(19) United States
(12) Patent Application Publication
(10) Pub. No.: US 2011/0117081 A1
(43) Pub. Date: May 19, 2011

(54) FUNCTIONALIZED PYRROLIDINES AND USE THEREOF AS IAP INHIBITORS

(75) Inventors: Alain Laurent, Montreal (CA); Melanie Proulx, Montreal (CA); James Jaquith, Pincourt (CA)

(73) Assignee: Aegera Therapeutics, Inc., Verdun, QC (CA)

(21) Appl. No.: 12/990,898

(22) PCT Filed: May 5, 2009
(86) PCT No.: PCT/IB09/05834

§ 371 Date: Dec. 8, 2010

Related U.S. Application Data
(60) Provisional application No. 61/071,541, filed on May 5, 2008.
(60) Provisional application No. 61/142,013, filed on Dec. 31, 2008.

(51) Int. Cl. A61K 39/395; C07D 403/12; A61K 31/4025; C07D 401/14; A61K 31/4545; A61K 31/4725; A61K 31/506; C07D 487/04; A61K 31/519; A61P 35/00; A61P 19/02; A61K 31/573; C12N 5/071

(52) U.S. Cl. 424/130.1; 548/518; 514/422; 546/187; 514/316; 546/140; 514/308; 544/296; 514/256; 544/281; 514/259.1; 548/455; 514/414; 514/171; 435/375

(57) ABSTRACT

A compound of Formula (1) or a salt thereof, methods for the preparation and use of such a compound, especially as an IAP inhibitor, and related compounds, compositions, and methods.

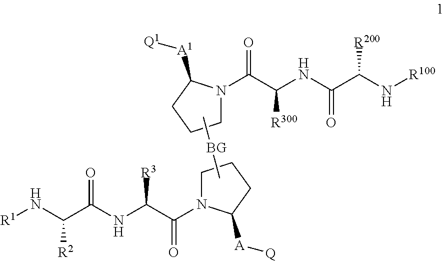

### BACKGROUND OF THE INVENTION

Apoptosis, or programmed cell death, typically occurs in the normal development and maintenance of healthy tissues in multicellular organisms. It is a complex process which results in the removal of damaged, diseased or developmentally redundant cells, in the absence of signs of inflammation or necrosis.

Intrinsic apoptotic pathways are known to be dysregulated in cancer and lymphoproliferative syndromes, as well as autoimmune disorders such as multiple sclerosis and rheumatoid arthritis, as well as in neurodegenerative diseases and inflammation. Additionally, alterations in a host apoptotic response have been described in the development or maintenance of viral and bacterial infections.

Apoptosis is the ordered dismantling of cellular components leading to cell death, which occurs as a normal part of development, the maintenance of normal cellular homeostasis, or as a consequence of injurious stimuli such as chemotherapy and radiation. Cancer cells, however, gain the ability to overcome or circumvent apoptosis and continue with inappropriate proliferation despite strong pro-apoptotic signals such as hypoxia, endogenous cytokines, radiation treatments and chemotherapy. In autoimmune disease, pathogenic effector cells can become resistant to normal apoptotic cues. Resistance results from numerous mechanisms, including alterations in the apoptotic machinery due to increased activity of anti-apoptotic pathways or expression of anti-apoptotic genes. Thus, approaches that reduce the threshold of apoptotic induction in cancer cells by overcoming innate resistance mechanisms may be of significant clinical utility.

The caspases are a family of proteolytic enzymes from the class of cysteine proteases which are known to initiate and execute apoptosis. In normal cells, the caspases are present as inactive zymogens, which are catalytically activated following external signals, for example those resulting from ligand driven Death Receptor activation, such as cytokines or immunological agents, or by release of mitochondrial factors, such as cytochrome C following genotoxic, chemotoxic, or radiation-induced cellular injury. The Inhibitors of Apoptosis Proteins (IAPs) constitute a family of proteins which are capable of binding to and inhibiting the caspases, thereby suppressing cellular apoptosis. Because of their central role in regulating caspase activity, the IAPs are capable of inhibiting programmed cell death from a wide variety of triggers, which include loss of homeostatic, or endogenous cellular growth control mechanisms, as well as chemotherapeutic drugs and irradiation.

The IAPs contain one to three homologous structural domains known as baculovirus IAP repeat (BIR) domains. They may also contain a RING zinc finger domain at the C-terminus, with a capability of inducing ubiquitinylation of IAP-binding molecules via its E3 ligase function. The human IAPs, XIAP, HIAP1 (also referred to as cIAP2), and HIAP2 (cIAP1) each have three BIR domains, and a carboxy terminal RING zinc finger. Another IAP, NAIP, has three BIR domains (BIR1, BIR2 and BIR3), but no RING domain, whereas Livin, TsIAP and MLIAP have a single BIR domain and a RING domain. The X chromosome-linked inhibitor of apoptosis (XIAP) is an example of an IAP which can inhibit the initiator caspase, known as caspase-9, and the effector caspases, Caspase-3 and Caspase-7, by direct binding. It can also induce the removal of caspases through the ubiquitylation-mediated proteasome pathway via the E3 ligase activity of a RING zinc finger domain. It is via the BIR3 domain that XIAP binds to and inhibits caspase-9. The linker-BIR2 domain of XIAP inhibits the activity of caspases-3 and -7. The BIR domains have also been associated with the interactions of IAPs with tumor necrosis factor-receptor associated factor (TRAFs)-1 and -2, and to TAB1, as adaptor proteins effecting survival signaling through NFkB activation. The IAPs thus function as a direct brake on the apoptosis cascade, by preventing the action of, or inhibiting active caspases and by re-directing cellular signaling to a pro-survival mode.

Cancer cells and cells involved in autoimmune disease may avoid apoptosis by the sustained over-expression of one or more members of the IAP family of proteins. For example, IAP overexpression has been demonstrated to be prognostic of poor clinical outcome in multiple cancers, and decreased IAP expression through RNA antisense or siRNA strategies sensitizes tumor cells to a wide variety of apoptotic insults including chemotherapy, radiotherapy and death receptor ligands. For XIAP this is shown in cancers as diverse as leukemia and ovarian cancer. Over expression of HIAP1 and HIAP2 resulting from the frequent chromosome amplification of the 11q21-q23 region, which encompasses both, has been observed in a variety of malignancies, including medulloblastomas, renal cell carcinomas, glioblastomas, and gastric carcinomas. Also, abnormally apoptotic resistant T-cells have been demonstrated in autoimmune diseases such as multiple sclerosis, rheumatoid arthritis, idiopathic thrombocytopenic purpura, and alopecia areata. Other abnormally apoptotic resistant cells also have been linked to autoimmune disease, such as fibroblast-like synoviocytes in rheumatoid arthritis (RA) and keratinocytes in psoriasis. Thus, IAPs are valid therapeutic targets and compounds that inhibit their expression or function may have significant utility in the treatment of proliferative diseases associated with dysregulated apoptosis, including cancer and autoimmune diseases.

### SUMMARY OF THE INVENTION

In one aspect of the invention, there is provided a compound represented by Formula 1:

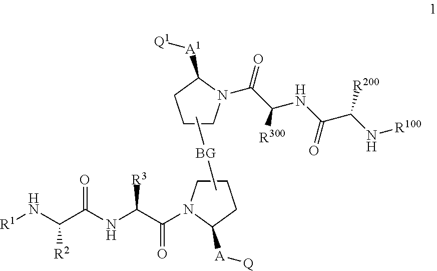

or a salt thereof, wherein
m is 0, 1 or 2;

### Y is NH, O or S;

### BG is —X-L-X¹—;

X and X¹ are independently

1) O,

2) NR¹²,

3) S,

4) —C₁-C₆ alkyl-,

5) —C₁-C₆ alkyl-O—,

6) —C₁-C₆ alkyl-NR¹²—,

7) —C₁-C₆ alkyl-S—,

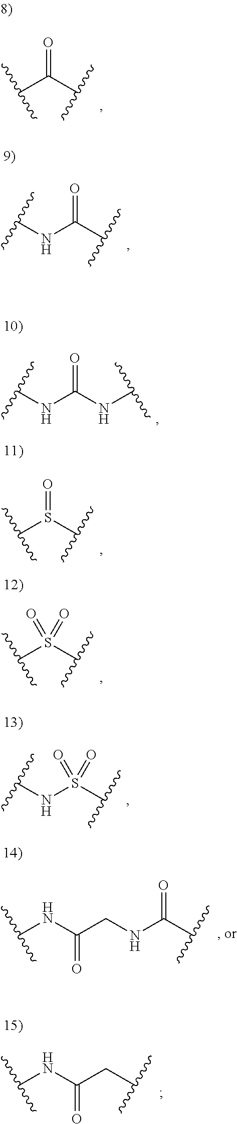

### L is

1) —C₁-C₂₀ alkyl-,

2) —C₂-C₆ alkenyl-,

3) —C₂-C₈ alkynyl-,

4) —C₃-C₇ cycloalkyl-,

5) -aryl-,

6) -biphenyl-,

7) -heteroaryl-,

8) -heterocyclyl-,

9) —C₁-C₆ alkyl-(C₂-C₆ alkenyl)-C₁-C₆ alkyl-,

10) —C₁-C₆ alkyl-(C₂-C₄ alkynyl)-C₁-C₆ alkyl-

11) —C₁-C₆ alkyl-(C₃-C₇ cycloalkyl)-C₁-C₆ alkyl-,

12) —C₁-C₆ alkyl-aryl-C₁-C₆ alkyl-,

13) —C₁-C₆ alkyl-biphenyl-C₁-C₆ alkyl-,

14) —C₁-C₆ alkyl-heteroaryl-C₁-C₆ alkyl-,

15) —C₁-C₆ alkyl-heterocycyl-C₁-C₆ alkyl-,

16) —C₁-C₆ alkyl-Y—C₁-C₆ alkyl-,

17) -aryl-Y-aryl-,

18) -heteroaryl-Y-heteroaryl-,

19) -heterocyclyl-Y-heterocyclyl-,

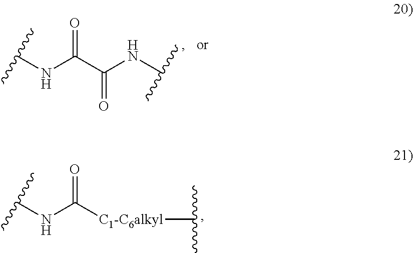

wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents;

### Q is

1) NR⁴R⁵,

2) OR¹¹,

3) S(O)ₘR¹¹,

4) aryl, or

5) heteroaryl,

wherein the aryl and the heteroaryl are optionally substituted with one or more R¹⁰ substituents;

Q¹ is

1) NR⁴⁰⁰R⁵⁰⁰,

2) OR¹¹⁰⁰,

3) S(O)ₘR¹¹⁰⁰,

4) aryl, or

5) heteroaryl,

wherein the aryl and the heteroaryl are optionally substituted with one or more R¹⁰ substituents;
A and A¹ are independently

1) C₁-C₃ alkylene, or

2) —C(O)—;

R¹ and R¹⁰⁰ are C₁-C₆ alkyl optionally substituted with one or more R⁶ substituents;
R² and R²⁰⁰ are independently

1) H,

2) C₁-C₆ alkyl optionally substituted with one or more R⁶ substituents, or

3) C₃-C₇ cycloalkyl optionally substituted with one or more R⁶ substituents;

R³ and R³⁰⁰ are independently

1) C₃-C₇ cycloalkyl,

2) C₃-C₇ cycloalkenyl,

3) aryl,

4) heteroaryl,

5) heterocyclyl, or

6) heterobicyclyl,

wherein the cycloalkyl, cycloalkenyl, heterocyclyl, and heterobicyclyl are optionally substituted with one or more R⁶ substituents; and wherein the aryl and heteroaryl are optionally substituted with one of more R¹⁰ substituents;
R⁴, R⁴⁰⁰, R⁵, R⁵⁰⁰ are each independently

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) C(O)—R¹¹,

13) C(O)O—R¹¹,

14) C(═Y)NR⁸R⁹, or

15) S(O)₂—R¹¹,

wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
or R⁴ and R⁵ taken together with the nitrogen to which they are attached, and R⁴⁰⁰ and R⁵⁰⁰ taken together with the nitrogen to which they are attached, form a C₃-C₇ heterocycloalkylene optionally substituted with C₁-C₆ alkyl, C₃-C₇ cycloalkyl, or C₆-C₁₀ aryl, wherein the aryl is optionally substituted with R¹⁰,

### R⁶ is

1) halogen,

2) NO₂,

3) CN,

4) haloalkyl,

5) C₁-C₆ alkyl,

6) C₂-C₆ alkenyl,

7) C₂-C₄ alkynyl,

8) C₃-C₇ cycloalkyl,

9) C₃-C₇ cycloalkenyl,

10) aryl,

11) heteroaryl,

12) heterocyclyl,

13) heterobicyclyl,

14) OR⁷,

15) S(O)ₘR⁷,

16) NR⁸R⁹,

17) NR⁸S(O)₂R¹¹,

18) COR⁷,

19) C(O)OR⁷,

20) CONR⁸R⁹,

21) S(O)₂NR⁸R⁹

22) OC(O)R⁷,

23) OC(O)Y—R¹¹,

24) SC(O)R⁷, or

25) NC(Y)NR⁸R⁹,

wherein the aryl, heteroaryl, heterocyclyl, and heterobicyclyl are optionally substituted with one or more R¹⁰ substituents;

### R⁷ is

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) —C(═Y)NR⁸R⁹, or

13) C₁-C₆ alkyl-C₂-C₄ alkenyl, or

14) C₁-C₆ alkyl-C₂-C₄ alkynyl,

wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
R⁸ and R⁹ are each independently

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) C(O)R¹¹,

13) C(O)Y—R¹¹, or

14) S(O)₂—R¹¹,

wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
or R⁸ and R⁹ together with the nitrogen atom to which they are attached form a five, six or seven membered heterocyclic ring optionally substituted with one or more R⁶ substituents;

### R¹⁰ is

1) halogen,

2) NO₂,

3) CN,

4) C₁-C₆ alkyl,

5) C₂-C₆ alkenyl,

6) C₂-C₄ alkynyl,

7) C₃-C₇ cycloalkyl,

8) C₃-C₇ cycloalkenyl,

9) haloalkyl,

10) OR⁷,

11) NR⁸R⁹,

12) SR⁷,

13) COR⁷,

14) C(O)OR⁷,

15) S(O)ₘR⁷,

16) CONR⁸R⁹,

17) S(O)₂NR⁸R⁹,

18) aryl,

19) heteroaryl,

20) heterocyclyl, or

21) heterobicyclyl,

wherein the alkyl, alkenyl, alkynyl, cycloalkyl, and cycloalkenyl are optionally substituted with one or more R⁶ substituents;

R¹¹ and R¹¹⁰⁰ are

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl, or

10) heterobicyclyl,

wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents. Methods of preparing a compound of Formula 1 also is provided herein.

In another aspect of the present invention, there is provided an intermediate compound represented by Formula 2:

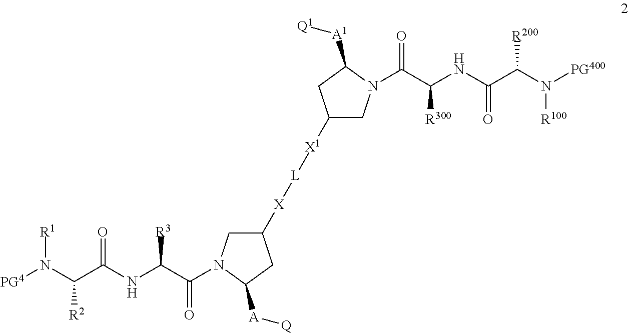

wherein PG⁴, PG⁴⁰⁰, L, X, X¹, R³, R³⁰⁰, R², R²⁰⁰, R¹, R¹⁰⁰, A, A¹, Q and Q¹ are as defined herein.

In another aspect of the present invention, there is provided an intermediate compound represented by Formula 3:

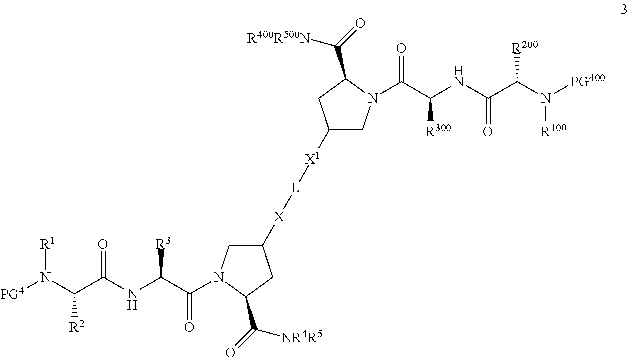

wherein PG⁴, PG⁴⁰⁰, L, X, X¹, R⁵, R⁵⁰⁰, R⁴, R⁴⁰⁰, R³, R³⁰⁰, R², R²⁰⁰, R¹ and R¹⁰⁰ are as defined herein.

In another aspect of the present invention, there is provided an intermediate compound represented by Formula 4:

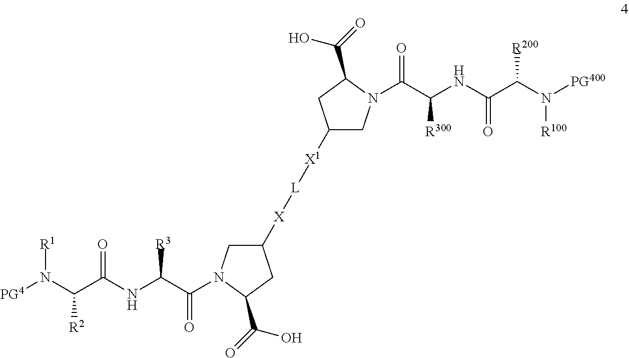

wherein PG⁴, PG⁴⁰⁰, L, X, X¹, R³, R³⁰⁰, R², R²⁰⁰, R¹ and R¹⁰⁰ are as defined herein.

In another aspect of the present invention, there is provided an intermediate compound represented by Formula 5:

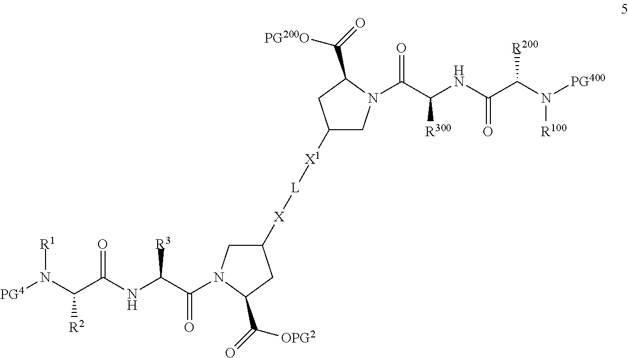

wherein PG⁴, PG⁴⁰⁰, PG², PG²⁰⁰, L, X, X¹, R³, R³⁰⁰, R², R²⁰⁰, R¹ and R¹⁰⁰ are as defined herein.

In another aspect of the present invention, there is provided an intermediate compound represented by Formula 6:

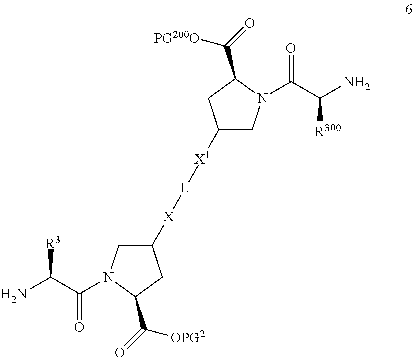

wherein PG², PG²⁰⁰, L, X, X¹, R³ and R³⁰⁰ are as defined herein.

In another aspect of the present invention, there is provided an intermediate compound represented by Formula 7:

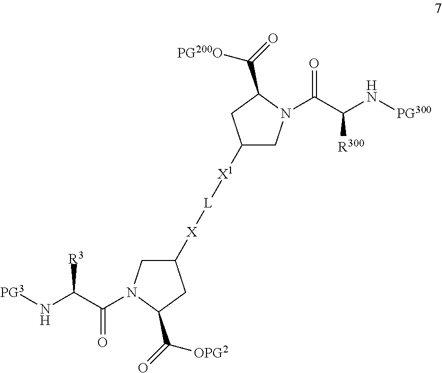

wherein PG³, PG³⁰⁰, PG², PG²⁰⁰, L, X, X¹, R³ and R³⁰⁰ are as defined herein.

In another aspect of the present invention, there is provided an intermediate compound represented by Formula 8:

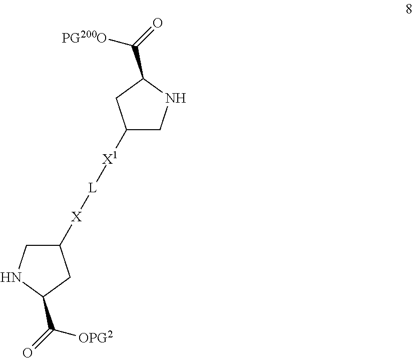

wherein PG², PG²⁰⁰, L, X and X¹ are as defined herein.

In another aspect of the present invention, there is provided an intermediate compound represented by Formula 9:

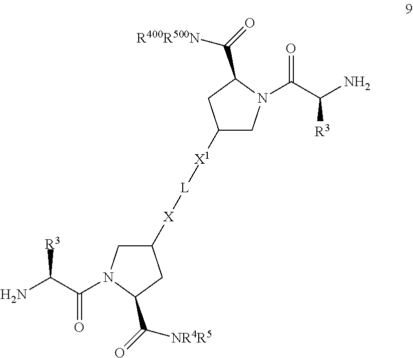

wherein L, X, X¹, R⁵, R⁵⁰⁰, R⁴, R⁴⁰⁰, R³ and R³⁰⁰ are as defined herein.

In another aspect of the present invention, there is provided an intermediate compound represented by Formula 10:

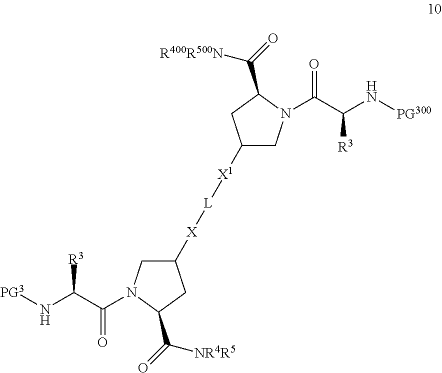

wherein PG³, PG³⁰⁰, L, X, X¹, R⁵, R⁵⁰⁰, R⁴, R⁴⁰⁰, R³ and R³⁰⁰ are as defined herein.

In another aspect of the present invention, there is provided an intermediate compound represented by Formula 11:

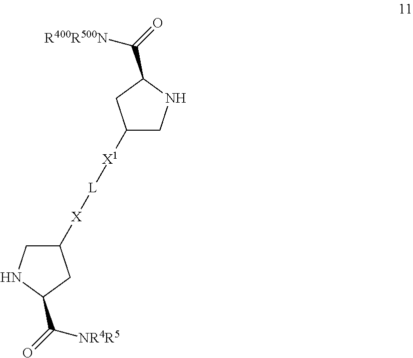

wherein L, X, X¹, R⁵, R⁵⁰⁰, R⁴ and R⁴⁰⁰ are as defined herein.

In another aspect of the present invention, there is provided an intermediate compound represented by Formula 12:

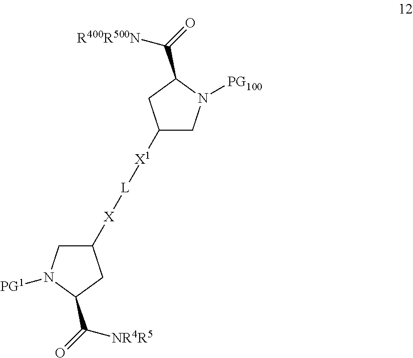

Wherein PG¹, PG¹⁰⁰, L, X, X¹, R⁵, R⁵⁰⁰, R⁴ and R⁴⁰⁰ are as defined herein.

In another aspect of the present invention, there is provided an intermediate compound represented by Formula 13:

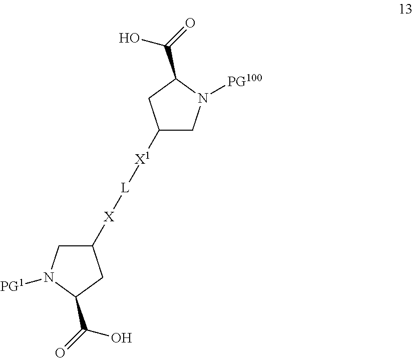

wherein PG¹, PG¹⁰⁰, L, X and X¹ are as defined herein.

In another aspect of the present invention, there is provided an intermediate compound represented by Formula 14:

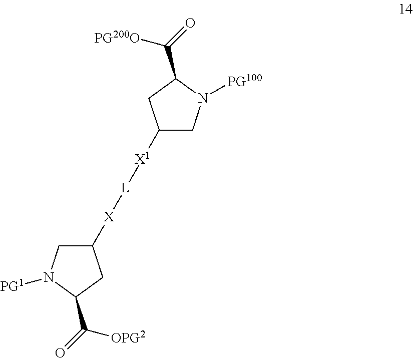

wherein PG², PG²⁰⁰, PG¹, PG¹⁰⁰, L, X and X¹ are as defined herein.

In another aspect of the present invention, there is provided an intermediate compound represented by Formula 15:

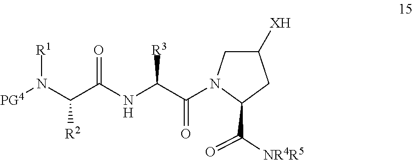

wherein PG⁴, X, R⁵, R⁴, R³, R² and R¹ are as defined herein.

In another aspect of the present invention, there is provided an intermediate compound represented by Formula 16:

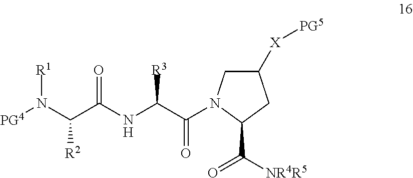

wherein PG⁵, PG⁴, X, R⁵, R⁴, R³, R² and R¹ are as defined herein.

In another aspect of the present invention, there is provided an intermediate compound represented by Formula 17:

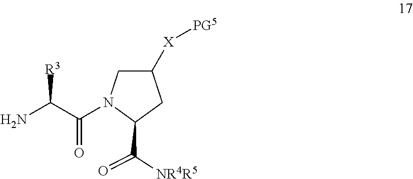

wherein PG⁵, X, R⁵, R⁴ and R³ are as defined herein.

In another aspect of the present invention, there is provided an intermediate compound represented by Formula 18:

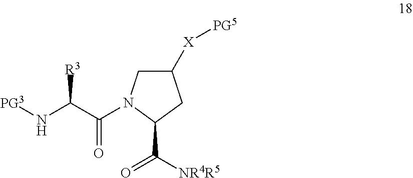

wherein PG⁵, PG³, X, R⁵, R⁴ and R³ are as defined herein.

In another aspect of the present invention, there is provided an intermediate compound represented by Formula 19:

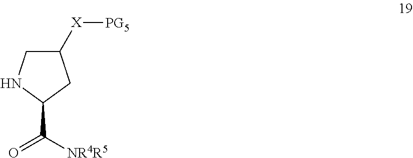

wherein PG⁵, X, R⁵ and R⁴ areas defined herein.

In another aspect of the present invention, there is provided an intermediate compound represented by Formula 20:

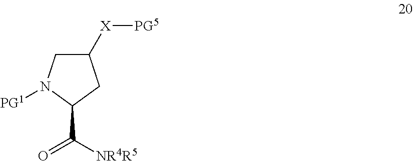

wherein PG⁵, PG¹, X, R⁵ and R⁴ are as defined herein.

In another aspect of the present invention, there is provided the following intermediates: 1-i, 1-ii, 1-iii, 1-iv, 1-v, 1-vi, 2-i, 2-ii, 4-i, 4-ii, 4-iii, 4-iv, 4-v, 4-vi and 4-vii.

In another aspect of the present invention, there is provided the following intermediates: 6-2, 6-3, 6-4, 6-5, 6-7, 6-8, 6-10, 7-2, 7-3, 7-5, 7-6, 7-8, 7-9, and 7-10.

In another aspect of the present invention, there is provided a method for the preparation of a pharmaceutically acceptable salt of a compound of formula 1. The method can comprise treating a compound of formula 1 with a pharmaceutically acceptable acid (e.g., 1 to 2 equivalents of a pharmaceutically acceptable acid), so as to form a pharmaceutically acceptable salt of a compound of Formula 1. Alternatively, the method can comprise treating an intermediate compound of formula 2-ii with a pharmaceutically acceptable acid so as to provide a pharmaceutically acceptable salt of a compound of Formula 1.

In another aspect of the present invention, there is provided a pharmaceutical composition comprising a compound of Formula 1 and a pharmaceutically acceptable carrier, diluent or excipient, as well as a method of preparing same comprising combining a compound of Formula 1 with a pharmaceutically acceptable carrier, diluents, or excipient.

In another aspect of the present invention, there is provided a method of treating a proliferative disorder or a disease state characterized by insufficient apoptosis, the method comprising: administering to a subject in need thereof, a therapeutically effective amount of a compound or pharmaceutical composition, as described above, so as to treat the proliferative disorder or disease state.

In another aspect of the present invention, there is provided a method of modulating IAP function, the method comprising: contacting a cell with a compound of the present invention so as to prevent binding of a BIR binding protein to an IAP BIR domain thereby modulating the IAP function.

In another aspect of the present invention, there is provided a probe, the probe being a compound of Formula 1 labeled with a detectable label or an affinity tag. In other words, the probe comprises a compound of Formula 1 and a detectable label.

In another aspect of the present invention, there is provided a method of identifying compounds that bind to an IAP BIR domain, the assay comprising:
a) contacting an IAP BIR domain with a probe, as described herein, to form a probe:BIR domain complex, the probe being displaceable by a test compound;
b) measuring a signal from the probe so as to establish a reference level;
c) incubating the probe:BIR domain complex with the test compound;
d) measuring the signal from the probe; and
e) comparing the signal from step d) with the reference level, a modulation of the signal (e.g., an increase or decrease in the signal relative to the reference level) being an indication that the test compound binds to the BIR domain.

In another aspect of the present invention, there is provided a method of detecting loss of function or suppression of IAPs in vivo, the method comprising: a) administering to a subject, a therapeutically effective amount of a pharmaceutical composition, as defined above; b) isolating a tissue sample from the subject; and c) detecting a loss of function or suppression of IAPs from the sample.

### DETAILED DESCRIPTION OF THE INVENTION

Provided herein is a compound of Formula 1:

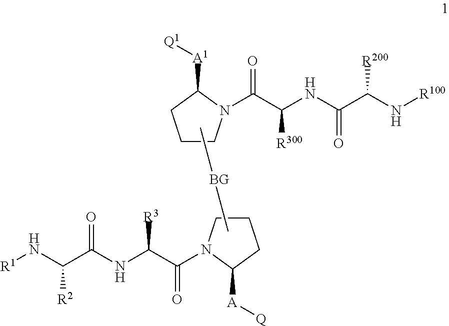

or a salt thereof. Compounds of Formula 1 also can be represented by the following formula, in which M1 and M2 represent independent BIR binding domains:

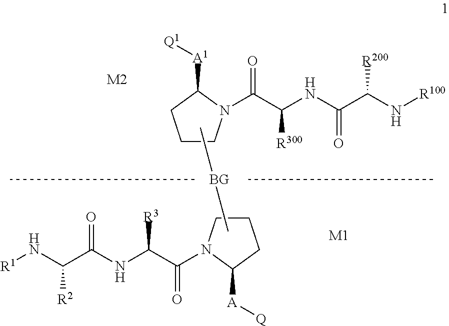

wherein R¹, R², R³, R¹⁰⁰, R²⁰⁰, R³⁰⁰, A, A¹, Q, Q¹ and BG are as defined herein, and the dotted line represents a hypothetical dividing line for comparing the substituents associated with M1 and M2.

In one subset of compounds of Formula 1, M1 is the same as M2 and the dotted line denotes a line of symmetry. In another subset, M1 is different from M2.

One skilled in the art will recognize that when M1 and M2 are the same, the R¹, R², R³, R⁴, R⁵, R⁶, R⁷, R⁸, R⁹, R¹⁰, R¹¹, R¹², R¹³, n, m, Y, A, Q, and X substituents in M1 have the same meaning as the R¹⁰⁰, R²⁰⁰, R³⁰⁰, R⁴⁰⁰, R⁵⁰⁰, R¹¹⁰⁰, R⁶, R⁷, R⁸, R⁹, R¹⁰, R¹¹, R¹², R¹³, n, m, Y, A¹, Q¹, and X¹ substituents respectively in M2. When M1 and M2 are different, at least one R¹, R², R³, R⁴, R⁵, R¹⁰⁰, R²⁰⁰, R³⁰⁰, R⁴⁰⁰, R⁵⁰⁰, R¹¹⁰⁰, R⁶, R⁷, R⁸, R⁹, R¹⁰, R¹¹, R¹², R¹³, n, m, Y, A, A¹, Q, Q¹, X, and X¹ substituent is different in either of M1 or M2.

Alternatively the substituents in M1 can be defined as R¹, R², R³, R⁴, R⁵, R⁶, R⁷, R⁸, R⁹, R¹⁰, R¹¹, R¹², R¹³, n, m, Y, A, Q, and X, and those in M2 can be defined as R¹⁰⁰, R²⁰⁰, R³⁰⁰, R⁴⁰⁰, R⁵⁰⁰, R¹¹⁰⁰, R⁶, R⁷, R⁸, R⁹, R¹⁰, R¹¹, R¹², R¹³, n, m, Y, A¹, Q¹ and X¹ respectively. In the case where M1 and M2 are the same, the R¹, R², R⁴, R⁵, R⁶, R⁷, R⁸, R⁹, R¹⁰, R¹¹, R¹², R¹³, n, m, Y, A, Q, and X substituents in M1 have the same meanings as R¹⁰⁰, R²⁰⁰, R³⁰⁰, R⁴⁰⁰, R⁵⁰⁰, R¹¹⁰⁰, R⁶, R⁷, R⁸, R⁹, R¹⁰, R¹¹, R¹², R¹³, n, m, Y, A¹, Q¹ and X¹ respectively in M2. In the case where M1 and M2 are different, at least one of the aforesaid substituents is different.

In a preferred aspect, the compounds of the present invention of Formula 1 are useful as BIR domain binding compounds in mammalian IAPs. The following embodiments further illustrate compounds according to Formula 1.

### A and A¹:

A and A¹ can be, independently, any group as hereinbefore defined, without limitation. However, in one subset of compounds of Formula 1, A and A¹ are both C₁-C₃ alkylene (e.g., CH₂CH₂ or CH₂), or are both C═O. In an alternative subset of compounds of Formula 1, A is C₁-C₃ alkylene (e.g., CH₂CH₂ or CH₂) and A¹ is C═O.

Any and each individual definition of A and A¹ as set out herein may be combined with any and each individual definition of Core, R¹, R², R¹⁰⁰, R²⁰⁰, R³, R³⁰⁰, Q, Q¹, and BG as set out herein.

### Core:

The compound of Formula 1 can be a compound of any of formulas 1A through 1C:

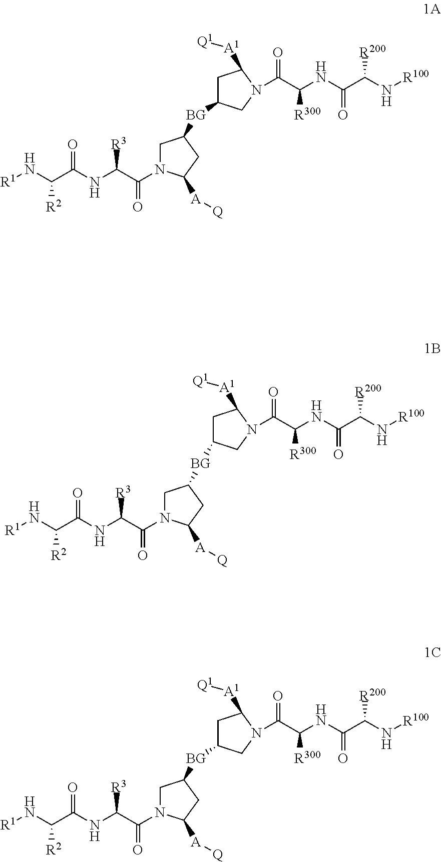

wherein BG, A, A¹, Q, Q¹, R¹, R¹⁰⁰, R², R²⁰⁰, R³, and R³⁰⁰ are as defined hereinabove and hereinafter.

Any and each individual definition of Core as set out herein may be combined with any and each individual definition of A, A¹, R¹, R², R¹⁰⁰, R²⁰⁰, R³, R³⁰⁰, Q, Q¹, and BG as set out herein.

### BG:

In compounds of Formula 1, BG is —X-L-X¹—. Such compounds can, for example, have the structure of any of Formulas 1a through 1c:

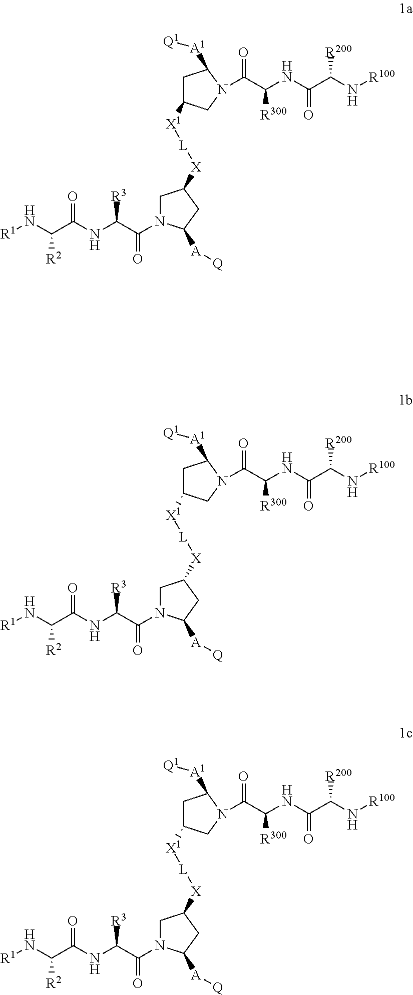

wherein L, X, X¹, A, A¹, Q, Q¹, R¹, R¹⁰⁰, R², R²⁰⁰, R³, and R³⁰⁰ are as defined hereinabove and hereinafter.

One further subset of the aforesaid compounds comprises compounds of any of Formulas 1.1a, 1.1a-1, 1.1a-2, 1.2 and 1.1c:

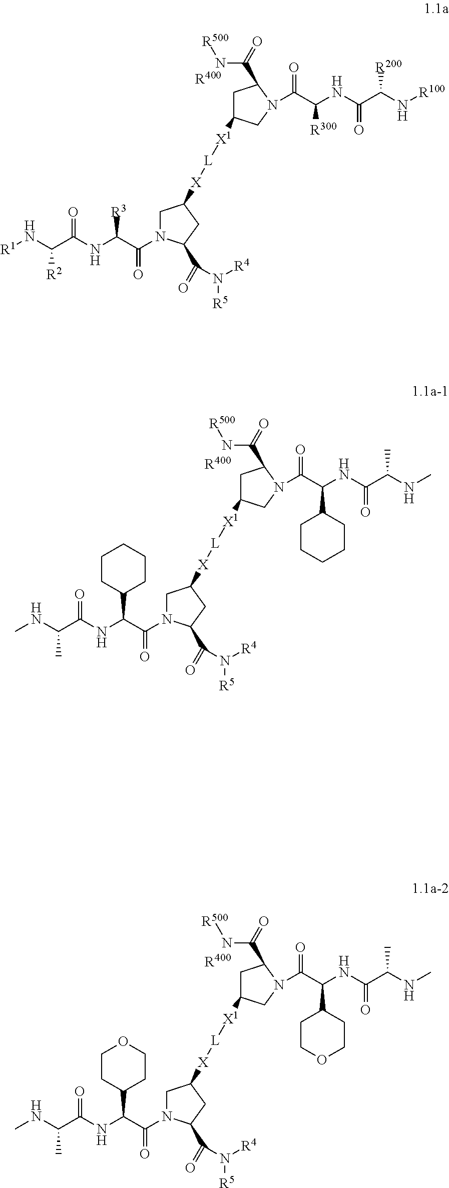

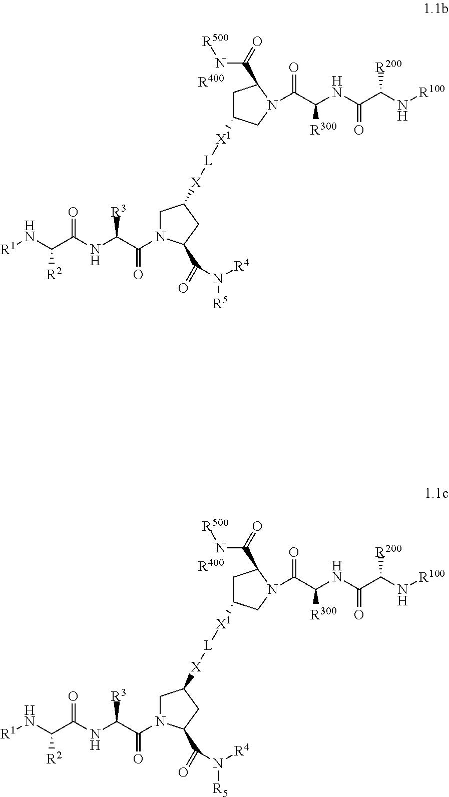

wherein L, X, X¹, A, A¹, R¹, R¹⁰⁰, R², R²⁰⁰, R³, R³⁰⁰, R⁴, R⁴⁰⁰, R⁵ and R⁵⁰⁰ are as defined hereinabove and hereinafter.

Any and each individual definition of BG as set out herein may be combined with any and each individual definition of Core, R¹, R², R¹⁰⁰, R²⁰⁰, R³, R³⁰⁰, A, A¹ Q, and Q¹ as set out herein.

### X and X¹:

X and X¹ can be, independently, any group as hereinbefore defined, without limitation. However, in one subset of the aforesaid compounds, X and X¹ are independently

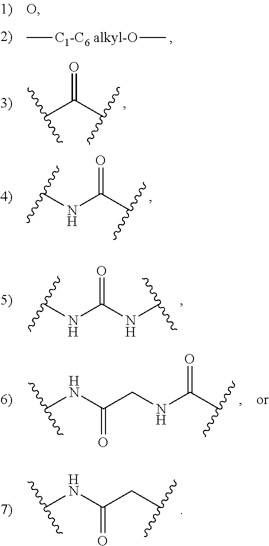

Any and each individual definition of X and X¹ as set out herein may be combined with any and each individual definition of Core, L, A, A¹, R¹, R², R¹⁰⁰, R²⁰⁰, R³, R³⁰⁰, Q, Q¹, and BG as set out herein.

### L:

L can be any group as hereinbefore defined, without limitation. However, in one subset of the aforesaid compounds, L is aryl (arylene) or biphenyl (biphenylene), which aryl can be at least disubstituted including, without limitation, disubstituted phenyl, disubstituted indanyl, disubstituted naphthyl, disubstituted anthracenyl, and disubstituted phenanthryl. The aryl(s) may be connected to X and X¹ at any two available positions on the aryl substituent, wherein the at least disubstituted aryl is optionally further substituted with one or more R¹⁰ substituents. In another subset, L is alkyl (alkylene) or cycloalkyl (cycloakylene). In still another subset, L is heteroarylene. Thus, by way of illustration, the compound of Formula 1 can be a compound

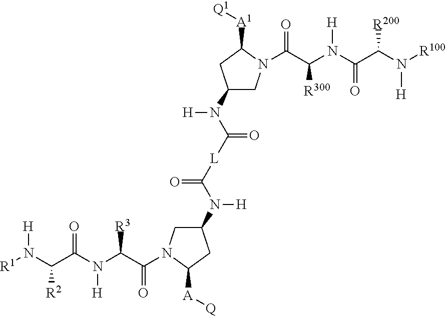

wherein L is (1) alkylene or cycloalkylene; (2) arylene or biphenylene; or (3) heteroarylene. Other non-limiting examples of suitable L groups include:

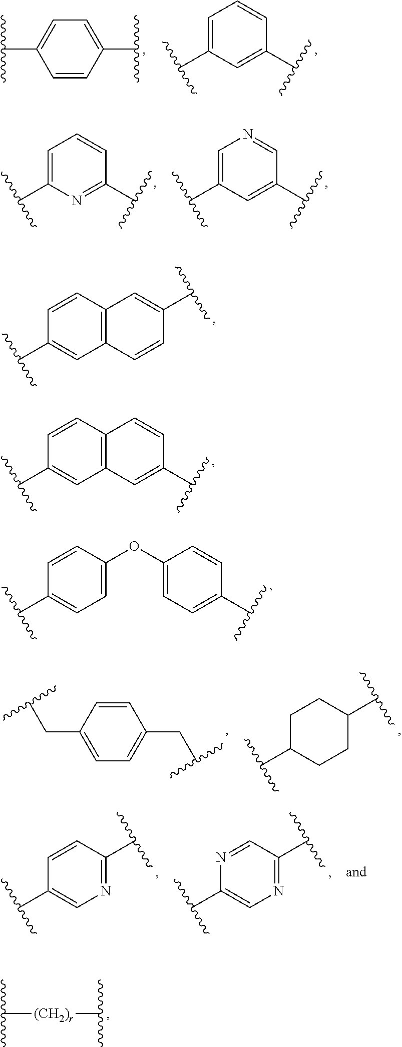

wherein r is 1, 2, 3, 4, 5, 6, 7, 8, 9, or 10.

Any and each individual definition of L as set out herein may be combined with any and each individual definition of Core, A, A¹, R¹, R², R¹⁰⁰, R²⁰⁰, R³, R³⁰⁰, X, X¹, Q, or Q¹, as set out herein.

In certain embodiments, the compound of Formula 1 can be a compound of any of Formulas 1.1 through 1.19:

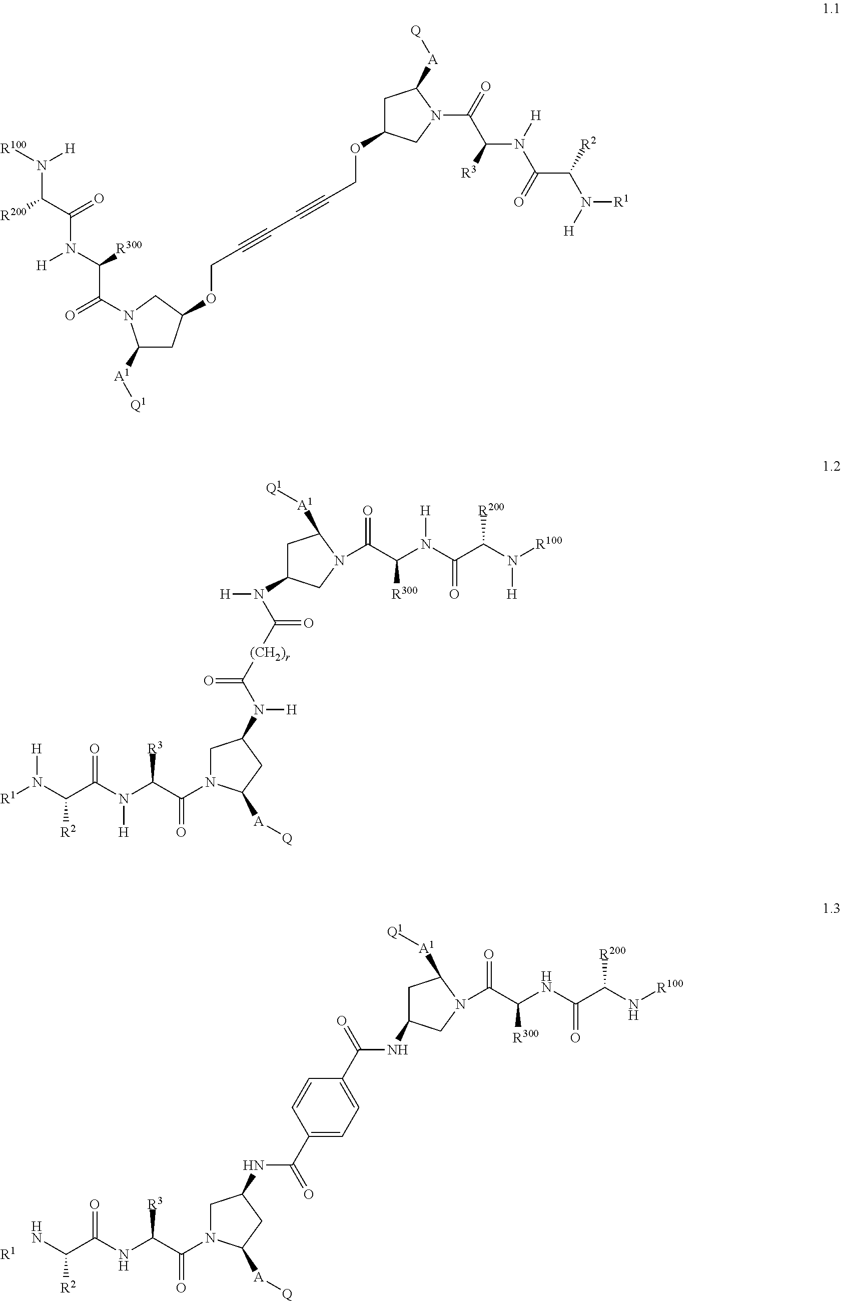

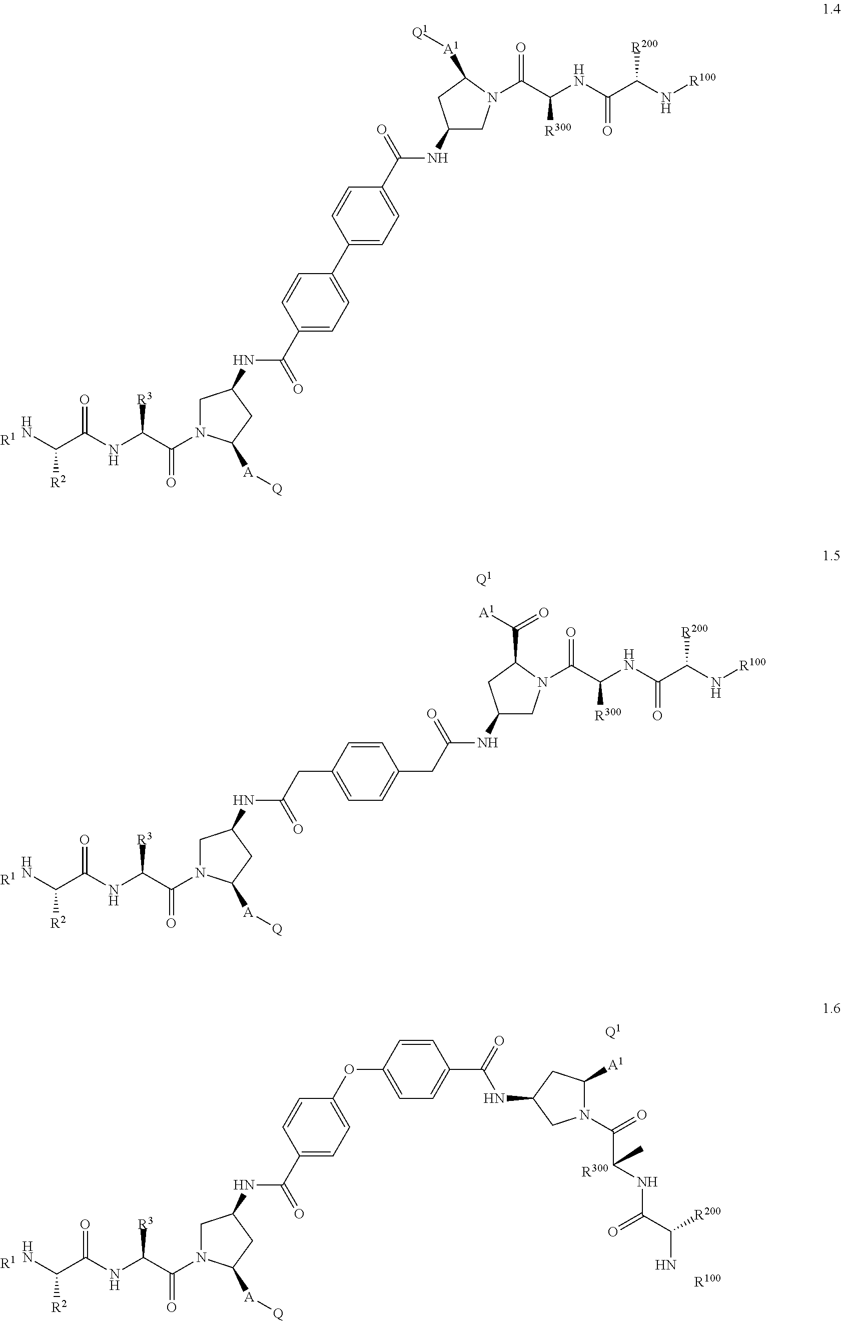

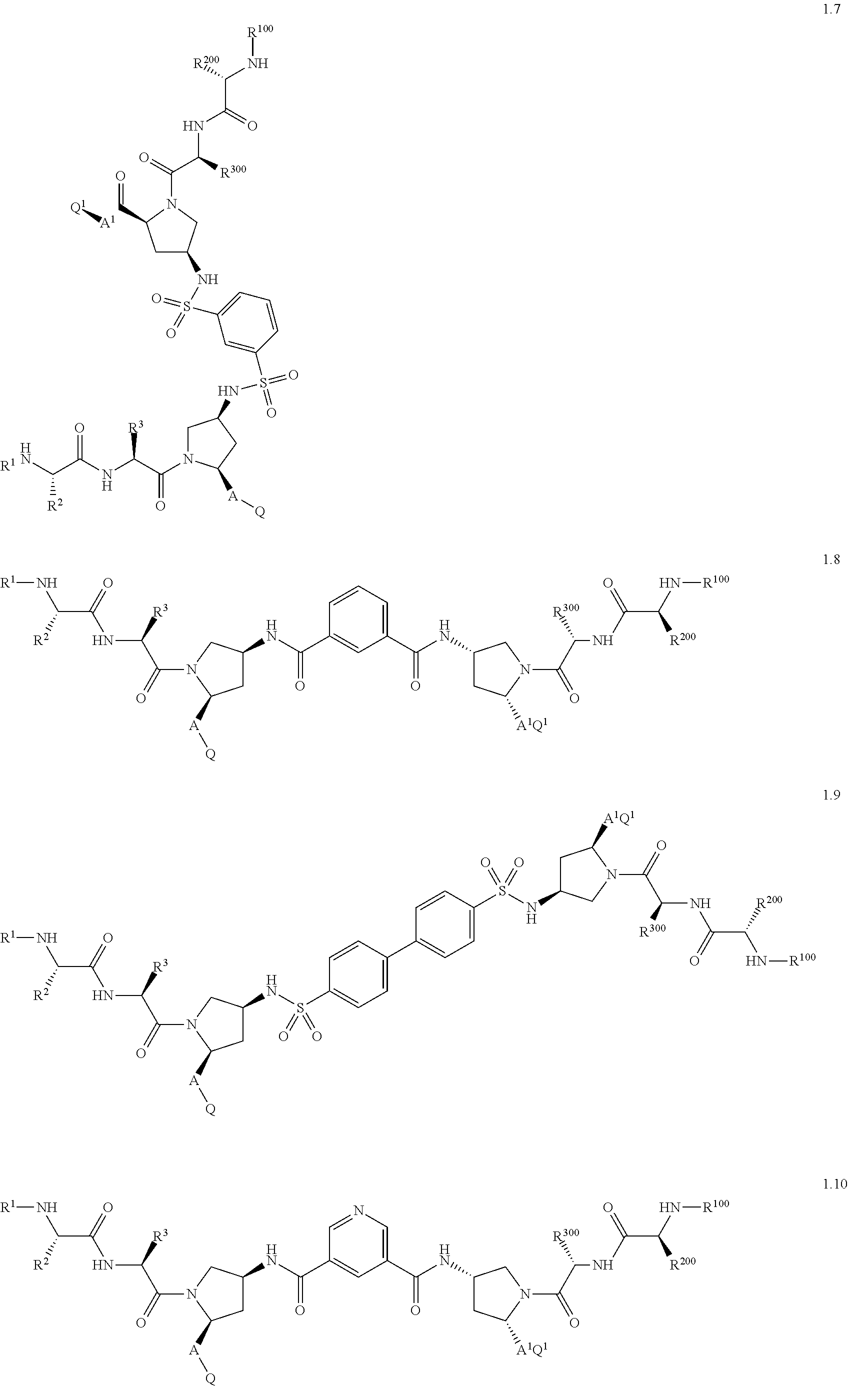

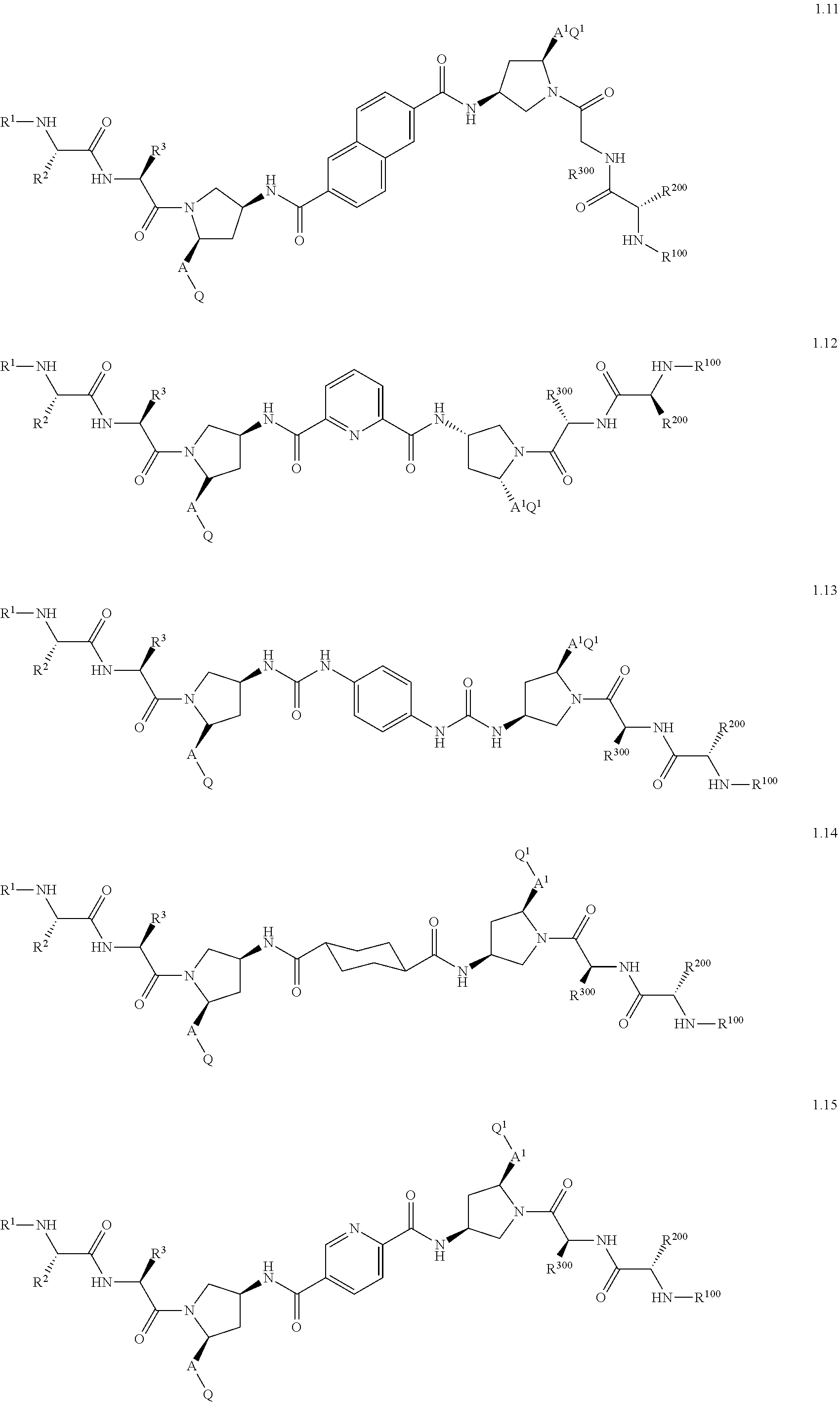

wherein r, A, A¹, Q, Q¹, R¹, R¹⁰⁰, R², R²⁰⁰, R³ and R³⁰⁰ are as defined hereinabove.

### R¹ and R¹⁰⁰:

R¹ and R¹⁰⁰ can be, independently, any group as hereinbefore defined, without limitation. In one subset of the aforesaid compounds R¹ and R¹⁰⁰ are both C₁-C₆ alkyl or C₁-C₃ alkyl. For example, R¹ and R¹⁰⁰ can both be CH₃ or CH₂CH₃.

Any and each individual definition of R¹ and R¹⁰⁰ as set out herein may be combined with any and each individual definition of Core, A, A¹, R², R²⁰⁰, R³, R³⁰⁰, Q, Q¹, and BG as set out herein.

### R² and R²⁰⁰:

R² and R²⁰⁰ can be, independently, any group as hereinbefore defined, without limitation. In one subset of the aforesaid compounds R² and R²⁰⁰ are both C₁-C₆ alkyl or C¹-C³ alkyl, for example, CH₃ or CH₂CH₃. In another subset of compounds, R² and R²⁰⁰ are both C₃-C₇ cycloalkyl, for example, cyclopropyl.

Any and each individual definition of R² and R²⁰⁰ as set out herein may be combined with any and each individual definition of Core, A, A¹, R¹, R¹⁰⁰, R³, R³⁰⁰, Q, Q¹, and BG as set out herein.

### R³ and R³⁰⁰:

R³ and R³⁰⁰ can be, independently, any group as hereinbefore defined, without limitation. In one subset of compounds of Formula 1, R³ and R³⁰⁰ can be

1) C₃-C₇ cycloalkyl,

2) C₃-C₇ cycloalkenyl,

3) heteroaryl,

4) heterocyclyl, or

5) heterobicyclyl,

wherein the cycloalkyl, cycloalkenyl, heterocyclyl, and heterobicyclyl are optionally substituted with one or more R⁶ substituents; and wherein the heteroaryl is optionally substituted with one of more R¹⁰ substituents. In another subset of compounds, R³ and R³⁰⁰ are both C₃-C₇ cycloalkyl or heterocyclyl. In yet another subset of compounds of Formula 1, R³ and R³⁰⁰ are both aryl. In still another subset of compounds of Formula 1, R³ and R³⁰⁰ are both heteroaryl. Examples of the aforesaid subsets include compounds of Formula 1, wherein R³ and R³⁰⁰ are both:

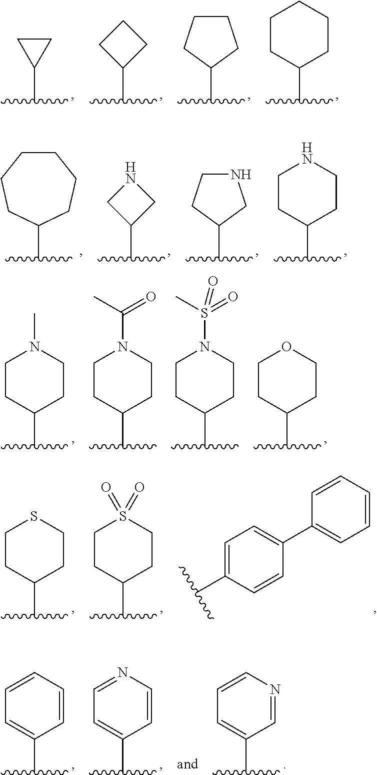

Any and each individual definition of R³ and R³⁰⁰ as set out herein may be combined with any and each individual definition of Core, A, A¹, R¹, R¹⁰⁰, R², R²⁰⁰, Q, Q¹, and BG as set out herein.

### Q and Q¹:

Q and Q¹ can be, independently, any group as hereinbefore defined, without limitation. In one subset of the aforesaid compounds, Q and Q¹ are NR⁴R⁵ and NR⁴⁰⁰R⁵⁰⁰, respectively, wherein R⁴, R⁴⁰⁰, R⁵ and R⁵⁰⁰ are as defined herein.

Any and each individual definition of Q and Q¹ as set out herein may be combined with any and each individual definition of Core, A, A¹, R¹, R¹⁰⁰, R², R²⁰⁰, R³, R³⁰⁰ and BG as set out herein.

### R⁴, R⁴⁰⁰, R⁵, and R⁵⁰⁰:

R⁴, R⁴⁰⁰, R⁵, and R⁵⁰⁰ can be, independently, any group as hereinbefore defined, without limitation. In one subset of the aforesaid compounds in which A and A¹ are both C═O, R⁴ and R⁴⁰⁰ are H and R⁵ and R⁵⁰⁰ are selected from

1) C₁-C₆ alkyl

2) C₂-C₆ alkenyl,

3) C₂-C₄ alkynyl,

4) C₃-C₇ cycloalkyl,

5) C₃-C₇ cycloalkenyl,

6) aryl,

7) heteroaryl,

8) heterocyclyl, or

9) heterobicyclyl,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents; wherein R⁶ and R¹⁰ are as defined herein. By way of further illustration, R⁵ and R⁵⁰⁰ can be, independently:

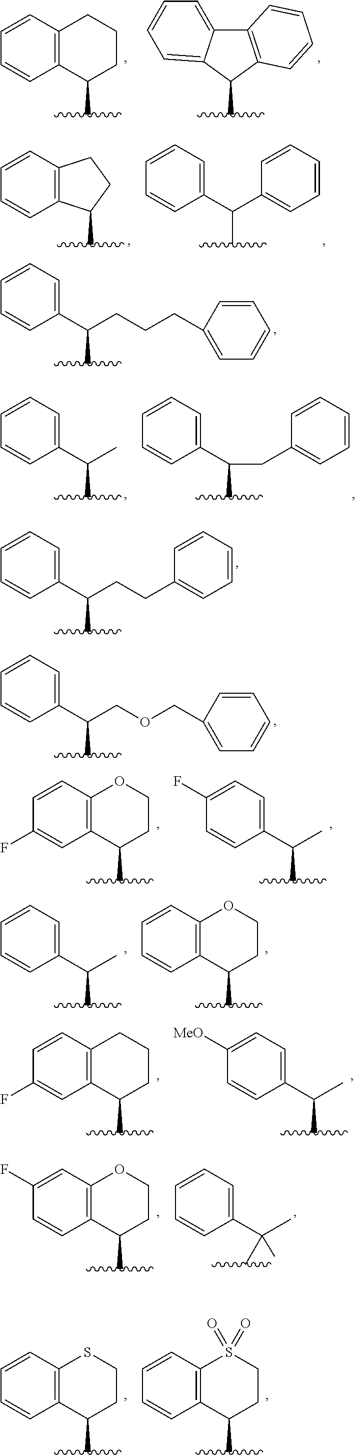

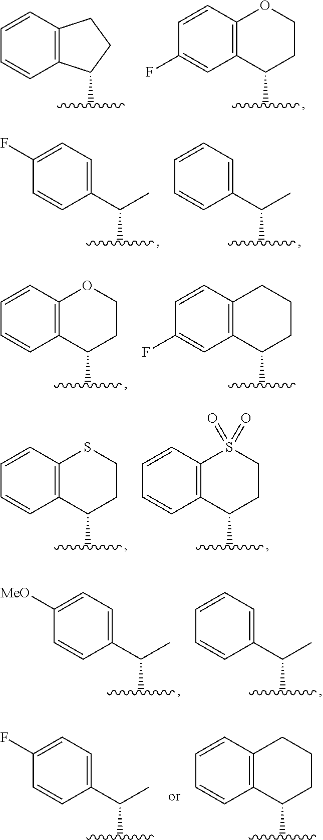

In an alternative subset of the aforesaid compounds in which A and A¹ are both CH₂, then R⁴, R⁴⁰⁰, R⁵, and R⁵⁰⁰ are each independently

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl,

10) heterobicyclyl,

11) C(O)—R¹¹,

12) C(O)O—R¹¹,

13) C(═Y)NR⁸R⁹, or

14) S(O)₂—R¹¹,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents; wherein Y, R⁶, R⁸, R⁹, R¹⁰ and R¹¹ are as defined herein.

In another alternative aspect of the invention, whether A and A¹ are both CH₂ or, desirably, C═O, R⁴ and R⁵ taken together and R⁴⁰⁰ and R⁵⁰⁰ taken together can form a C₃-C₇ alkylene optionally substituted with C₁-C₆ alkyl, C₃-C₇ cycloalkyl, or C₆-C₁₀ aryl. In other words, when taken together with the nitrogen to which they are attached, R⁴ and R⁵ (and R⁴⁰⁰ and R⁵⁰⁰) can form a C₃-C₇ heterocycloalkylene optionally substituted with C₁-C₆ alkyl, C₃-C₇ cycloalkyl, or C₆-C₁₀ aryl, wherein the aryl is optionally substituted with R¹⁰. Furthermore, the aryl can optionally be substituted with one or more R¹⁰ groups. By way of non-limiting example, R⁴ and R⁵ taken together and R⁴⁰⁰ and R⁵⁰⁰ taken together can, independently, form a group:

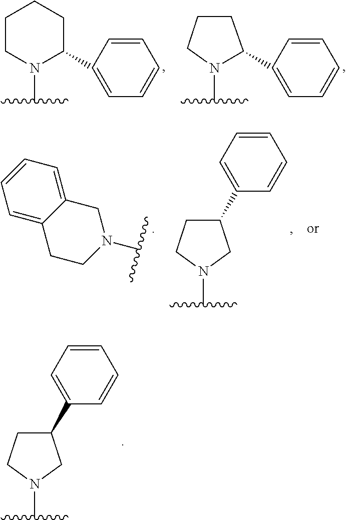

### R⁶:

R⁶ can be any group as hereinbefore defined, without limitation. In one subset of the aforesaid compounds, R⁶ is

1) halogen,

2) NO₂,

3) CN,

4) aryl,

5) heteroaryl,

6) heterocyclyl,

7) heterobicyclyl,

8) OR⁷,

9) SR⁷, or

10) NR⁸R⁹,

wherein the aryl, heteroaryl, heterocyclyl, and heterobicyclyl is optionally substituted with one or more R¹⁰ substituents; and wherein R⁷, R⁸, R⁹ and R¹⁰ are as defined herein.

Any and each individual definition of R⁶ as set out herein may be combined with any and each individual definition of Core, A, A¹, R¹, R¹⁰⁰, R², R²⁰⁰, R³, R³⁰⁰, R⁴, R⁵, and BG as set out herein.

### R⁸ and R⁹:

R⁸ and R⁹ can be, independently, any group as hereinbefore defined, without limitation. In one subset of the aforesaid compounds, R⁸ and R⁹ are each independently

1) H,

2) haloalkyl,

3) C₁-C₆ alkyl,

4) C₂-C₆ alkenyl,

5) C₂-C₄ alkynyl,

6) C₃-C₇ cycloalkyl, or

7) C₃-C₇ cycloalkenyl,

wherein the alkyl, alkenyl, alkynyl, cycloalkyl, cycloalkenyl is optionally substituted with one or more R⁶ substituents; and wherein the R⁶ substituents are as defined herein.

Any and each individual definition of R⁸ and R⁹ as set out herein may be combined with any and each individual definition of Core, A, A¹, R¹, R¹⁰⁰, R², R²⁰⁰, R³, R³⁰⁰, R⁴, R⁵ and BG as set out herein.

### R¹⁰:

R¹⁰ can be any group as hereinbefore defined, without limitation. In one aspect of the aforesaid compounds, R¹⁰ is

1) halogen,

2) NO₂,

3) CN,

4) haloalkyl,

5) OR⁷,

6) NR⁸R⁹, or

7) SR⁷.

wherein R⁷, R⁸, and R⁹ are as defined herein.

Any and each individual definition of R¹⁰ as set out herein may be combined with any and each individual definition of Core, A, A¹, R¹, R¹⁰⁰, R², R²⁰⁰, R³, R³⁰⁰, R⁴, R⁵, and BG as set out herein.

If any variable, such as R⁶, R⁶⁰⁰, R¹⁰, R¹⁰⁰⁰ and the like, occurs more than one time in any constituent structure, the definition of the variable at each occurrence is independent at every other occurrence unless otherwise specified. If a substituent is itself substituted with one or more substituents, it is to be understood that that the one or more substituents may be attached to the same carbon atom or different carbon atoms. Combinations of substituents and variables defined herein are allowed only if they produce chemically stable compounds.

One skilled in the art will understand that substitution patterns and substituents on compounds of the present invention may be selected to provide compounds that are chemically stable and can be readily synthesized using the chemistry set forth in the examples and chemistry techniques well known in the art using readily available starting materials.

It is to be understood that many substituents or groups described herein have functional group equivalents, which means that the group or substituent may be replaced by another group or substituent that has similar electronic, hybridization or bonding properties.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are included but that other elements are optional and may or may not be present.

As used herein, the term “consisting of” is intended to mean including and limited to whatever follows the phrase “consisting of.” Thus the phrase “consisting of” indicates that the listed elements are included and that no other elements may be present.

As used herein, the term “alkyl” is intended to include both branched and straight chain saturated aliphatic hydrocarbon groups having the specified number of carbon atoms, for example, C₁-C₆ as in C₁-C₆-alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement, and for example, C₁-C₂₀ as in C₁-C₂₀-alkyl is defined as including groups having 1, 2, 3, 4, 5, 6, 7, 8, 9, 10, 11, 12, 13, 14, 15, 16, 17, 18, 19 or 20 carbons in a linear or branched arrangement, Examples of C₁-C₆-alkyl and C₁-C₄ alkyl as defined above include, but are not limited to, methyl, ethyl, n-propyl, i-propyl, n-butyl, t-butyl, i-butyl, pentyl and hexyl. For the purposes of describing the invention, the term “alkyl” encompasses an “alkylene.”

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regiochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like. For the purposes of describing the invention, the term “alkenyl” encompasses an “alkenylene.”

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like. For the purposes of describing the invention, the term “alkynyl” encompasses an “alkynylene.”

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl. For the purposes of describing the invention, the term “cycloalkyl” encompasses a “cycloalkylene.”

As used herein, the term “cycloalkenyl” is intended to mean a monocyclic unsaturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl. For the purposes of describing the invention, the term “cycloalkenyl” encompasses a “cycloalkenylene.”

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second or a third 5- or 6-membered carbocyclic group which may be aromatic, saturated or unsaturated. Aryl includes, but is not limited to, phenyl, indanyl, 1-naphthyl, 2-naphthyl, tetrahydronaphthyl, 1-anthracenyl, 2-anthracenyl, 9-anthracenyl, 1-phenanthryl, 2-phenanthryl, 3-phenanthryl, 4-phenanthryl, and 5-phenanthryl. The aryls may be connected to another group either at a suitable position on the cycloalkyl ring or the aromatic ring. For example:

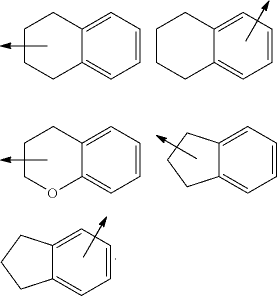

Arrowed lines drawn from the ring system indicate that the bond may be attached to any of the suitable ring atoms. For the purposes of describing the invention, the term “aryl” encompasses an “arylene.”

As used herein, the term “biphenyl” is intended to mean two phenyl groups bonded together at any one of the available sites on the phenyl ring. For example:

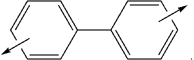

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and fluoroscein derivatives such as:

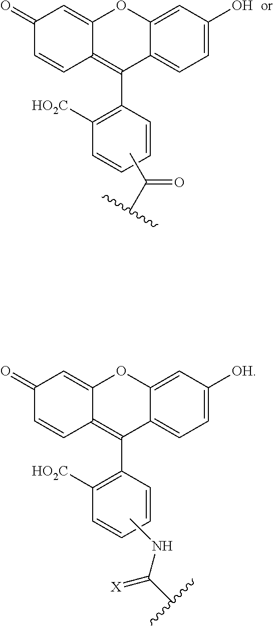

For the purposes of describing the invention, the term “heteroaryl” encompasses a “heteroarylene.”

As used herein, the term “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl, and

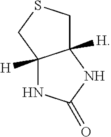

For the purposes of describing the invention, the term “heterocyclyl” encompasses a “heterocyclylene.”

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzo[d][1,3]dioxole, 2,3-dihydrobenzo[b][1,4]dioxine and 3,4-dihydro-2H-benzo[b][1,4]dioxepine.

As used herein, the term “heteroatom” is intended to mean O, S or N.

As used herein, the term “activated diacid” is intended to mean a diacid wherein the carboxylic acid moieties have been transformed to, for example, but not limited to, acid halides, a succinate esters, or HOBt esters, either in situ or in a separate synthetic step. For example, succinyl chloride and terephthaloyl chloride are examples of “diacid chlorides”. HOBt esters can be formed in situ by the treatment of a diacid with a dehydrating agent such as DCC, EDC, HBTU, or others, a base such as DIPEA, and HOBt in an appropriate solvent. The reaction of an activated diacid with an amine will result in the conversion of the acid functionality to amide functionality.

As used herein, the term “detectable label” is intended to mean a group that may be linked to a compound of the present invention to produce a probe or to an IAP BIR domain, such that when the probe is associated with the BIR domain, the label allows either direct or indirect recognition of the probe so that it may be detected, measured and quantified.

As used herein, the term “affinity tag” is intended to mean a ligand or group, which is linked to either a compound of the present invention or to an IAP BIR domain to allow another compound to be extracted from a solution to which the ligand or group is attached.

As used herein, the term “probe” is intended to mean a compound of Formula I which is labeled with either a detectable label or an affinity tag, and which is capable of binding, either covalently or non-covalently, to an IAP BIR domain. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

As used herein, the term “subject” is intended to mean humans and non-human mammals such as primates, cats, dogs, swine, cattle, sheep, goats, horses, rabbits, rats, mice and the like.

As used herein, the term “prodrug” is intended to mean a compound that may be converted under physiological conditions or by solvolysis to a biologically active compound of the present invention. Thus, the term “prodrug” refers to a precursor of a compound of the invention that is pharmaceutically acceptable. A prodrug may be inactive or display limited activity when administered to a subject in need thereof, but is converted in vivo to an active compound of the present invention. Typically, prodrugs are transformed in vivo to yield the compound of the invention, for example, by hydrolysis in blood or other organs by enzymatic processing. The prodrug compound often offers advantages of solubility, tissue compatibility or delayed release in the subject (see, Bundgard, H., Design of Prodrugs (1985), pp. 7-9, 21-24 (Elsevier, Amsterdam). The definition of prodrug includes any covalently bonded carriers which release the active compound of the invention in vivo when such prodrug is administered to a subject. Prodrugs of a compound of the present invention may be prepared by modifying functional groups present in the compound of the invention in such a way that the modifications are cleaved, either in routine manipulation or in vivo, to a parent compound of the invention.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “BIR domain binding” is intended to mean the action of a compound of the present invention upon an IAP BIR domain, which blocks or diminishes the binding of IAPs to BIR binding proteins or is involved in displacing BIR binding proteins from an IAP. Examples of BIR binding proteins include, but are not limited to, caspases and mitochondrially derived BIR binding proteins such as Smac, Omi/WTR2A and the like.

As used herein, the term “insufficient apoptosis” is intended to mean a state wherein a disease is caused or continues because cells deleterious to the subject have not apoptosed. This includes, but is not limited to, cancer cells that survive in a subject without treatment, cancer cells that survive in a subject during or following anti-cancer treatment, or immune cells whose action is deleterious to the subject, and includes, neutrophils, monocytes, B-cells and auto-reactive T-cells.

As used herein, the term “therapeutically effective amount” is intended to mean an amount of a compound of Formula 1 which, when administered to a subject is sufficient to effect treatment for a disease-state associated with insufficient apoptosis. The amount of the compound of Formula 1 will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression or alleviation of the condition or any symptom thereof.

As used herein, the term “treating cancer” is intended to mean the administration of a pharmaceutical composition of the present invention to a subject, preferably a human, which is afflicted with cancer to cause an alleviation of the cancer (i.e., any symptom of the cancer) by killing, inhibiting the growth, or inhibiting the metastasis of the cancer cells.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of pathogenic-cell survival in conditions that lead to diseases such as asthma, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “BIR domain” or “BIR” are used interchangeably throughout and are intended to mean a domain which is characterized by a number of invariant amino acid residue including conserved cysteines and one conserved hisitidine residue within the sequence Cys-(Xaa1)₂Cys-(Xaa1)₁₆His-(Xaa1)₆₋₈Cys. The BIR domain residues are listed below (see Genome Biology (2001) 1-10):

| | XIAP | HIAP-1 | HIAP-2 |
|---|---|---|---|
| BIR1 | 21-93 | 41-113 | 24-96 |
| BIR2 | 159-230 | 179-250 | 164-235 |
| BIR3 | 258-330 | 264-336 | 250-322 |
| Seq. # | P98170 | XP-006266 | XP-006267 |

As used herein, the term “ring zinc finger” or “RZF” is intended to mean a domain having the amino acid sequence of the consensus sequence: Glu-Xaa1-Xaa1-Xaa1-Xaa1-Xaa1-Xaa-1-Xaa2-Xaa1-Xaa1-Xaa1-Cys-Lys-Xaa3-Cys-Met-Xaa1-Xaa1-Xaa1-Xaa1-Xaa1-Xaa3-X-aa1-Phe-Xaa1-Pro-Cys-Gly-His-Xaa1-Xaa1-Xaa1-Cys-Xaa1-Xaa1-Cys-Ala-Xaa1-Xaa-1-Xaa1-Xaa1-Xaa1-Cys-Pro-Xaa1-Cys, wherein Xaa1 is any amino acid, Xaa2 is Glu or Asp, and Xaa3 is Val or Ile.

As used herein, the term “IAP” is intended to mean a polypeptide or protein, or fragment thereof, encoded by an IAP gene. Examples of IAPs include, but are not limited to human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6) (see for example U.S. Pat. Nos. 6,107,041; 6,133,437; 6,156,535; 6,541,457; 6,656,704; 6,689,562; Deveraux and Reed, Genes Dev. 13, 239-252, 1999; Kasof and Gomes, J. Biol. Chem., 276, 3238-3246, 2001; Vucic et al., Curr. Biol. 10, 1359-1366, 2000; Ashab et al. FEBS Lett., 495, 56-60, 2001, the contents of which are hereby incorporated by reference).

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity (preferably 95% or greater sequence identity or 100% sequence identity) to at least one of human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6). The region of sequence over which identity is measured is a region encoding at least one BIR domain and a ring zinc finger domain. Mammalian IAP genes include nucleotide sequences isolated from any mammalian source.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

As used herein, the term “modulate” or “modulating” is intended to mean the treatment, prevention, suppression, enhancement or induction of a function or condition using the compounds of the present invention. For example, the compounds of the present invention can modulate IAP function in a subject, thereby enhancing apoptosis by significantly reducing, or essentially eliminating the interaction of activated apoptotic proteins, such as caspase-3, 7 and 9, with the BIR domains of mammalian IAPs or by inducing the loss of XIAP protein in a cell.

As used herein, the term “enhancing apoptosis” is intended to mean increasing the number of cells that apoptose in a given cell population either in vitro or in vivo. Examples of cell populations include, but are not limited to, ovarian cancer cells, colon cancer cells, breast cancer cells, lung cancer cells, pancreatic cancer cells, or T cells and the like. It will be appreciated that the degree of apoptosis enhancement provided by an apoptosis-enhancing compound of the present invention in a given assay will vary, but that one skilled in the art can determine the statistically significant change in the level of apoptosis that identifies a compound that enhances apoptosis otherwise limited by an IAP. Preferably “enhancing apoptosis” means that the increase in the number of cells undergoing apoptosis is at least 25%, more preferably the increase is 50%, and most preferably the increase is at least one-fold. Preferably the sample monitored is a sample of cells that normally undergo insufficient apoptosis (i.e., cancer cells). Methods for detecting the changes in the level of apoptosis (i.e., enhancement or reduction) are described in the Examples and include methods that quantitate the fragmentation of DNA, methods that quantitate the translocation phosphatoylserine from the cytoplasmic to the extracellular side of the membrane, determination of activation of the caspases and methods quantitate the release of cytochrome C and the apoptosis inhibitory factor into the cytoplasm by mitochondria.

As used herein, the term “proliferative disease” or “proliferative disorder” is intended to mean a disease that is caused by or results in inappropriately high levels of cell division, inappropriately low levels of apoptosis, or both. For example, cancers and autoimmune disorders are all examples of proliferative diseases.

As used herein, the term “death receptor agonist” is intended to mean an agent capable of stimulating by direct or indirect contact the pro apoptotic response mediated by the death-receptors. For example, an agonist TRAIL receptor antibody would bind to TRAIL receptor (S) and trigger an apoptotic response. On the other hand, other agents such as interferon-α could trigger the release of endogeneous TRAIL and/or up regulate the TRAIL receptors in such a way that the cell pro-apoptotic response is amplified.

The compounds of the present invention, or their pharmaceutically acceptable salts, may contain one or more asymmetric centers, chiral axes and chiral planes. These compounds may, thus, give rise to enantiomers, diastereomers, and other stereoisomeric forms and may be defined in terms of absolute stereochemistry, such as (R)- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

### Utilities

The compounds of the present invention can be used for any purpose. However, compounds of Formula 1 as provided herein are believed to be especially useful as IAP BIR domain binding compounds. As such the compounds, compositions and method of the present invention include application to the cells or subjects afflicted with or having a predisposition towards developing a particular disease state, which is characterized by insufficient apoptosis. Thus, the compounds, compositions and methods of the present invention can be used to treat cellular proliferative diseases/disorders, which include, but are not limited to, i) cancer, ii) autoimmune disease, iii) inflammatory disorders, iv) proliferation induced post medical procedures, including, but not limited to, surgery, angioplasty, and the like. Accordingly, the invention provides a method of treating a proliferative disorder or other disease state characterized by insufficient apoptosis comprising administering to a subject in need thereof a therapeutically effective amount of a compound of the invention (e.g., a compound of Formula 1) or pharmaceutical composition comprising same, so as to treat the proliverative disorder or disease state characterized by insufficient apoptosis.

The compounds of the present invention may be particularly useful in the treatment of diseases in which there is a defect in the programmed cell-death or the apoptotic machinery (TRAIL, FAS, apoptosome), such as multiple sclerosis, artherosclerosis, inflammation, autoimmunity, rheumatoid arthritis (RA) and the like. Without wishing to be bound by any particular theory, it is believed that the compounds of the present invention act in combination with endogenous cell-death ligands, such as Fas, to induce apoptosis in synoviocytes (e.g., human synoviocytes). Thus, in another aspect, the invention provides a method of inducing apoptosis in a synoviocyte, especially human synoviocytes, comprising administering to the synoviocyte a compound of the invention alone or in combination, simultaneously or sequentially, with a cell-death ligand including, but not limited to, Fas. The synoviocyte can be in a tissue or a subject, for example, a tissue or subject afflicted with a disease associated with a defect in the programmed cell-death or the apoptotic machinery (TRAIL, FAS, apoptosome) of a synoviocyte, especially an autoimmune disease such as RA.

In particular, the compounds, compositions and methods of the present invention can be used for the treatment of cancer including solid tumors such as skin, breast, brain, lung, testicular carcinomas, and the like. Cancers that may be treated by the compounds, compositions and methods of the invention include, but are not limited to the following:

| Tissue | Example |
|---|---|
| Adrenal gland | neuroblastoma |
| Bone | osteogenic sarcoma (osteosarcoma), fibrosarcoma, malignant |
| | fibrous histiocytoma, chondrosarcoma, Ewing's sarcoma, |
| | malignant lymphoma (reticulum cell sarcoma), multiple |
| | myeloma, malignant giant cell tumor chordoma, |
| | osteochronfroma (osteocartilaginous exostoses), benign |
| | chondroma, chondroblastoma, chondromyxofibroma, osteoid |
| | osteoma and giant cell tumors |
| Cardiac | sarcoma (angiosarcoma, fibrosarcoma, rhabdomyosarcoma, |
| | liposarcoma), myxoma, rhabdomyoma, fibroma, lipoma and teratoma |
| Gastrointestinal | esophagus (squamous cell carcinoma, adenocarcinoma, |
| | leiomyosarcoma, lymphoma), stomach (carcinoma, lymphoma, |
| | leiomyosarcoma), pancreas (ductal adenocarcinoma, |
| | insulinoma, glucagonoma, gastrinoma, carcinoid tumors, |
| | vipoma), small bowel (adenocarcinoma, lymphoma, carcinoid |
| | tumors, Karposi's sarcoma, leiomyoma, hemangioma, lipoma, |
| | neurofibroma, fibroma), large bowel (adenocarcinoma, tubular |
| | adenoma, villous adenoma, hamartoma, leiomyoma) |
| Genitourinary tract | kidney (adenocarcinoma, Wilm's tumor [nephroblastoma], |
| | lymphoma, leukemia), bladder and urethra (squamous cell |
| | carcinoma, transitional cell carcinoma, adenocarcinoma), |
| | prostate (adenocarcinoma, sarcoma), testis (seminoma, |
| | teratoma, embryonal carcinoma, teratocarcinoma, |
| | choriocarcinoma, sarcoma, interstitial cell carcinoma, fibroma, |
| | fibroadenoma, adenomatoid tumors, lipoma) |
| Gynecological | uterus (endometrial carcinoma), cervix (cervical carcinoma, |
| | pre-tumor cervical dysplasia), ovaries (ovarian carcinoma |
| | [serous cystadenocarcinoma, mucinous cystadenocarcinoma, |
| | unclassified carcinoma], granulosa-thecal cell tumors, Sertoli- |
| | Leydig cell tumors, dysgerminoma, malignant teratoma), vulva |
| | (squamous cell carcinoma, intraepithelial carcinoma, |
| | adenocarcinoma, fibrosarcoma, melanoma), vagina (clear cell |
| | carcinoma, squamous cell carcinoma, botryoid sarcoma |
| | (embryonal rhabdomyosarcoma), fallopian tubes (carcinoma) |
| Hematologic | blood (myeloid leukemia [acute and chronic], acute |
| | lymphoblastic leukemia, chronic lymphocytic leukemia, |
| | myeloproliferative diseases, multiple myeloma, myelodysplastic |
| | syndrome), Hodgkin's disease, non-Hodgkin's lymphoma |
| | [malignant lymphoma] |
| Liver | hepatoma (hepatocellular carcinoma), cholangiocarcinoma, |
| | hepatoblastoma, angiosarcoma, hepatocellular adenoma, |
| | hemangioma |
| Lung | bronchogenic carcinoma (squamous cell, undifferentiated small |
| | cell, undifferentiated large cell, adenocarcinoma), alveolar |
| | (bronchiolar) carcinoma, bronchial adenoma, sarcoma, |
| | lymphoma, chondromatous hamartoma, mesothelioma |
| Nervous system | skull (osteoma, hemangioma, granuloma, xanthoma, osteitis |
| | deformans), meninges (meningioma, meningiosarcoma, |
| | gliomatosis), brain (astrocytoma, medulloblastoma, glioma, |
| | ependymoma, germinoma [pinealoma], glioblastoma multiform, |
| | oligodendroglioma, schwannoma, retinoblastoma, congenital |
| | tumors), spinal cord neurofibroma, meningioma, glioma, |
| | sarcoma) |
| Skin | malignant melanoma, basal cell carcinoma, squamous cell |
| | carcinoma, Karposi's sarcoma, moles dysplastic nevi, lipoma, |
| | angioma, dermatofibroma, keloids |

The compounds of the present invention, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions of the present invention can be prepared by mixing a compound of the present invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins, including, but not limited to, α, β, or δ-hydroxypropylcyclodextins or Captisol; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 10% by weight of the compound of the present invention. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others.

The pharmaceutical composition of the present invention may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the present invention from about 0.1 to about 10% w/v (weight per unit volume).

The pharmaceutical composition of the present invention may be used for rectal administration to treat for example, colon cancer, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the present invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical composition of the present invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

### Combination Therapy

The compounds of the present invention, or pharmaceutically acceptable salts thereof, may also be administered simultaneously with, prior to, or after administration of one or more additional therapeutic agents described herein. Such combination therapy may include administration of a single pharmaceutical dosage formulation which contains a compound of the present invention and one or more additional agents given below, as well as administration of the compound of the present invention in a pharmaceutical dosage formulation separate from one or more additional therapeutic agents. For example, a compound of the present invention and a chemotherapeutic agent, such as taxol (paclitaxel), taxotere, etoposide, cisplatin, vincristine, vinblastine, and the like, can be administered to the patient either together in a single oral dosage composition such as a tablet or capsule, or each agent administered in separate oral dosage formulations or via intravenous injection. Where separate dosage formulations are used, the compounds of the present invention and one or more additional agents can be administered at essentially the same time, i.e., concurrently, or at separately staggered times, i.e., sequentially; combination therapy is understood to include all these regimens. In addition, these compounds may synergize with molecules that may stimulate the death receptor apoptotic pathway through a direct or indirect manner. Accordingly, the compounds of the present invention may be used in combination with soluble TRAIL, an anti-TRAIL receptor antibody, or any agent or procedure that can cause an increase in circulating level of TRAIL, such as interferon-alpha or radiation.

Thus, the present invention also encompasses the use of the compounds of the present invention in combination with radiation therapy and/or one or more additional agents such as those described in WO 03/099211 (PCT/US03/15861), which is hereby incorporated by reference. Examples of such additional agents include, but are not limited to the following:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug.
s) a proteasome inhibitor such as Velcade and MG132 (7-Leu-Leu-aldehyde) (see He at al. in Oncogene (2004) 23, 2554-2558);
t) an HDAC inhibitor, such as sodium butyrate, phenyl butyrate, hydroamic acids, cyclin tetrapeptide and the like (see Rosato et al., Molecular Cancer Therapeutics 2003, 1273-1284);′
u) an inhibitor of the chimotrypsin-like activity in the proteasome;
v) E3 ligase inhibitors;
w) a modulator of the immune system such as interferon-alpha and ionizing radiation (UVB) that can induce the release of cytokines, such as the interleukins, TNF, or induce release of Death receptor Ligands such as TRAIL;
x) a modulator of death receptors, including TRAIL and TRAIL receptor agonists such as the humanized antibodies HGS-ETR1 and HGS-ETR2.

Additional combinations may also include agents which reduce the toxicity of the aforesaid agents, such as hepatic toxicity, neuronal toxicity, nephprotoxicity and the like.

### TRAIL Receptor Agonists

In one example, co-administration of one of the compounds of Formula I of the present invention with a death receptor agonist such as TRAIL, such as a small molecule or an antibody that mimics TRAIL may cause an advantageous synergistic effect. Moreover, the compounds of the present invention may be used in combination with any compounds that cause an increase in circulating levels of TRAIL. Agonist antibodies directed against the death receptors TRAIL-R1 and/or TRAIL-R2 can be used in combination with compounds of the invention. Exemplary agonist antibodies that may be used in combination with compounds of the invention include those described in U.S. Pat. No. 7,244,429; in U.S. Patent Application Publication Nos. 2007/0179086, 2002/0004227, 2006/0269554, 2005/0079172, 2007/0292411, 2006/0270837, 2006/0269555, 2004/0214235, and 2007/0298039; and in International Patent Publications WO2006/017961 and WO98/51793. Each of these publications is hereby incorporated by reference in its entirety. In preferred embodiments, compounds of the invention are used in combination with one or more of these TRAIL receptor agonist antibodies for the treatment of cancer and other neoplasms.

### Vinca Alkaloids and Related Compounds

Vinca alkaloids that can be used in combination with the nucleobase oligomers of the invention to treat cancer and other neoplasms include vincristine, vinblastine, vindesine, vinflunine, vinorelbine, and anhydrovinblastine.

Dolastatins are oligopeptides that primarily interfere with tubulin at the vinca alkaloid binding domain. These compounds can also be used in combination with the compounds of the invention to treat cancer and other neoplasms. Dolastatins include dolastatin-10 (NCS 376128), dolastatin-15, ILX651, TZT-1027, symplostatin 1, symplostatin 3, and LU103793 (cemadotin).

Cryptophycins (e.g., cryptophycin 1 and cryptophycin 52 (LY355703)) bind tubulin within the vinca alkaloid-binding domain and induce G2/M arrest and apoptosis. Any of these compounds can be used in combination with the compounds of the invention to treat cancer and other neoplasms.

Other microtubule disrupting compounds that can be used in conjunction with the compounds of the invention to treat cancer and other neoplasms are described in U.S. Pat. Nos. 6,458,765; 6,433,187; 6,323,315; 6,258,841; 6,143,721; 6,127,377; 6,103,698; 6,023,626; 5,985,837; 5,965,537; 5,955,423; 5,952,298; 5,939,527; 5,886,025; 5,831,002; 5,741,892; 5,665,860; 5,654,399; 5,635,483; 5,599,902; 5,530,097; 5,521,284; 5,504,191; 4,879,278; and 4,816,444, and U.S. patent application Publication Nos. 2003/0153505 A1; 2003/0083263 A1; and 2003/0055002 A1, each of which is hereby incorporated by reference.

### Taxanes and Other Micortubule Stabilizing Compounds

Taxanes such as paclitaxel, doxetaxel, RPR 109881A, SB-T-1213, SB-T-1250, SB-T-101187, BMS-275183, BRT 216, DJ-927, MAC-321, IDN5109, and IDN5390 can be used in combination with the compounds of the invention to treat cancer and other neoplasms. Taxane analogs (e.g., BMS-184476, BMS-188797) and functionally related non-taxanes (e.g., epothilones (e.g., epothilone A, epothilone B (EPO906), deoxyepothilone B, and epothilone B lactam (BMS-247550)), eleutherobin, discodermolide, 2-epi-discodermolide, 2-des-methyldiscodermolide, 5-hydroxymethyldiscoder-molide, 19-des-aminocarbonyldiscodermolide, 9(13)-cyclodiscodermolide, and laulimalide) can also be used in the methods and compositions of the invention.

Other microtubule stabilizing compounds that can be used in combination with the compounds of the invention to treat cancer and other neoplasms are described in U.S. Pat. Nos. 6,624,317; 6,610,736; 6,605,599; 6,589,968; 6,583,290; 6,576,658; 6,515,017; 6,531,497; 6,500,858; 6,498,257; 6,495,594; 6,489,314; 6,458,976; 6,441,186; 6,441,025; 6,414,015; 6,387,927; 6,380,395; 6,380,394; 6,362,217; 6,359,140; 6,306,893; 6,302,838; 6,300,355; 6,291,690; 6,291,684; 6,268,381; 6,262,107; 6,262,094; 6,147,234; 6,136,808; 6,127,406; 6,100,411; 6,096,909; 6,025,385; 6,011,056; 5,965,718; 5,955,489; 5,919,815; 5,912,263; 5,840,750; 5,821,263; 5,767,297; 5,728,725; 5,721,268; 5,719,177; 5,714,513; 5,587,489; 5,473,057; 5,407,674; 5,250,722; 5,010,099; and 4,939,168; and U.S. patent application Publication Nos. 2003/0186965 A1; 2003/0176710 A1; 2003/0176473 A1; 2003/0144523 A1; 2003/0134883 A1; 2003/0087888 A1; 2003/0060623 A1; 2003/0045711 A1; 2003/0023082 A1; 2002/0198256 A1; 2002/0193361 A1; 2002/0188014 A1; 2002/0165257 A1; 2002/0156110 A1; 2002/0128471 A1; 2002/0045609 A1; 2002/0022651 A1; 2002/0016356 A1; 2002/0002292 A1, each of which is hereby incorporated by reference.

Other chemotherapeutic agents that may be administered with a compound of the present invention are listed in the following Table:

| | | |
|---|---|---|
| Alkylating | cyclophosphamide | mechlorethamine |
| agents | lomustine | thiotepa |
| | busulfan | streptozocin |
| | procarbazine | chlorambucil |
| | ifosfamide | temozolomide |
| | altretamine | dacarbazine |
| | melphalan | semustine |
| | estramustine phosphate | carmustine |
| | hexamethylmelamine | |
| Platinum | cisplatin | tetraplatin |
| agents | carboplatinum | BBR-3464 (Hoffmann-La Roche) |
| | oxaliplatin | Ormiplatin |
| | ZD-0473 (AnorMED) | SM-11355 (Sumitomo) |
| | spiroplatinum | iproplatin |
| | lobaplatin (Aeterna) | AP-5280 (Access) |
| | carboxyphthalatoplatinum | |
| | satraplatin (Johnson Matthey) | |
| Antimetabolites | azacytidine | 6-mercaptopurine |
| | tomudex | hydroxyurea |
| | gemcitabine | 6-thioguanine |
| | trimetrexate | decitabine (SuperGen) |
| | capecitabine | cytarabin |
| | deoxycoformycin | clofarabine (Bioenvision) |
| | 5-fluorouracil | 2-fluorodeoxy |
| | fludarabine | cytidine |
| | floxuridine | irofulven (MGI Pharma) |
| | pentostatin | methotrexate |
| | 2-chlorodeoxyadenosine | DMDC (Hoffmann-La Roche) |
| | raltitrexed | idatrexate |
| | | ethynylcytidine (Taiho) |
| Topoisomerase | amsacrine | TAS-103 (Taiho) |
| inhibitors | rubitecan (SuperGen) | Topotecan |
| | epirubicin | elsamitrucin (Spectrum) |
| | exatecan mesylate (Daiichi) | dexrazoxanet (TopoTarget) |
| | etoposide | J-107088 (Merck & Co) |
| | quinamed (ChemGenex) | pixantrone (Novuspharma) |
| | teniposide or mitoxantrone | BNP-1350 (BioNumerik) |
| | gimatecan (Sigma-Tau) | rebeccamycin analogue (Exelixis) |
| | irinotecan (CPT-11) | CKD-602 (Chong Kun Dang) |
| | diflomotecan (Beaufour-Ipsen) | BBR-3576 (Novuspharma) |
| | 7-ethyl-10-hydroxy-camptothecin | KW-2170 (Kyowa Hakko) |
| Antitumor | dactinomycin (actinomycin D) | bleomycinic acid |
| antibiotics | amonafide | idarubicin |
| | doxorubicin (adriamycin) | bleomycin A |
| | azonafide | rubidazone |
| | deoxyrubicin | bleomycin B |
| | anthrapyrazole | plicamycinp |
| | valrubicin | mitomycin C |
| | oxantrazole | porfiromycin |
| | daunorubicin (daunomycin) | MEN-10755 (Menarini) |
| | losoxantrone | cyanomorpholinodoxorubicin |
| | epirubicin | GPX-100 (Gem Pharmaceuticals) |
| | bleomycin sulfate (blenoxane) | mitoxantrone (novantrone) |
| | therarubicin | |
| Antimitotic | paclitaxel | RPR 109881A (Aventis) |
| agents | SB 408075 (GlaxoSmithKline) | ZD 6126 (AstraZeneca) |
| | docetaxel | TXD 258 (Aventis) |
| | E7010 (Abbott) | PEG-paclitaxel (Enzon) |
| | Colchicines | epothilone B (Novartis) |
| | PG-TXL (Cell Therapeutics) | AZ10992 (Asahi) |
| | vinblastine | T 900607 (Tularik) |
| | IDN 5109 (Bayer) | IDN-5109 (Indena) |
| | Vincristine | T 138067 (Tularik) |
| | A 105972 (Abbott) | AVLB (Prescient NeuroPharma) |
| | Vinorelbine | cryptophycin 52 (Eli Lilly) |
| | A 204197 (Abbott) | azaepothilone B (BMS) |
| | Vindesine | vinflunine (Fabre) |
| | LU 223651 (BASF) | BNP-7787 (BioNumerik) |
| | dolastatin 10 (NCI) | auristatin PE (Teikoku Hormone) |
| | D 24851 (ASTAMedica) | CA-4 prodrug (OXiGENE) |
| | rhizoxin (Fujisawa) | BMS 247550 (BMS) |
| | ER-86526 (Eisai) | dolastatin-10 (NIH) |
| | mivobulin (Warner-Lambert) | BMS 184476(BMS) |
| | combretastatin A4 (BMS) | CA-4 (OXiGENE) |
| | cemadotin (BASF) | BMS 188797 (BMS) |
| | isohomohalichondrin-B | taxoprexin (Protarga) |
| | (PharmaMar) | |
| Aromatase | Aminoglutethimide | anastrazole |
| inhibitors | Exemestane | YM-511 (Yamanouchi) |
| | Letrozole | formestane |
| | atamestane (BioMedicines) | |
| Thymidylate | pemetrexed (Eli Lilly) | ZD-9331 (BTG) |
| synthase | nolatrexed (Eximias) | CoFactor ™ (BioKeys) |
| inhibitors | | |
| DNA | trabectedin (PharmaMar) | albumin + 32P (Isotope Solutions) |
| antagonists | mafosfamide (Baxter International) | O6 benzyl guanine (Paligent) |
| | glufosfamide (Baxter International) | thymectacin (NewBiotics) |
| | apaziquone (Spectrum | edotreotide (Novartis) |
| | Pharmaceuticals) | |
| Farnesyltransferase | arglabin (NuOncology Labs) | perillyl alcohol (DOR BioPharma) |
| inhibitors | tipifarnib (Johnson & Johnson) | BAY-43-9006 (Bayer) |
| | lonafarnib (Schering-Plough) | |
| Pump | CBT-1 (CBA Pharma) | tariquidar (Xenova) |
| inhibitors | zosuquidar trihydrochloride (Eli | biricodar dicitrate (Vertex) |
| | Lilly) | MS-209 (Schering AG) |
| Histone | tacedinaline (Pfizer) | depsipeptide (Fujisawa) |
| acetyltransferase | pivaloyloxymethyl butyrate (Titan) | MS-275 (Schering AG) |
| inhibitors | SAHA (Aton Pharma) | |
| Metalloproteinase | Neovastat (Aeterna Laboratories) | marimastat (British Biotech) BMS- |
| inhibitors | CMT-3 (CollaGenex) | 275291 (Celltech) |
| Ribonucleoside | gallium maltolate (Titan) | triapine (Vion) |
| reductase | tezacitabine (Aventis) | didox (Molecules for Health) |
| inhibitors | | |
| TNF alpha | virulizin (Lorus Therapeutics) | CDC-394 (Celgene) |
| agonists/antagonists | revimid (Celgene) | |
| Endothelin A | atrasentan (Abbott) | ZD-4054 (AstraZeneca) |
| receptor | YM-598 (Yamanouchi) | |
| antagonist | | |
| Retinoic acid | fenretinide (Johnson & Johnson) | LGD-1550 (Ligand) |
| receptor | alitretinoin (Ligand) | |
| agonists | | |
| Immuno- | Interferon | norelin (Biostar) |
| modulators | dexosome therapy (Anosys) | IRX-2 (Immuno-Rx) |
| | oncophage (Antigenics) | BLP-25 (Biomira) |
| | pentrix (Australian Cancer | PEP-005 (Peplin Biotech) |
| | Technology) | MGV (Progenics) |
| | GMK (Progenics) | synchrovax vaccines (CTL |
| | ISF-154 (Tragen) | Immuno) |
| | adenocarcinoma vaccine | beta.-alethine (Dovetail) |
| | (Biomira) cancer vaccine | melanoma vaccine (CTL Immuno) |
| | (Intercell) | CLL therapy (Vasogen) |
| | CTP-37 (A VI BioPharma) | p21 RAS vaccine (GemVax) |
| Hormonal and | estrogens | bicalutamide |
| antihormonal | Prednisone | testosterone propionate; |
| agents | conjugated estrogens | fluoxymesterone |
| | methylprednisolone | flutamide |
| | ethinyl estradiol | methyltestosterone |
| | prednisolone | octreotide |
| | chlortrianisen | diethylstilbestrol |
| | aminoglutethimide | nilutamide |
| | idenestrol | megestrol |
| | leuprolide | mitotane tamoxifen |
| | hydroxyprogesterone caproate | P-04 (Novogen) |
| | goserelin | Toremofine |
| | medroxyprogesterone | 2-methoxyestradiol (EntreMed) |
| | leuporelin | dexamethasone |
| | testosterone | arzoxifene (Eli Lilly) |
| Photodynamic | talaporfin (Light Sciences) | motexafin |
| agents | Pd-bacteriopheophorbide (Yeda) | gadolinium (Pharmacyclics) |
| | Theralux (Theratechnologies) | hypericin |
| | lutetium texaphyrin | |
| | (Pharmacyclics) | |
| Tyrosine | imatinib (Novartis) | C225 (ImClone) |
| Kinase | kahalide F (PharmaMar) | ZD4190 (AstraZeneca) |
| Inhibitors | leflunomide (Sugen/Pharmacia) | rhu-Mab (Genentech) |
| | CEP-701 (Cephalon) | ZD6474 (AstraZeneca) |
| | ZD1839 (AstraZeneca) | MDX-H210 (Medarex) |
| | CEP-751 (Cephalon) | vatalanib (Novartis) |
| | erlotinib (Oncogene Science) | 2C4 (Genentech) |
| | MLN518 (Millenium) | PKI166 (Novartis) |
| | canertinib (Pfizer) | MDX-447 (Medarex) |
| | PKC412 (Novartis) | GW2016 (GlaxoSmithKline) |
| | squalamine (Genaera) | ABX-EGF (Abgenix) |
| | phenoxodiol | EKB-509 (Wyeth) |
| | SU5416 (Pharmacia) | IMC-1C11 (ImClone) |
| | trastuzumab (Genentech) | EKB-569 (Wyeth) |
| | SU6668 (Pharmacia) | CI-1033 |
| | Sorafenib | EKB-569 |
| | Herceptin | Semaxanib |
| | Cetuximab | ZD6474 |
| | ZD1839 | PTK-787 |
| | PKI 166 | INC-1C11 |

Miscellaneous agents

| | |
|---|---|
| SR-27897 (CCK A inhibitor, Sanofi- | gemtuzumab (CD33 antibody, Wyeth |
| Synthelabo) | Ayerst) |
| BCX-1777 (PNP inhibitor, BioCryst) | CCI-779 (mTOR kinase inhibitor, Wyeth) |
| tocladesine (cyclic AMP agonist, | PG2 (hematopoiesis enhancer, |
| Ribapharm) | Pharmagenesis) |
| ranpirnase (ribonuclease stimulant, Alfacell) | exisulind (PDE V inhibitor, Cell Pathways) |
| alvocidib (CDK inhibitor, Aventis) | Immunol ™ (triclosan oral rinse, Endo) |
| galarubicin (RNA synthesis inhibitor, Dong- | CP-461 (PDE V inhibitor, Cell Pathways) |
| A) | triacetyluridine (uridine prodrug, Wellstat) |
| CV-247 (COX-2 inhibitor, Ivy Medical) | AG-2037 (GART inhibitor, Pfizer) |
| tirapazamine (reducing agent, SRI | SN-4071 (sarcoma agent, Signature |
| International) | BioScience) WX-UK1 (plasminogen |
| P54 (COX-2 inhibitor, Phytopharm) | activator inhibitor, Wilex) |
| N-acetylcysteine (reducing agent, Zambon) | TransMID-107 .TM. (immunotoxin, KS |
| CapCell ™ (CYP450 stimulant, Bavarian | Biomedix) |
| Nordic) | PBI-1402 (PMN stimulant, ProMetic |
| R-flurbiprofen (NF-kappaB inhibitor, | LifeSciences) |
| Encore) | PCK-3145 (apoptosis promotor, Procyon) |
| GCS-100 (gal3 antagonist, GlycoGenesys) | bortezomib (proteasome inhibitor, |
| 3CPA (NF-kappaB inhibitor, Active Biotech) | Millennium) |
| G17DT immunogen (gastrin inhibitor, | doranidazole (apoptosis promotor, Pola) |
| Aphton) | SRL-172 (T cell stimulant, SR Pharma) |
| seocalcitol (vitamin D receptor agonist, Leo) | CHS-828 (cytotoxic agent, Leo) |
| efaproxiral (oxygenator, Allos Therapeutics) | TLK-286 (glutathione S transferase |
| 131-I-TM-601 (DNA antagonist, | inhibitor, Telik) |
| TransMolecular) | trans-retinoic acid (differentiator, NIH) |
| PI-88 (heparanase inhibitor, Progen) | PT-100 (growth factor agonist, Point |
| eflornithine (ODC inhibitor, ILEX Oncology) | Therapeutics) |
| tesmilifene (histamine antagonist, YM | MX6 (apoptosis promotor, MAXIA) |
| BioSciences) | midostaurin (PKC inhibitor, Novartis) |
| minodronic acid (osteoclast inhibitor, | apomine (apoptosis promotor, ILEX |
| Yamanouchi) | Oncology) |
| histamine (histamine H2 receptor agonist, | bryostatin-1 (PKC stimulant, GPC Biotech) |
| Maxim) | urocidin (apoptosis promotor, Bioniche) |
| indisulam (p53 stimulant, Eisai) | CDA-II (apoptosis promotor, Everlife) |
| tiazofurin (IMPDH inhibitor, Ribapharm) | Ro-31-7453 (apoptosis promotor, La |
| aplidine (PPT inhibitor, PharmaMar) | Roche) |
| cilengitide (integrin antagonist, Merck | SDX-101 (apoptosis promotor, Salmedix) |
| KGaA) | brostallicin (apoptosis promotor, |
| rituximab (CD20 antibody, Genentech) | Pharmacia) |
| SR-31747 (IL-1 antagonist, Sanofi- | ceflatonin (apoptosis promotor, |
| Synthelabo) | ChemGenex) |

Additional combinations may also include agents which reduce the toxicity of the aforesaid agents, such as hepatic toxicity, neuronal toxicity, nephprotoxicity and the like.

Additional combinations may be used in the treatment of RA such as non-steroidal anti-inflammatory drugs (NSAIDs), analgesics, corticosteroids and disease-modifying antirheumatic drugs. Further combinations may include Kineret, Actemra, Hydroxychloroquine (Plaquenil™), Sulfasalazine (Azulfidine™), Leflunomide (Arava™), Tumor Necrosis Factor Inhibitors such as etanercept (Enbrel™, adalimumab (Humira™), and infliximab (Remicade™), T-cell costimulatory blocking agents such as abatacept (Orencia™) B cell depleting agents such as rituximab (Rituxan™), Interleukin-1 (IL-1) receptor antagonist therapy such as anakinra (Kineret™), intramuscular gold and other immunomodulatory and cytotoxic agents such as azathioprine (Imuran™), cyclophosphamide and cyclosporine A (Neoral™, Sandimmune™).

Other cotherapies for the treatment of RA include Methotrexate, Campath (alemtuzumab), anti-RANKL MAb (denosumab), anti-Blys MAb LymphoStat-B™ (belimumab), Cimzia (certolizumab pegol), p38 inhibitors, JAK inhibitors, anti-TNF agents, anti-CD20 MAbs, anti-IL/ILR targeting agents such as those which target IL-1, IL-5, IL-6 (toclizumab), 11-4, IL-13, and IL-23.

Additional combinations may be used in the treatment of MS such as Remicade™, Enbrel™, Humaira™, Kineret™, Orencia™, Rituxan™ and TYSABRI™ (natalizumab).

### Screening Assays

The compounds of the present invention may also be used in a method to screen for other compounds that bind to an IAP BIR domain. Generally speaking, to use the compounds of the invention in a method of identifying compounds that bind to an IAP BIR domain, the IAP is bound to a support, and a compound of the invention is added to the assay. Alternatively, the compound of the invention may be bound to the support and the IAP is added.

There are a number of ways in which to determine the binding of a compound of the present invention to the BIR domain. In one way, the compound of the invention, for example, may be fluorescently or radioactively labeled and binding determined directly. For example, this may be done by attaching the IAP to a solid support, adding a detectably labeled compound of the invention, washing off excess reagent, and determining whether the amount of the detectable label is that present on the solid support. Numerous blocking and washing steps may be used, which are known to those skilled in the art.

In some cases, only one of the components is labeled. For example, specific residues in the BIR domain may be labeled. Alternatively, more than one component may be labeled with different labels; for example, using I¹²⁵ for the BIR domain, and a fluorescent label for the probe.

The compounds of the invention may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the IAP biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an IAP BIR domain and a probe to form a probe:BIR domain complex in a first sample followed by adding a test compound from a second sample. The binding of the test is determined, and a change or difference in binding between the two samples indicates the presence of a test compound capable of binding to the BIR domain and potentially modulating the IAP's activity.

In one case, the binding of the test compound is determined through the use of competitive binding assays. In this embodiment, the probe is labeled with a fluorescent label. Under certain circumstances, there may be competitive binding between the test compound and the probe. Test compounds which display the probe, resulting in a change in fluorescence as compared to control, are considered to bind to the BIR region.

In one case, the test compound may be labeled. Either the test compound, or a compound of the present invention, or both, is added first to the IAP BIR domain for a time sufficient to allow binding to form a complex.

Formation of the probe:BIR domain complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the BIR domain.

In one case, the probe is added first, followed by the test compound. Displacement of the probe is an indication the test compound is binding to the BIR domain and thus is capable of binding to, and potentially modulating, the activity of IAP. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the BIR domain with a higher affinity. Thus, if the probe is detected on the support, coupled with a lack of test compound binding, may indicate the test compound is capable of binding to the BIR domain.

Modulation is tested by screening for a test compound's ability to modulate the activity of IAP and includes combining a test compound with an IAP BIR domain, as described above, and determining an alteration in the biological activity of the IAP. Therefore in this case, the test compound should both bind to the BIR domain (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays in this invention include a fluorescent label such as Fluorescein, Oregon green, dansyl, rhodamine, tetramethyl rhodamine, texas red, Eu³⁺; a chemiluminescent label such as luciferase; colorimetric labels; enzymatic markers; or radioisotopes such as tritium, I¹²⁵ and the like. Affinity tags, which may be useful in performing the screening assays of the present invention include be biotin, polyhistidine and the like.

### Synthesis and Methodology

General methods for the synthesis of the compounds of the present invention are shown below and are disclosed merely for the purpose of illustration and are not meant to be interpreted as limiting the processes to make the compounds by any other methods. Those skilled in the art will readily appreciate that a number of methods are available for the preparation of the compounds of the present invention.

### General Procedures

Scheme 1, 2, 3, 4, 6, and 7 illustrate various general synthetic procedures for the preparation of compounds of the instant invention. As used in Schemes 1, 2, 3, 4, 6, and 7, L is as defined herein and L¹ is a group defined by the structure: —C(O)-L-C(O)—.

### Method A

Protected amino-proline derivative 1-i is treated with LG-L¹-LG to provide intermediate 1-ii. Intermediate 1-ii is then deprotected at PG¹ to yield intermediate 1-iii. Intermediate 1-iii is converted to intermediate 1-v by an amino acid coupling/deprotection sequences. A second amino acid coupling step converts intermediate 1-v to intermediate 1-vi. Deprotection of 1-vi at PG² yields the diacid intermediate 2-i. Treatment of 2-i with amino acid coupling reagents, followed by R⁴R⁵NH yields intermediate 2-ii, which upon deprotection of PG⁴ provided compound 2-iii.

Thus, the invention provides a method of preparing a compound of Formula 1 comprising the steps of Method A. Furthermore, each of the individual steps of Method A, the intermediates involved, and methods of preparing the intermediates, are considered to be additional aspects of the invention. Thus, by way of illustration, the method can comprise (a) deprotecting combining 1-I with LG-L¹-LG to provide intermediate 1-ii. Alternatively, or in addition, the method can comprise (b) deprotecting PG1 to yield intermediate 1-iii. Alternatively, or in addition, the method can comprise (c) converting intermediate 1-iii to intermediate 1-v by combining intermediate 1-iii with a coupling agent as illustrated in Scheme 1. Alternatively, or in addition, the method can comprise (d) converting intermediate 1-v to intermediate 1-vi by combining intermediate 1-v with a coupling agent as illustrated in Scheme 1. Alternatively or in addition, the method can comprise (e) deprotecting 1-vi at PG² to provide intermediate 2-i. Alternatively, or in addition, the method can comprise (f) treatment of 2-I with a coupling agent and R⁴R⁵NH to provide intermediate 2-ii. Alternatively, or in addition, the method can comprise deprotecting intermediate 2-ii to provide compound 2-iii.

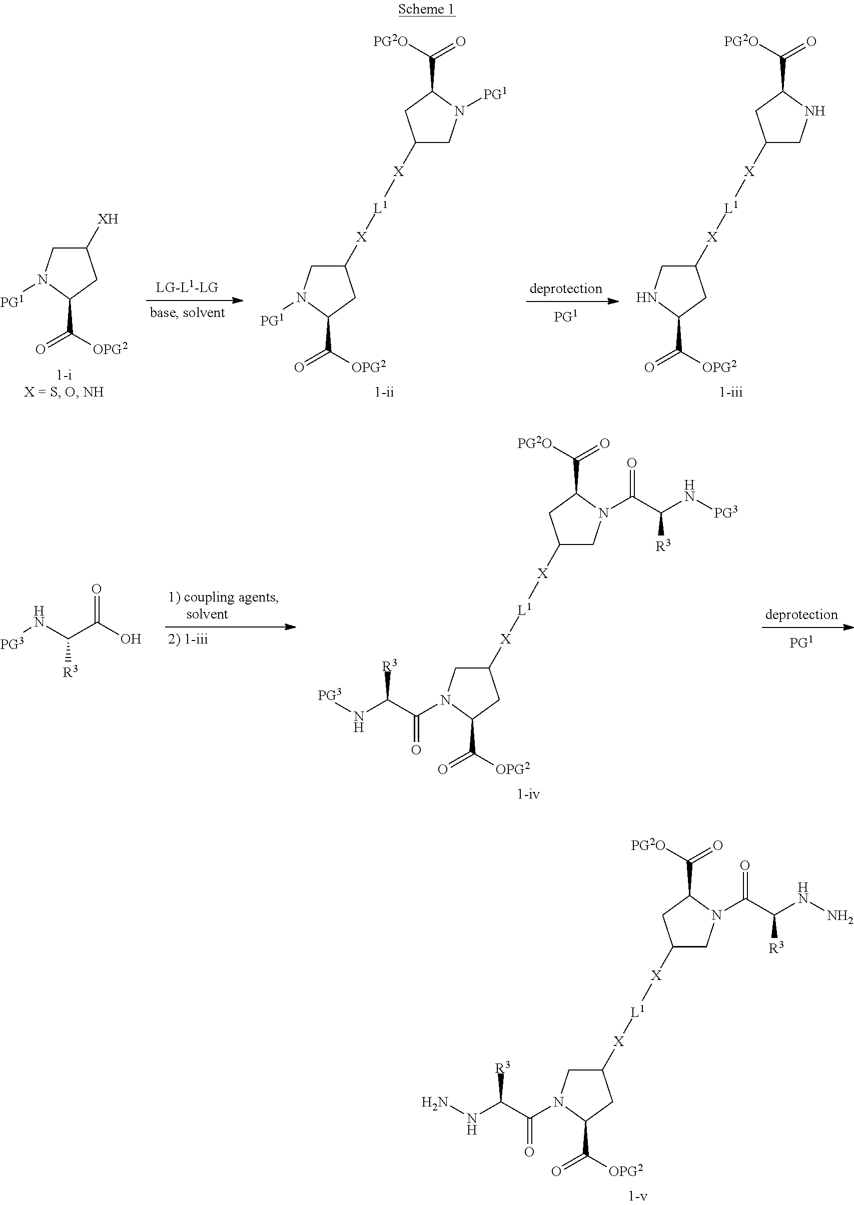

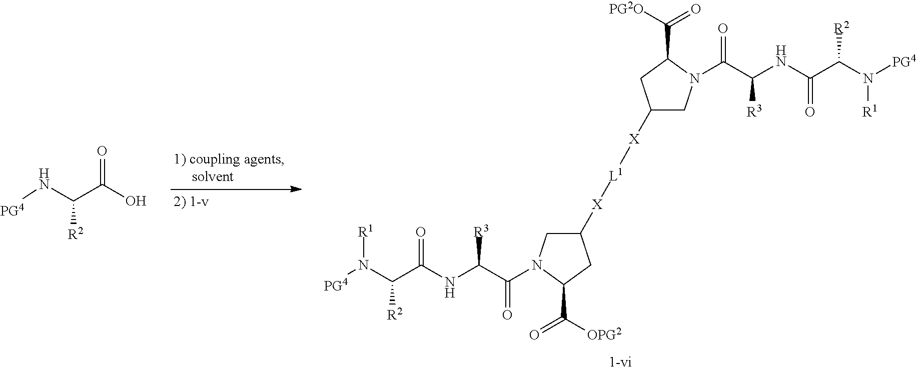

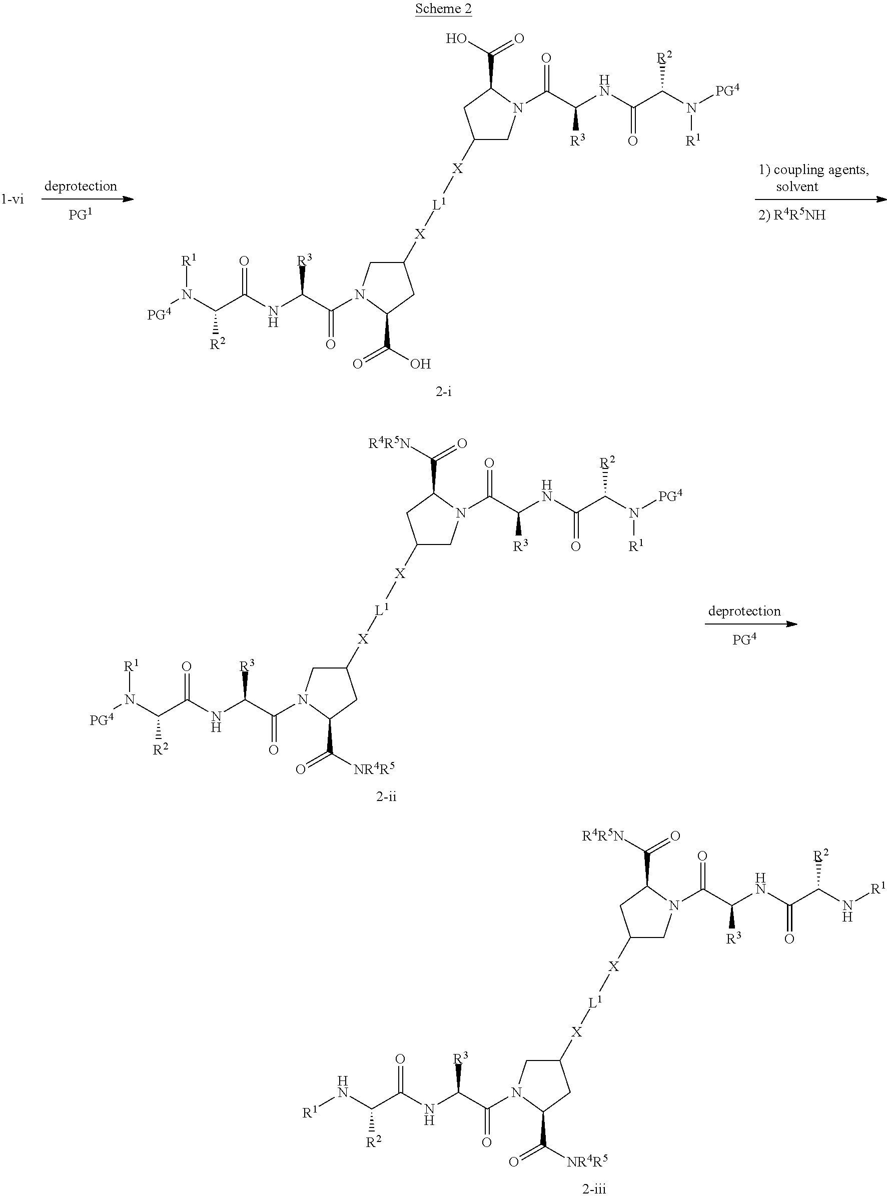

### Method B

PG² deprotection of intermediate 1-ii yields the diacid 3-i. Treatment of 3-i with amino acid coupling reagents, followed by R⁵R⁴NH yields intermediate 3-ii, which upon deprotection of PG¹ yields intermediate 3-iii. Intermediate 3-iii is converted to intermediate 3-v by an amino acid coupling/deprotection sequence. A second amino acid coupling/deprotection sequence converts intermediate 3-v to compound 2-iii.

Thus, the invention provides a method of preparing a compound of Formula 1 comprising the steps of Method B. Furthermore, each of the individual steps of Method B, the intermediates involved, and methods of preparing the intermediates, are considered to be additional aspects of the invention. By way of illustration, the method can comprise (a) PG² deprotection of intermediate 1-ii to provide diacid 3-i. Alternatively, or in addition, the method can comprise (b) combining 3-i with an amino acid coupling agent and R⁵R⁴NH to provide intermediate 3-ii. Alternatively, or in addition, the method can comprise (c) combining 3-ii with an amino acid coupling agent to provide 3-iv, as illustrated in Scheme 3. Alternatively, or in addition, the method can comprise (d) deprotecting PG³ of 3-iv to provide 3-v. Alternatively, or in addition, the method can comprise (e) combining 3-v with an amino acid coupling agent to provide 2-ii as illustrated in scheme 3. Alternatively, or in addition, the method can comprise (f) deprotecting 2-ii at PG⁴ to provide 2-iii.

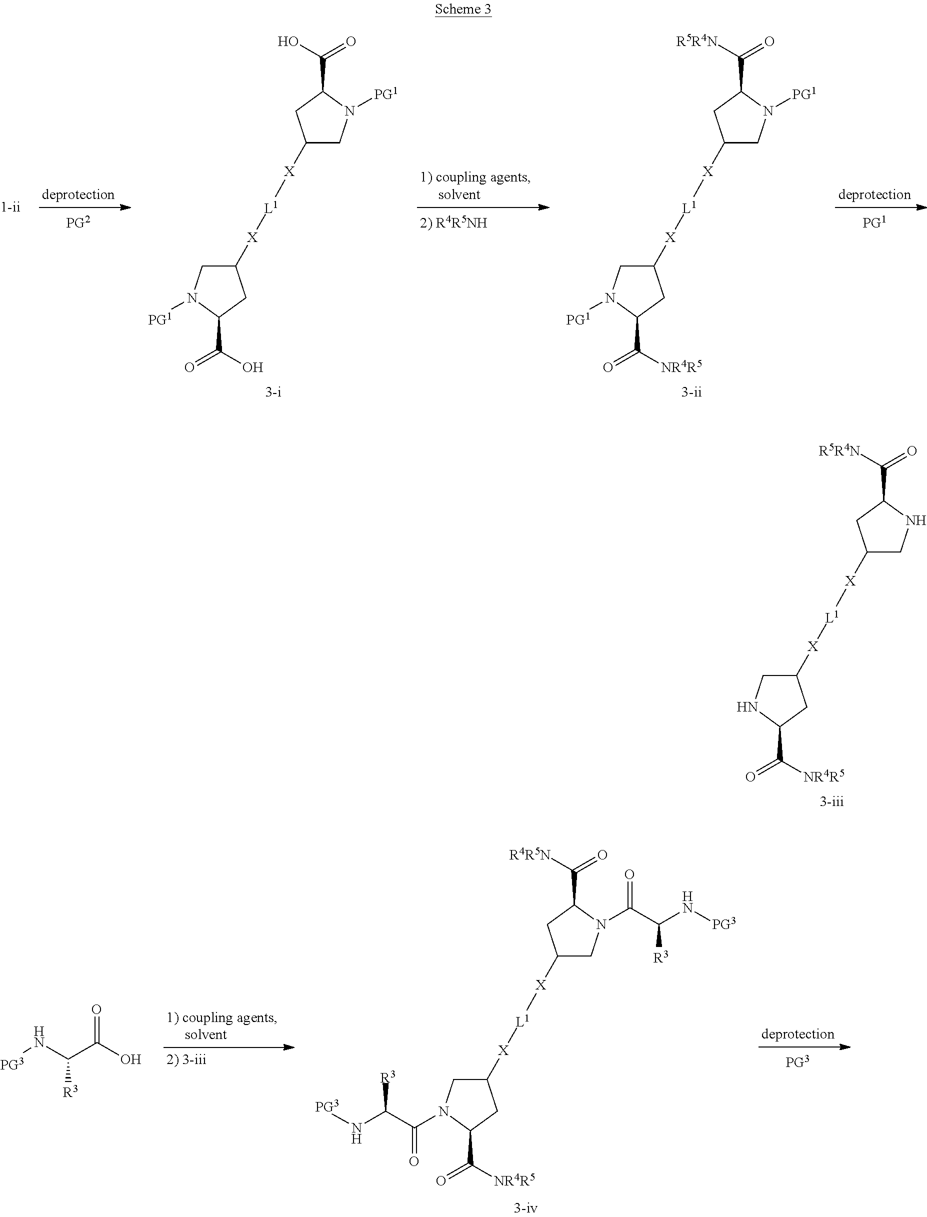

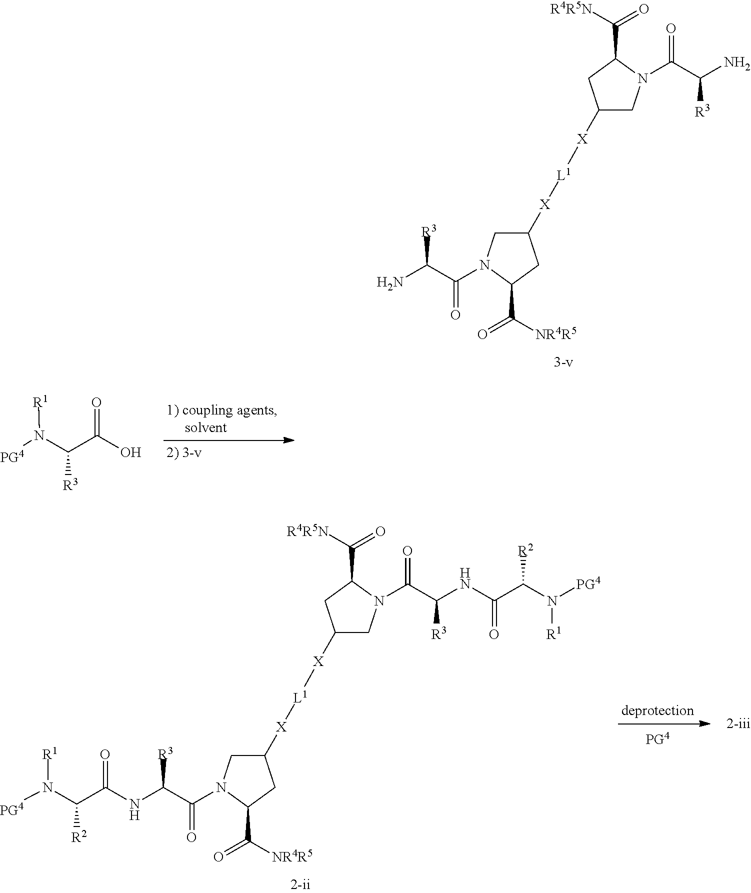

### Method C

Treatment of protected amino-proline derivative 4-i with amino acid coupling reagents, followed by R⁵R⁴NH yields intermediate 4-ii, which upon deprotection of PG¹ yields intermediate 4-iii. Intermediate 4-iii is converted to intermediate 4-v by an amino acid coupling/deprotection sequence. A second amino acid coupling step converts intermediate 4-v to intermediate 4-iv. Deprotection at PG⁵ yields intermediate 4-vii. Intermediate 4-vii is treated with LG-L¹-LG to provide intermediate 3-vi which upon deprotection at PG⁴ provided compound 2-iii.

Thus, the invention provides a method of preparing a compound of Formula 1 comprising the steps of Method C. Furthermore, each of the individual steps of Method C, the intermediates involved, and methods of preparing the intermediates, are considered to be additional aspects of the invention. By way of illustration, the method can comprise (a) combining 4-I with an amino acid coupling agent and R⁵R⁴NH to provide intermediate 4-ii. Alternatively, or in addition, the method can comprise (b) deprotecting PG¹ of 4-ii to provide 4-iii. Alternatively, or in addition, the method can comprise (c) combining 4-iii with an amino acid coupling agent to provide 4-iv, as illustrated in Scheme 4. Alternatively, or in addition, the method can comprise (d) deprotecting PG³ of 4-iv to provide 4-v. Alternatively, or in addition, the method can comprise (e) combining 4-v with an amino acid coupling agent to provide 4-iv, as illustrated in scheme 4. Alternatively, or in addition, the method can comprise (f) deprotecting 4-iv at PG⁵ to provide 4-vii. Alternatively, or in addition, the method can comprise combining 4-vii with LG-L¹-LG to provide intermediate 3-vi. Alternatively, or in addition, the method can comprise deprotecting 3-vi at PG⁴ to provide compound 2-iii.

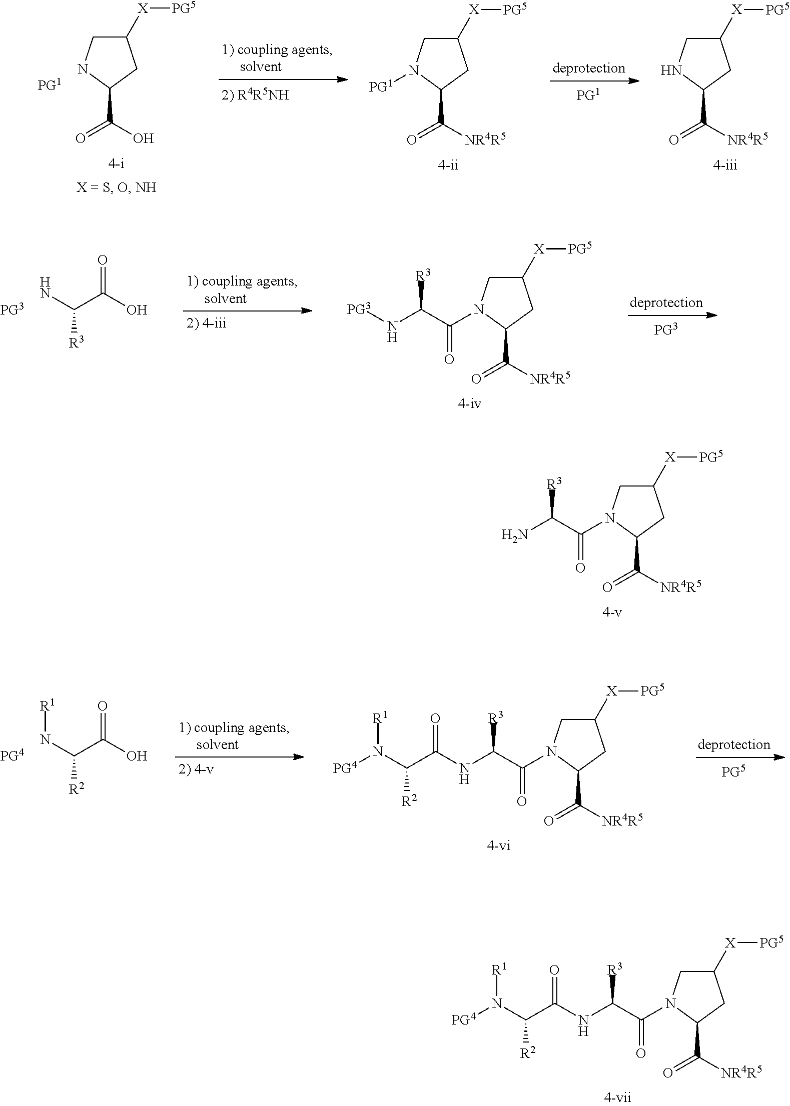

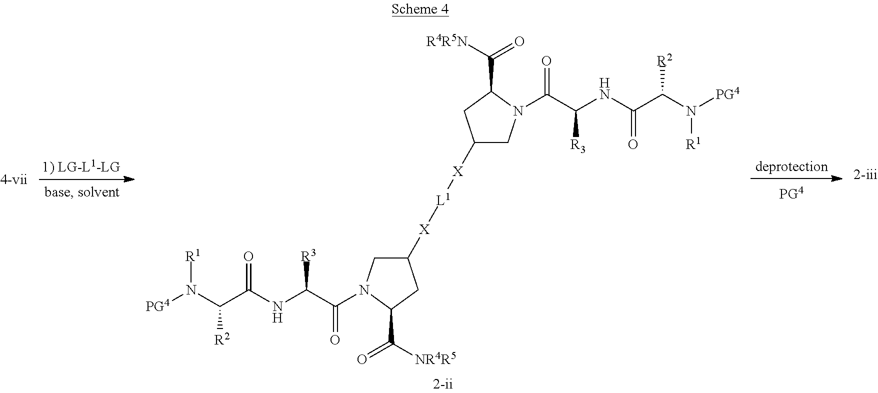

### EXAMPLES

The following abbreviations are used throughout:

Boc: t-butoxycarbonyl;
CBz: benzyloxycarbonyl;
DCM: dichloromethane, CH₂Cl₂;
DIPEA: diisopropylethylamine;
DMAP: 4-(dimethylamino)pyridine;

### DMF: N,N-dimethylformamide;

DTT: dithiothreitol;
EDC: 3-dimethylaminopropyl-3-ethylcarbodiimide hydrochloride;
EDTA: ethylenediaminetetracetic acid;
Fmoc: N-(9-fluorenylmethoxycarbonyl);
HBTU: O-(benzotriazol-1-yl)-N,N,N′,N′-tetramethyluronium hexafluorophosphate;
HCl: hydrochloric acid;
HOAc: acetic acid;
HOBt: 1-hydroxybenzotriazole;
HPLC: high performance liquid chromatography;
LCMS: liquid chromatography-mass spectrometer;
MeOH: methanol;
MgSO₄: magnesium sulfate;
MS: mass spectrum;
NaHCO₃: sodium hydrogen carbonate;
Pd/C: palladium on carbon;
TEA: triethylamine;
THF: tetrahydrofuran; and

### TMEDA: N,N,N,N-tetramethylethylenediamine.

LG: Leaving group
PG: Protective group

### Synthetic Methods

Synthesis of compound 1

The synthesis of compound 1 is illustrated in Scheme 6. N-Boc-cis-4-amino-L-proline methyl ester 6-1 was treated with terephthaloyl chloride to provide intermediate 6-2 which was further saponified using 2N LiOH to yield intermediate 6-3. Intermediate 6-3 was coupled to (R)-(−)-1,2,3,4-Tetrahydro-1-naphthylamine using HBTU and HOBt to provide intermediate 6-4, TFA deprotection yielded intermediate 6-5•2TFA. Intermediate 6-5•2TFA was coupled to Boc-cyclohexyl-Gly-OH 7-1 using HBTU and HOBt to provide intermediate 7-2, HCl deprotection yielded intermediate 7-3•2HCl. Intermediate 7-3•2HCl was coupled to Boc-N-MeAla-OH 7-4 using HBTU and HOBt to provide intermediate 7-5, HCl deprotection yielded compound 1.

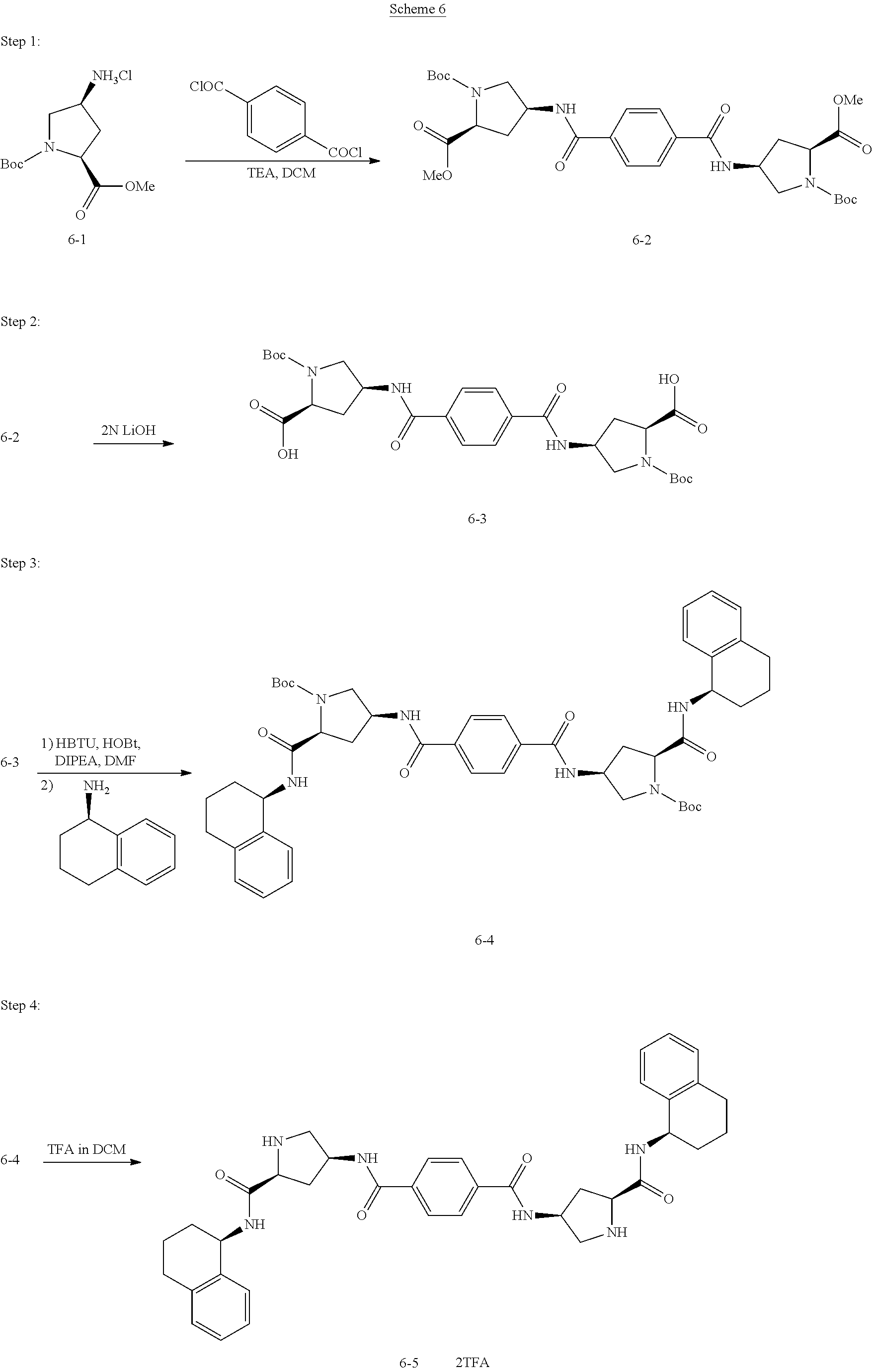

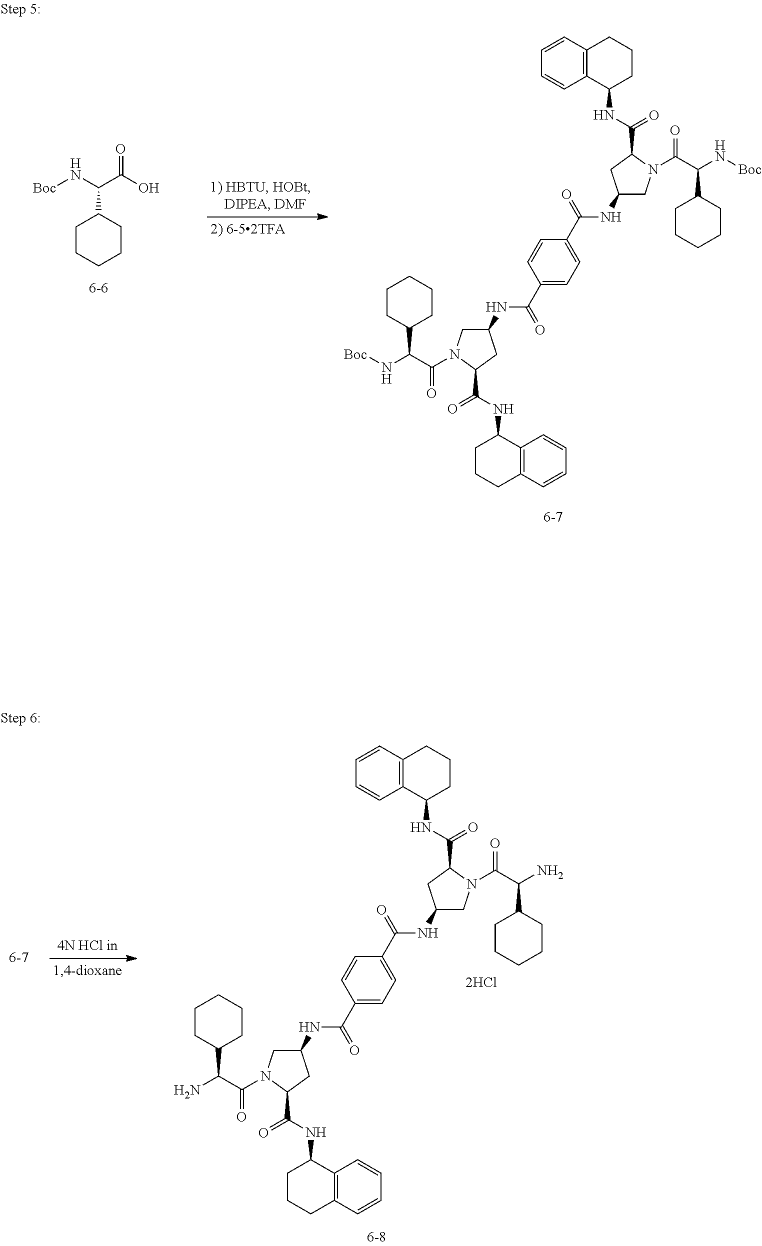

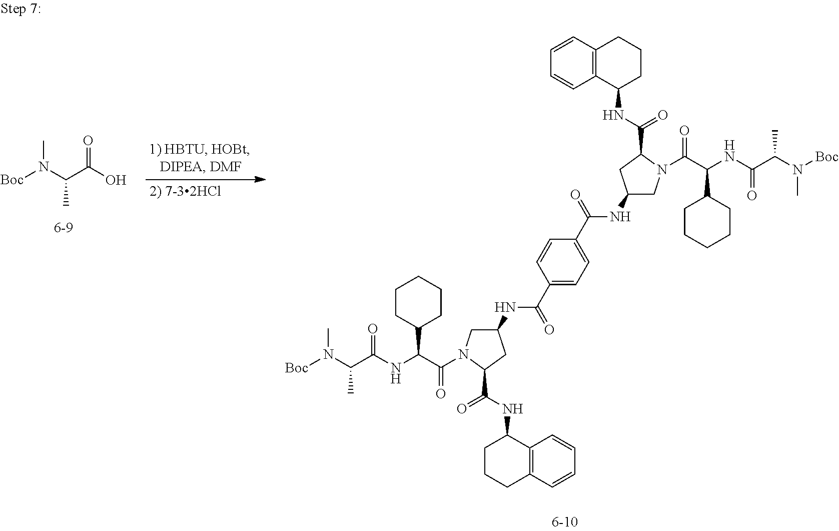

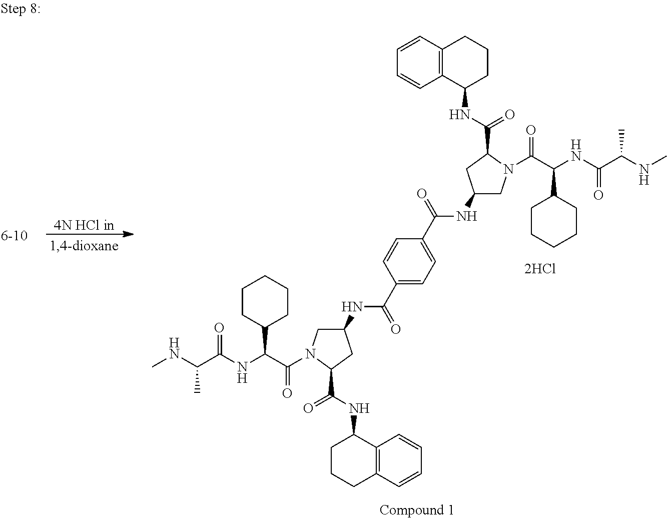

### Step 1: Intermediate 6-2

To a solution of N-Boc-cis-4-amino-L-proline methyl ester hydrochloride, 6-1, (25.0 g, 89.2 mmol), in CH₂Cl₂ cooled to 0° C., were sequentially added triethylamine (50.0 mL, 356.8 mmol), DMAP (545 mg, 4.46 mmol) and terephthaloyl chloride (8.69 g, 42.8 mmol). The reaction was stirred overnight at room temperature. Water and ethyl acetate were added, the organic layer was separated, washed with 10% citric acid, aqueous NaHCO₃ and brine, dried over anhydrous MgSO₄, filtered and concentrated in vacuo. Purification by silica gel chromatography provided intermediate 6-2 as a pale yellow solid.

### Step 2: Intermediate 6-3

To a solution of intermediate 6-2 (26.5 g, 42.9 mmol) in THF (600 mL) and MeOH (60 mL) cooled to 0° C. was added 2N aqueous LiOH (107 mL, 215 mmol) and the reaction was stirred overnight at room temperature. The pH was adjusted to 3 with 10% citric acid and ethyl acetate was added. The organic layer was separated and the aqueous phase was extracted two times with ethyl acetate. The combined organic layers were washed with brine, dried over anhydrous MgSO₄, filtered and concentrated in vacuo to provide intermediate 6-3 as a white solid.

### Step 3: Intermediate 6-4

To a solution of intermediate 6-3 (22.6 g, 38.3 mmol) in DMF cooled to 0° C. were sequentially added DIPEA (67.0 mL, 383 mmol), HOBt (12.9 g, 95.7 mmol) and HBTU (36.3 g, 95.7 mmol). After stirring for 10 minutes 1,2,3,4-(R)-tetrahydro-1-naphthylamine (12.4 g, 84.2 mmol) was added and the reaction mixture was stirred overnight at room temperature. Water and ethyl acetate were added, the organic layer was separated, washed with 10% citric acid, aqueous NaHCO₃ and brine, dried over anhydrous MgSO₄, filtered and concentrated in vacuo to provide intermediate 6-4 as a yellow solid.

### Step 4: Intermediate 6-5•2TFA

Intermediate 6-4 (38.3 mmol) was dissolved in a mixture of CH₂Cl₂ (200 mL) and TFA (200 mL) at 0° C. The solution was stirred for 7 hours at room temperature. Volatiles were removed under reduced pressure and the residue was triturated with diethyl ether to provide intermediate 6-5•2TFA as a white solid. MS (m/z) M+H=499.4.

### Step 5: Intermediate 6-7

To a solution of Boc-Chg-OH, 6-6, (2.00 g, 7.80 mmol) in DMF cooled to 0° C. were sequentially added, DIPEA (5.24 ml, 30.0 mmol), HOBt (1.21 g, 9.00 mmol) and HBTU (3.41 g, 9.00 mmol). After stirring for 10 minutes intermediate 6-5•2TFA (2.63 g, 3.00 mmol) was added and the reaction mixture was stirred overnight at room temperature. Water and ethyl acetate were added, the organic layer was separated, washed with 10% citric acid, saturated NaHCO₃, and brine, dried over anhydrous MgSO₄, filtered and concentrated in vacuo. Purification by silica gel chromatography provided intermediate 6-7 as a white solid.

### Step 6: Intermediate 6-8•2HCl

4N HCl in 1,4-dioxane (5.0 ml) was added to intermediate 6-7 (1.70 g, 1.51 mmol) and the solution was stirred at 0° C. for 2 hours. Volatiles were removed under reduced pressure and the residue was triturated with diethyl ether to provide intermediate 6-8•2HCl as a white solid.

MS (m/z) M+1=927.4.

### Step 7: Intermediate 6-10

To a solution of Boc-NMe-Ala-OH, 6-9, (0.68 g, 3.38 mmol) in DMF cooled to 0° C. were sequentially added, DIPEA (2.26 ml, 13.00 mmol), HOBt (0.52 g, 3.90 mmol) and HBTU (1.47 g, 3.90 mmol). After stirring for 10 minutes intermediate 6-8•2HCl (1.3 g, 1.30 mmol) was added and the reaction mixture was stirred overnight at room temperature. Water and ethyl acetate were added; the organic layer was separated, washed with 10% citric acid, saturated NaHCO₃, and brine, dried over anhydrous MgSO4, filtered and concentrated in vacuo. Purification by silica gel chromatography provided intermediate 6-10 as a white solid.

### Step 8: Compound 1

4N HCl in 1,4-dioxane (2.5 ml) was added to intermediate 7-5 (0.80 g, 0.62 mmol) and the solution was stirred at 0° C. for 2 hours. Volatiles were removed under reduced pressure and the residue was triturated with diethyl ether to provide compound 1•2HCl as a white solid. MS (m/z) M+H=1097.6.

### Synthesis of Compound 3

The synthesis of compound 3 is illustrated in Scheme 7. N-Boc-cis-4-amino-L-proline methyl ester 7-1 was treated with trans-1,4-cyclohexyldicarbonyl dichloride to provide intermediate 7-2. Deprotection using TFA/CH₂Cl₂ yielded intermediate 7-3•2TFA. Intermediate 7-3•2TFA was coupled to Boc-cyclohexyl-Gly-OH 7-4 using HBTU and HOBt to provide intermediate 7-5. Deptrotection using TFA/CH₂Cl₂ yielded intermediate 7-6•2TFA. Intermediate 7-6•2TFA was coupled to Boc-N-MeAla-OH, 7-7, using HBTU and HOBt to provide intermediate 7-8. Saponification of 7-8 using 2N LiOH provided intermediate 7-9. Intermediate 7-9 was coupled to (R)-(−)-1-aminoindan using HBTU and HOBt to provide intermediate 7-10. Boc-deprotection using 2M HCl in 1,4-dioxane provided compound 3•2HCl.

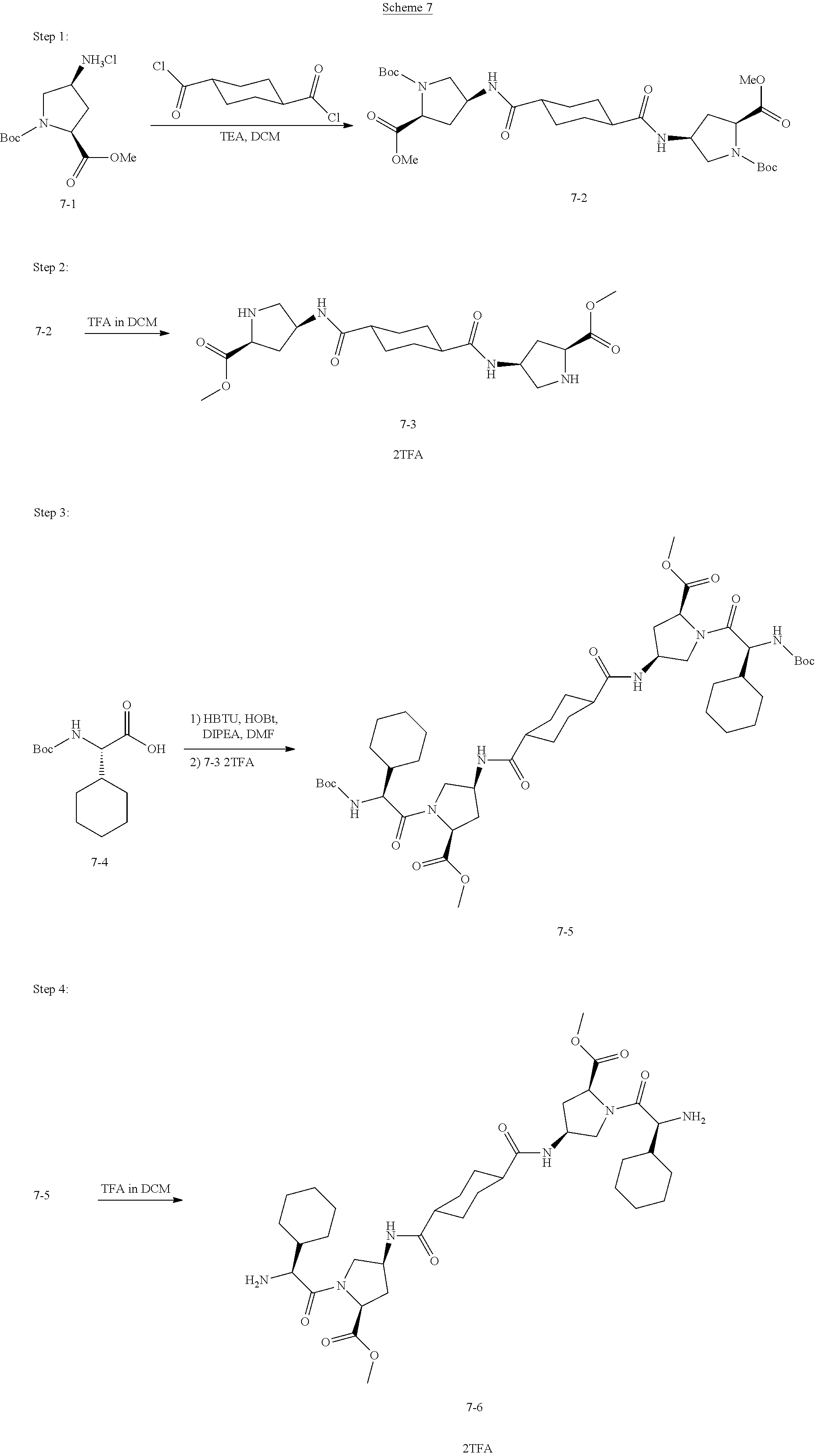

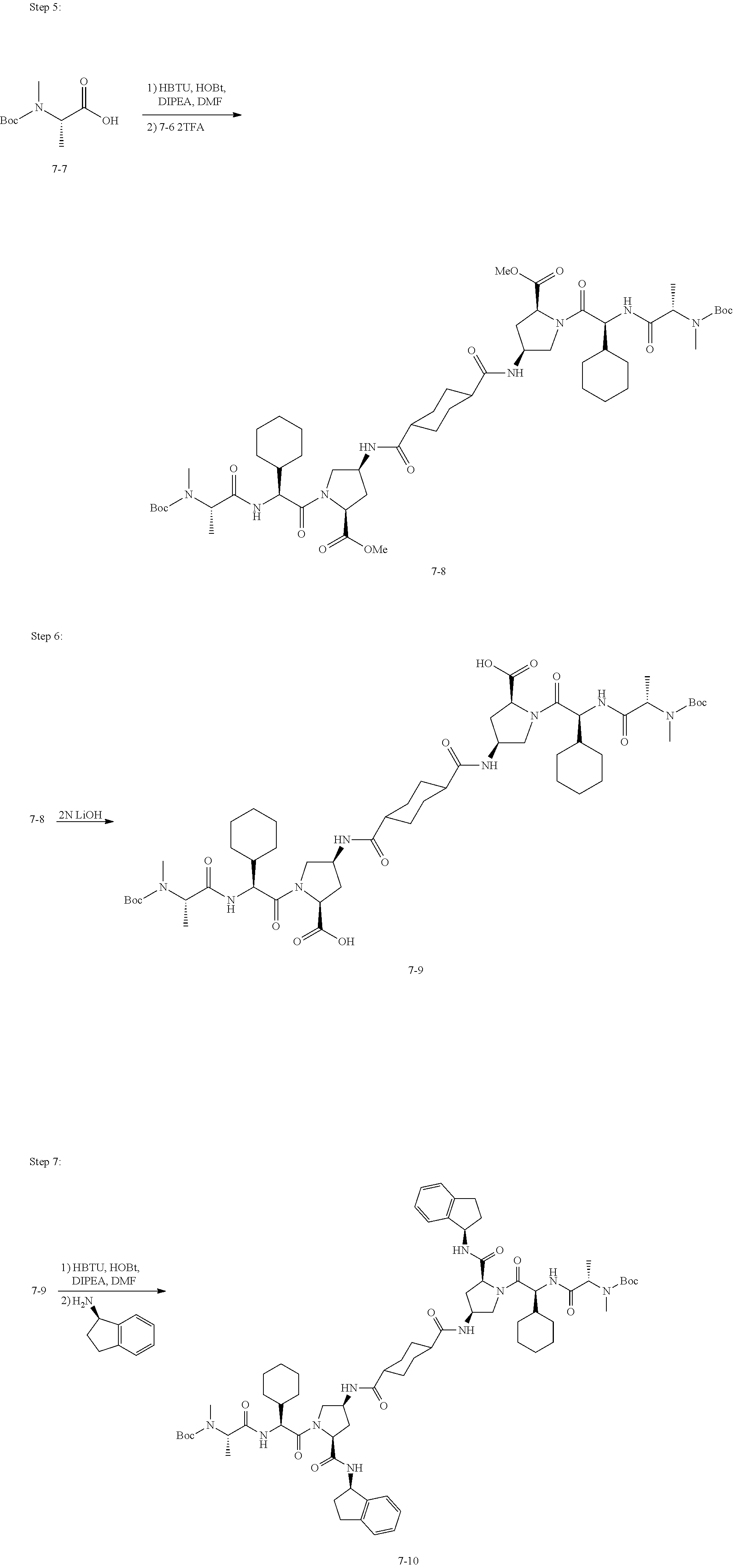

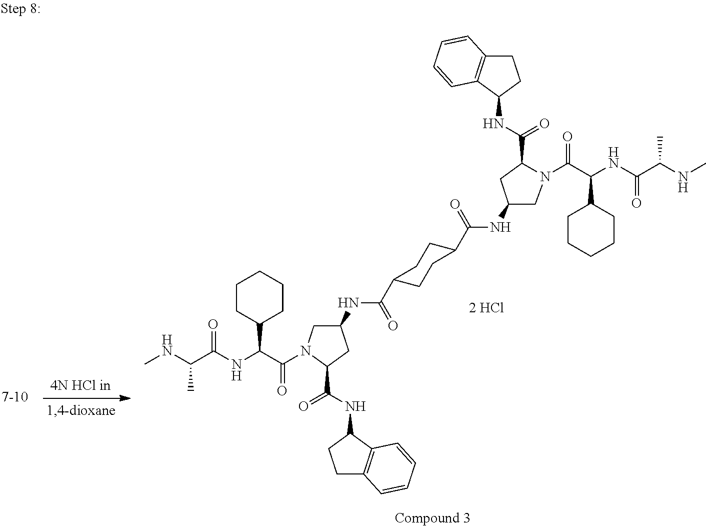

### Step 1: Intermediate 7-2

To a suspension of trans-1,4-cyclohexyldicarboxylic acid (3.37 g, 19.59 mmol) in CH₂Cl₂ cooled to 0° C. was added oxalyl chloride (29.4 mL, 58.80 mmol) and DMF (0.30 mL, 3.92 mmol), the mixture was stirred at 0° C. for 5 minutes and at room temperature for 2 hours. Volatiles were removed under reduced pressure to provide crude trans-1,4-cyclohexyldicarbonyl dichloride, which was added to a solution of N-Boc-cis-4-amino-L-proline methyl ester hydrochloride, 7-1, (11.0 g, 39.2 mmol), and triethylamine (16.38 mL, 118.0 mmol) in CH₂Cl₂ cooled to 0° C. The resulting mixture was stirred overnight at room temperature. Aqueous NaHCO₃ was added, the organic layer was separated and the aqueous phase was extracted with CH₂Cl₂. The combined organic extracts were washed with brine, dried over anhydrous MgSO₄, filtered and concentrated in vacuo. Purification by silica gel chromatography provided intermediate 7-2 as a pale yellow solid.

### Step 2: Intermediate 7-3•2TFA

Intermediate 7-2 (10.0 g, 16.01 mmol) was dissolved in a mixture of CH₂Cl₂ (64 mL) and TFA (50.6 mL) at 0° C. The solution was stirred for 15 minutes at 0° C. hours and for 2.5 hours at room temperature. Volatiles were removed under reduced pressure and the residue was triturated with diethyl ether to provide intermediate 7-3•2TFA as a white solid.

### Step 3: Intermediate 7-5

To a solution of Boc-Chg-OH, 7-4, (9.06 g, 35.2 mmol) in DMF cooled to 0° C. were sequentially added, DIPEA (28.0 ml, 160.0 mmol), HOBt (6.19 g, 45.8 mmol) and HBTU (17.36 g, 45.8 mmol). After stirring for 10 minutes intermediate 7-3•2TFA (10.0 g, 16.01 mmol) was added and the reaction mixture was stirred overnight at room temperature. Water and ethyl acetate were added, the organic layer was separated and washed with 10% citric acid, saturated NaHCO₃, and brine, dried over anhydrous MgSO₄, filtered and concentrated in vacuo. Purification by silica gel chromatography provided intermediate 7-5 as a white solid.

### Step 4: Intermediate 7-6•2TFA

Intermediate 7-5 (12.71 g, 14.07 mmol) was dissolved in a mixture of CH₂Cl₂ (56 mL) and TFA (44.5 mL) at 0° C. The solution was stirred for 2 hours at 0° C. Volatiles were removed under reduced pressure and the residue was triturated with diethyl ether to provide intermediate 7-6•2TFA as a white solid. MS (m/z) M+H=697.2

### Step 5: Intermediate 7-8

To a solution of Boc-NMe-Ala-OH, 7-7, (6.29 g, 31.0 mmol) in DMF cooled to 0° C. were sequentially added, DIPEA (24.57 mL, 141.0 mmol), HOBt (5.44 g, 40.2 mmol) and HBTU (15.26 g, 40.2 mmol). After stirring for 10 minutes at 0° C. intermediate 7-6•2TFA (13.10 g, 14.07 mmol) was added and the reaction mixture was stirred overnight at room temperature. Water and ethyl acetate were added; the organic layer was separated and washed with 10% citric acid, saturated NaHCO₃, and brine, dried over anhydrous MgSO4, filtered and concentrated in vacuo. Purification by silica gel chromatography provided intermediate 7-8 as a white solid.

### Step 6: Intermediate 7-9

To a solution of intermediate 7-8 (3.29 g, 3.07 mmol) in THF (15 mL) cooled to 0° C. was added 2N aqueous LiOH (15.33 mL, 30.7 mmol) and the reaction was stirred overnight at room temperature. The pH was adjusted to 3 with 10% citric acid and ethyl acetate was added. The organic layer was separated and the aqueous phase was extracted two times with ethyl acetate. The combined organic layers were washed with brine, dried over anhydrous MgSO₄, filtered and concentrated in vacuo to provide intermediate 7-9 as a white solid.

### Step 7: Intermediate 7-10

To a solution of intermediate 7-9 (400 mg, 0.38 mmol) in DMF cooled to 0° C. were sequentially added DIPEA (668 uL, 3.83 mmol), HOBt (155 mg, 1.14 mmol) and HBTU (435 mg, 1.14 mmol). After stirring for 10 minutes (R)-(−)-1-aminoindan (128 uL, 0.99 mmol) was added and the reaction mixture was stirred overnight at room temperature. Water and ethyl acetate were added, the organic layer was separated and washed with 10% citric acid, aqueous NaHCO₃ and brine, dried over anhydrous MgSO₄, filtered and concentrated in vacuo to provide intermediate 7-10 as a white solid.

### Step 8: Compound 3•2HCl

4N HCl in 1,4-dioxane (3.21 ml) was added to intermediate 7-10 (231 mg, 0.18 mmol) and the solution was stirred at 0° C. for 1.5 hour. Volatiles were removed under reduced pressure and the residue was triturated with diethyl ether to provide compound 3•2HCl as a white solid. MS (m/z) M+H=1075.6

Representative compounds of the present invention prepared generally in accordance with the above procedures and are illustrated in Table 1:

TABLE 1

| COM- | | |
|---|---|---|
| POUND | STRUCTURE | MS (m/z) |
| 1 | 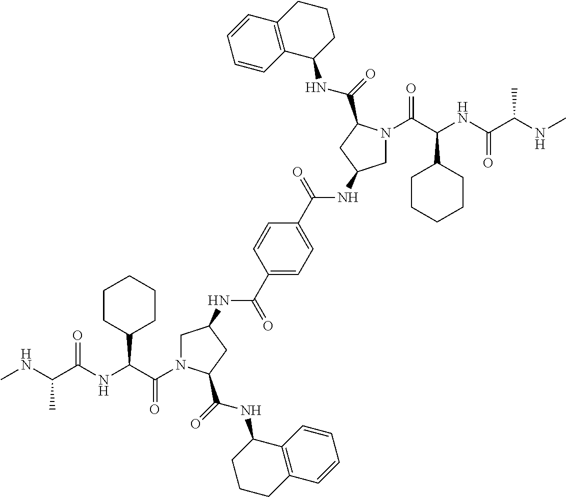 | [M + H]⁺ = 1097.6 [M + 2 H]²⁺ = 549.4 |
| 2 | 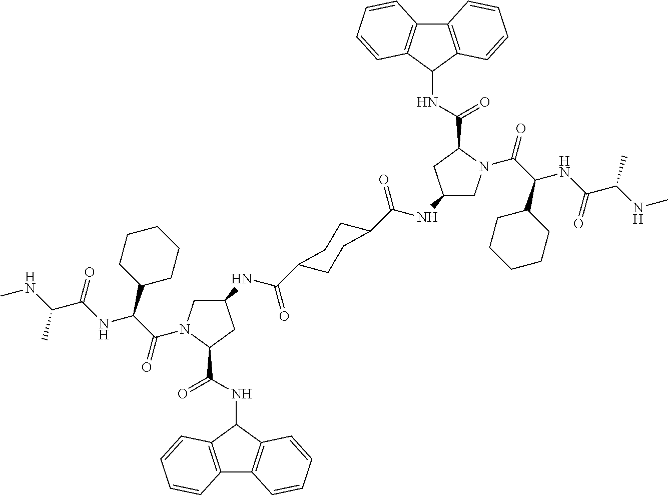 | [M + H]⁺ = 1171.2 [M + 2 H]²⁺ = 586.4 |
| 3 | 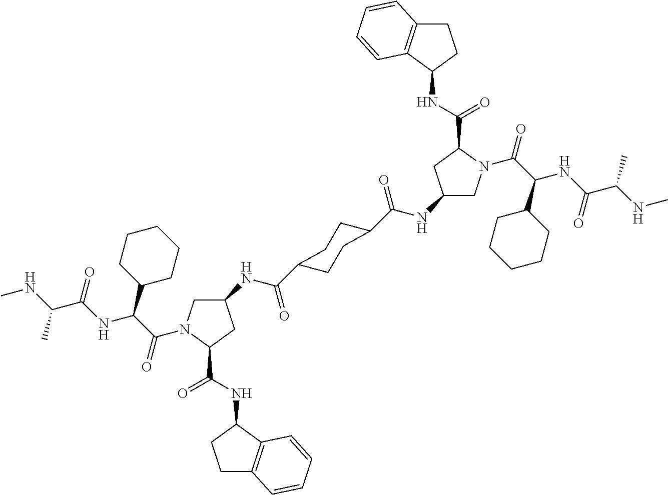 | [M + H]⁺ = 1075.6 [M + 2 H]²⁺ = 537.4 |
| 4 | 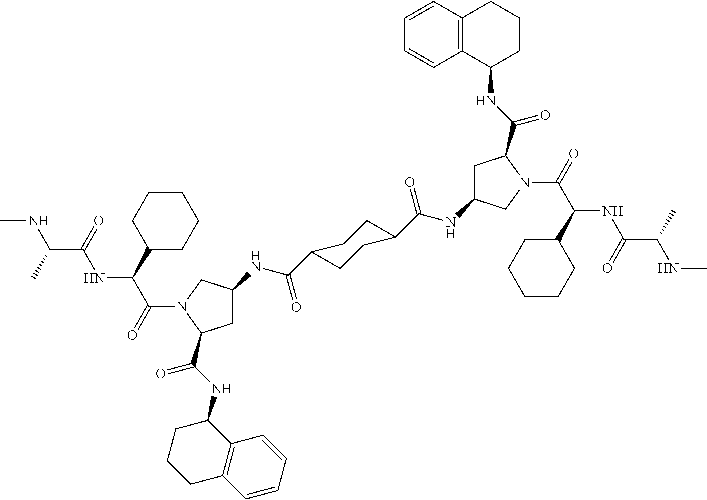 | [M + H]⁺ = 1103.6 [M + 2 H]²⁺ = 552.4 |
| 5 | 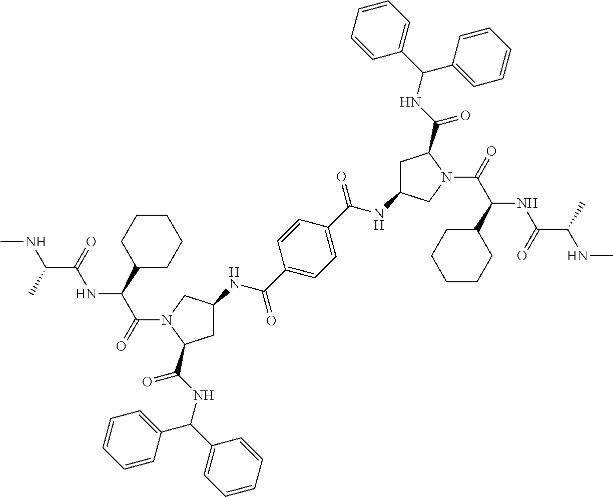 | [M + H]⁺ = 1169.6 [M + 2 H]²⁺ = 585.4 |
| 6 | 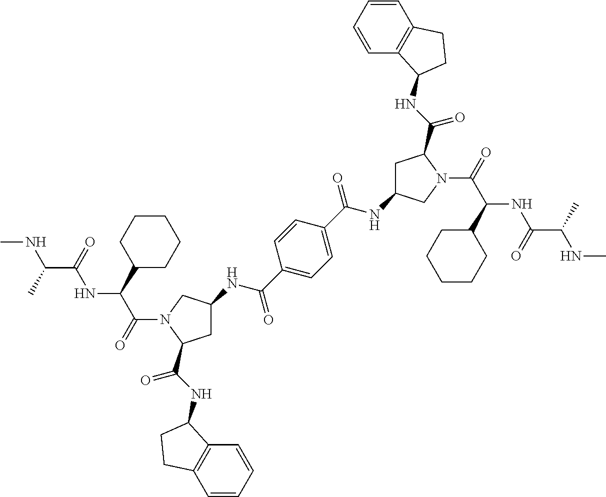 | [M + H]⁺ = 1069.4 [M + 2 H]²⁺ = 535.4 |
| 7 | 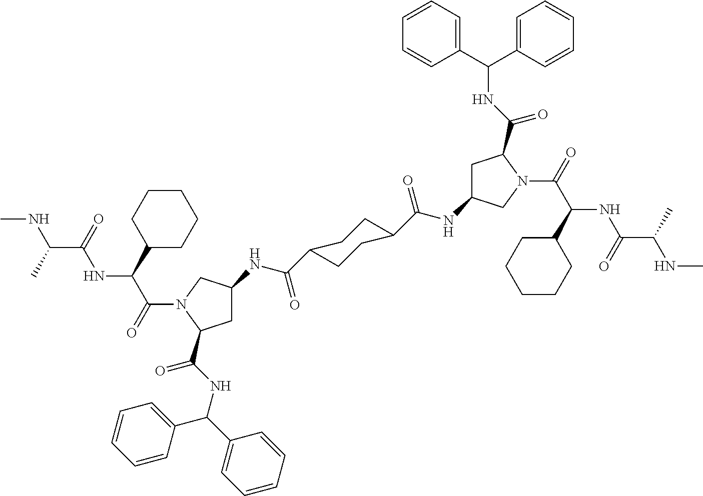 | [M + H]⁺ = 1175.6 [M + 2 H]²⁺ = 588.2 |
| 8 | 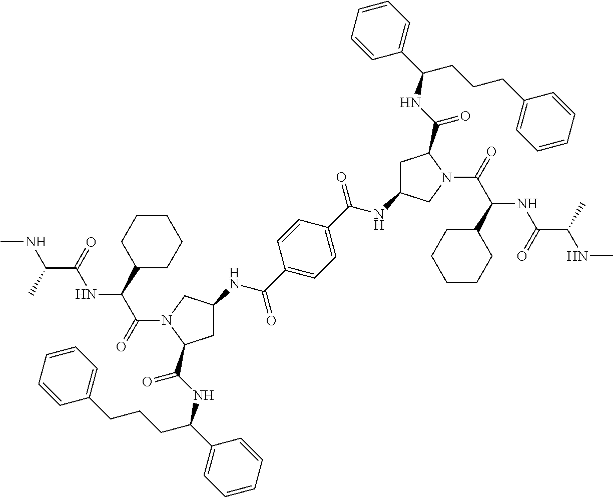 | [M + H]⁺ = 1253.6 [M + 2 H]²⁺ = 627.3 |
| 9 | 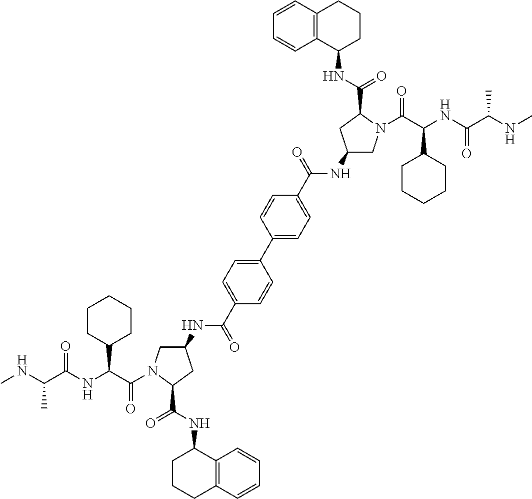 | [M + 2 H]²⁺ = 587.7 |
| 10 | 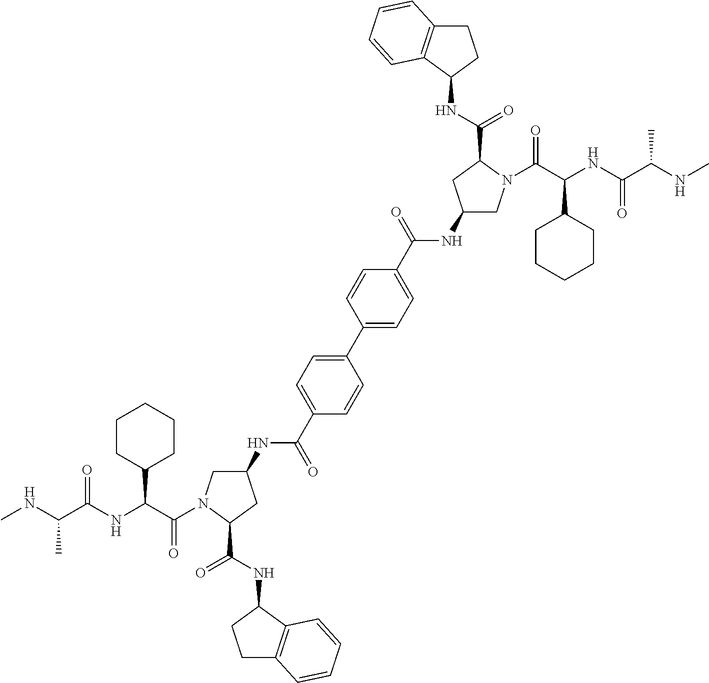 | [M + H]⁺ = 1146.6 [M + 2 H]²⁺ = 573.6 |
| 11 | 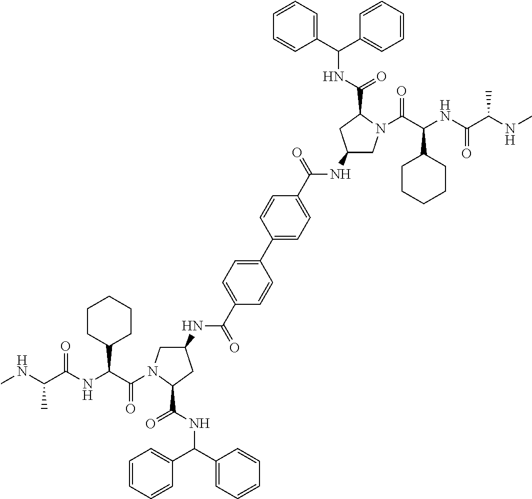 | [M + 2 H]²⁺ = 623.8 |
| 12 | 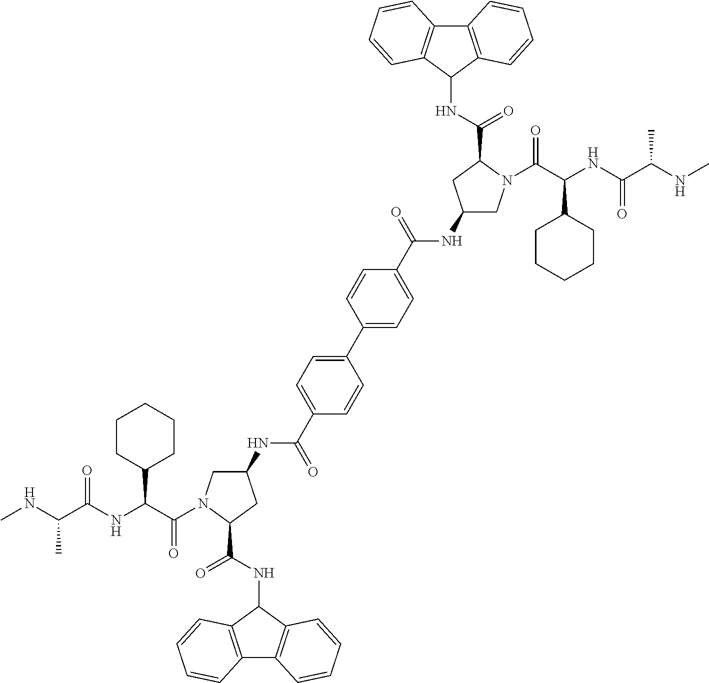 | [M + H]⁺ = 1242.3 [M + 2 H]²⁺ = 621.4 |
| 13 | 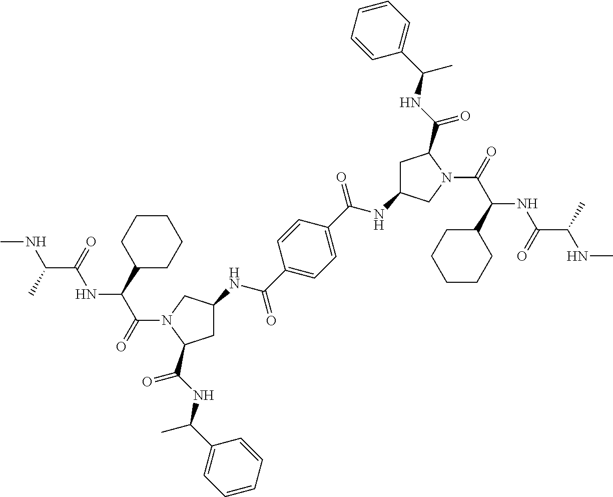 | [M + H]⁺ = 1095.6 [M + 2 H]²⁺ = 523.4 |
| 14 | 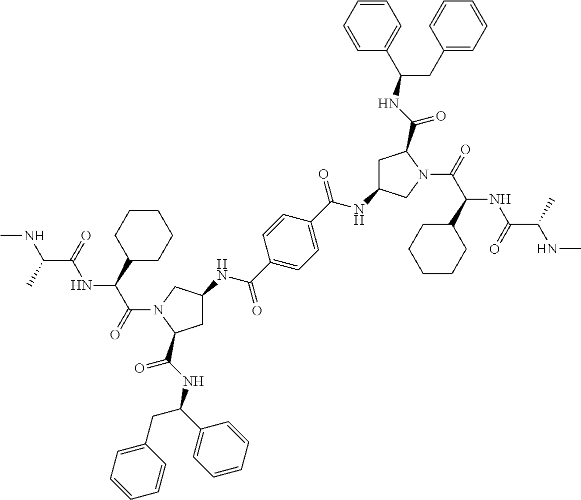 | [M + 2 H]²⁺ = 599.4 |
| 15 | 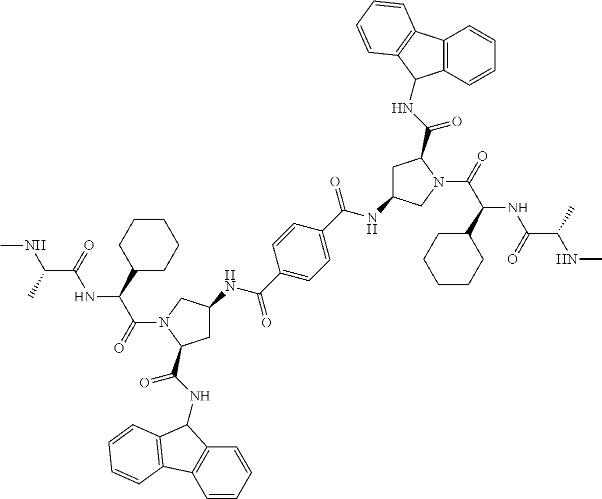 | [M + H]⁺ = 1166.4 [M + 2 H]²⁺ = 583.4 |
| 16 | 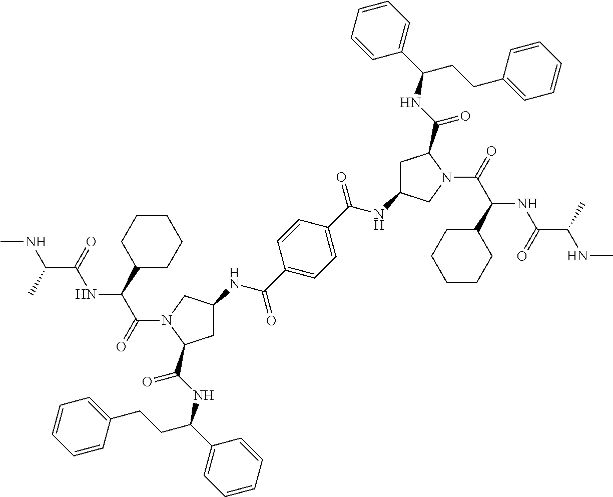 | [M + 2 H]²⁺ = 613.4 |
| 17 | 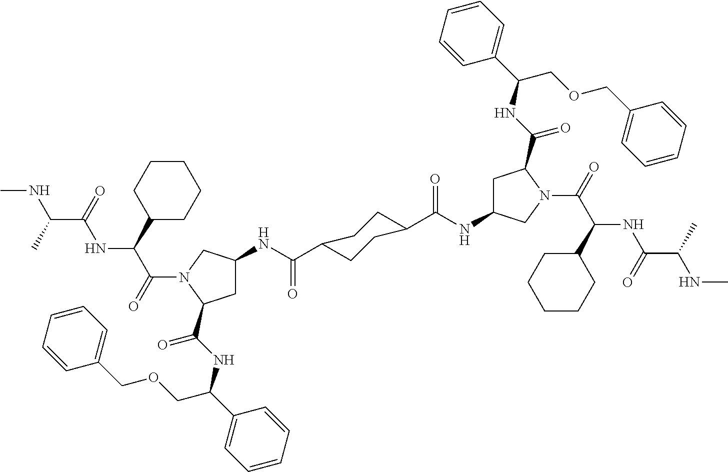 | [M + H]⁺ = 1263.8 [M + 2 H]²⁺ = 632.4 |
| 18 | 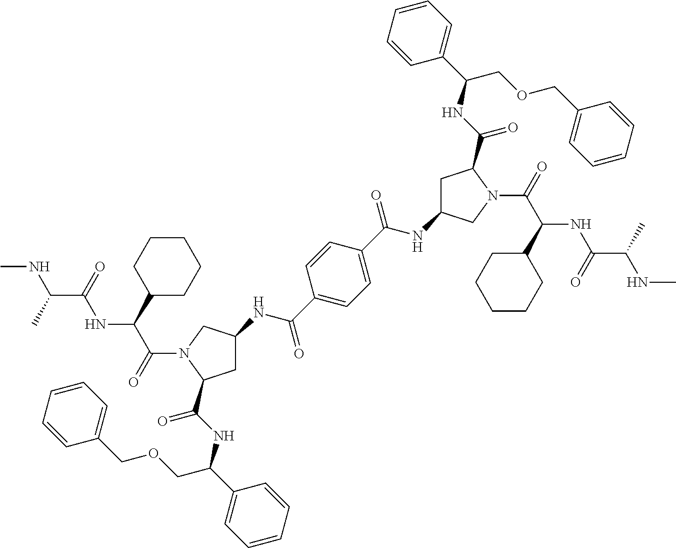 | [M + H]⁺ = 1257.8 [M + 2 H]²⁺ = 629.8 |
| 19 | 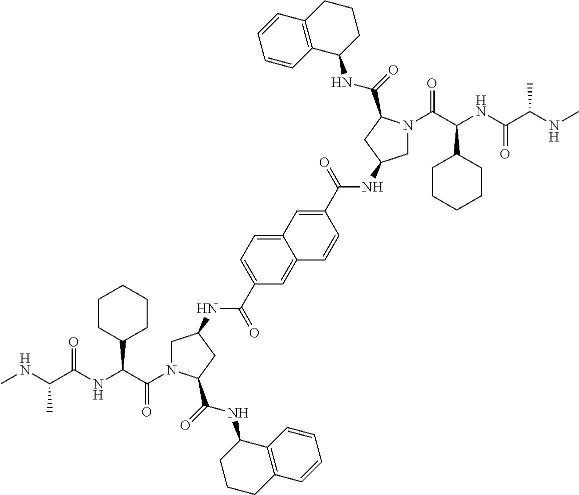 | [M + 2 H]²⁺ = 574.6 |
| 20 | 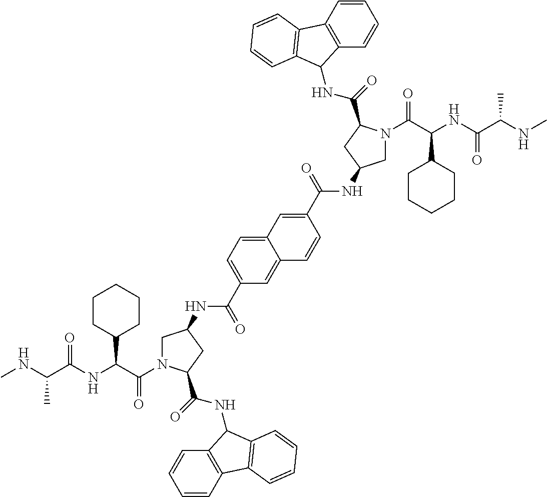 | [M + H]⁺ = 1215.7 [M + 2 H]²⁺ = 608.6 |
| 21 | 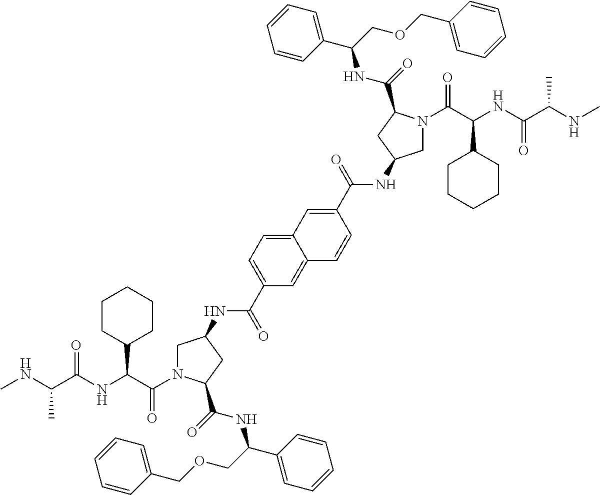 | [M + H]⁺ = 1308.8 [M + 2 H]²⁺ = 654.7 |
| 22 | 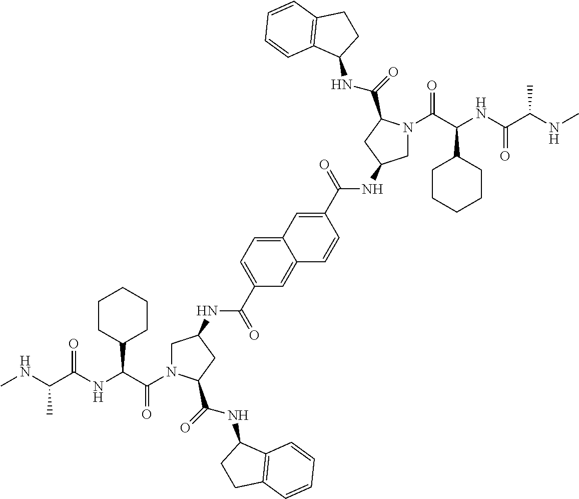 | [M + H]⁺ = [M + 2 H]²⁺ = |
| 23 | 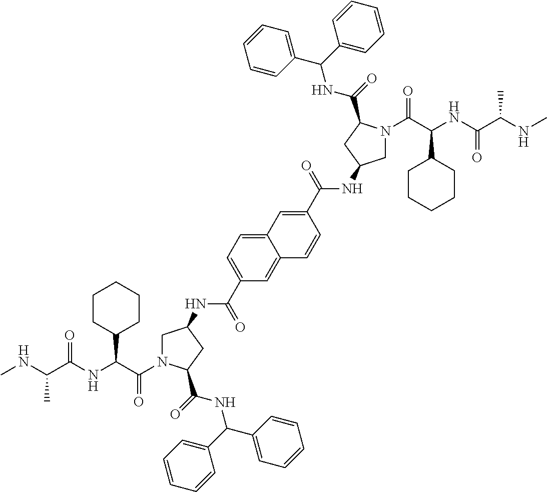 | [M + H]⁺ = 1219.7 [M + 2 H]²⁺ = 610.6 |
| 24 | 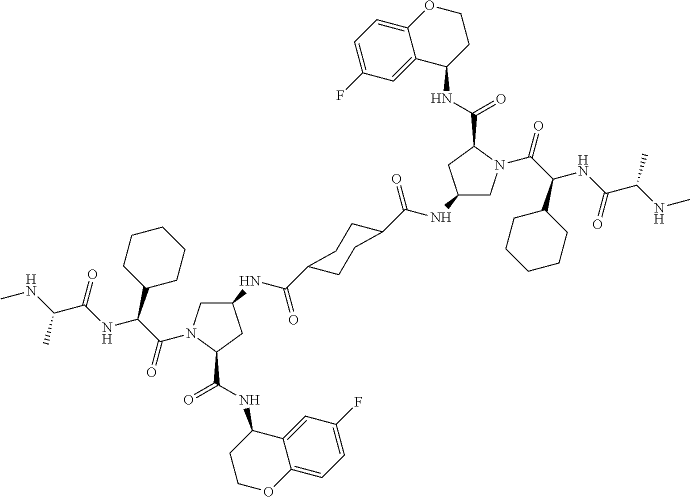 | [M + H]⁺ = 1144.6 [M + 2 H]²⁺ = 572.8 |
| 25 | 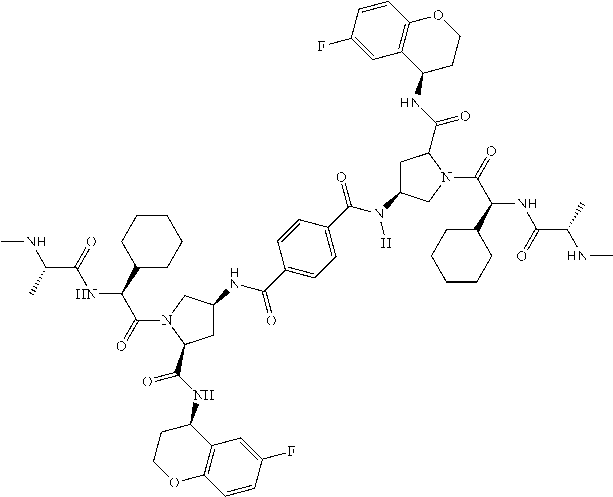 | [M + H]⁺ = 1138.5 [M + 2 H]²⁺ = 569.8 |
| 26 | 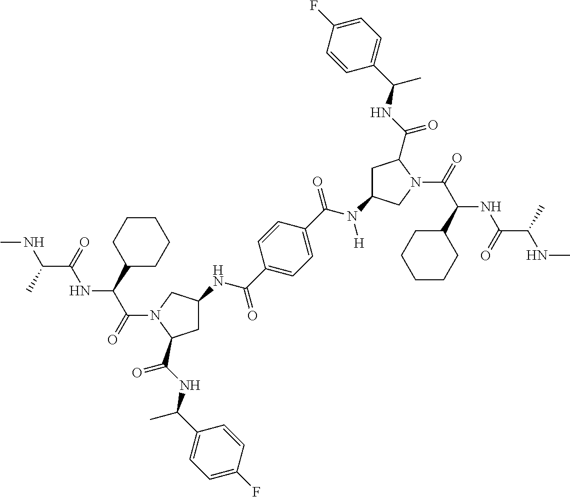 | [M + H]⁺ = 1081.7 [M + 2 H]²⁺ = 541.5 |
| 27 | 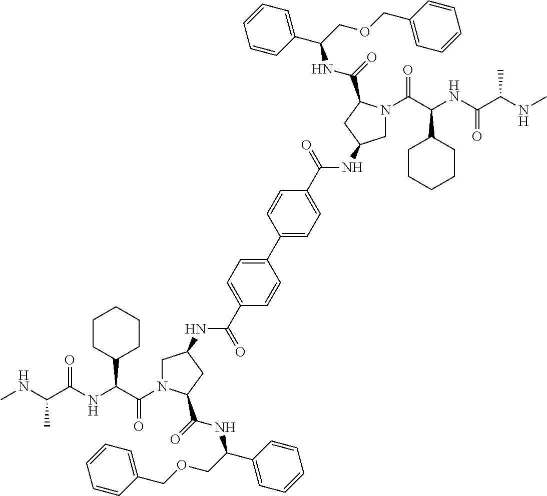 | [M + 2 H]²⁺ = 667.7 |
| 28 | 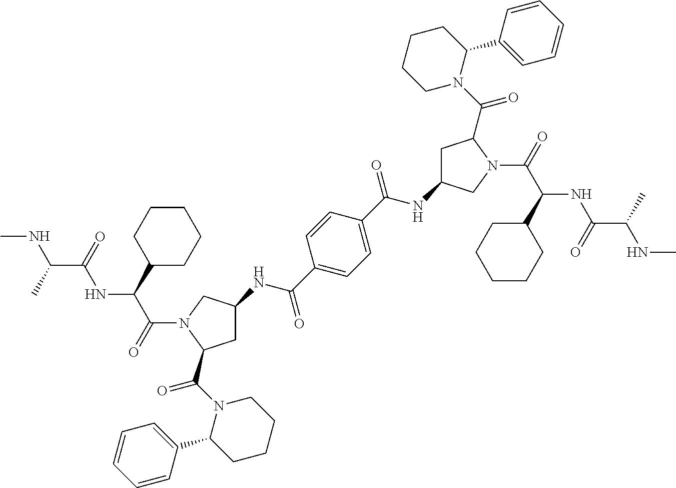 | [M + H]⁺ = 1125.9 [M + 2 H]²⁺ = 563.6 |
| 29 | 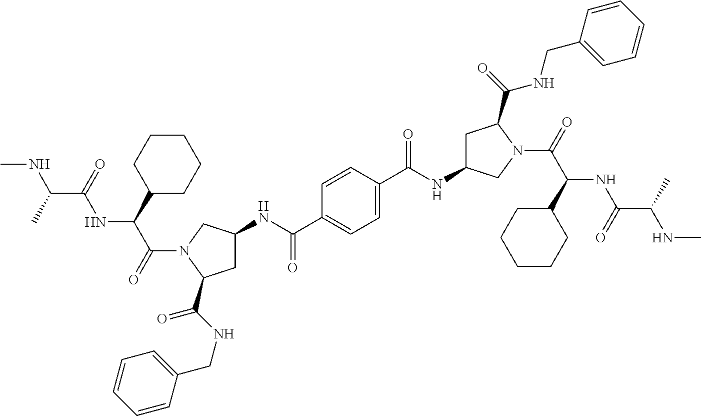 | [M + H]⁺ = 1017.6 [M + 2 H]²⁺ = 509.5 |
| 30 | 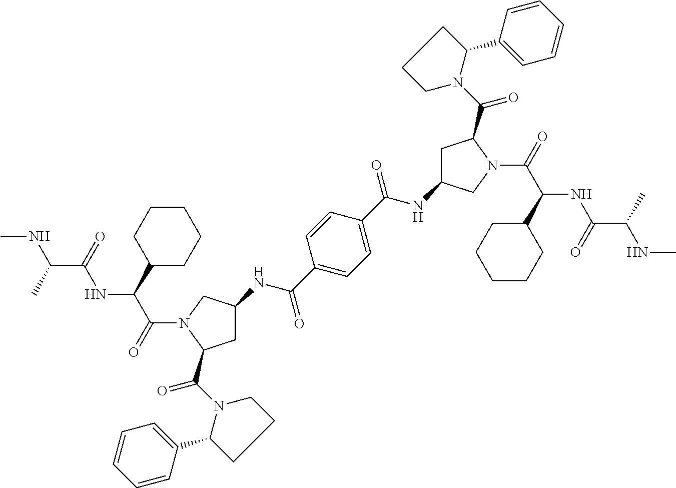 | [M + H]⁺ = 1097.8 [M + 2 H]²⁺ = 549.6 |
| 31 | 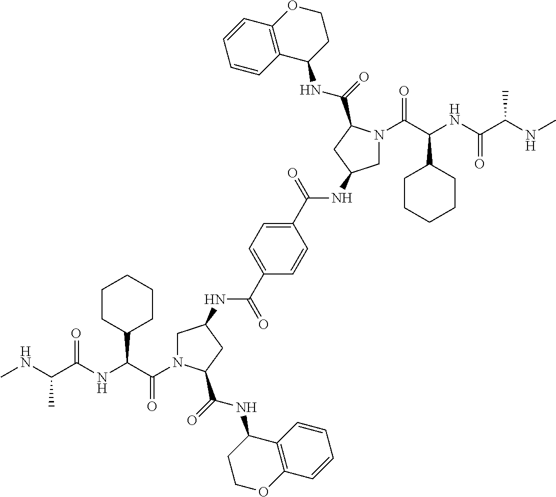 | [M + H]⁺ = 1101.7 [M + 2 H]²⁺ = 551.7 |
| 32 | 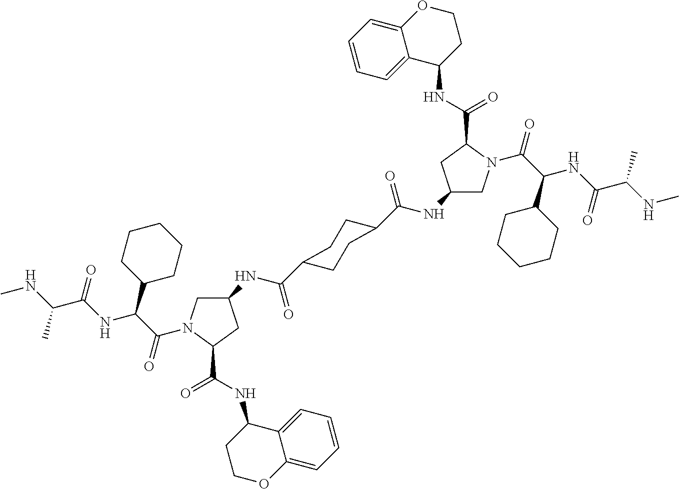 | [M + H]⁺ = 1107.8 [M + 2 H]²⁺ = 554.6 |
| 33 | 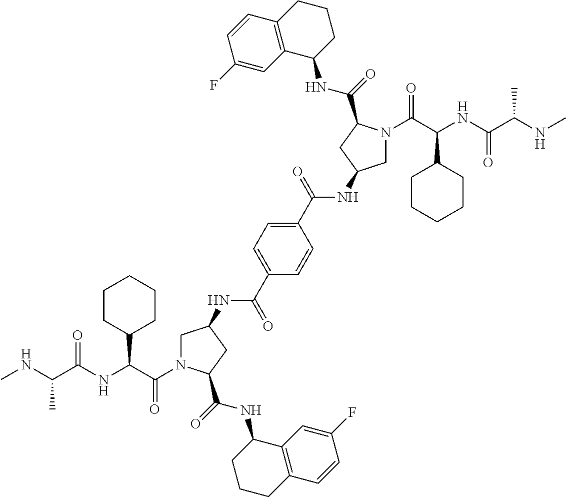 | [M + H]⁺ = 1133.8 [M + 2 H]²⁺ = 567.6 |
| 34 | 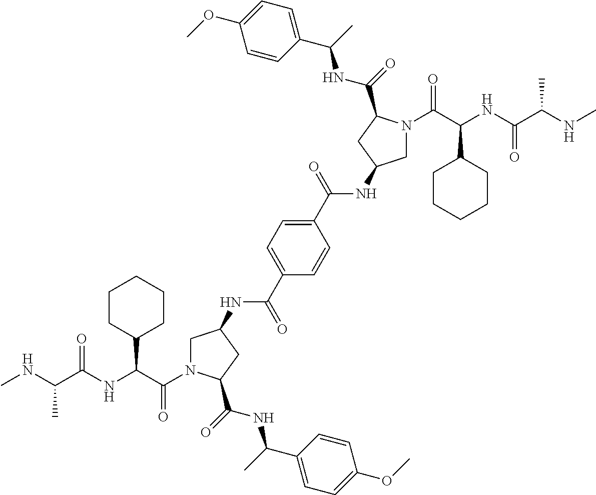 | [M + H]⁺ = 1105.8 [M + 2 H]²⁺ = 553.6 |
| 35 | 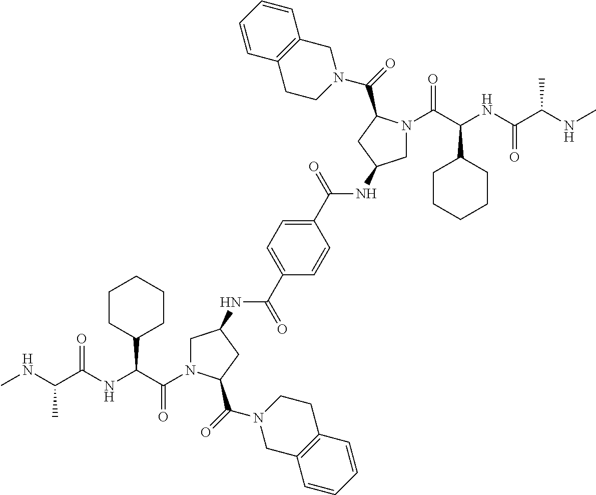 | [M + H]⁺ = 1069.8 [M + 2 H]²⁺ = 535.6 |
| 36 | 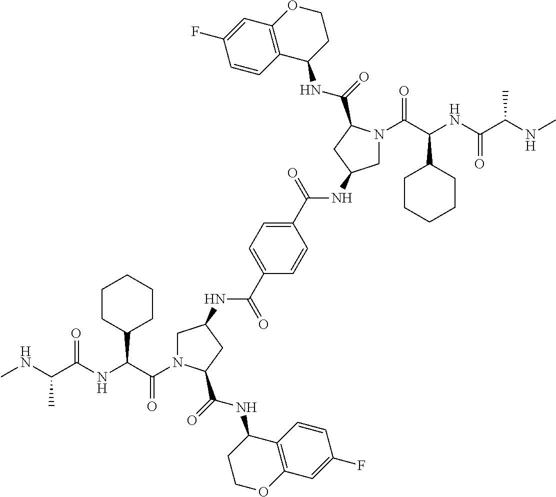 | [M + H]⁺ = 1137.6 [M + 2 H]²⁺ = 569.6 |
| 37 | 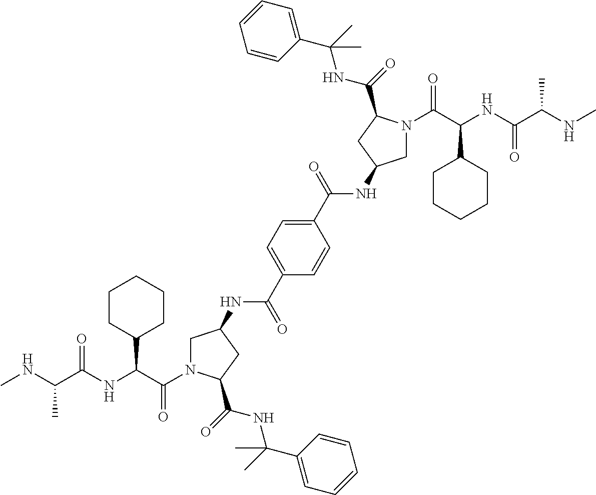 | [M + H]⁺ = 1073.7 [M + 2 H]²⁺ = 537.6 |
| 38 | 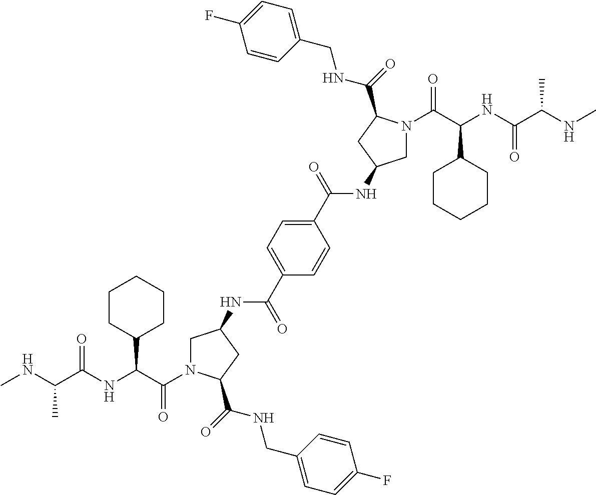 | [M + H]⁺ = 1053.7 [M + 2 H]²⁺ = 527.6 |
| 39 | 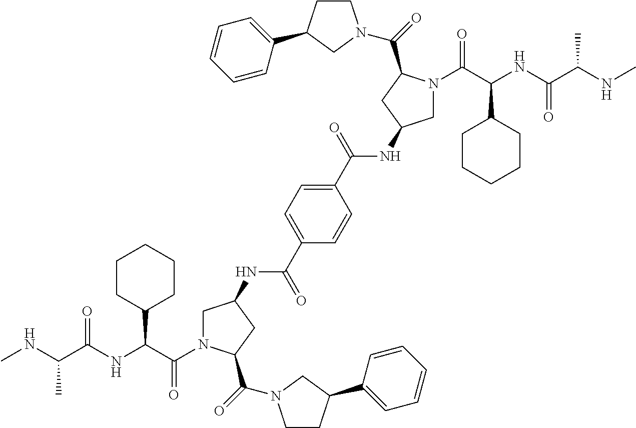 | [M + H]⁺ = 1098.8 [M + 2 H]²⁺ = 549.7 |
| 40 | 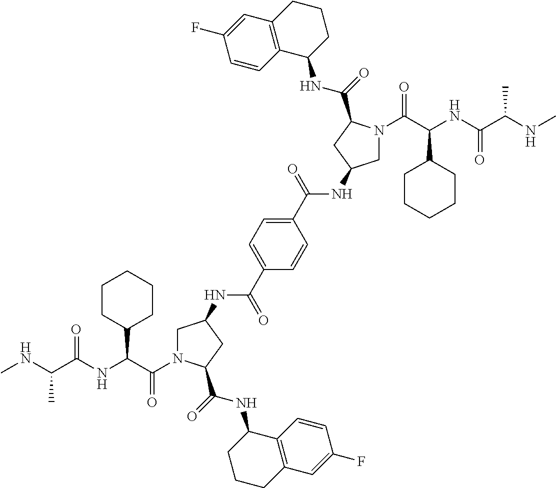 | [M + H]⁺ = 1133.8 [M + 2 H]²⁺ = 567.6 |
| 41 | 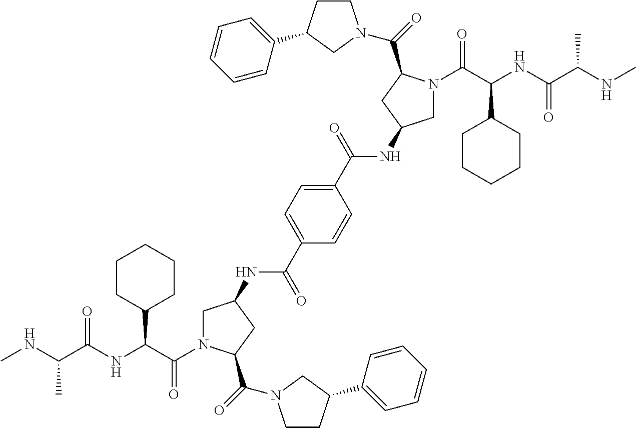 | [M + H]⁺ = 1097.8 [M + 2 H]²⁺ = 549.6 |
| 42 | 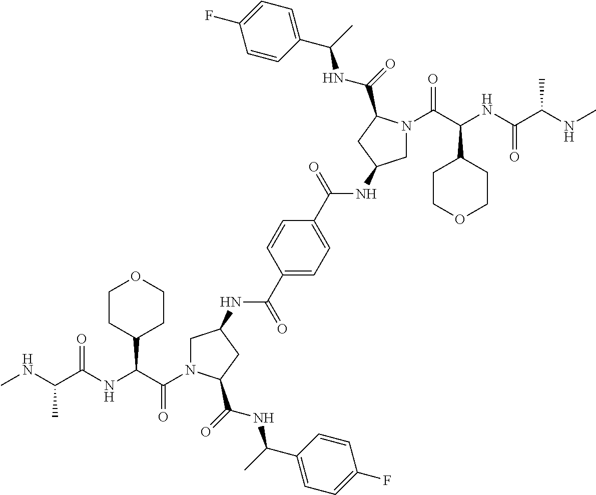 | [M + H]⁺ = 1085.5 [M + 2 H]²⁺ = 554.5 |
| 43 | 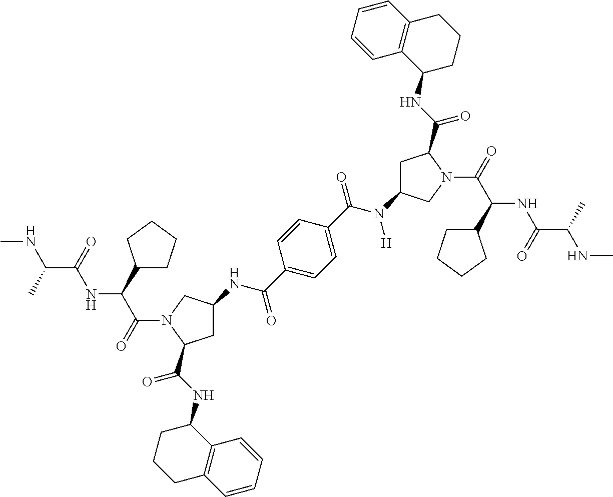 | [M + H]⁺ = 1069.6 [M + 2 H]²⁺ = 535.5 |
| 44 | 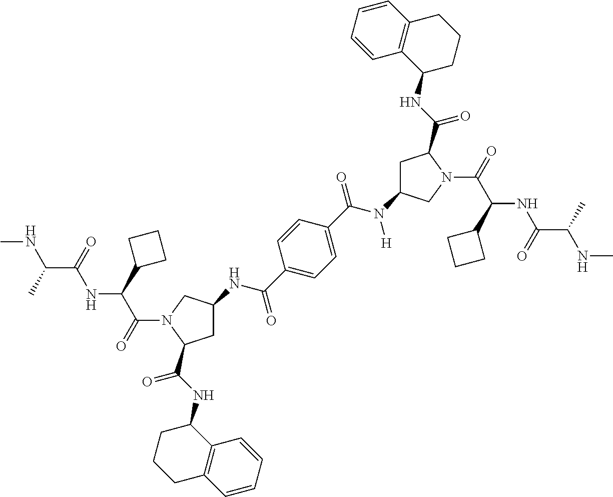 | [M + H]⁺ = 1041.5 [M + 2 H]²⁺ = 521.4 |
| 45 | 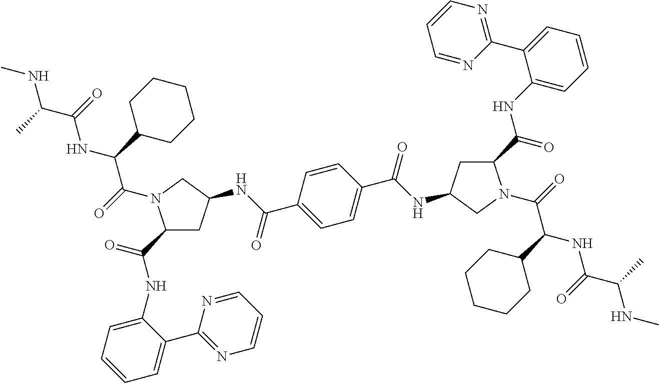 | [M + H]⁺ = 1145.7 [M + 2 H]²⁺ = 573.4 |
| 46 | 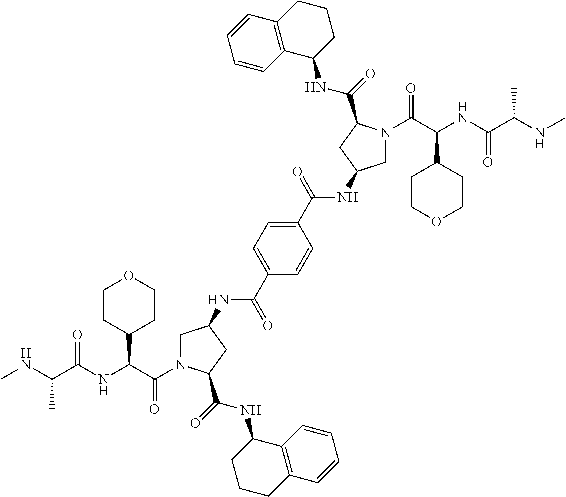 | [M + H]⁺ = 1101.6 [M + 2 H]²⁺ = 551.5 |
| 47 | 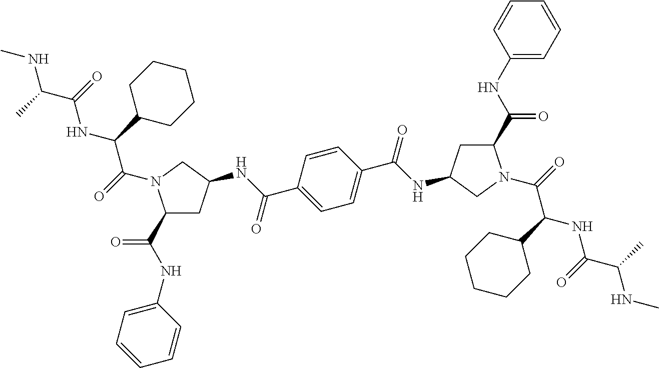 | [M + H]⁺ = 989.5 [M + 2 H]²⁺ = 495.5 |
| 48 | 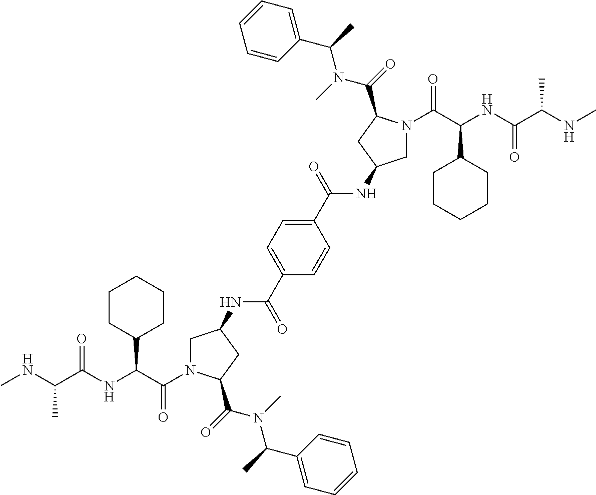 | [M + H]⁺ = 1073.7 |
| 49 | 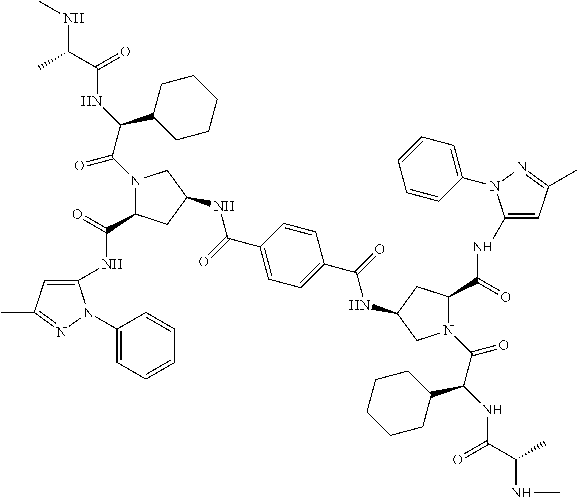 | [M + H]⁺ = 1149.6 [M + 2 H]²⁺ = 575.5 |
| 50 | 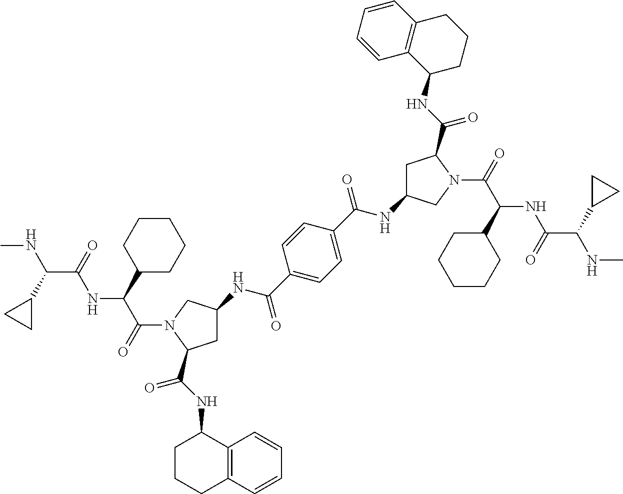 | [M + H]⁺ = 1149.6 [M + 2 H]²⁺ = 575.6 |
| 51 | 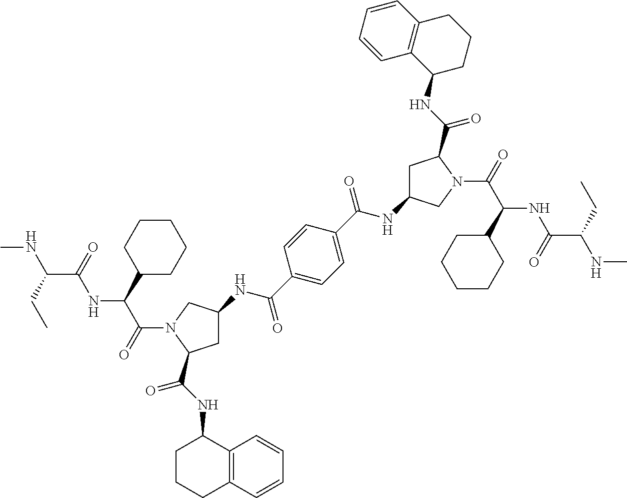 | [M + H]⁺ = 1126.6 [M + 2 H]²⁺ = 563.6 |
| 52 | 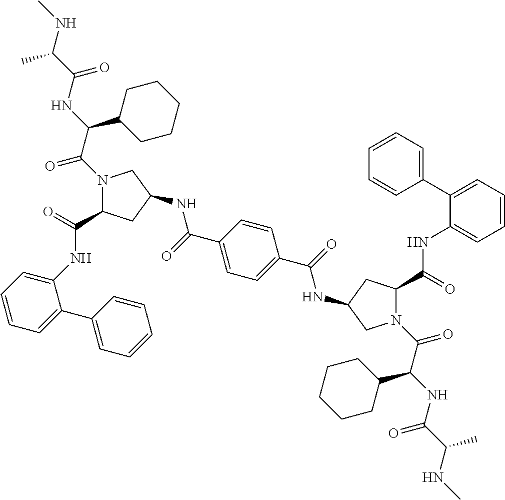 | [M + H]⁺ = 1141.6 [M + 2 H]²⁺ = 571.6 |
| 53 | 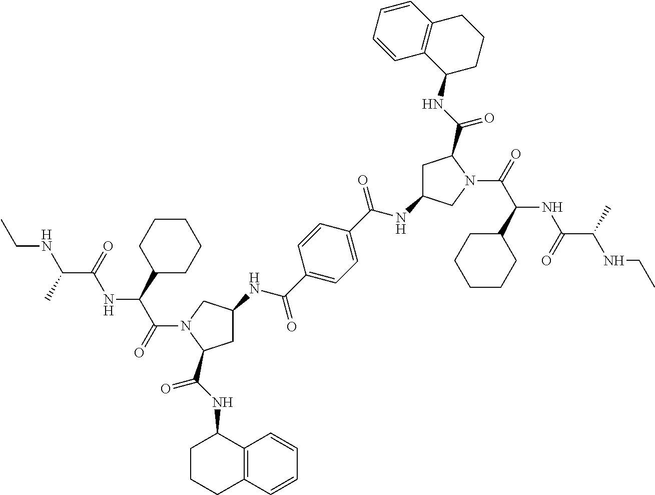 | [M + H]⁺ = 1125.6 [M + 2 H]²⁺ = 563.5 |
| 54 | 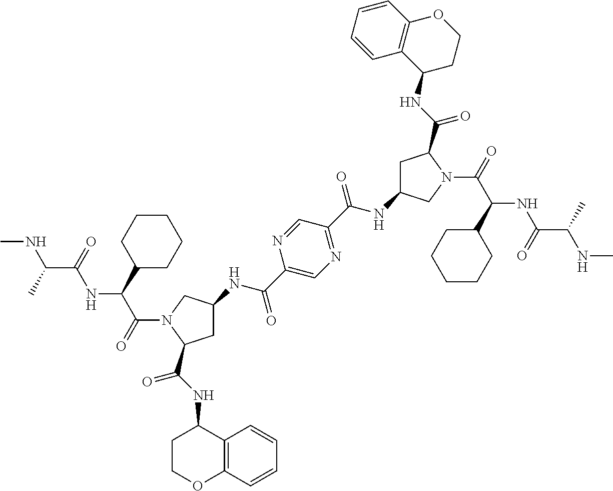 | [M + H]⁺ = 1103.4 [M + 2 H]²⁺ = 552.5 |
| 55 | 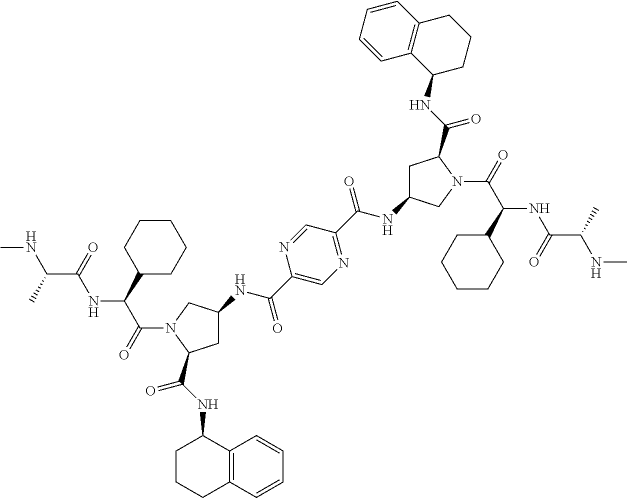 | [M + H]⁺ = 1099.5 [M + 2 H]²⁺ = 550.5 |
| 56 | 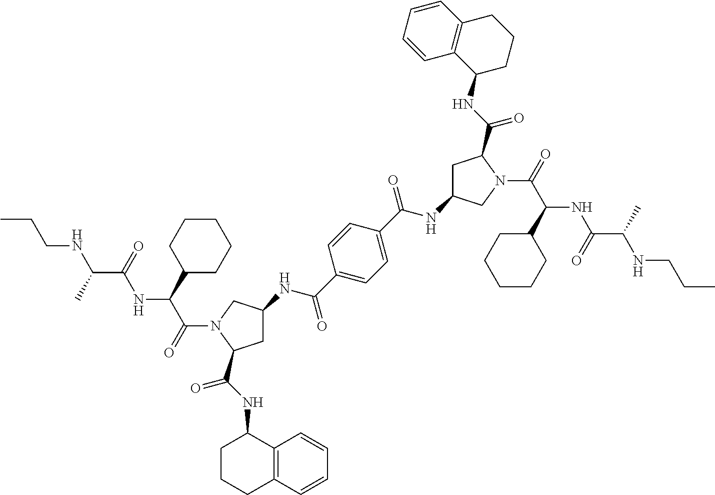 | [M + H]⁺ = 1153.6 [M + 2 H]²⁺ = 577.6 |
| 57 | 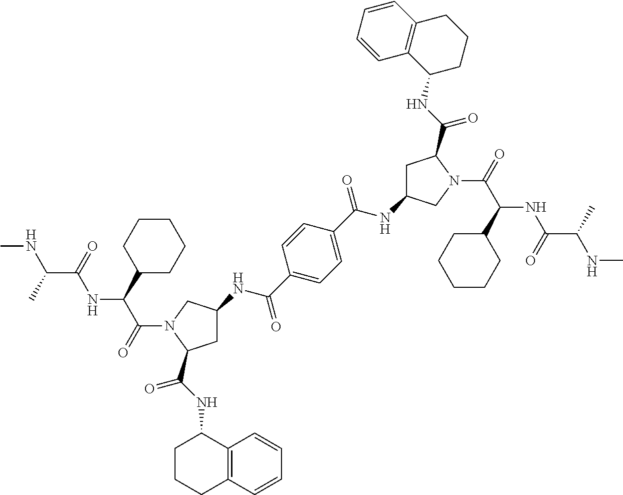 | [M + H]⁺ = 1097.6 [M + 2 H]²⁺ = 549.6 |
| 58 | 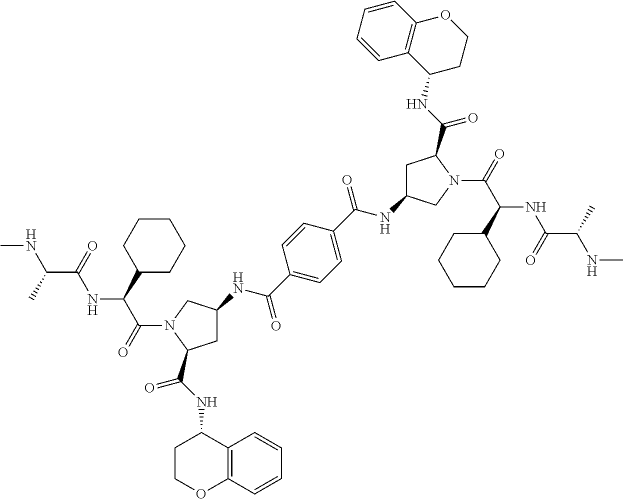 | [M + H]⁺ = 1101.6 [M + 2 H]²⁺ = 551.6 |

Other compounds of the instant invention include those of Table 1.1:

TABLE 1.1

| Compound | STRUCTURE |
|---|---|
| 59 | 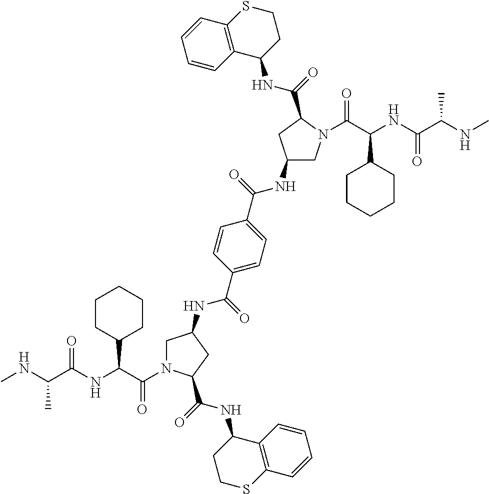 |
| 60 | 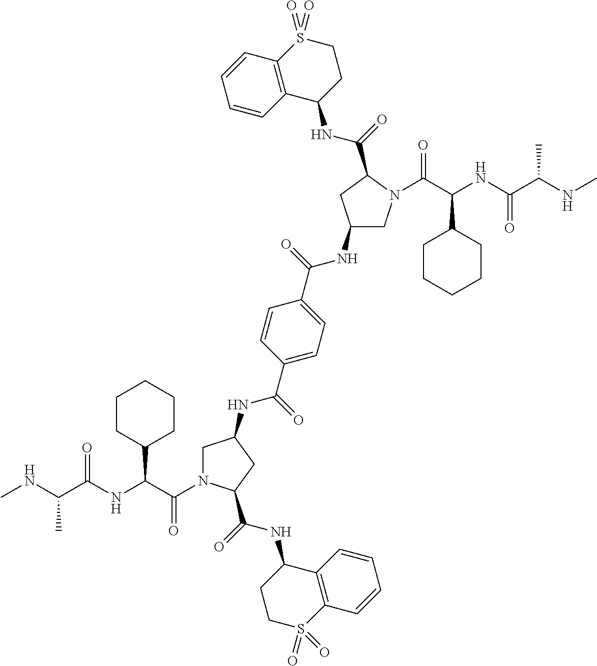 |
| 61 | 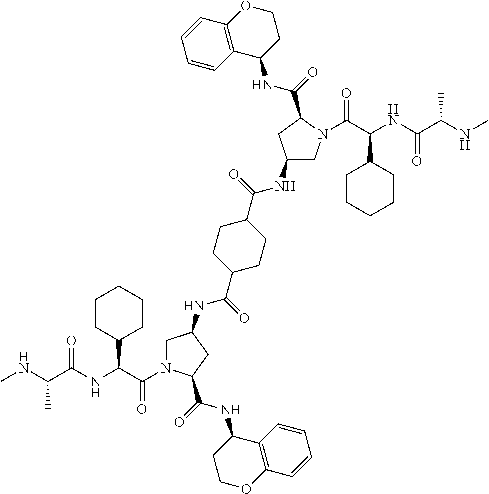 |
| 62 | 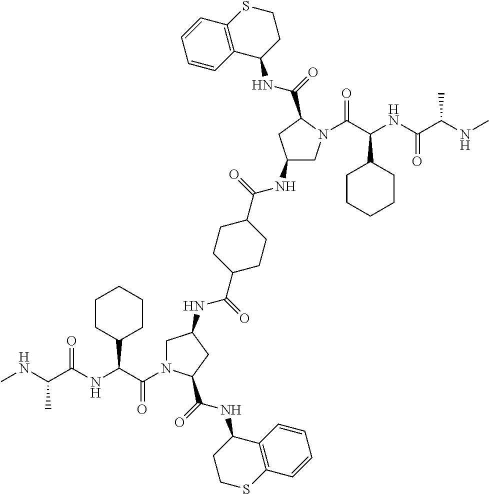 |
| 63 | 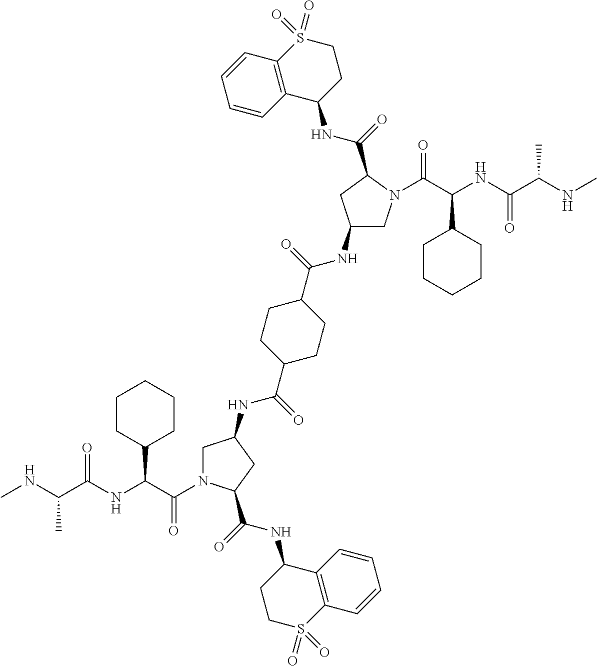 |
| 64 | 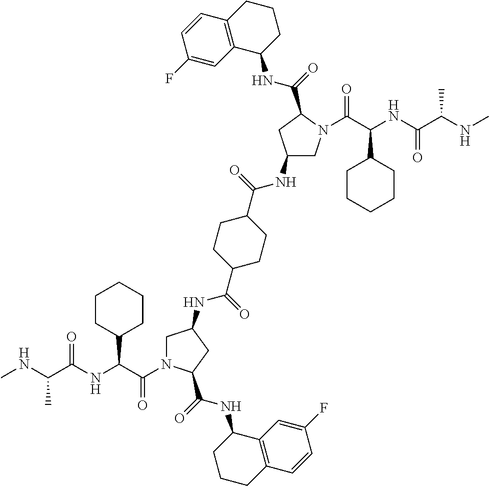 |
| 65 | 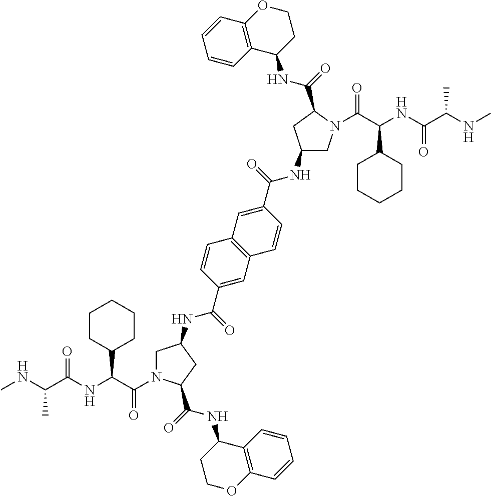 |
| 66 | 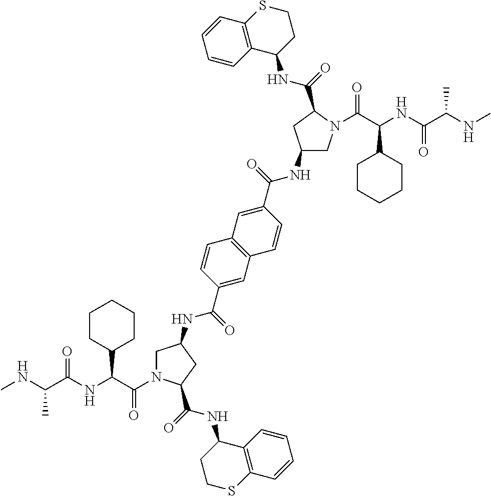 |
| 67 | 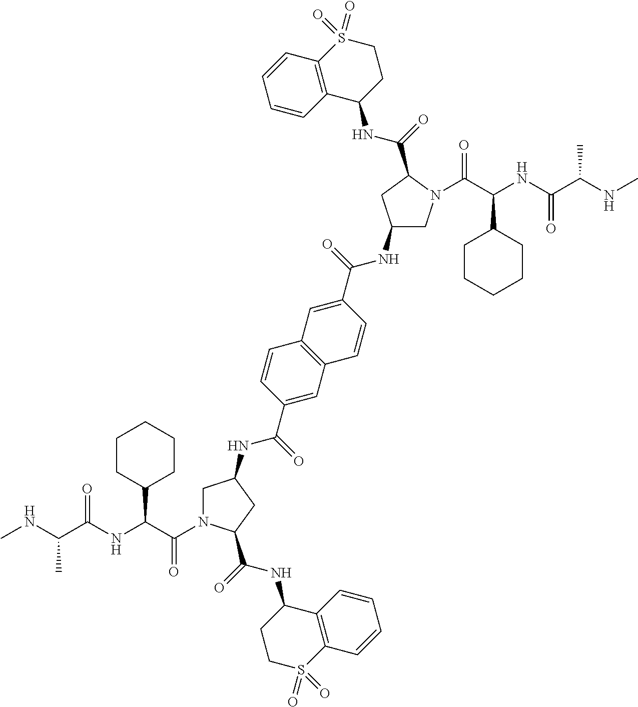 |
| 68 | 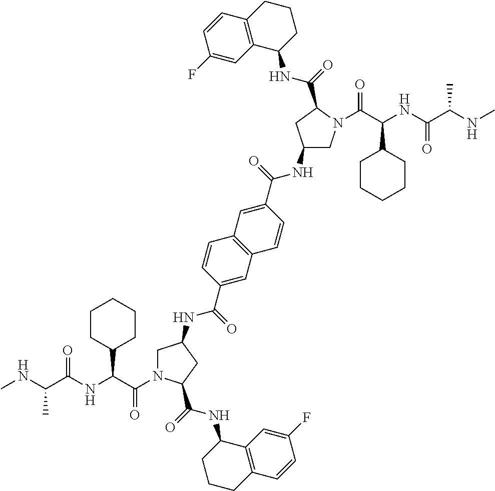 |
| 69 | 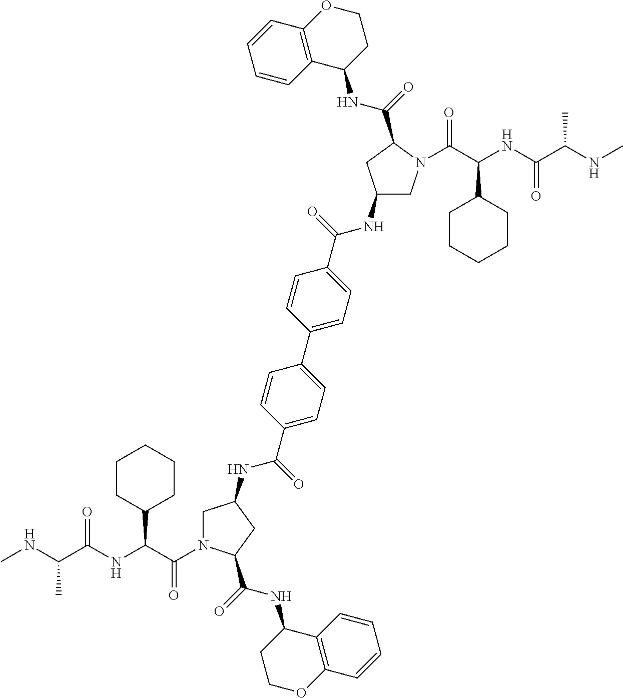 |
| 70 | 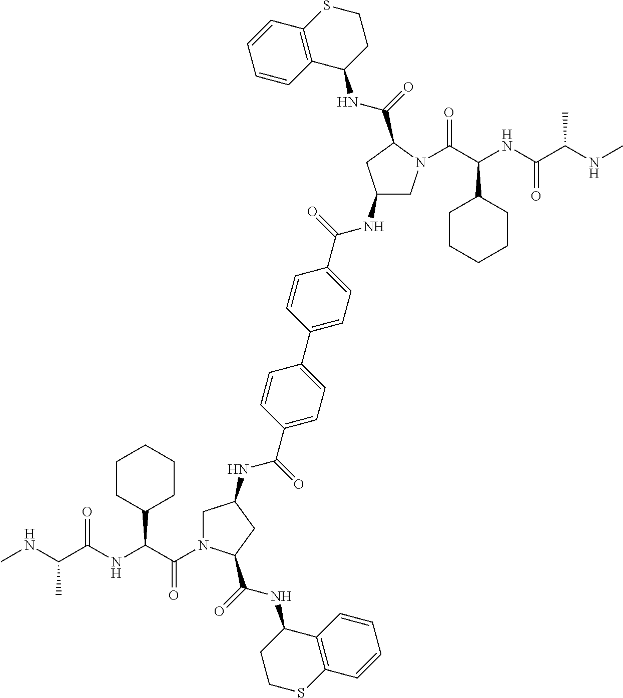 |
| 71 | 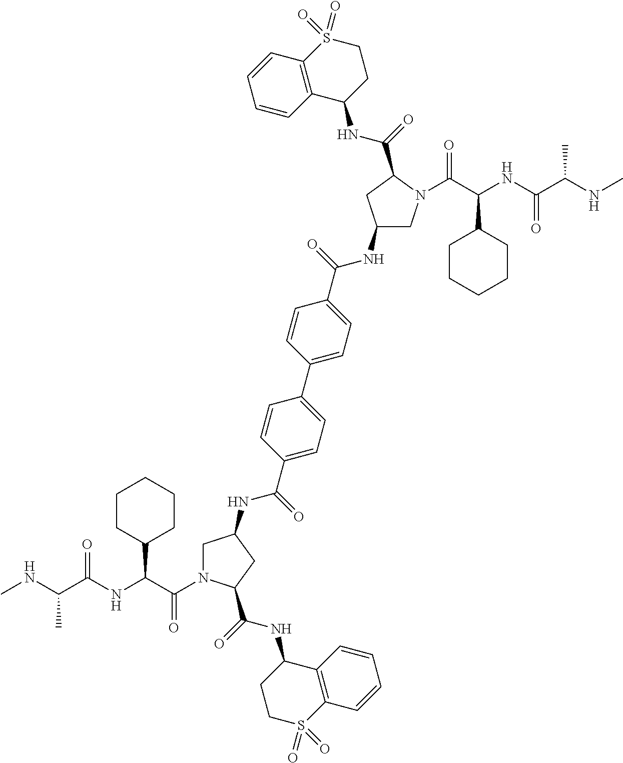 |
| 72 | 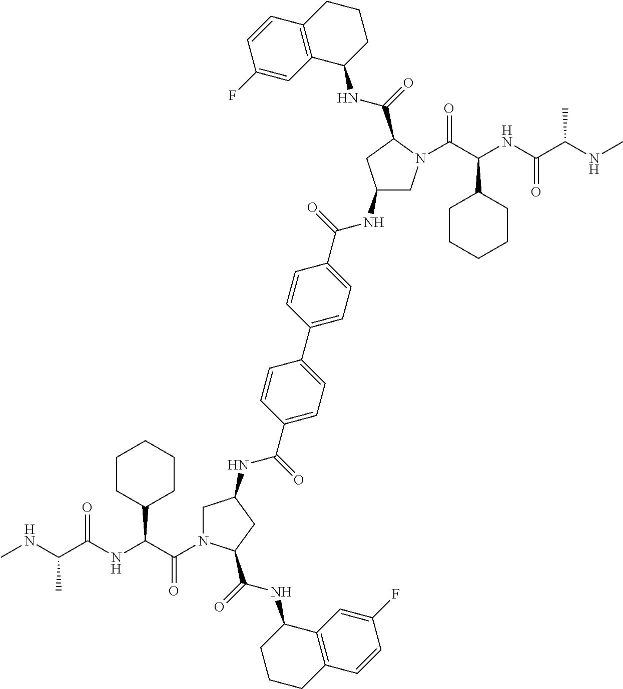 |
| 73 | 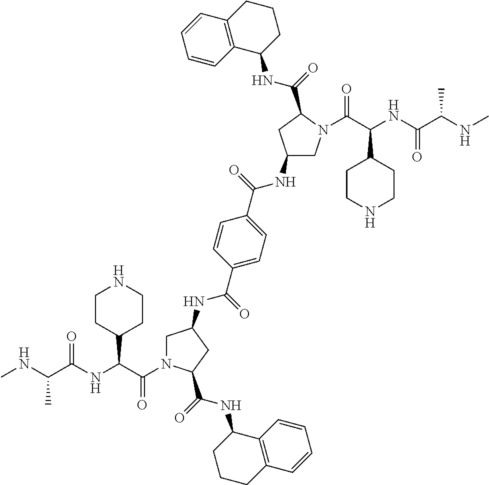 |
| 74 | 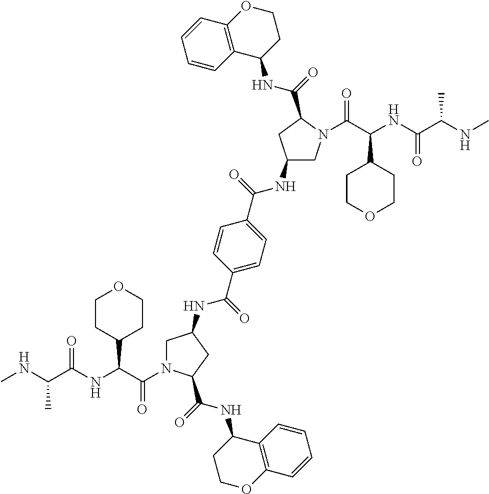 |
| 75 | 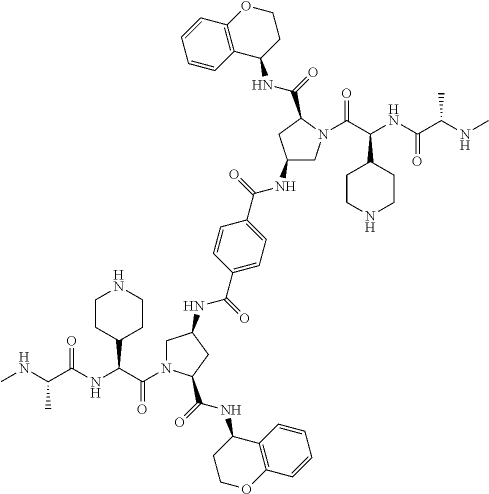 |
| 76 | 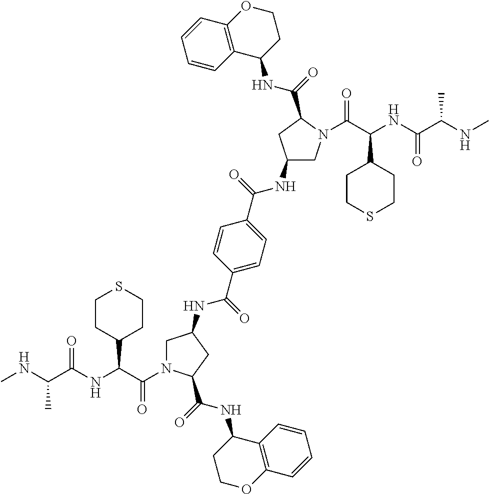 |
| 78 | 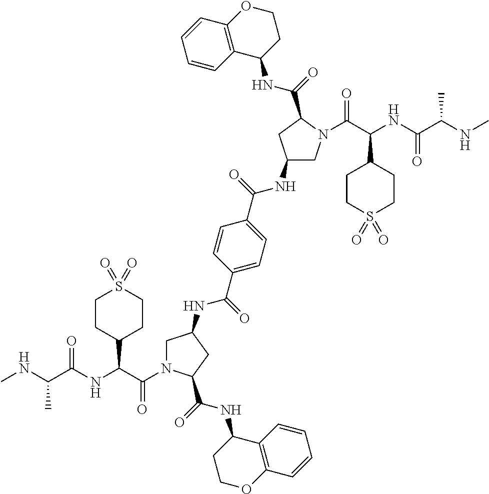 |
| 79 | 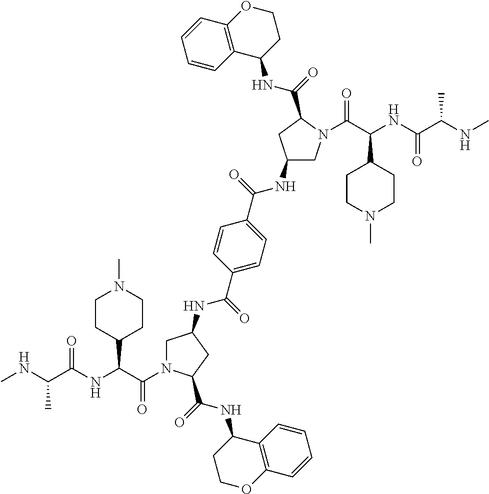 |
| 80 | 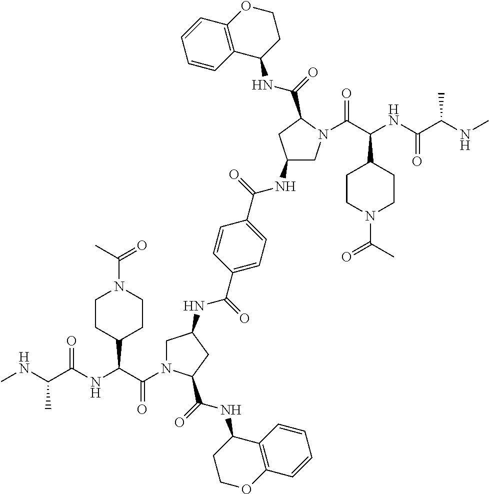 |
| 81 | 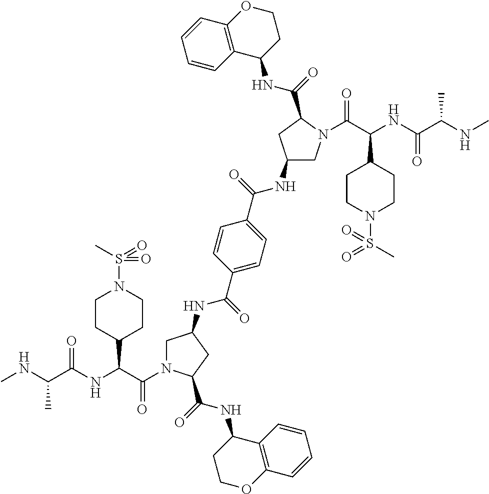 |
| 82 | 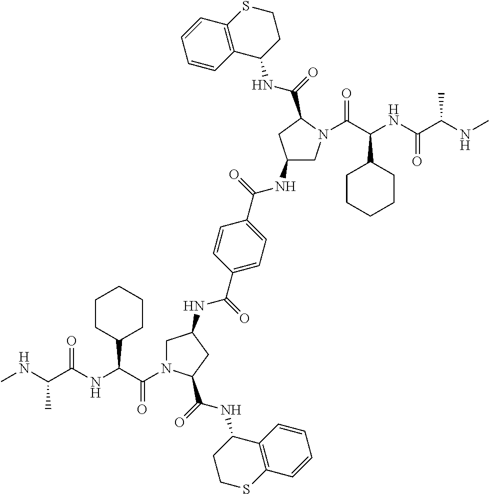 |
| 83 | 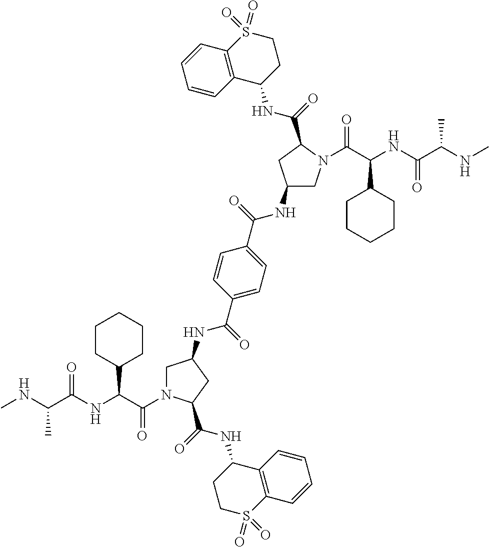 |
| 84 | 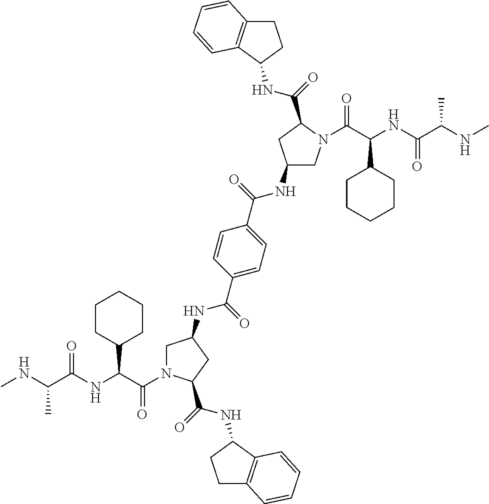 |
| 85 | 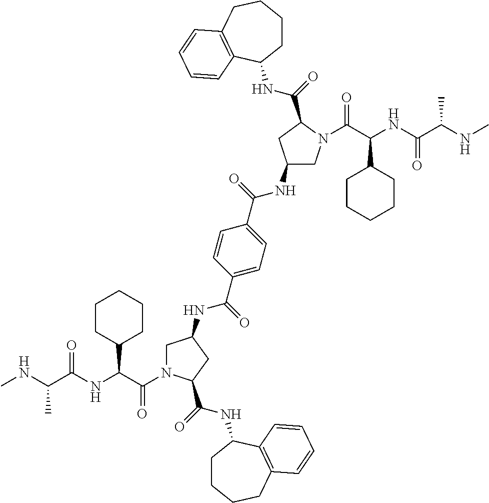 |
| 86 | 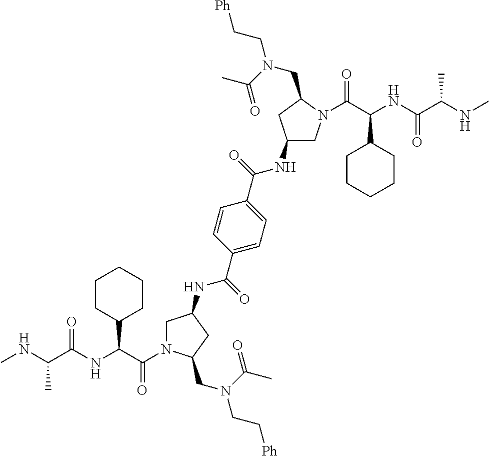 |
| 87 | 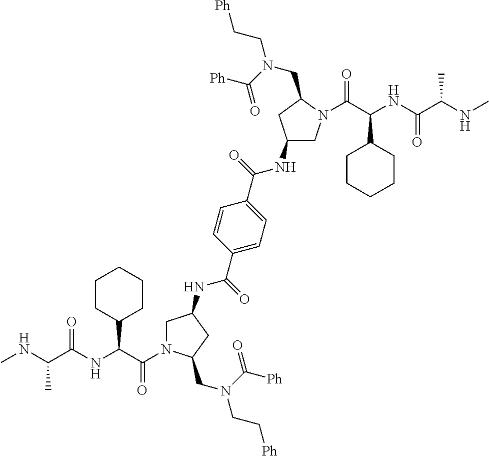 |
| 88 | 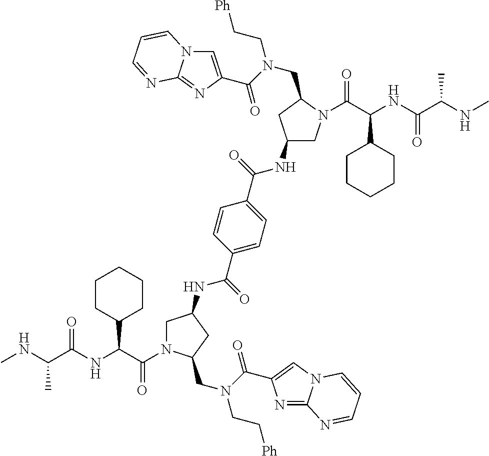 |
| 89 | 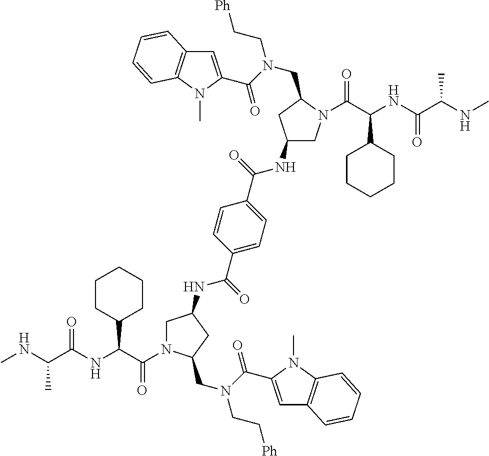 |
| 90 | 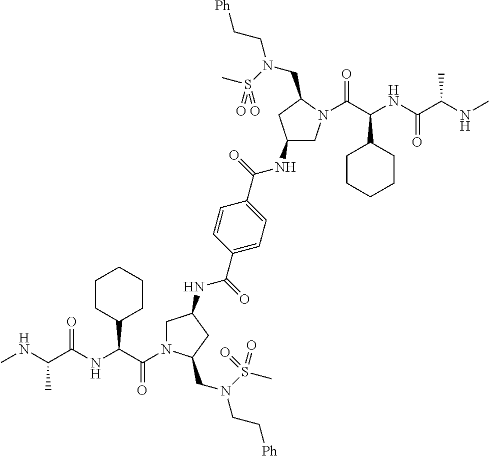 |
| 91 | 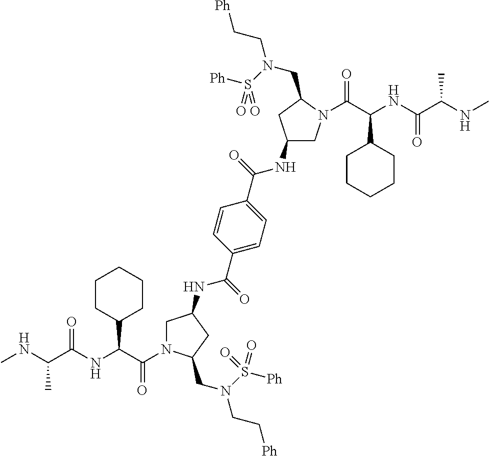 |
| 92 | 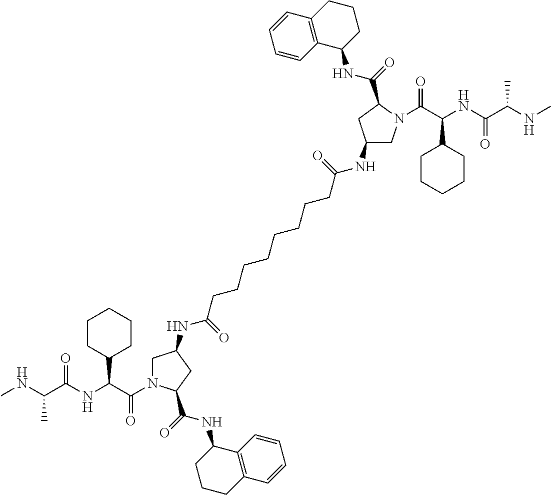 |
| 93 | 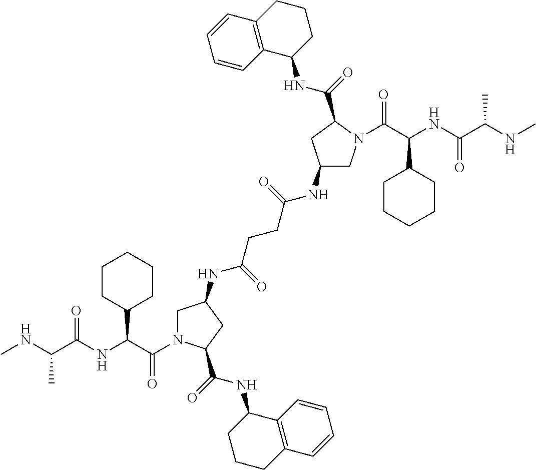 |

Representative compounds of the present invention which can be prepared by simple modification of the above procedures are illustrated below:

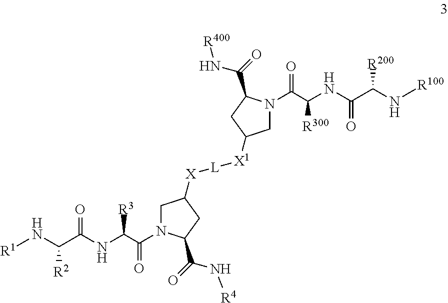

R¹, R², R³, R⁴, R¹⁰⁰, R²⁰⁰, R³⁰⁰, and R⁴⁰⁰ are defined as hereinabove,
—X-L-X¹— is chosen from:

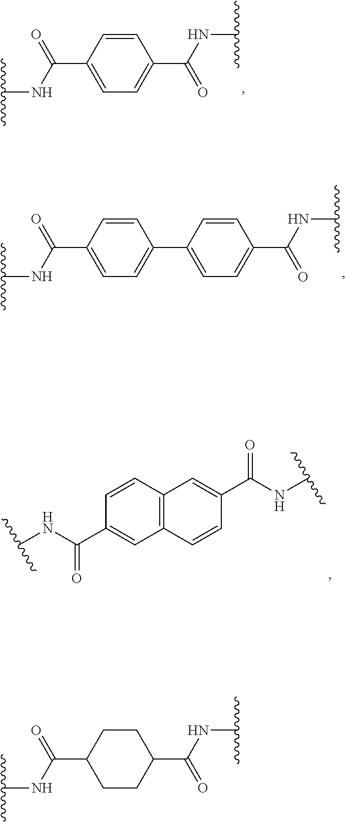

R³ and R³⁰⁰ are chosen from:

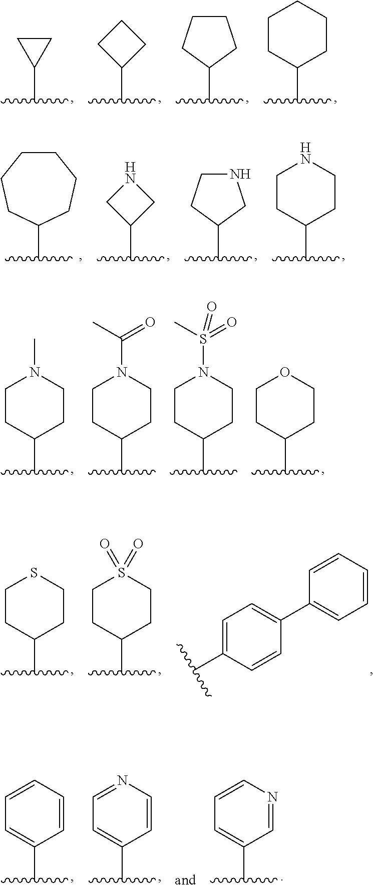

R⁴ and R⁴⁰⁰ are chosen from:

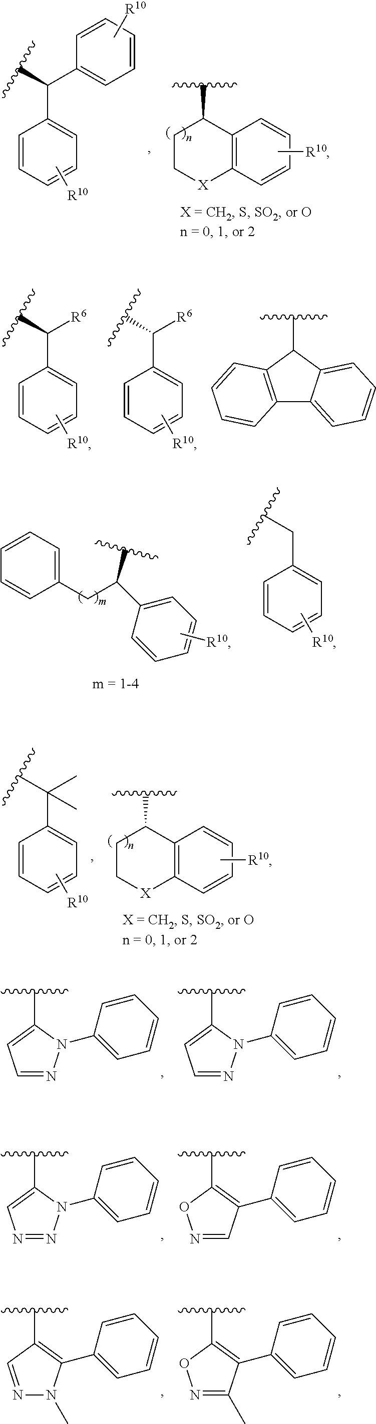

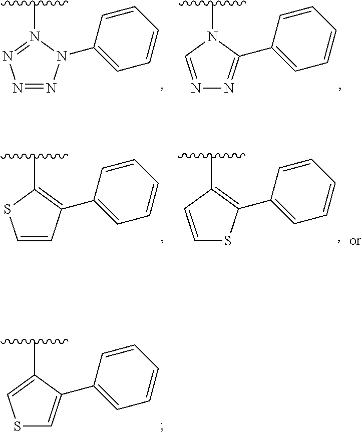

wherein R¹⁰ is optional, the aryl moieties may be substituted by R¹⁰ as defined hereinabove, and the alkyl may be substituted by R⁶ as defined hereinabove.

### Assays

Various in vitro assays were developed to demonstrate binding of compounds to the selected IAPs. The proteins were expressed using protein expression systems and purified by affinity using glutathione-derived beads for GST fusion proteins (Ge Health Care) and Maltose derived beads (New England Biolabs) for MBP fusion proteins. The details on each construction coding for the selected IAP protein are listed below:

GST-XIAP BIR3RING: AEG plasmid number 26: XIAP coding sequence amino acids 246-497 cloned into plasmid PGEX2T1.
GST-HIAP2 (cIAP-1) BIR 3: AEG plasmid number 104: HIAP2 coding sequence amino acids 251-363 cloned into PGex4T3.
GST-HIAP1(cIAP-2) BIR 3: AEG plasmid number 105: HIAP1 coding sequence amino acids 236-349, cloned into PGex4T3.
GST-XIAP-linker BIR 2 BIR3Ring: AEG plasmid number 219: XIAP coding sequence from amino acids 93-497 cloned into PGex4T1.
MBP-CIAP1-linker BIR 2 BIR3-Linker-Card: AEG plasmid number 291: cIAP-1 coding sequence amino acids 122-545 cloned into plasmid pMal-c4X.
MBP-CIAP1-linker BIR 2 BIR3-Linker: AEG plasmid number 294: cIAP-1 coding sequence amino acids 122-422 cloned into plasmid pMal-c4X.
MPB-CIAP2-linker BIR 2 BIR3-Linker: AEG plasmid number 289: cIAP-2 coding sequence amino acids 99-431 cloned into plasmid pMal-c4X.
GST-Full-length human XIAP, AEG plasmid number 23. XIAP coding sequence amino acids 1-497 cloned into plasmid PGex4T1.
GST-Livin full-length human livin, AEG plasmid number 224 coding sequence amino acids cloned into plasmid pGex4T3.

### Synthesis of Fluorescent Probe P1

A fluorescent peptide probe, Fmoc-Ala-Val-Pro-Phe-Tyr(t-Bu)-Leu-Pro-Gly(t-Bu)-Gly-OH was prepared using standard Fmoc chemistry on 2-chlorotrityl chloride resin (see Int. J. Pept. Prot. Res. 38:555-561, 1991). Cleavage from the resin was performed using 20% acetic acid in dichloromethane (DCM), which left the side chain still blocked. The C-terminal protected carboxylic acid was coupled to 4′-(aminomethy) fluorescein (Molecular Probes, A-1351; Eugene, Oreg.) using excess diisopropylcarbodiimide (DIC) in dimethylformamide (DMF) at room temperature and was purified by silica gel chromatography (10% methanol in DCM). The N-terminal Fmoc protecting group was removed using piperidine (20%) in DMF, and purified by silica gel chromatography (20% methanol in DCM, 0.5% HOAc). Finally, the t-butyl side chain protective groups were removed using 95% trifluoroacetic acid containing 2.5% water and 2.5% triisopropyl silane, to provide probe P1 (>95% pure, HPLC).

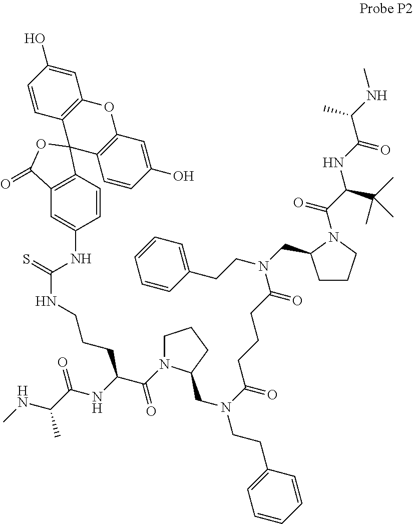

Probe P2 was prepared using methods as described in WO 2007/131,366.

### Binding Assay

### Fluorescence Polarization-Based Competition Assay

For all assays, the fluorescence and fluorescence-polarization was evaluated using a Tecan instrument with the excitation filter set at 485 nm and the emission filter set at 535 nm. For each assay, the concentration of the target protein was first established by titration of the selected protein in order to produce a dose-response signal when incubated alone in the presence of the fluorescent probe P1 or P2. Upon establishing these conditions, the compounds potency (IC₅₀) and selectivity, was assessed in the presence of a fix defined-amount of target protein and fluorescent probe and various concentrations (10-12 points) of the selected compounds and the fluorescence polarization evaluated.

For each assay the relative polarization-fluorescence units were plotted against the final concentrations of compound and the IC₅₀ calculated using the Grad pad prism software. The ki value were derived from the calculated IC₅₀ value as described above and according to the equation described in Nikolovska-Coleska, Z. (2004) Anal Biochem 332, 261-273.

Compounds of the invention displayed ki values of less than 1 μM in the XIAP, cIAP1 and cIAP2 BIR2-BIR3 FP assays described above (using probe P2),

### Caspase-3 Full Length XIAP, Linker BIR2 or Linker-BIR2-BIR3-RING Derepression Assay

In order to determine the relative activity of the selected compound against XIAP-Bir2, an in vitro assay can be employed using caspase-3 and GST fusion proteins of XIAP linker-Bir2, XIAP Linker Bir2-Bir3-RING or full-length XIAP. For example, Caspase 3 (0.125 ul) and 12.25-34.25 nM (final concentration) of GST-XIAP fusion protein (GST-Bir2, GST-Bir2Bir3RING or full-length XIAP) can be co-incubated with serial dilutions of compound (200 uM-5 pM). Caspase 3 activity can then be measured by overlaying 25 uL of a 0.4 mM DEVD-AMC solution. Final reaction volume was 100 uL. All dilutions can be performed using caspase buffer (50 mM Hepes pH 7.4, 100 mM NaCl, 10% sucrose, 1 mM EDTA, 10 mM DTT, 0.1% CHAPS) (Stennicke, H. R., and Salvesen, G. S. (1997), Biochemical characteristics of caspase-3, -6, -7, and -8. J. Biol. Chem. 272, 25719-25723).

The fluorescent AMC released from the caspase-3 hydrolysis of the substrate can be measured in a TECAN spectrophotometer at 360 nm excitation and 444 nm emission, after 15 minutes of incubation at room temperature. IC₅₀ values can be calculated on a one or two-site competition model using GraphPad v4.0, using the fluorescence values after 15 minutes of incubation.

### Cell Culture and Cell Viability Assays

### SKOV3

Ovarian adenocarcinoma SKOV3 cells (ATCC# HTB-77) were cultured as monolayers in McCoy's 5a medium (HyClone) supplemented with 2.2 g/L sodium bicarbonate (Gibco), 10% FBS (HyClone) and 1% penicillin/streptomycin (HyClone). Cells were seeded in tissue culture-treated 96 well plates at 5000 cells/well in 150 ul of media. After 24 hours, triplicate wells of cells were treated with various concentrations of compound (0.01 to 10000 nM) diluted in 50 ul of culture media. Cells were incubated at 37° C. (5% CO₂) in the presence of compound for 72 hours. Metabolic viability of remaining cells was assessed by MTT (thiazolyl blue tetrazolium bromide, Sigma) assay. 20 ul of MTT reagent (10 mg/ml) was added per well and plates were incubated for 4 hours at 37° C. (5% CO₂). The supernatant was then removed from the plate. The converted MTT product was solubilized with 100 uL of isopropanol and the absorbance was read at 570 nm using a Tecan spectrophotometer.

### HCT116+ETR1

Colorectal carcinoma HCT116 cells (ATCC# CCL-247) were cultured as monolayers in 96 well plates at a density of 2000 cells per well in 100 ul of McCoy's 5a medium (HyClone) supplemented with 2.2 g/L sodium bicarbonate (Gibco), 10% FBS (HyClone) and 1% penecillin/streptomycin (HyClone) for 24 hours. Triplicate wells of cells were treated for 72 hrs at 37° C. (5% CO₂) with 50 ul of HGS agonistic Trail receptor antibody, ETR1 (40 ng/ml) in combination with 50 ul of diluted compound (0.01 to 10000 nM). Metabolic viability of remaining cells was assessed by MTT assay.

### Human Synoviocytes

Rheumatoid arthritis human fibroblast-like synoviocytes (HFLS-RA, Cell Applications Inc.) were seeded in 96-well plates at 3000 cells per well in 100 ul of complete synoviocyte growth medium (Cell Applications Inc.) one day prior to treatment. Triplicate wells of cells were treated by the addition of 50 ul of diluted compound (0.01 to 100 nM) in combination with 50 ul of anti-human CD95 (Fas) antibody (300 ng/ml; clone CH-11, Beckman Coulter/Immunotech) and incubated at 37° C. (5% CO2) for 72 hrs. Cell viability was measured by Cell Titer-Glo Luminescent Assay (Promega). Briefly, 100 ul of media was removed from wells and an equal volume of Cell Titer-Glo Reagent was added to the cells. The plates were mixed for 2 minutes on an orbital shaker and after a 10-minute incubation period at room temperature, the emitted luminescence was detected using a Tecan Infinite F200 spectrophotometer.

### Determination of EC₅₀ Values

The percentage viability of compound-treated cells was expressed as a fraction of the absorbance/luminescence signal obtained from non-treated cells. EC₅₀ values (corresponding to 50% cell survival in the presence of compound as compared to untreated controls) were calculated from MTT survival curves (HCT116 and SKOV3) using BioAssay software (CambridgeSoft) and from Cell Titer-Glo survival curves (synovioycte and Jurkat cells) using GraphPad Prism (Graph Pad Software Inc.). In the below chart, EC50 values are summarized as follows: a=EC₅₀ of less than 10 nM; b=EC₅₀ of 10-100 nM; and c=EC₅₀ of greater than 100 nM.

| | EC₅₀ | EC₅₀ | EC₅₀ |
|---|---|---|---|
| | SKOV3 | HCT116 + ETR1 | synoviocyctes + |
| Compound | (nM) | (nM) | FAS (nM) |
| 1 | a | a | a |
| 2 | b | a | — |
| 3 | a | a | a |
| 4 | a | a | — |
| 5 | a | a | — |
| 6 | a | a | — |
| 7 | a | a | a |
| 8 | c | b | — |
| 9 | a | a | — |
| 10 | a | a | — |
| 11 | b | a | — |
| 12 | b | a | — |
| 13 | a | a | — |
| 14 | b | a | — |
| 15 | a | a | — |
| 16 | b | b | — |
| 17 | b | a | — |
| 18 | b | a | — |
| 19 | a | a | — |
| 20 | a | a | — |
| 21 | b | a | — |
| 22 | a | a | — |
| 23 | a | a | — |
| 24 | b | a | — |
| 25 | b | a | — |
| 26 | a | a | — |
| 27 | b | a | — |
| 28 | b | b | — |
| 29 | a | a | — |
| 30 | c | c | — |
| 31 | a | a | — |
| 32 | a | a | — |
| 33 | — | a | — |
| 34 | — | a | — |
| 35 | — | b | — |
| 36 | — | a | — |
| 37 | — | a | — |
| 38 | — | a | — |
| 39 | — | b | — |
| 40 | — | a | — |
| 41 | — | b | b |
| 42 | — | b | — |
| 43 | — | a | — |
| 44 | — | a | — |
| 45 | — | b | — |
| 46 | — | b | — |
| 47 | — | a | — |
| 48 | — | b | — |
| 49 | — | b | — |
| 50 | — | b | — |
| 51 | — | a | a |
| 52 | — | a | — |
| 53 | — | a | — |
| 54 | — | a | — |
| 55 | — | a | — |
| 56 | — | a | — |
| 57 | — | b | — |
| 58 | — | b | — |

Tumor Suppressive Effect of Compound in Combination with Taxotere using an H460 Xenograft Model

Female, CD-1, nude mice received 1×10⁶ H460 cells (in 100 uL of serum-free media) subcutaneously at the right flank. When average tumor size reached ˜100 mm³ groups were formed using a balanced design based on tumor size, and treatment commenced. Tumor bearing mice were treated with either vehicle (5% D5W; 5 mL/kg, IV) or compound (1 mg/kg, IV) using a 5 on/2off treatment schedule. Taxotere (30 mg/kg, IP) was given twice, one week apart commencing one day after IAP inhibitory compounds.

Compound 1 (1 mg/kg) provided a 54% tumor growth suppression relative to vehicle treated controls after 3 weeks of treatment.

Tumor Suppressive Effect of Compound in Combination with ETR1 using an HCT-116 Xenograft Model

Female, CD-1, nude mice received 1.5×10⁶ HCT-116 cells (in 100 uL of serum-free media) subcutaneously at the right flank. When average tumor size reached ˜150 mm³ groups were formed using a balanced design based on tumor size, and treatment commenced. Tumor bearing mice were treated with either vehicle (5% D5W; 5 mL/kg, IV) or compound (1 mg/kg, IV) using a 5 on/2off treatment schedule. Mapatumamab (10 mg/kg, IP) was given twice weekly for the duration of the experiment, commencing one day after IAP inhibitory compounds.

Compound 1 (1 mg/kg) provided a 59% tumor growth suppression relative to vehicle treated controls after 3 weeks of treatment.

### Other Embodiments

From the foregoing description, it will be apparent to one of ordinary skill in the art that variations and modifications may be made to the invention described herein to adapt it to various usages and conditions. Such embodiments are also within the scope of the present invention.

1. A compound of Formula 1: 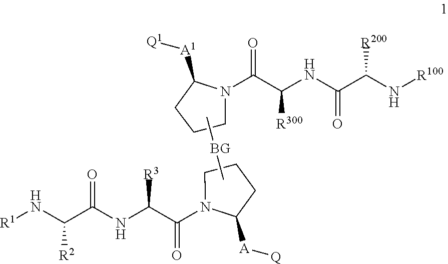
or a salt thereof, wherein
m is 0, 1 or 2;
Y is NH, O or S;
BG is —X-L-X¹—;
X and X¹ are independently
1) O,
2) NR¹²,
3) S,
4) —C₁-C₆ alkyl-,
5) —C₁-C₆ alkyl-O—,
6) —C₁-C₆ alkyl-NR¹²—,
7) —C₁-C₆ alkyl-S—, 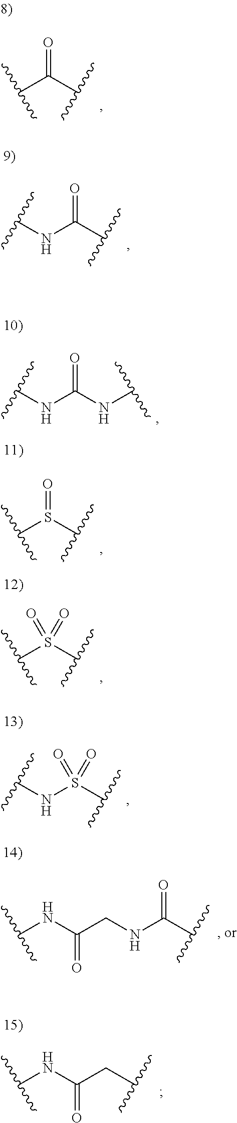
L is
1) —C₁-C₂₀ alkyl-,
2) —C₂-C₆ alkenyl-,
3) —C₂-C₈ alkynyl-,
4) —C₃-C₇ cycloalkyl-,
5) -aryl-,
6) -biphenyl-,
7) -heteroaryl-,
8) -heterocyclyl-,
9) —C₁-C₆ alkyl-(C₂-C₆ alkenyl)-C₁-C₆ alkyl-,
10) —C₁-C₆ alkyl-(C₂-C₄ alkynyl)-C₁-C₆ alkyl-
11) —C₁-C₆ alkyl-(C₃-C₇ cycloalkyl)-C₁-C₆ alkyl-,
12) —C₁-C₆ alkyl-aryl-C₁-C₆ alkyl-,
13) —C₁-C₆ alkyl-biphenyl-C₁-C₆ alkyl-,
14) —C₁-C₆ alkyl-heteroaryl-C₁-C₆ alkyl-,
15) —C₁-C₆alkyl-heterocycyl-C₁-C₆ alkyl-,
16) —C₁-C₆ alkyl-Y—C₁-C₆ alkyl-,
17) -aryl-Y-aryl-,
18) -heteroaryl-Y-heteroaryl-,
19) -heterocyclyl-Y-heterocyclyl-, 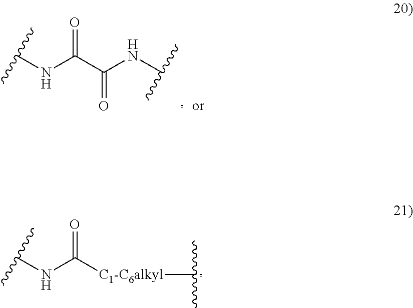
wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents;
Q is
1) NR⁴R⁵,
2) OR¹¹,
3) S(O)ₘR¹¹;
4) aryl, or
5) heteroaryl,
wherein the aryl and the heteroaryl are optionally substituted with one or more R¹⁰ substituents;
Q¹ is
1) NR⁴⁰⁰R⁵⁰⁰,
2) OR¹¹⁰⁰,
3) S(O)ₘR¹¹⁰⁰,
4) aryl, or
5) heteroaryl,
wherein the aryl and the heteroaryl are optionally substituted with one or more R¹⁰ substituents;
A and A¹ are independently
1) C₁-C₃ alkylene, or
2) —C(O)—;
R¹ and R¹⁰⁰ are C₁-C₆ alkyl optionally substituted with one or more R⁶ substituents;
R² and R²⁰⁰ are independently
1) H,
2) C₁-C₆ alkyl optionally substituted with one or more R⁶ substituents; or
3) C₃-C₇ cycloalkyl optionally substituted with one or more R⁶ substituents.
R³ and R³⁰⁰ are independently
1) C₃-C₇ cycloalkyl,
2) C₃-C₇ cycloalkenyl,
3) aryl,
4) heteroaryl,
5) heterocyclyl, or
6) heterobicyclyl,
wherein the cycloalkyl, cycloalkenyl, heterocyclyl, and heterobicyclyl are optionally substituted with one or more R⁶ substituents; and wherein the aryl and heteroaryl are optionally substituted with one of more R¹⁰ substituents;
R⁴, R⁴⁰⁰, R⁵, and R⁵⁰⁰ are each independently
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)—R¹¹,
13) C(O)O—R¹¹,
14) C(═Y)NR⁸R⁹, or
15) S(O)₂—R¹¹,
wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
or R⁴ and R⁵ taken together with the nitrogen to which they are attached, and R⁴⁰⁰ and R⁵⁰⁰ taken together with the nitrogen to which they are attached, form a C₃-C₇ heterocycloalkylene optionally substituted with C₁-C₆ alkyl, C₃-C₇ cycloalkyl, or C₆-C₁₀ aryl, wherein the aryl is optionally substituted with R¹⁰,
R⁶ is
1) halogen,
2) NO₂,
3) CN,
4) haloalkyl,
5) C₁-C₆ alkyl,
6) C₂-C₆ alkenyl,
7) C₂-C₄ alkynyl,
8) C₃-C₇ cycloalkyl,
9) C₃-C₇ cycloalkenyl,
10) aryl,
11) heteroaryl,
12) heterocyclyl,
13) heterobicyclyl,
14) OR⁷,
15) S(O)ₘR⁷,
16) NR⁸R⁹,
17) NR⁸S(O)₂R¹¹,
18) COR⁷,
19) C(O)OR⁷,
20) CONR⁸R⁹,
21) S(O)₂NR⁸R⁹
22) OC(O)R⁷,
23) OC(O)Y—R¹¹,
24) SC(O)R⁷, or
25) NC(Y)NR⁸R⁹,
wherein the aryl, heteroaryl, heterocyclyl, and heterobicyclyl are optionally substituted with one or more R¹⁰ substituents;
R⁷ is
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) —C(═Y)NR⁸R⁹, or
13) C₁-C₆ alkyl-C₂-C₄ alkenyl, or
14) C₁-C₆ alkyl-C₂-C₄ alkynyl,
wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
R⁸ and R⁹ are each independently
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents;
or R⁸ and R⁹ together with the nitrogen atom to which they are attached form a five, six or seven membered heterocyclic ring optionally substituted with one or more R⁶ substituents;
R¹⁰ is
1) halogen,
2) NO₂,
3) CN,
4) C₁-C₆ alkyl,
5) C₂-C₆ alkenyl,
6) C₂-C₄ alkynyl,
7) C₃-C₇ cycloalkyl,
8) C₃-C₇ cycloalkenyl,
9) haloalkyl,
10) OR⁷,
11) NR⁸R⁹,
12) SR⁷,
13) COR⁷,
14) C(O)OR⁷,
15) S(O)ₘR⁷,
16) CONR⁸R⁹,
17) S(O)₂NR⁸R⁹,
18) aryl,
19) heteroaryl,
20) heterocyclyl, or
21) heterobicyclyl,
wherein the alkyl, alkenyl, alkynyl, cycloalkyl, and cycloalkenyl are optionally substituted with one or more R⁶ substituents;
R¹¹ and R¹¹⁰⁰ are
1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) heteroaryl,
9) heterocyclyl, or
10) heterobicyclyl,
wherein the alkyl, alkenyl, alkynyl, cycloalkyl, and cycloalkenyl are optionally substituted with one or more R⁶ substituents; and wherein the aryl, heteroaryl, heterocyclyl, and heterobicyclyl are optionally substituted with one or more R¹⁰ substituents.

2. The compound according to claim 1, wherein A and A¹ are both CH₂ or both C═O and R³ and R³⁰⁰ are bot
1) C₃-C₇ cycloalkyl,
2) C₃-C₇ cycloalkenyl,
3) heteroaryl,
4) heterocyclyl, or
5) heterobicyclyl,
wherein the cycloalkyl, cycloalkenyl, heterocyclyl, and heterobicyclyl are optionally substituted with one or more R⁶ substituents; and wherein the heteroaryl is optionally substituted with one of more R¹⁰ substituents.

3. A compound according to claim 1 of any one of Formulas 1a through 1c:

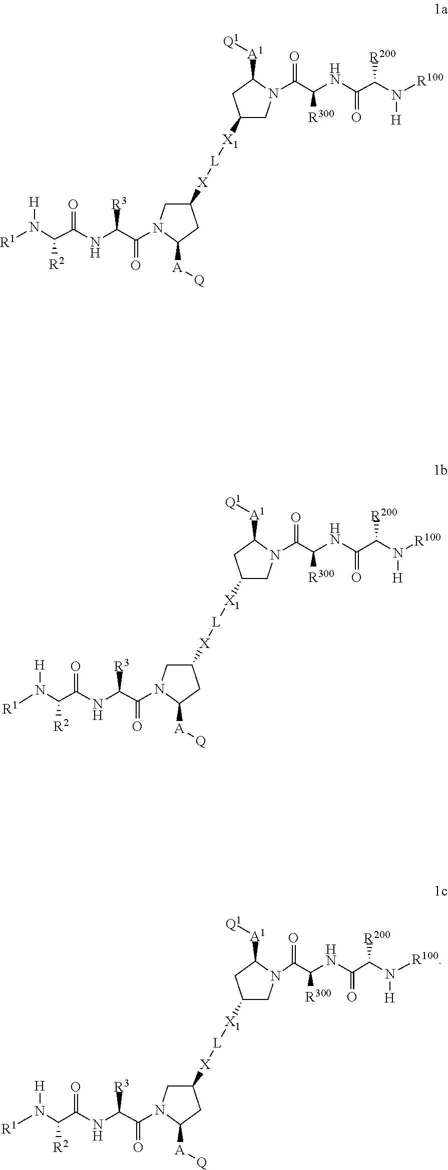

4. A compound according to claim 1 of Formula 1.1a through 1.1c:

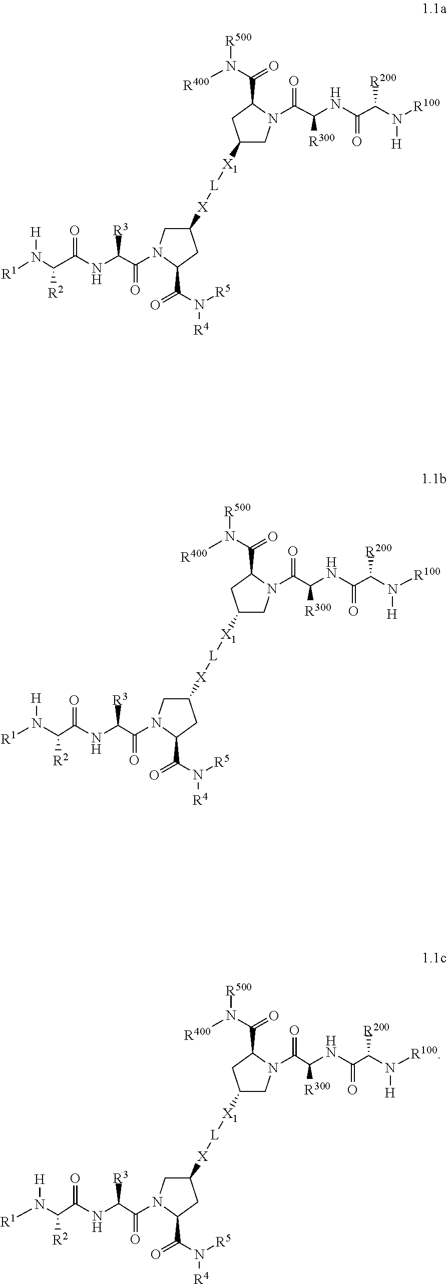

5. The compound according to claim 1, wherein X and X¹ are:

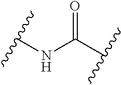

6. The compound according to claim 1, wherein L is
1) —C₁-C₂₀ alkyl-,
2) —C₃-C₇ cycloalkyl-,
3) -aryl-,
4) -biphenyl-,
5) -heteroaryl-,
6) —C₁-C₆ alkyl-(C₂-C₄ alkynyl)-C₁-C₆ alkyl-
7) —C₁-C₆ alkyl-aryl-C₁-C₆ alkyl-,
8) —C₁-C₆ alkyl-biphenyl-C₁-C₆ alkyl-,
9) —C₁-C₆ alkyl-heteroaryl-C₁-C₆ alkyl-,
10) —C₁-C₆ alkyl-heterocycyl-C₁-C₆ alkyl-,
11) —C₁-C₆ alkyl-Y—C₁-C₆ alkyl-, 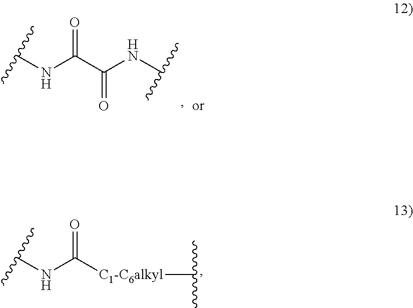
wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents

7. The compound according to claim 6, wherein L is: 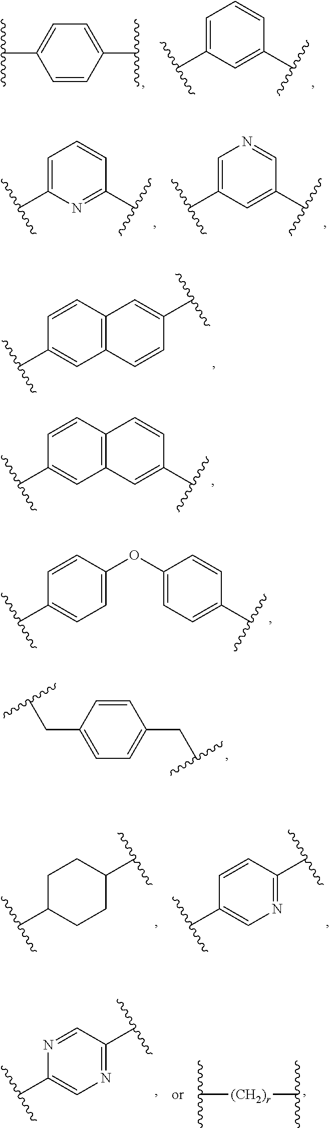
wherein r is 1, 2, 3, 4, 5, 6, 7, 8, 9, or 10.

8. A compound according to claim 1 having the formula 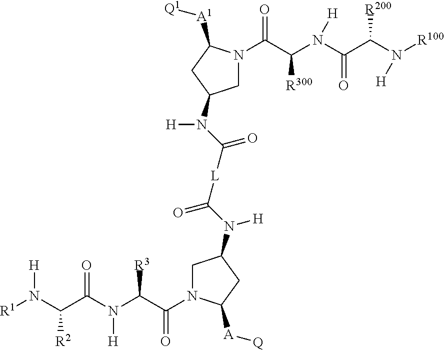
wherein L is
1) alkylene or cycloalkylene;
2) arylene or biphenylene; or
3) heteroarylene.

9. The compound according to claim 1, wherein R¹ and R¹⁰⁰ are both C₁-C₆ alkyl.

10. The compound according to claim 1, wherein R² and R²⁰⁰ are both C₁-C₆ alkyl.

11. The compound according to claim 1, wherein R³ and R³⁰⁰ are independently C₃-C₆ cycloalkyl or heterocyclyl, wherein the cycloalkyl or heterocyclyl is optionally substituted with one or more R⁶ substituents.

12. The compound according to claim 1, wherein Q is NR⁴R⁵ and Q¹ is NR⁴⁰⁰R⁵⁰⁰.

13. The compound according to claim 1, wherein A and A¹ are both C═O, Q is NR⁴R⁵, Q¹ is NR⁴⁰⁰R⁵⁰⁰, R⁴ and R⁴⁰⁰ is H, and R⁵ and R⁵⁰⁰ are independently:
1) —C₁-C₆ alkyl,
2) —C₃-C₇ cycloalkyl
3)-heterocyclyl, or
4)-heterobicyclyl,
wherein the alkyl, cycloalkyl, heterocyclyl, and heterobicyclyl are optionally substituted with one or more R⁶ substituents.

14. The compound according to claim 13, wherein R⁵ and R⁵⁰⁰ are independently:

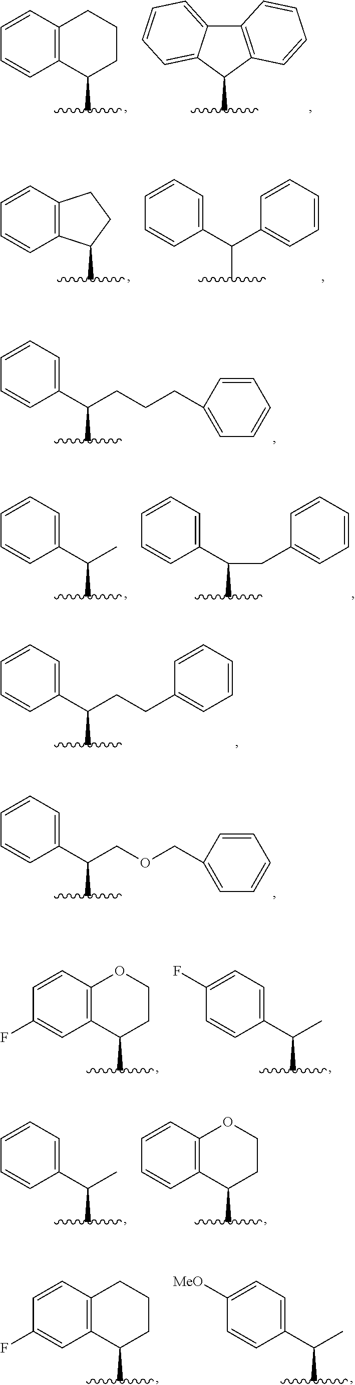

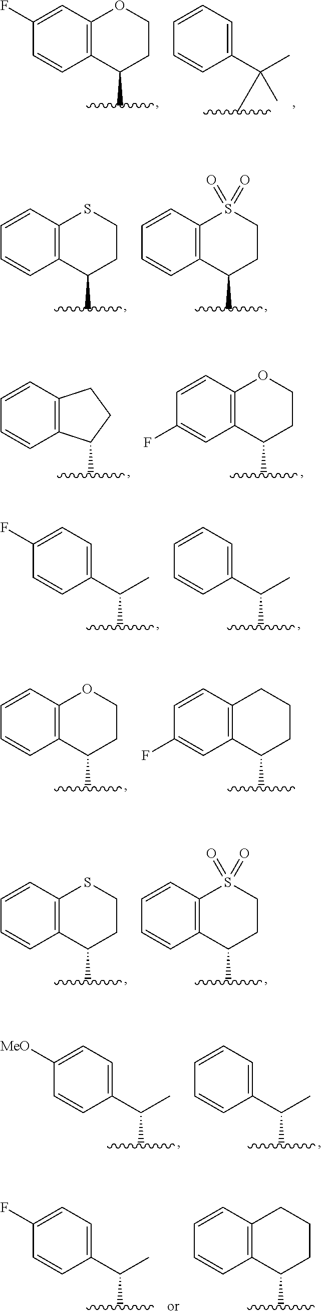

15-16. (canceled)

17. A compound according to claim 1, wherein the compound is:

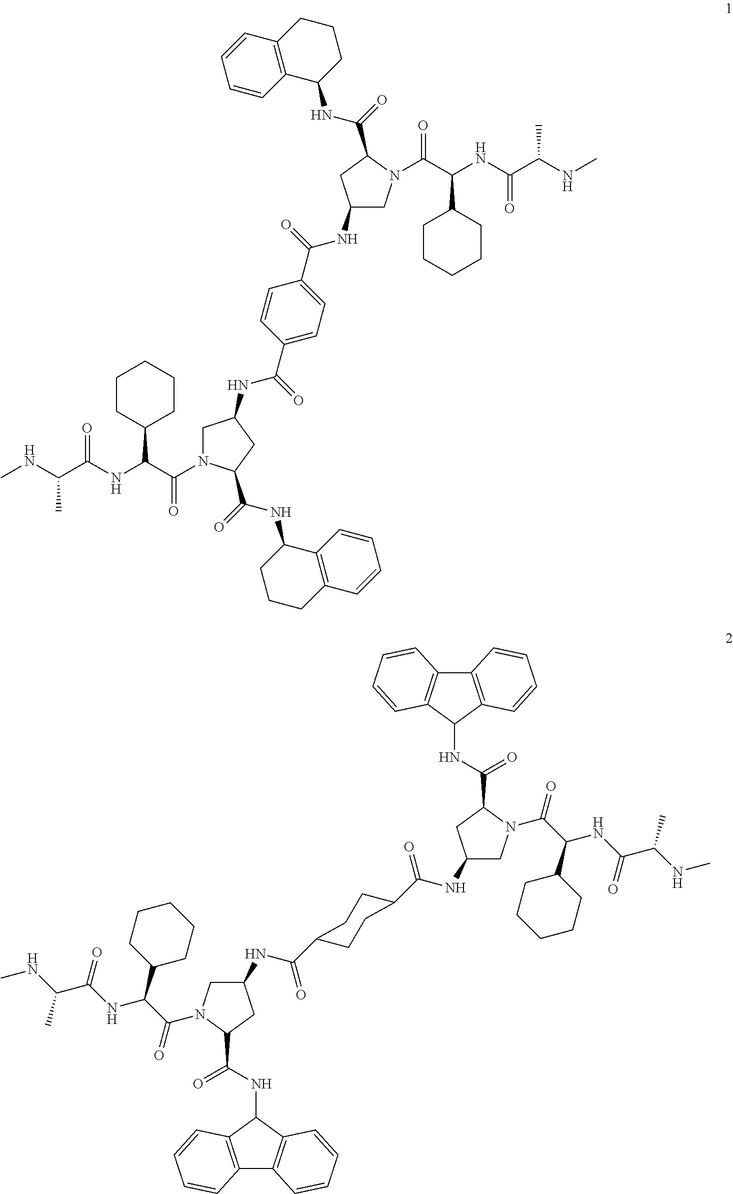

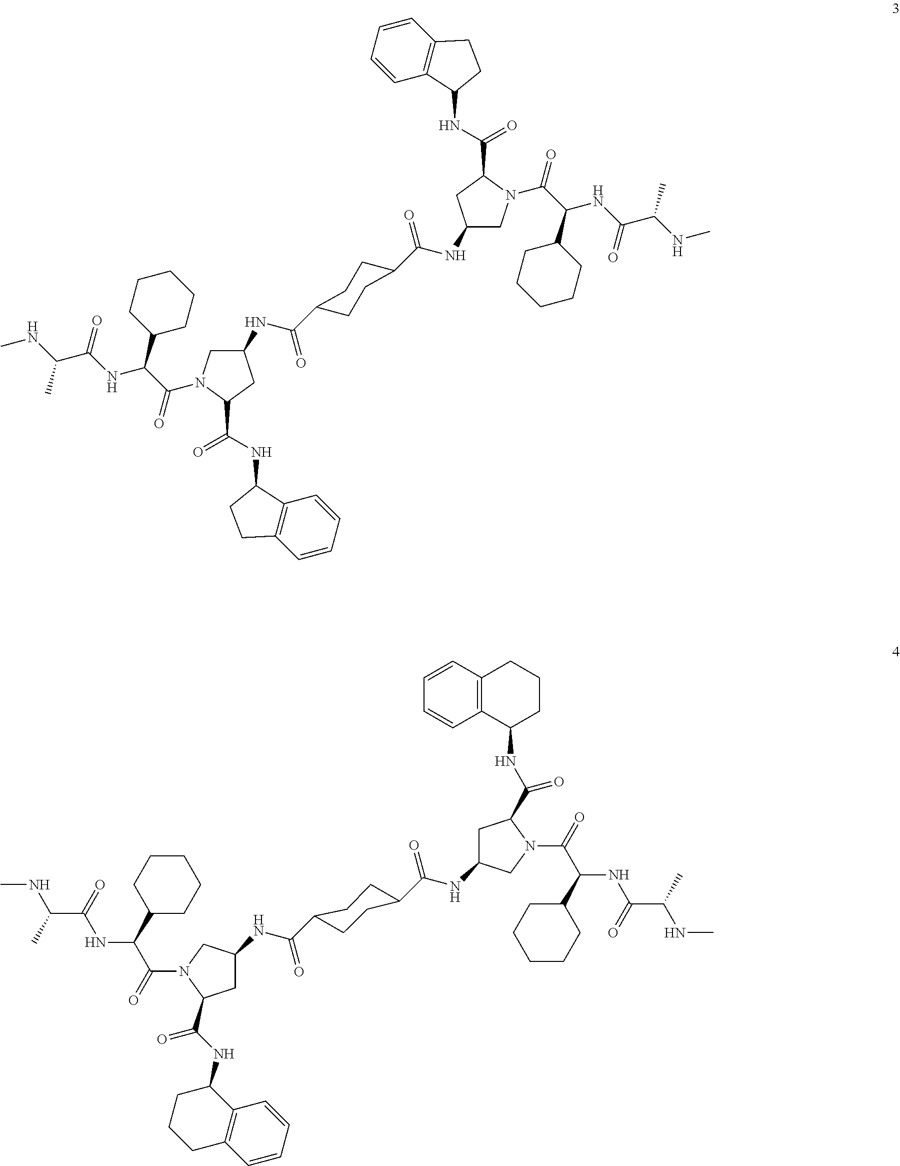

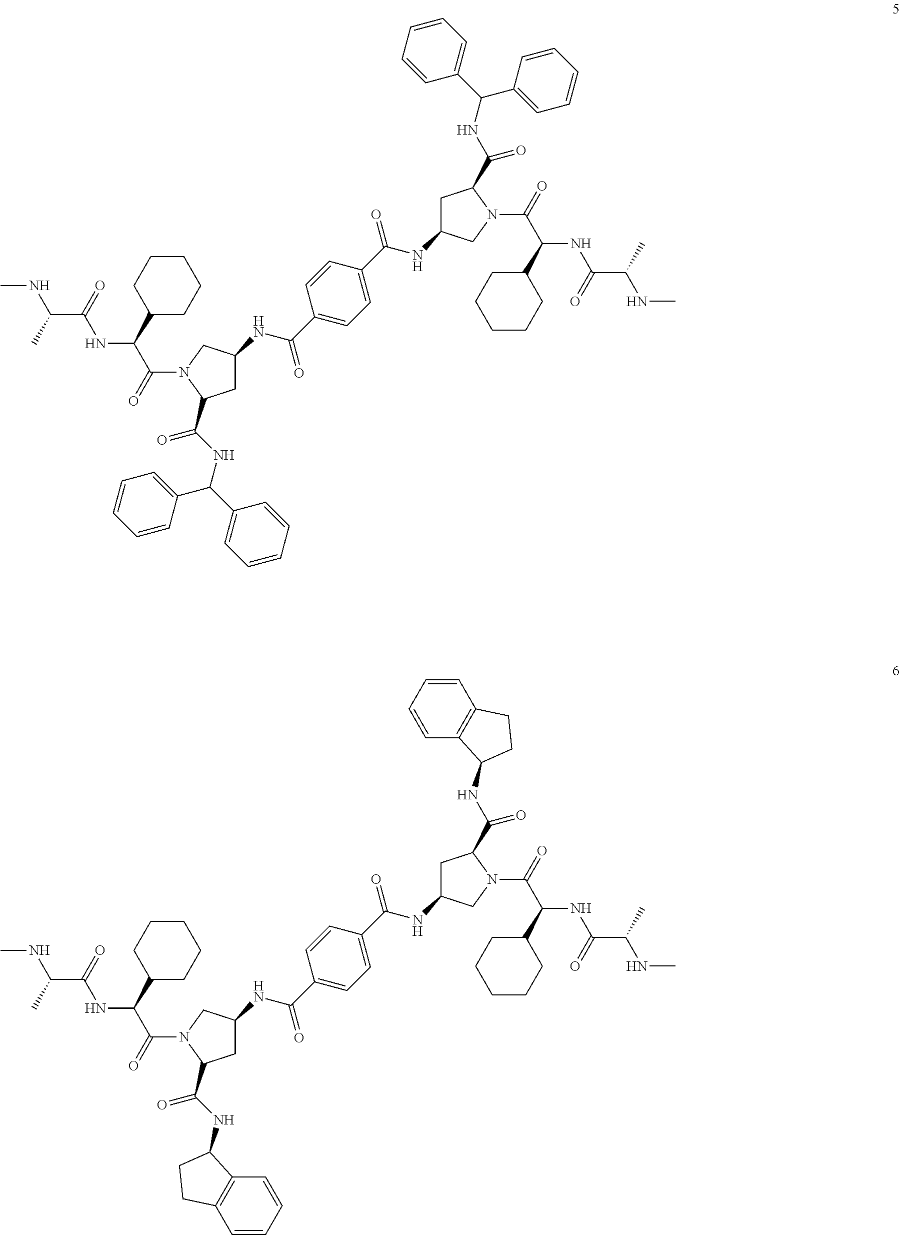

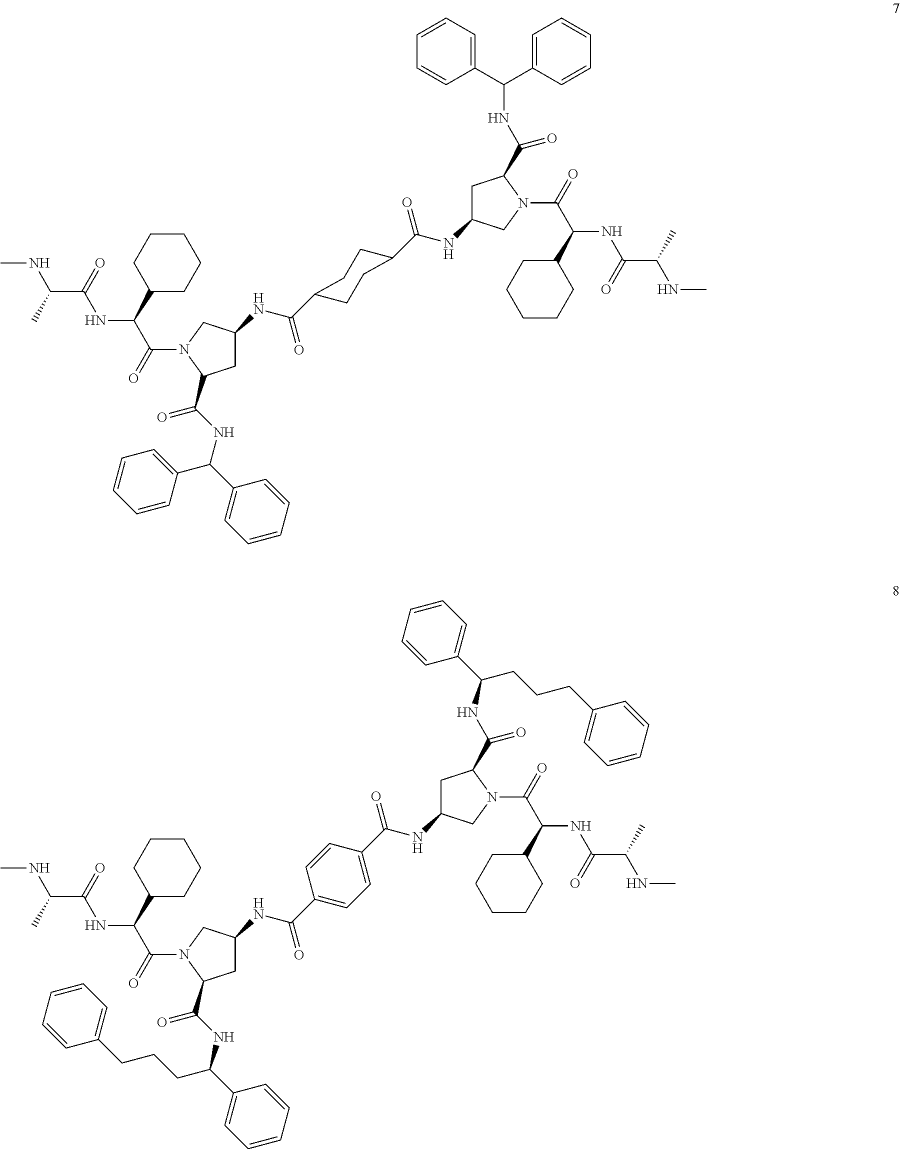

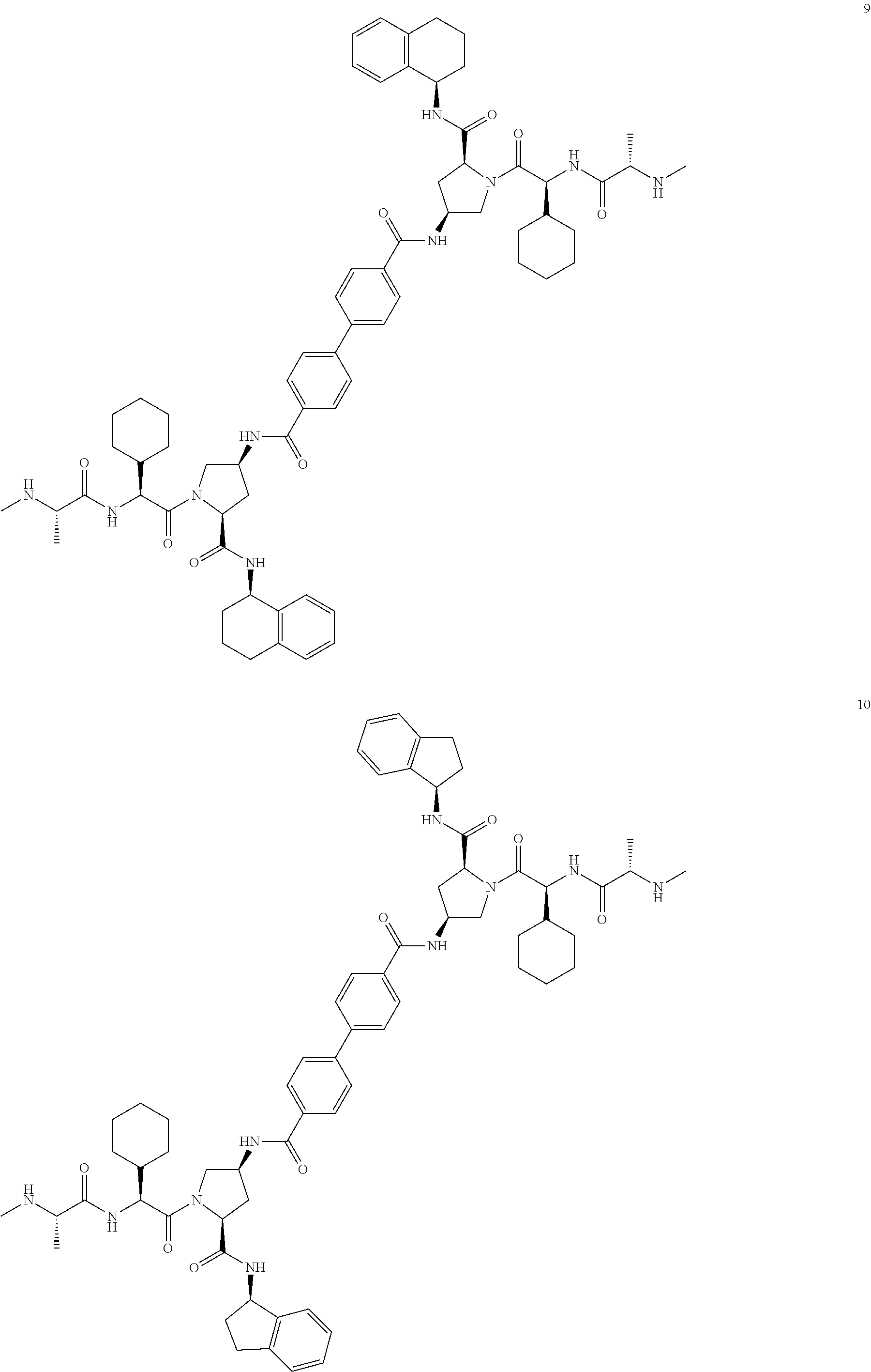

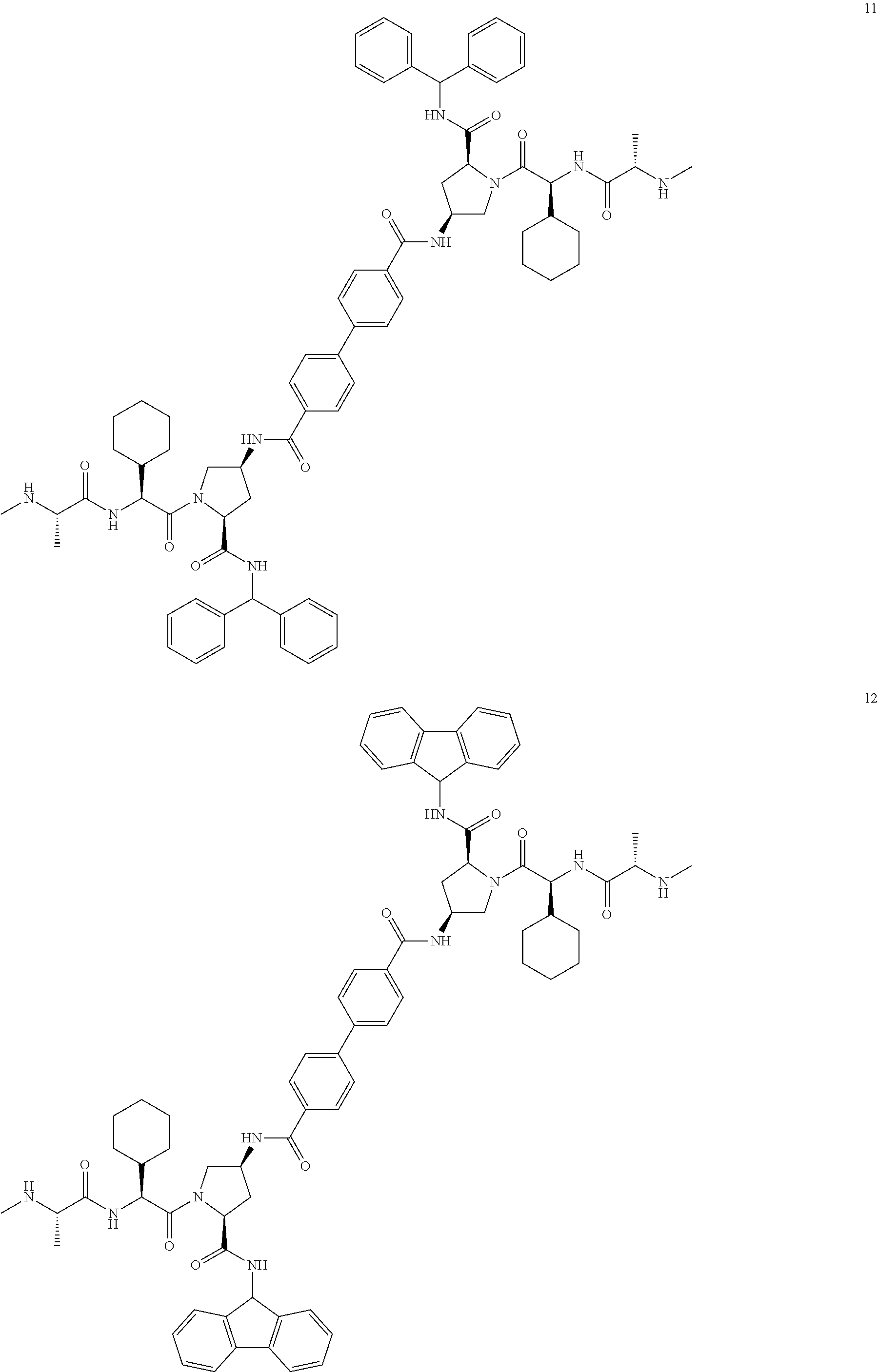

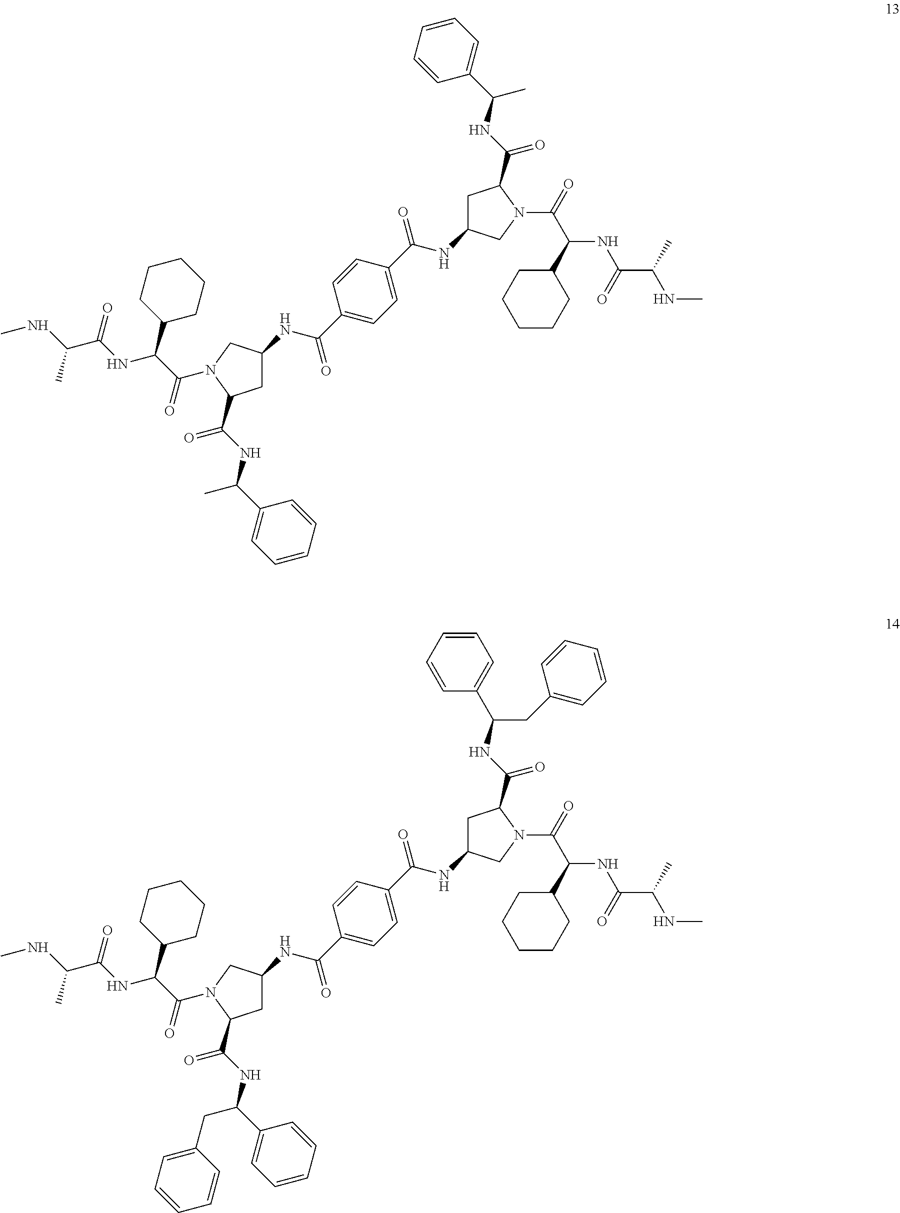

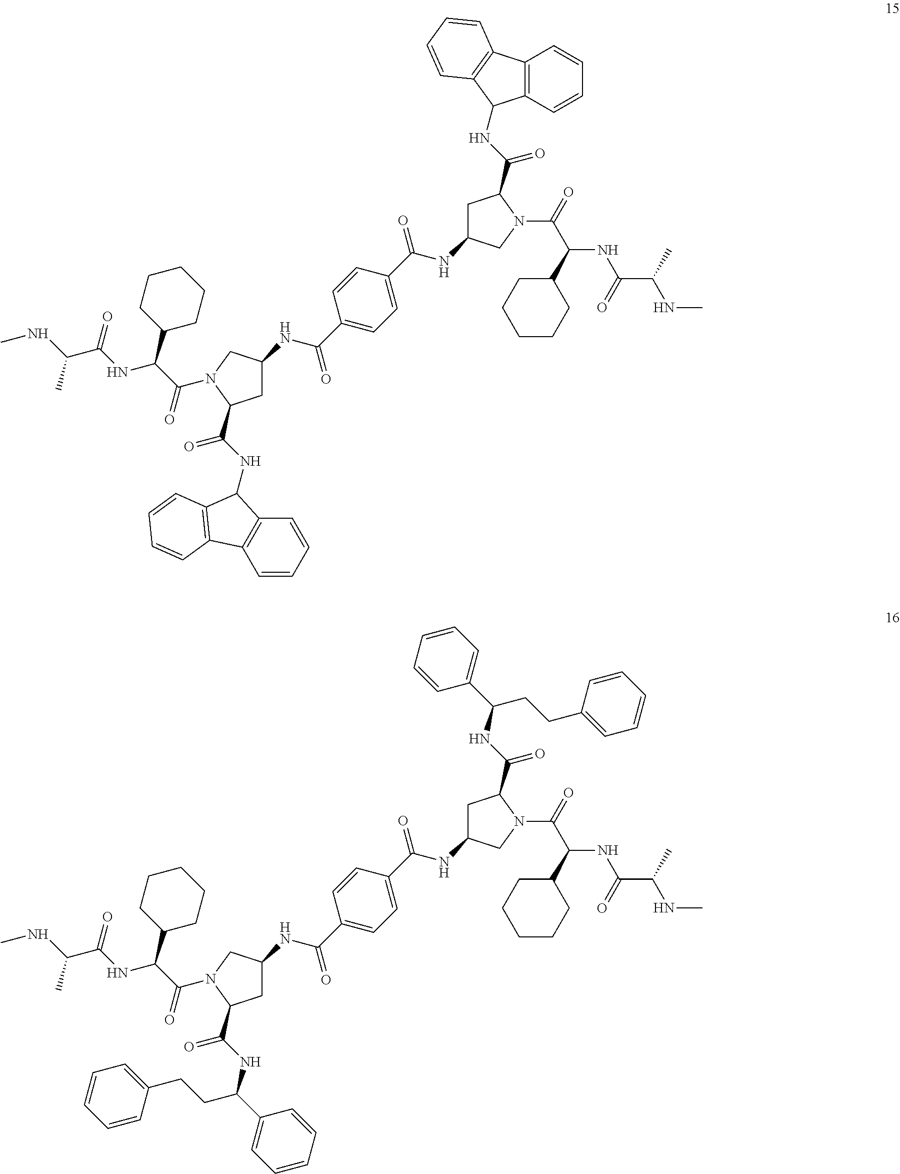

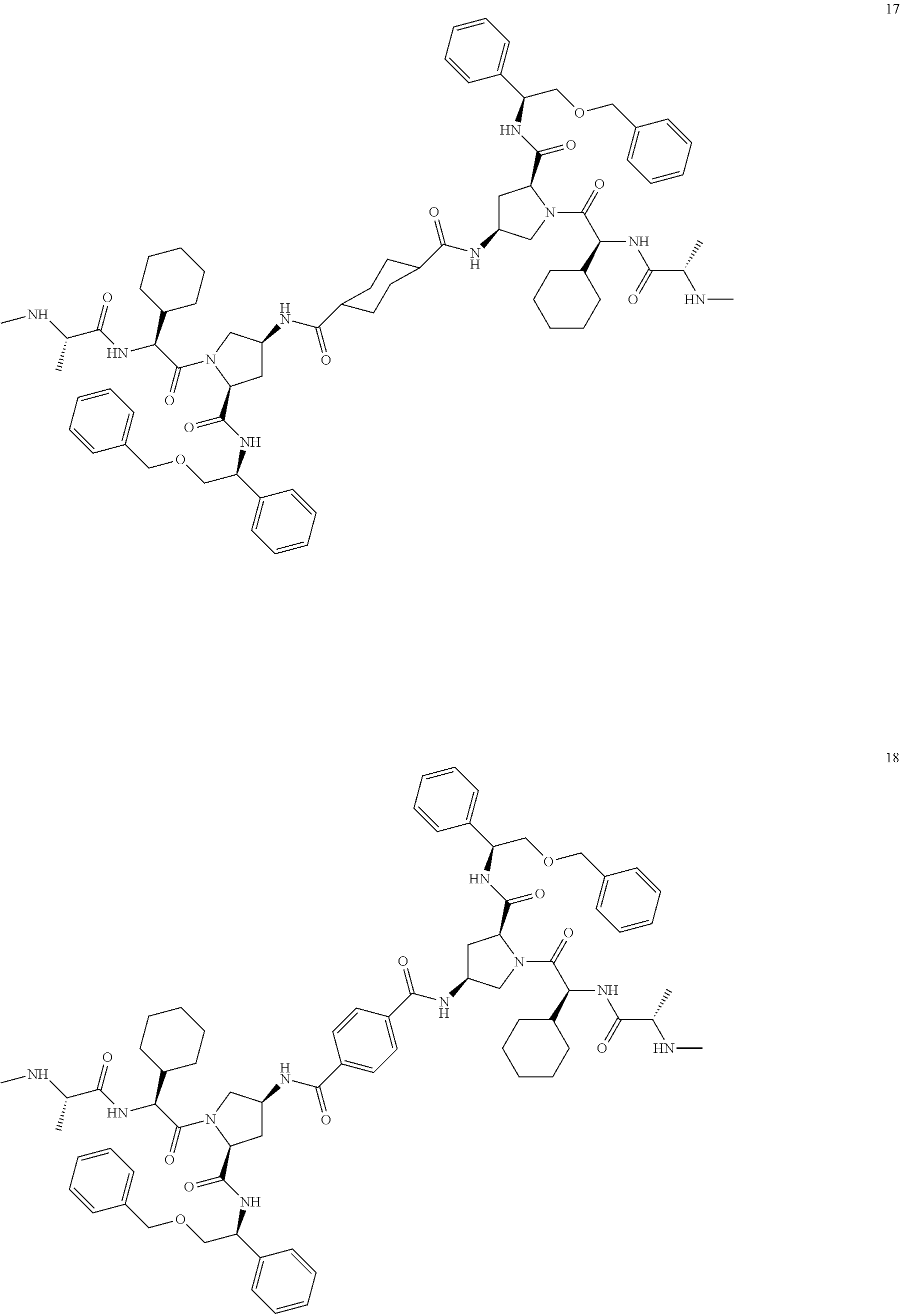

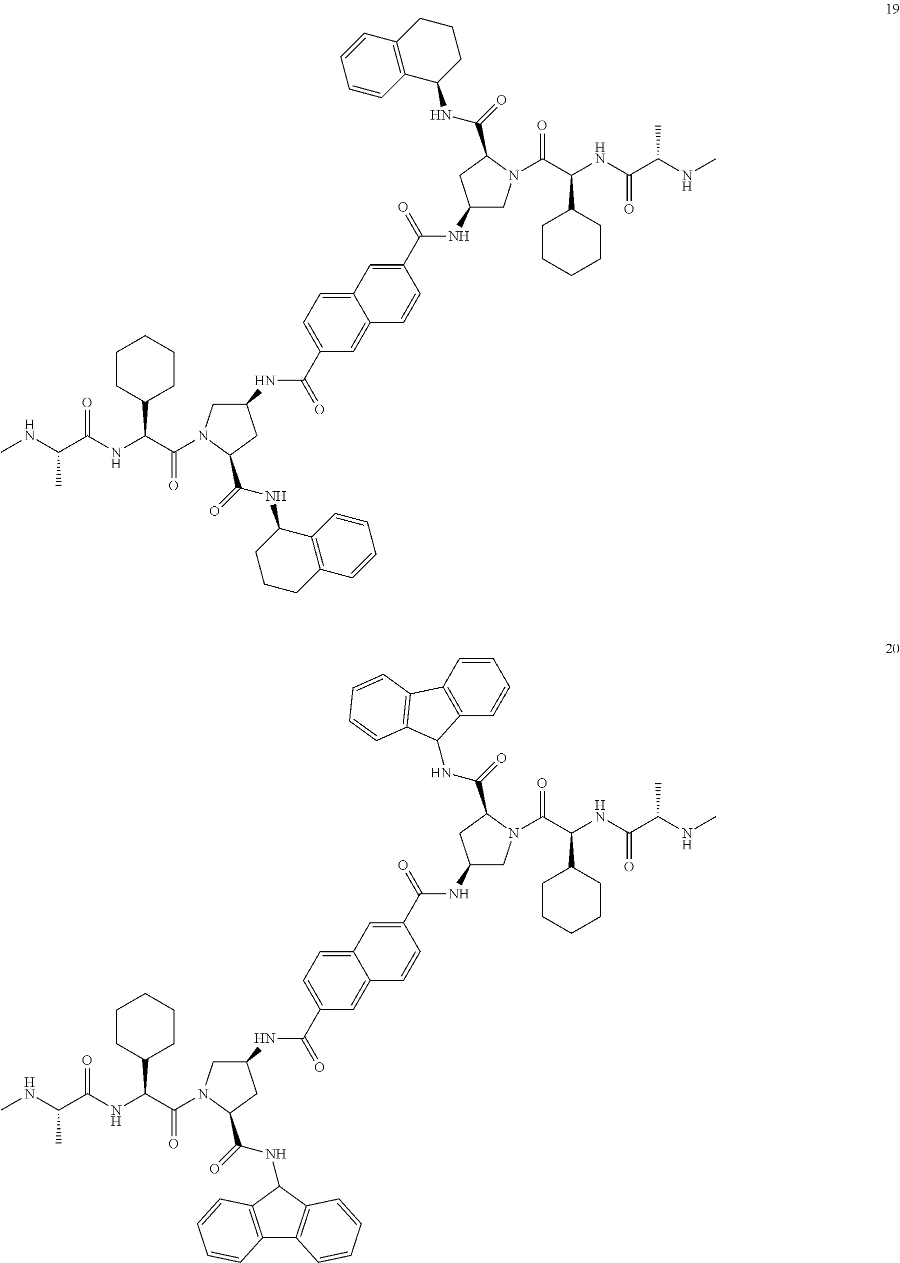

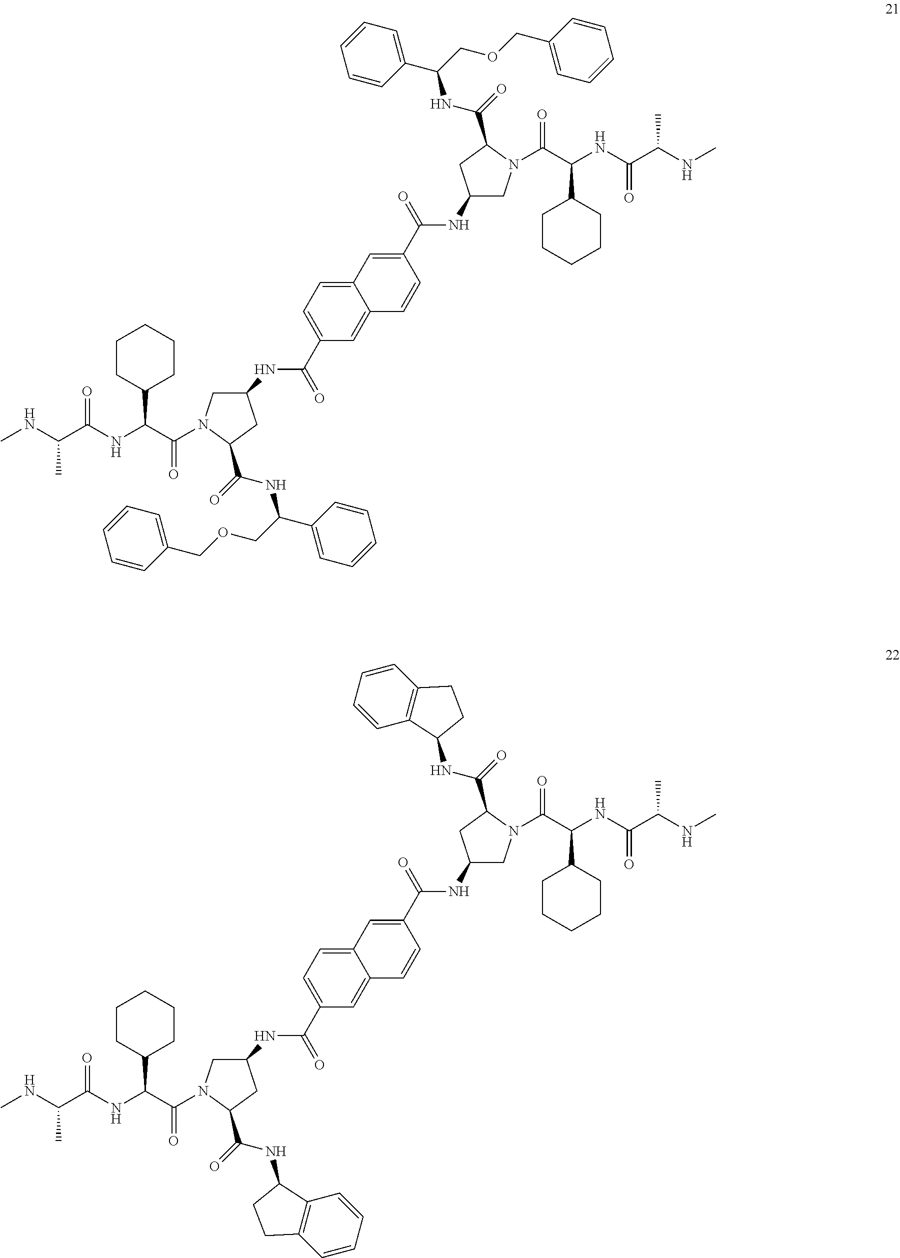

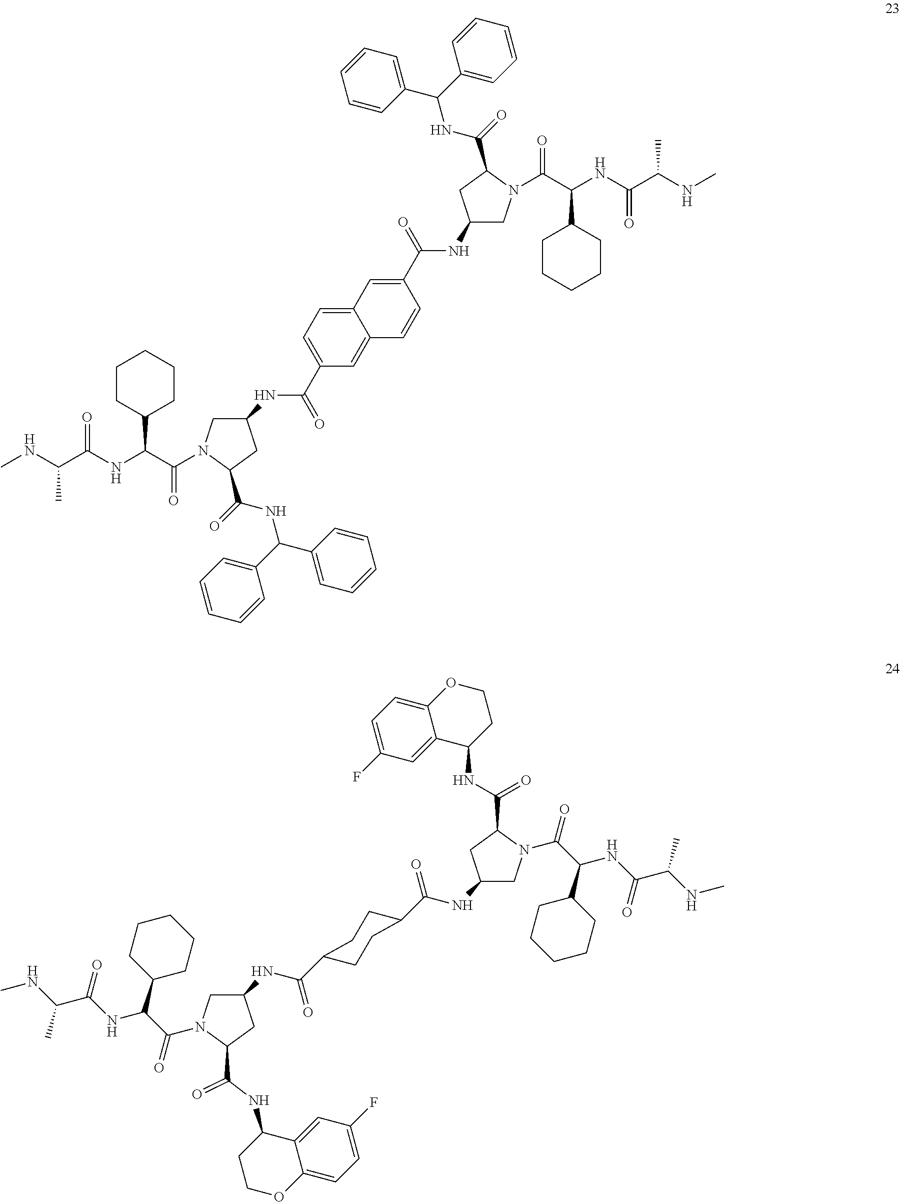

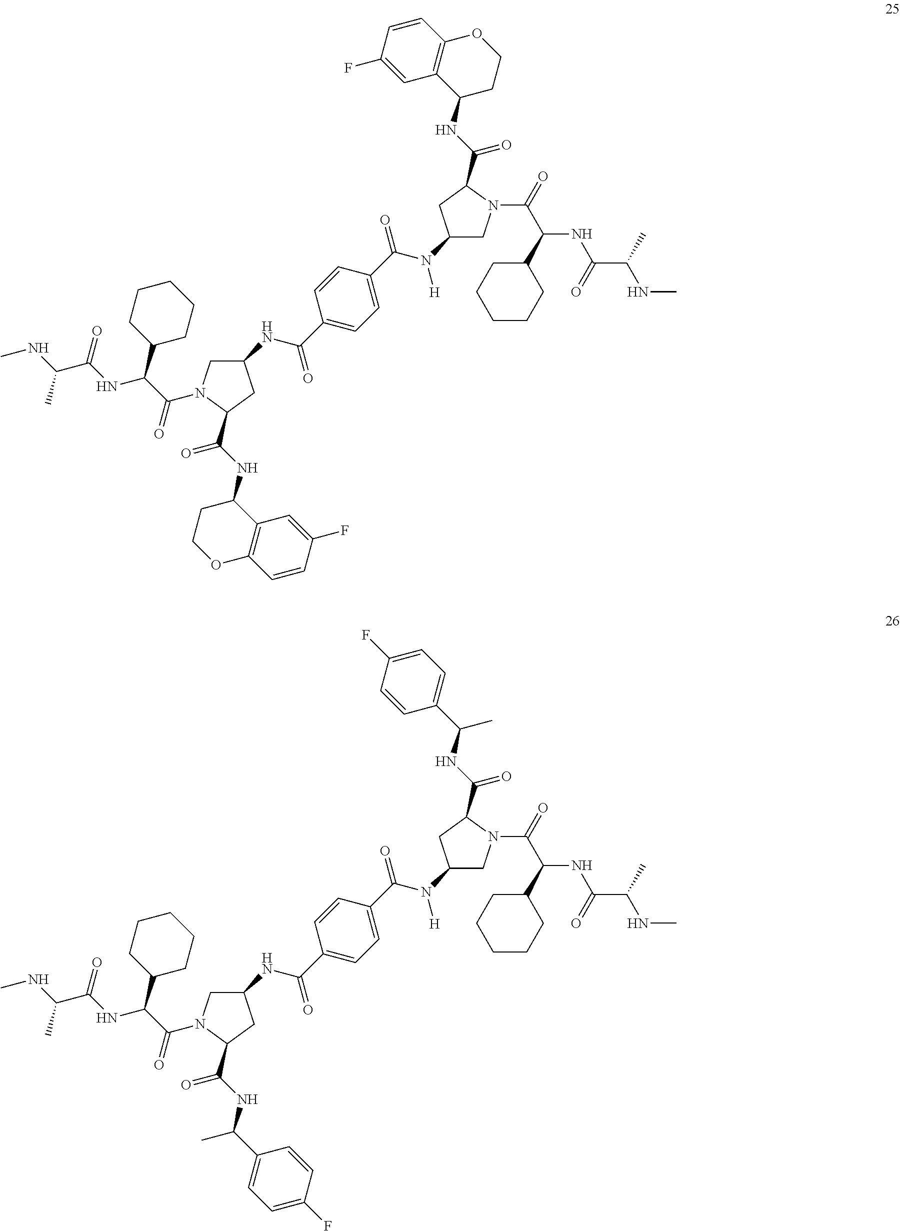

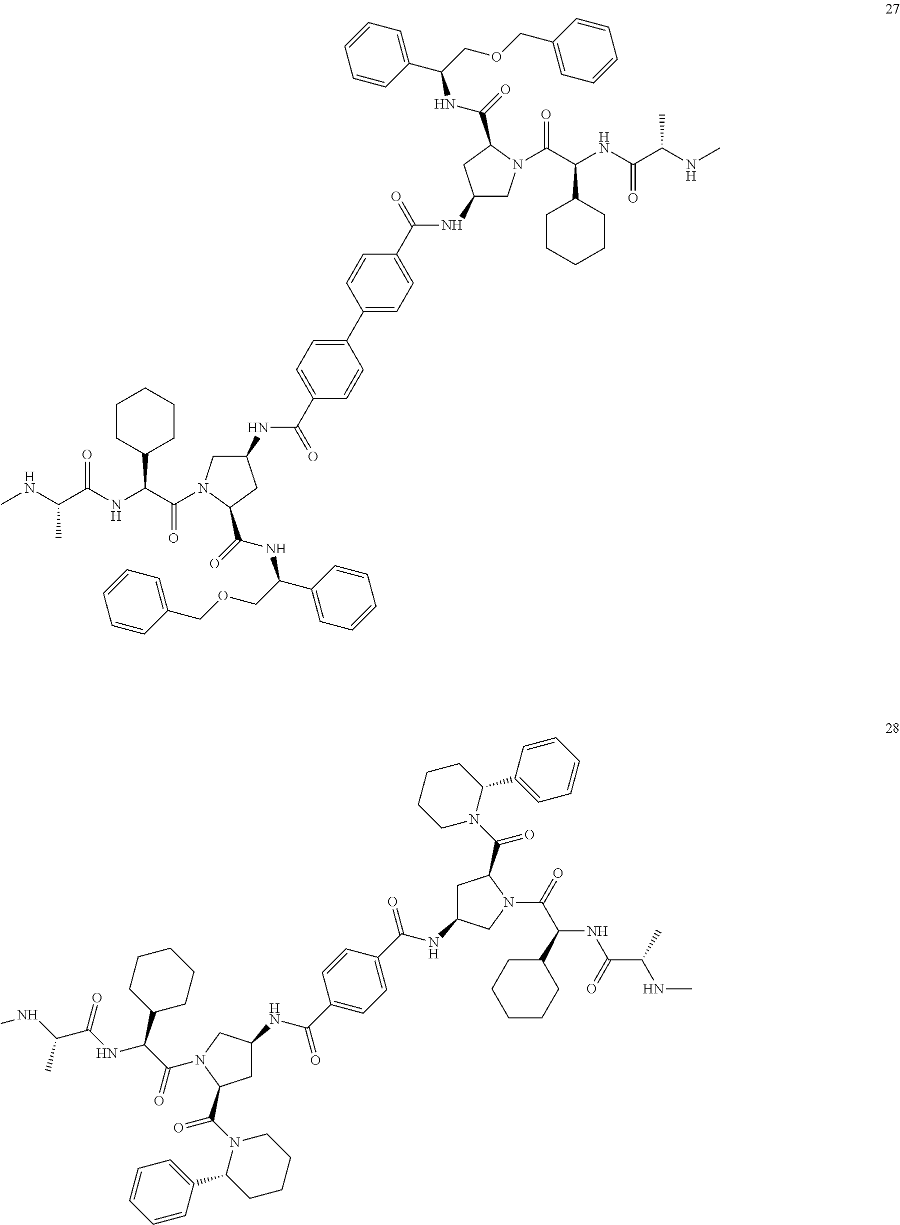

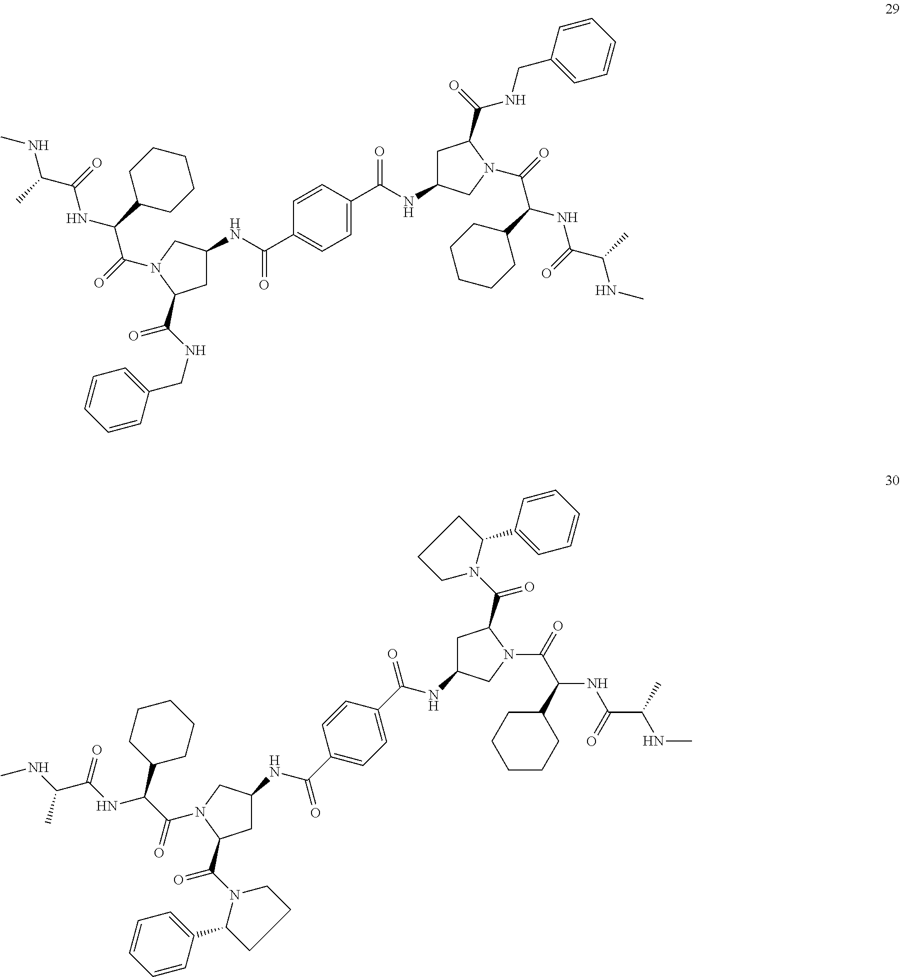

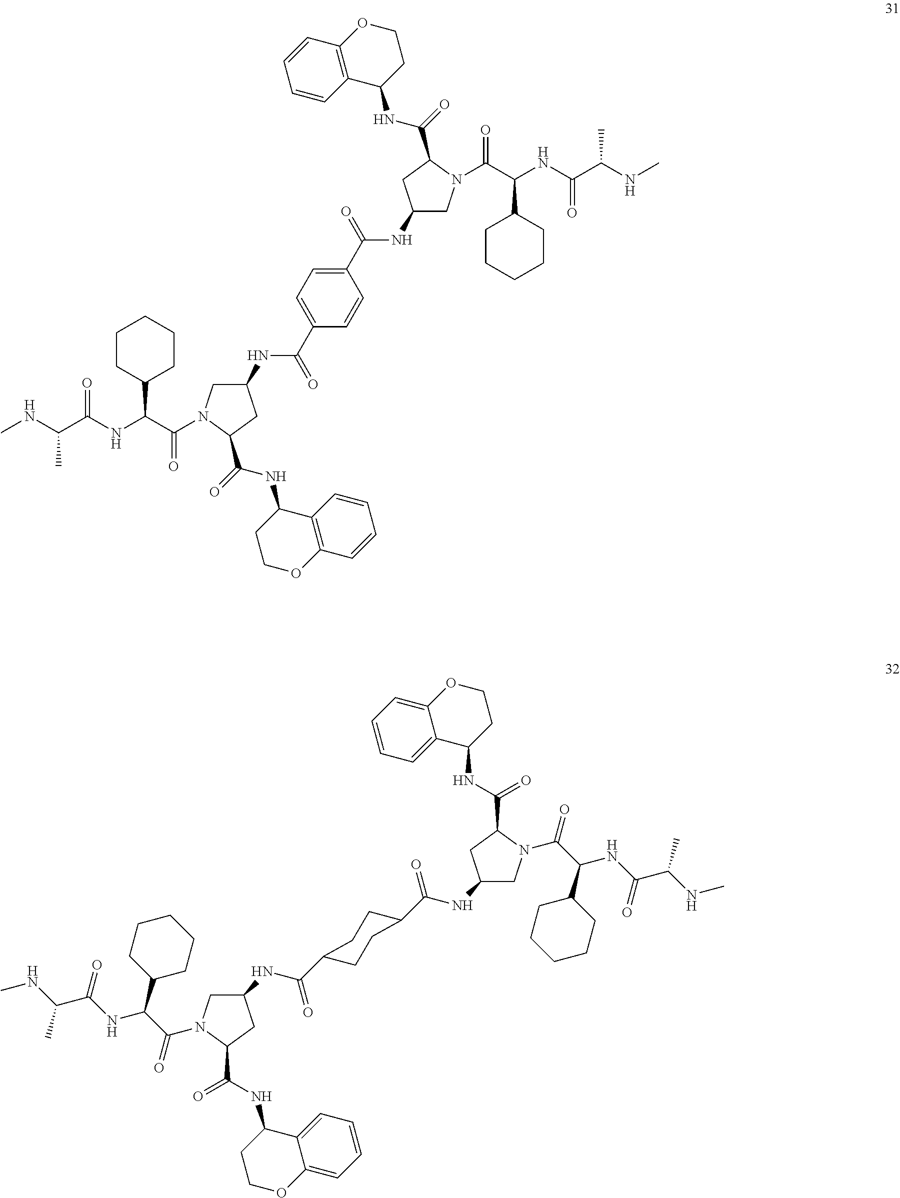

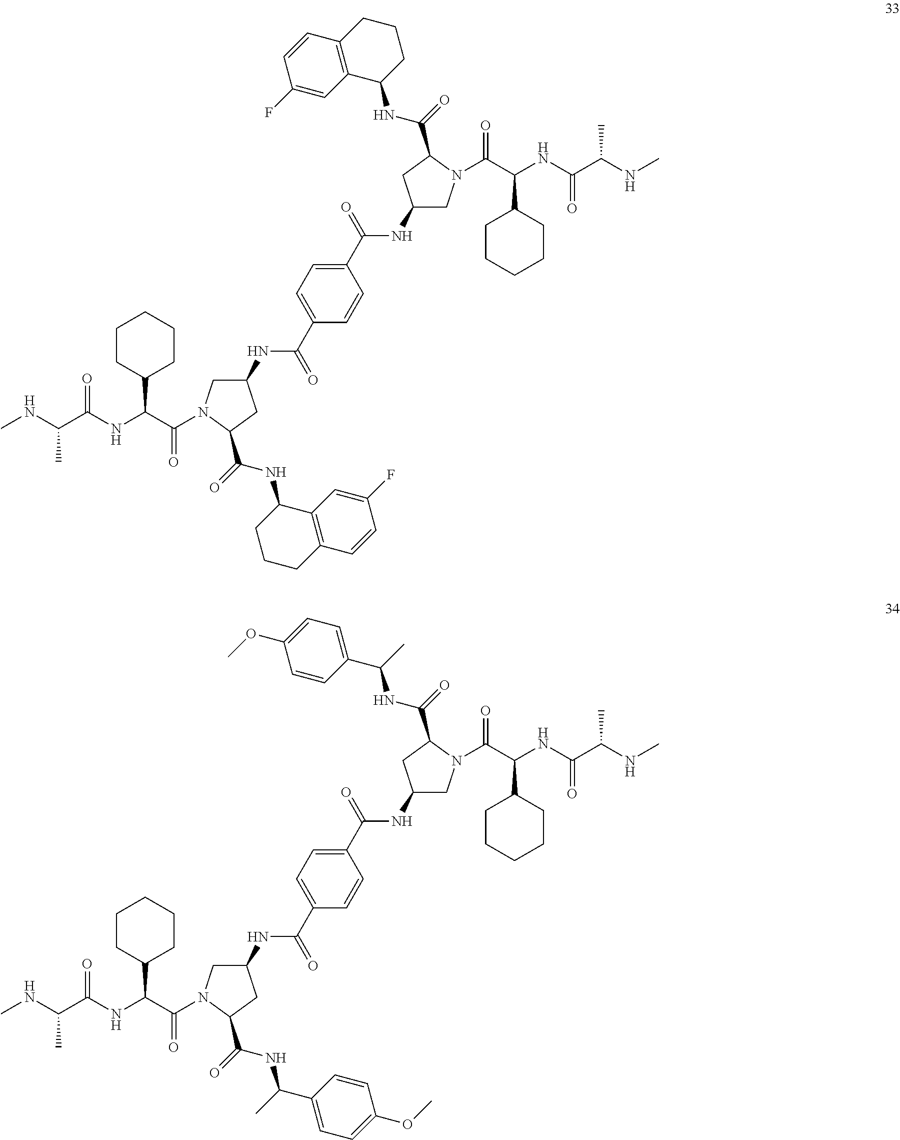

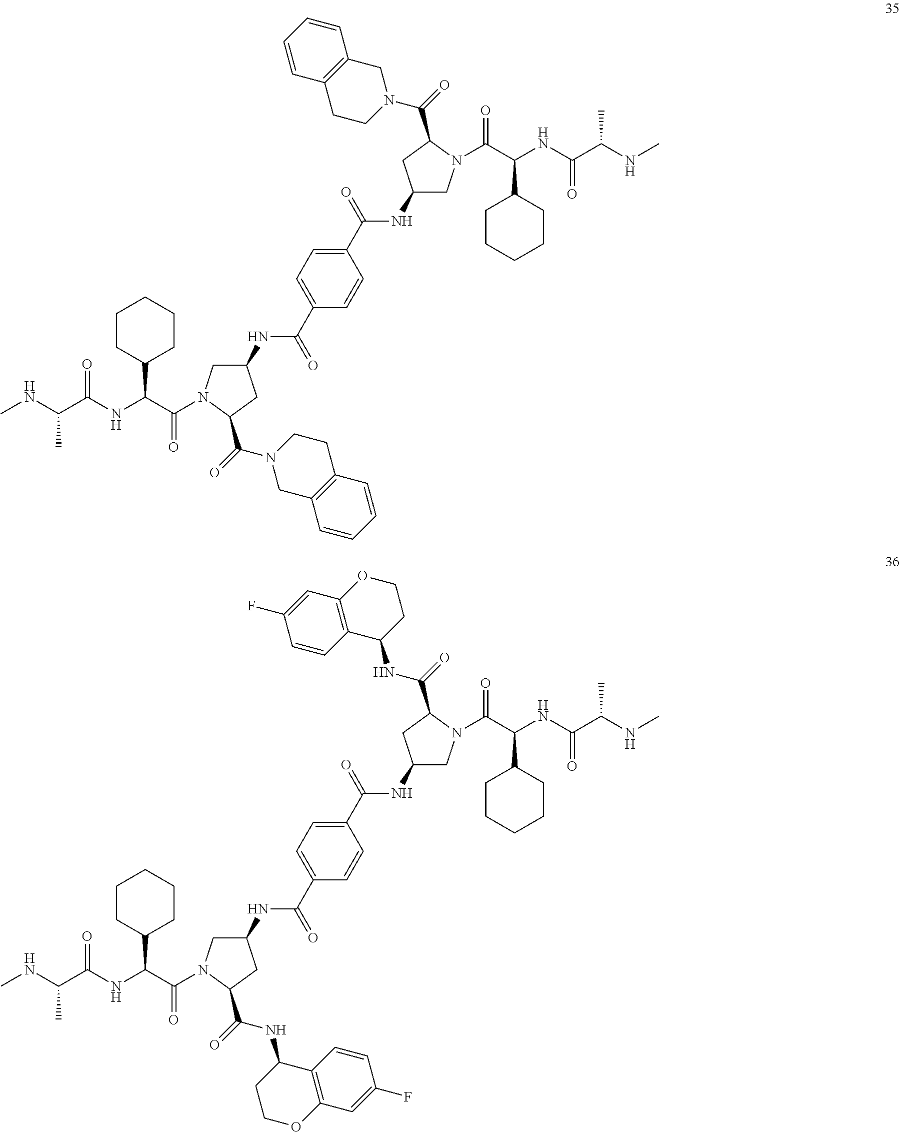

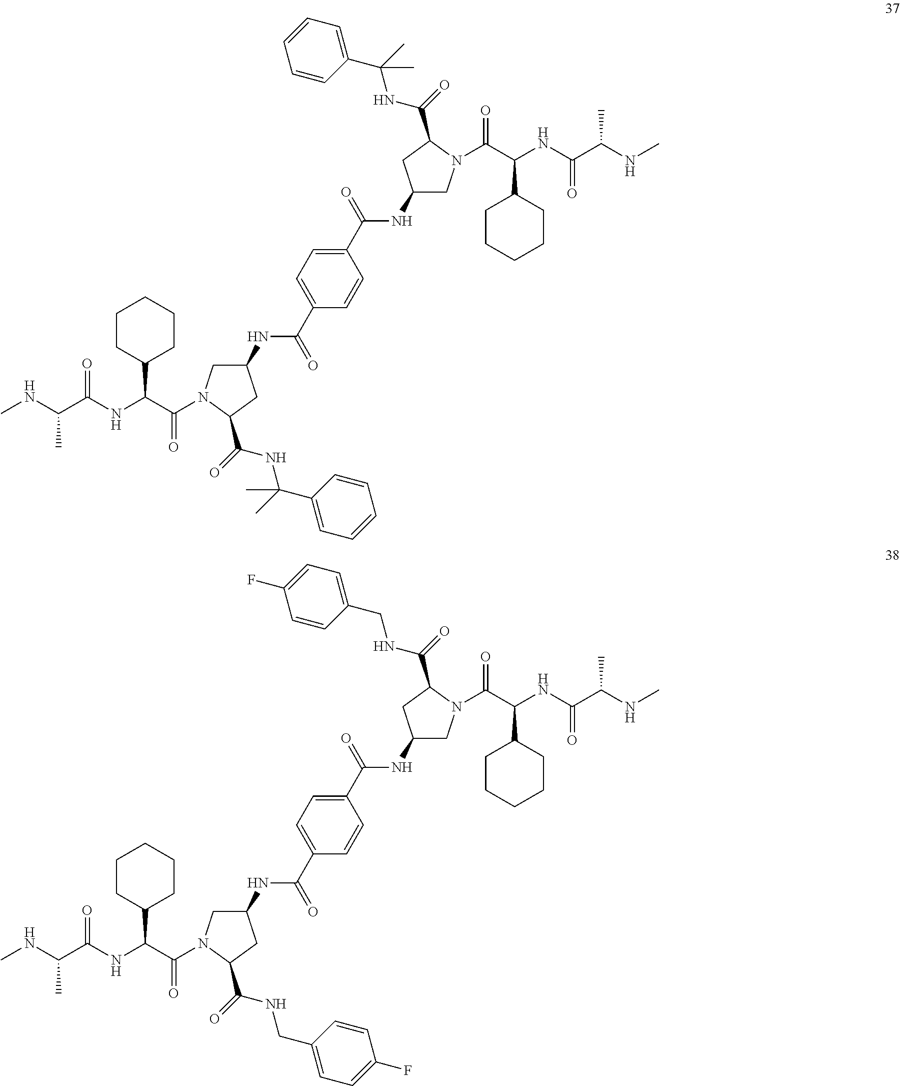

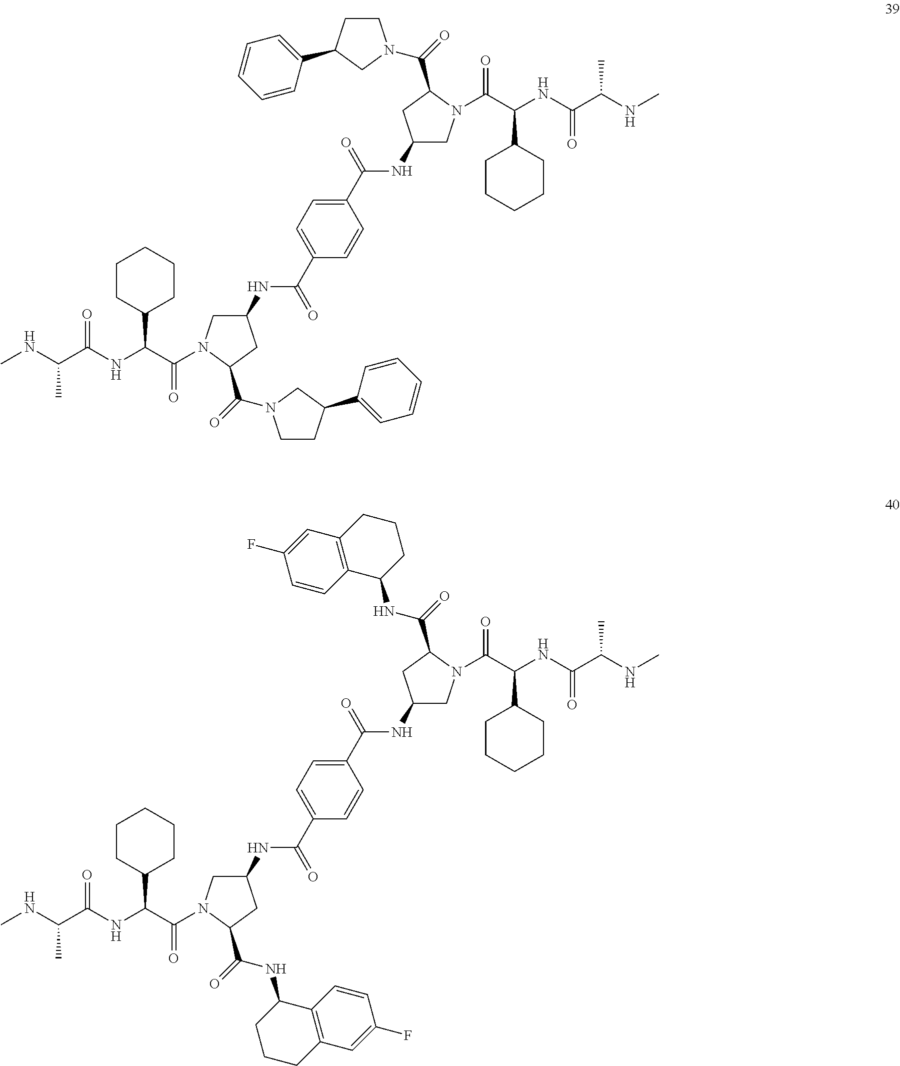

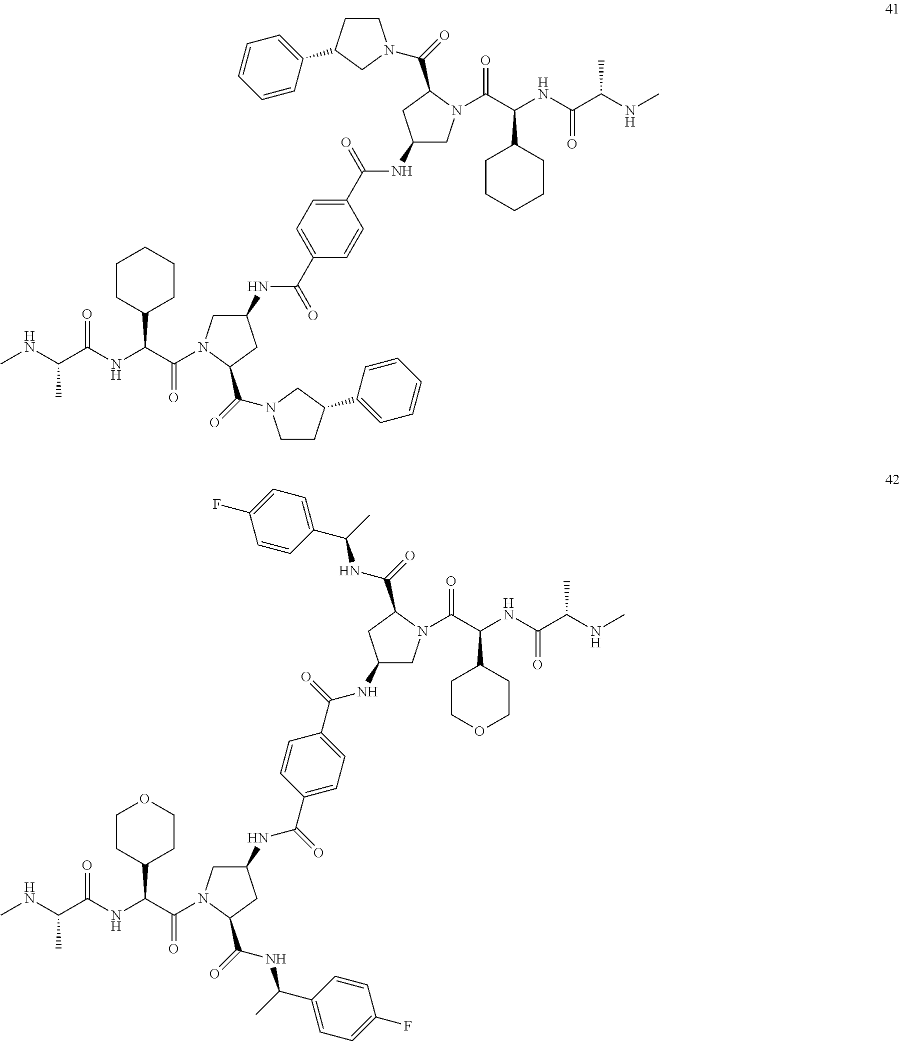

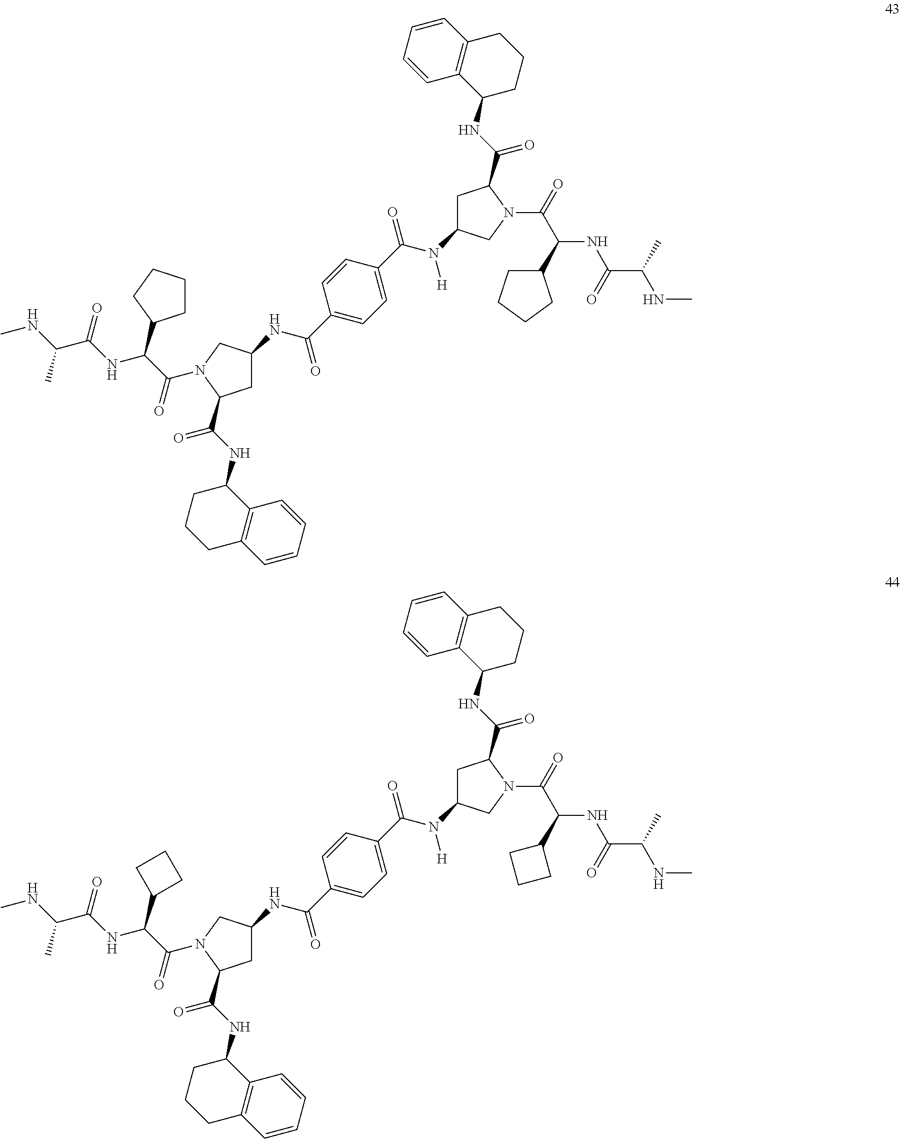

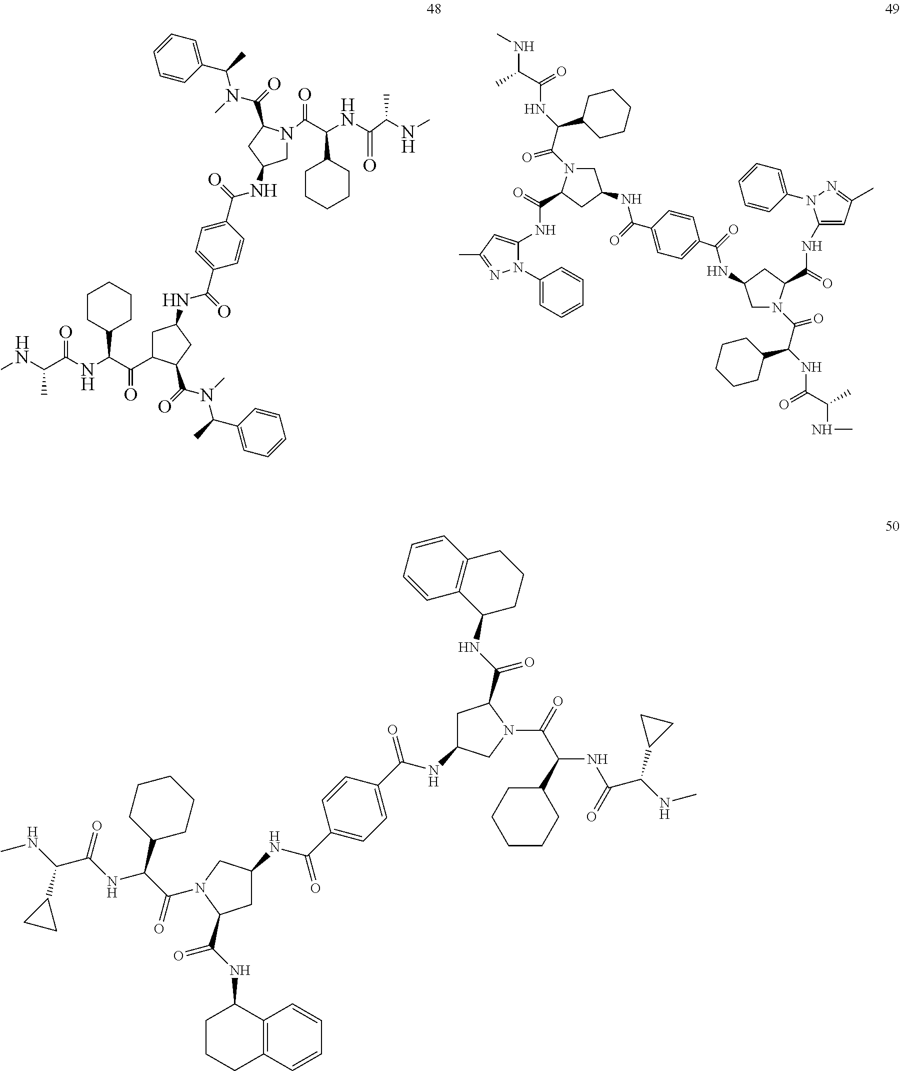

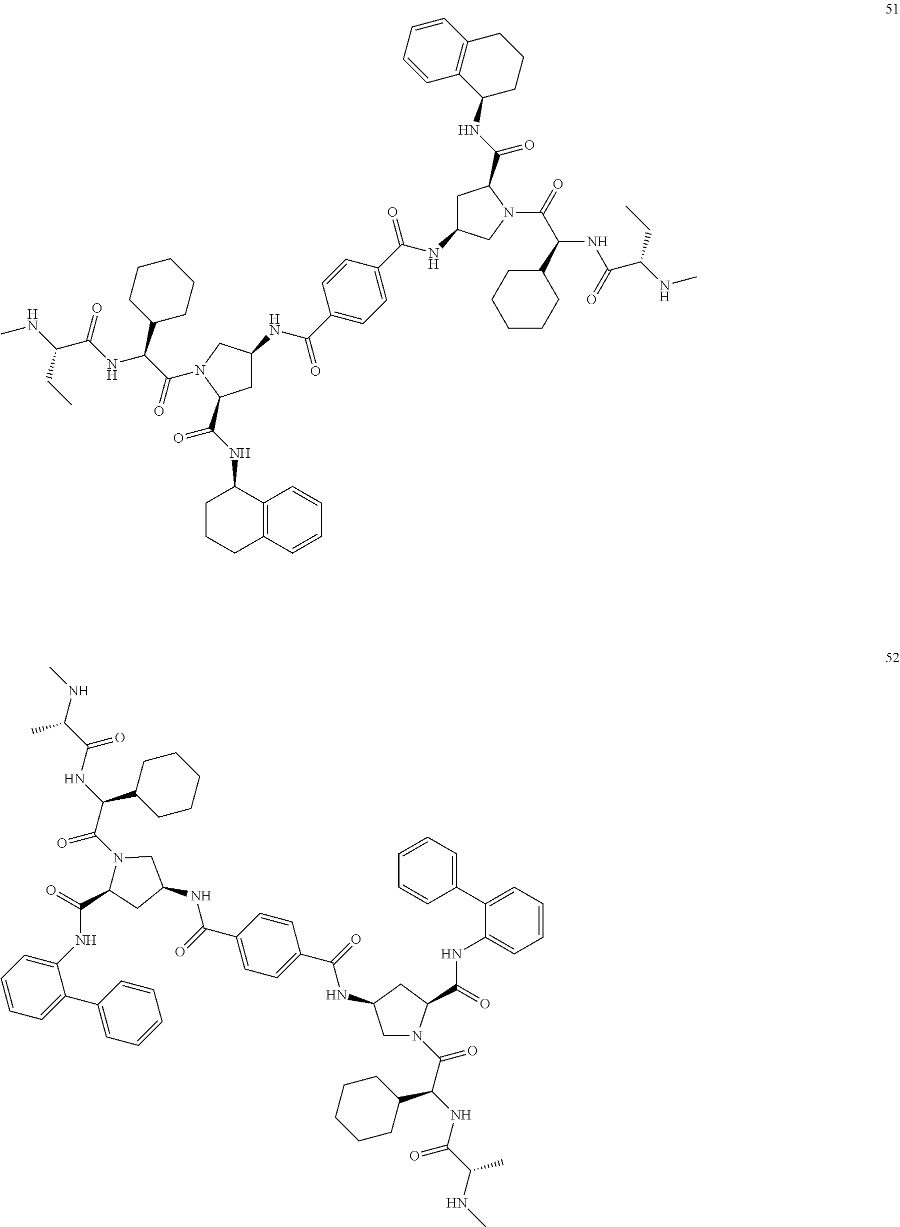

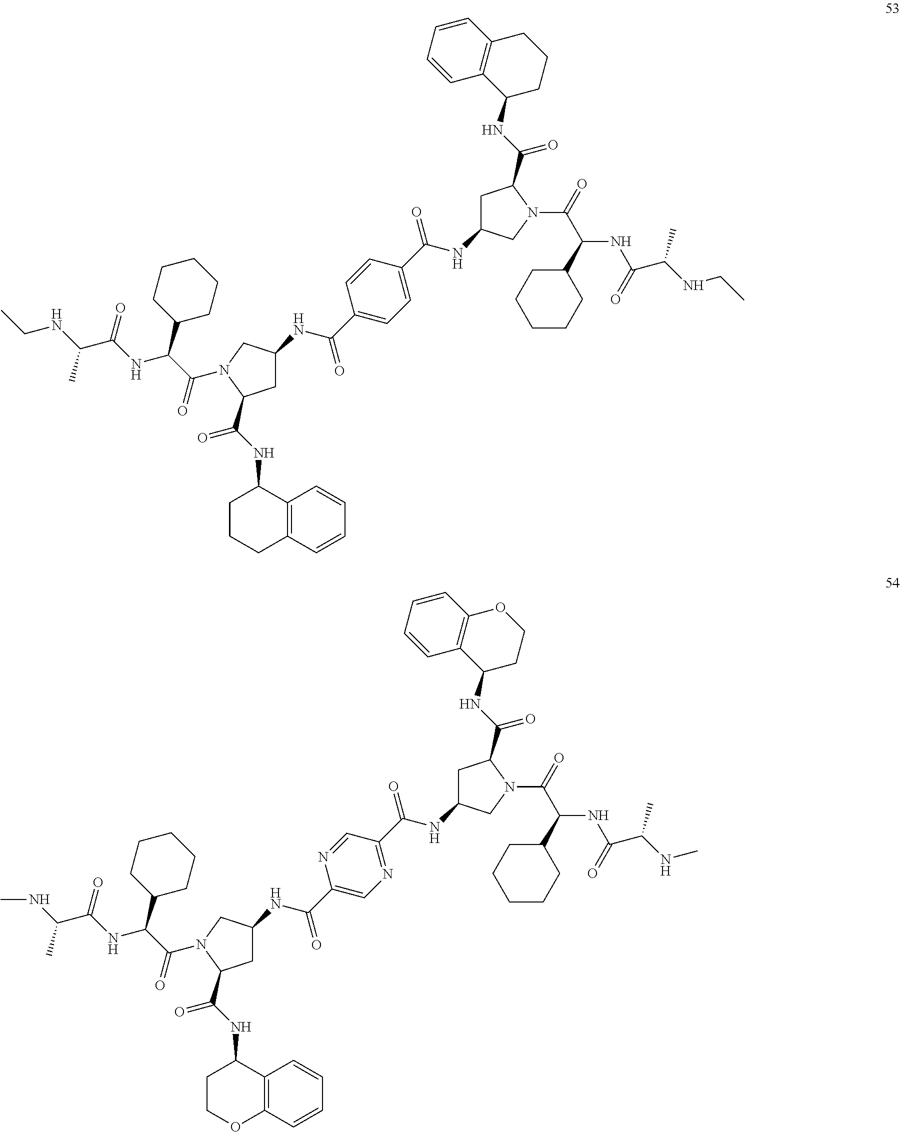

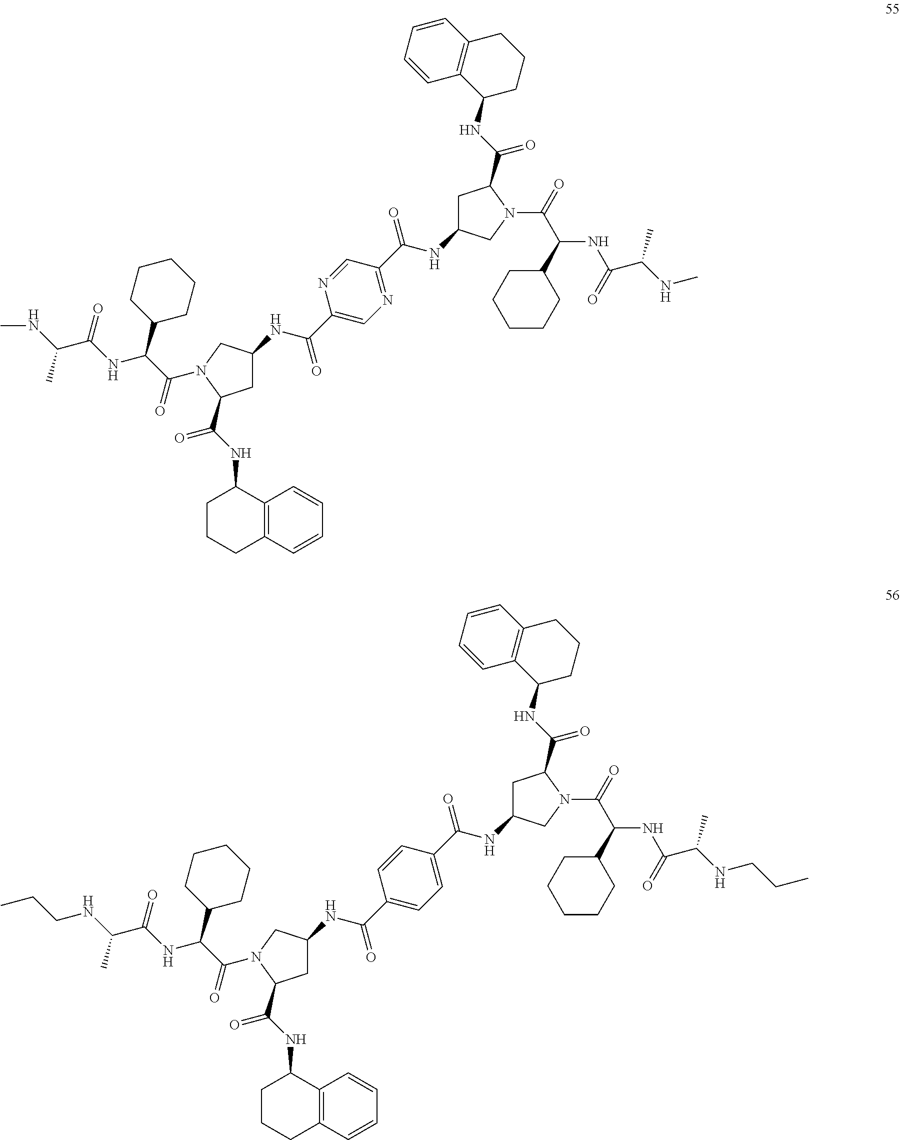

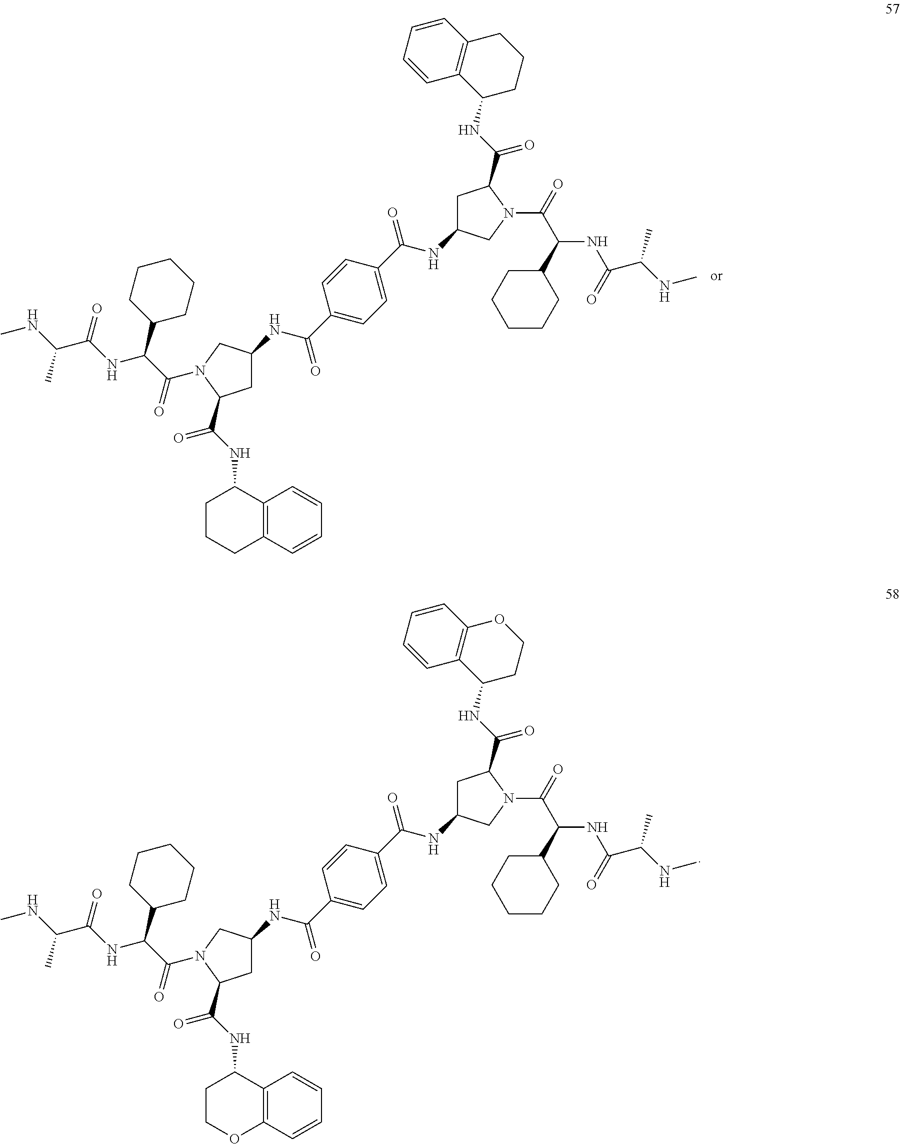

18. A compound according to claim 1, wherein the compound is:

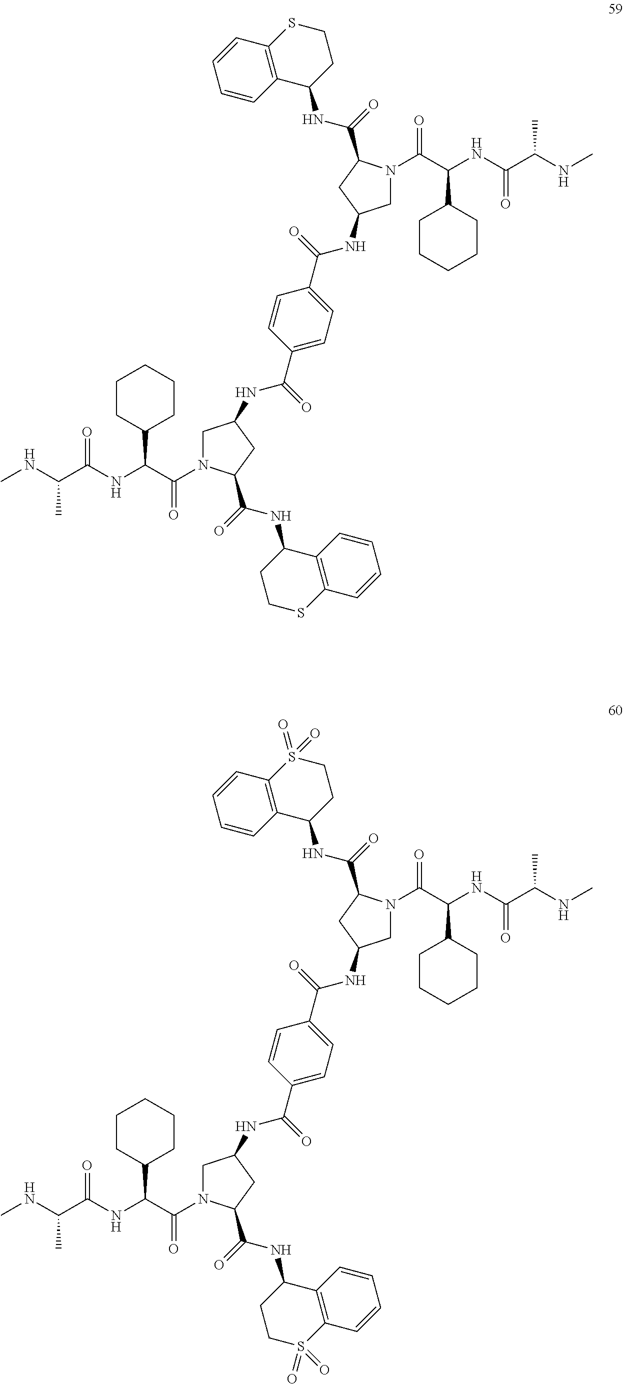

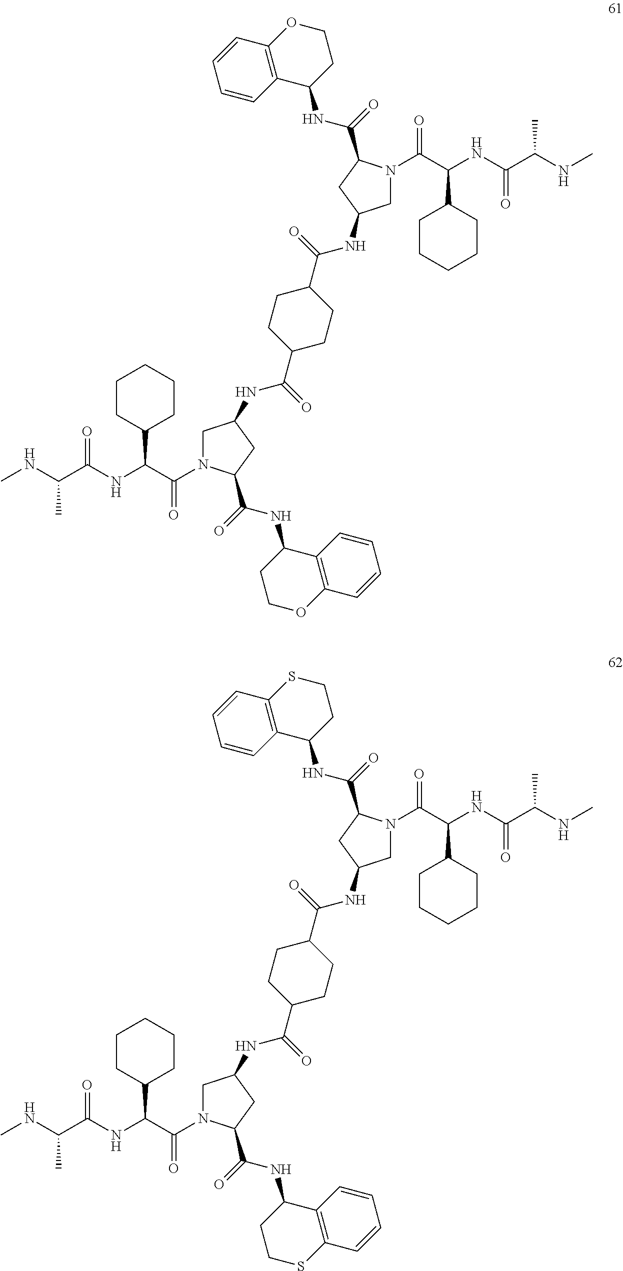

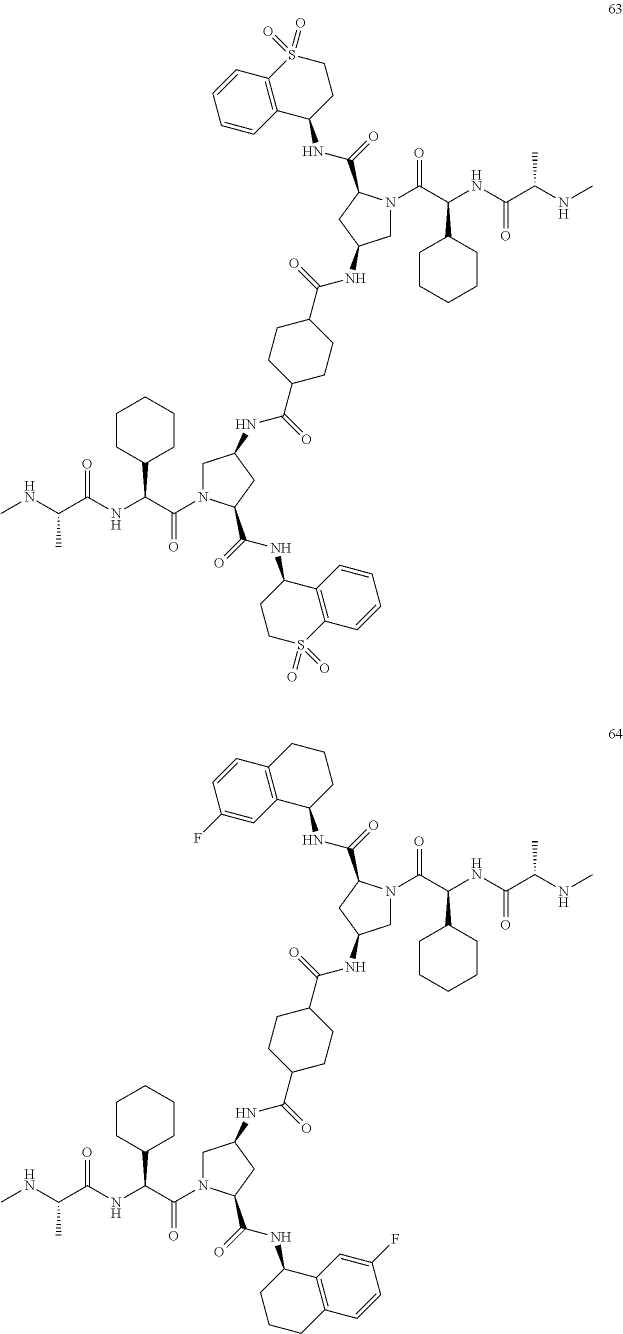

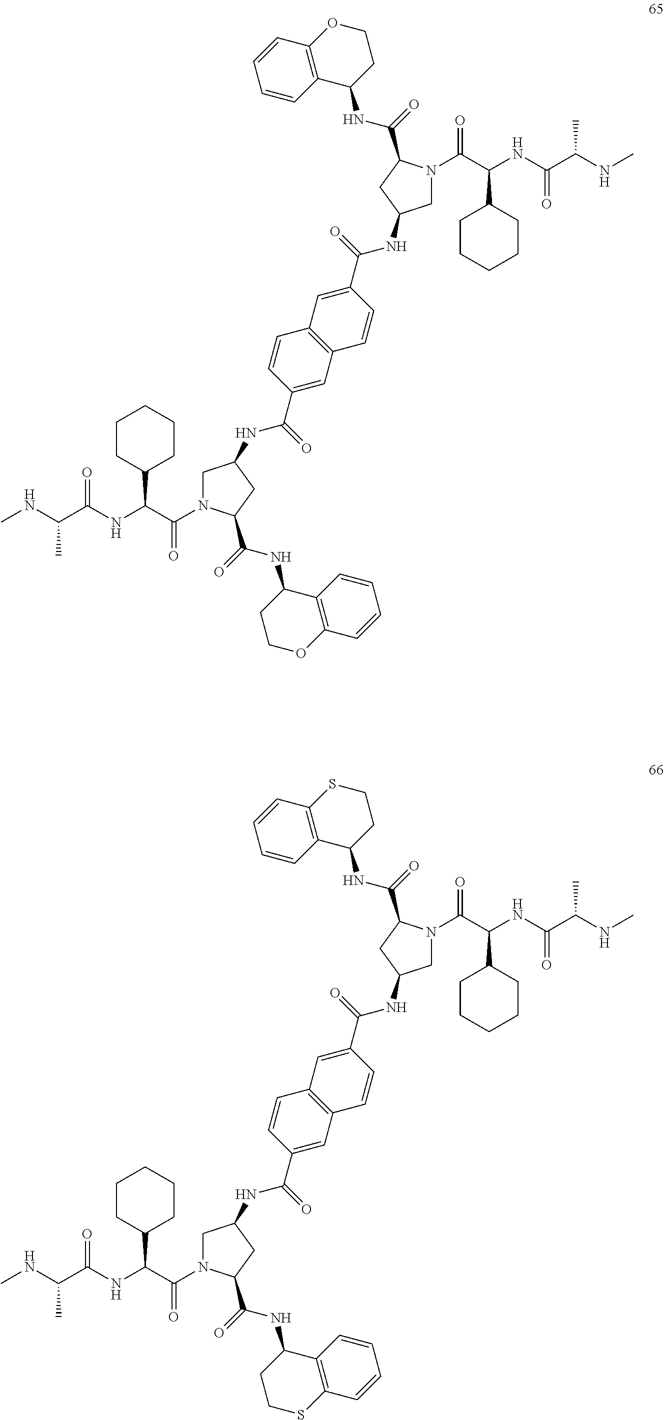

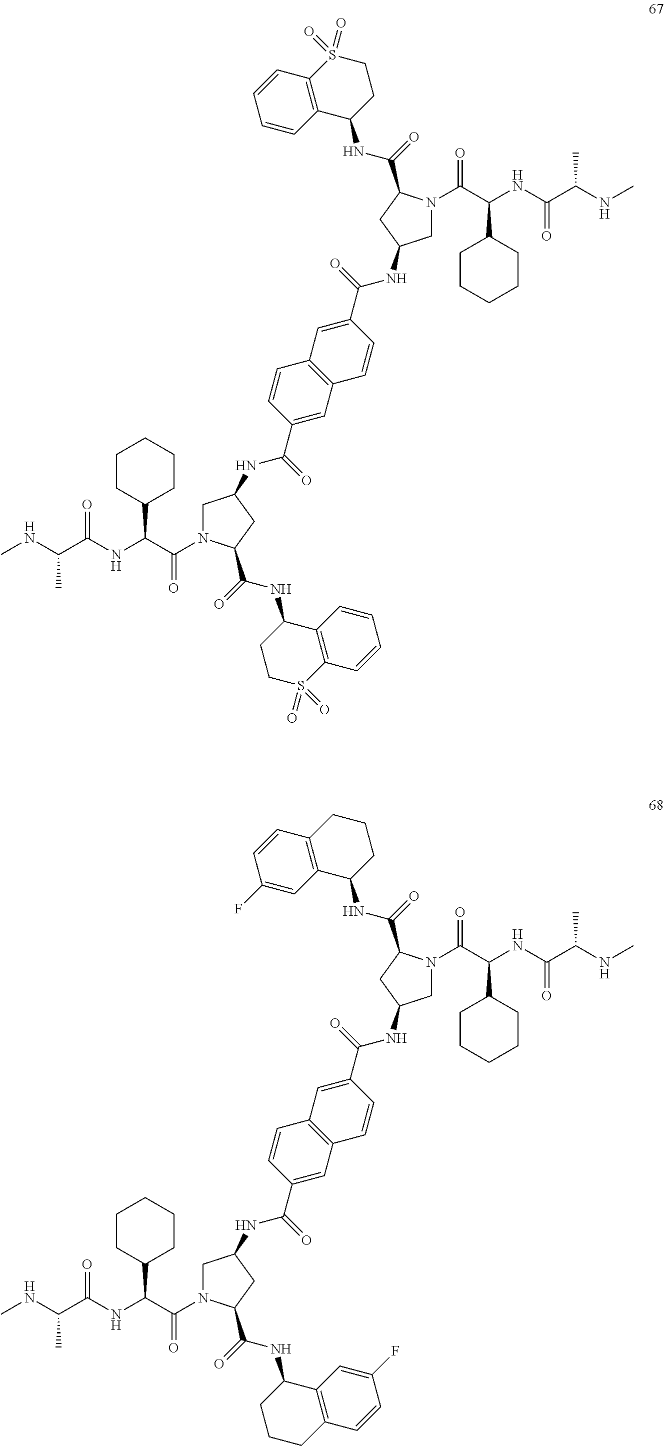

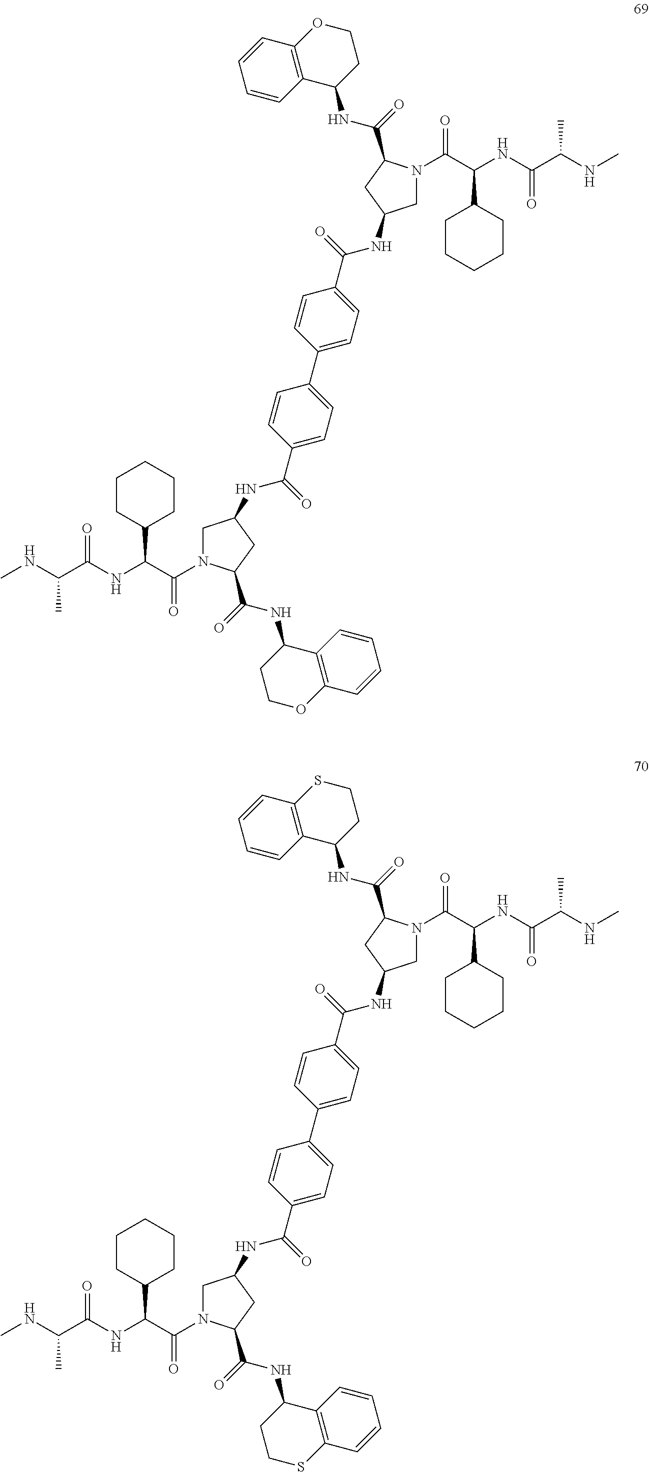

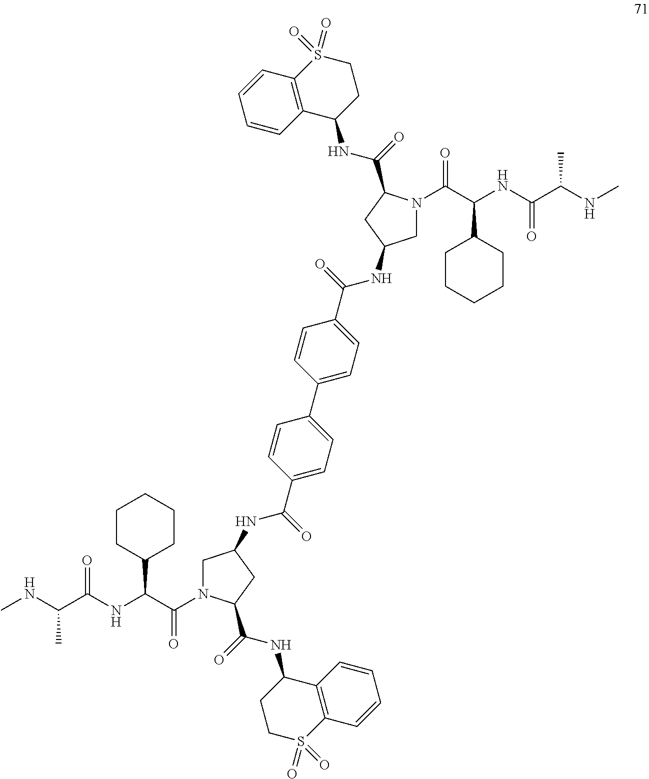

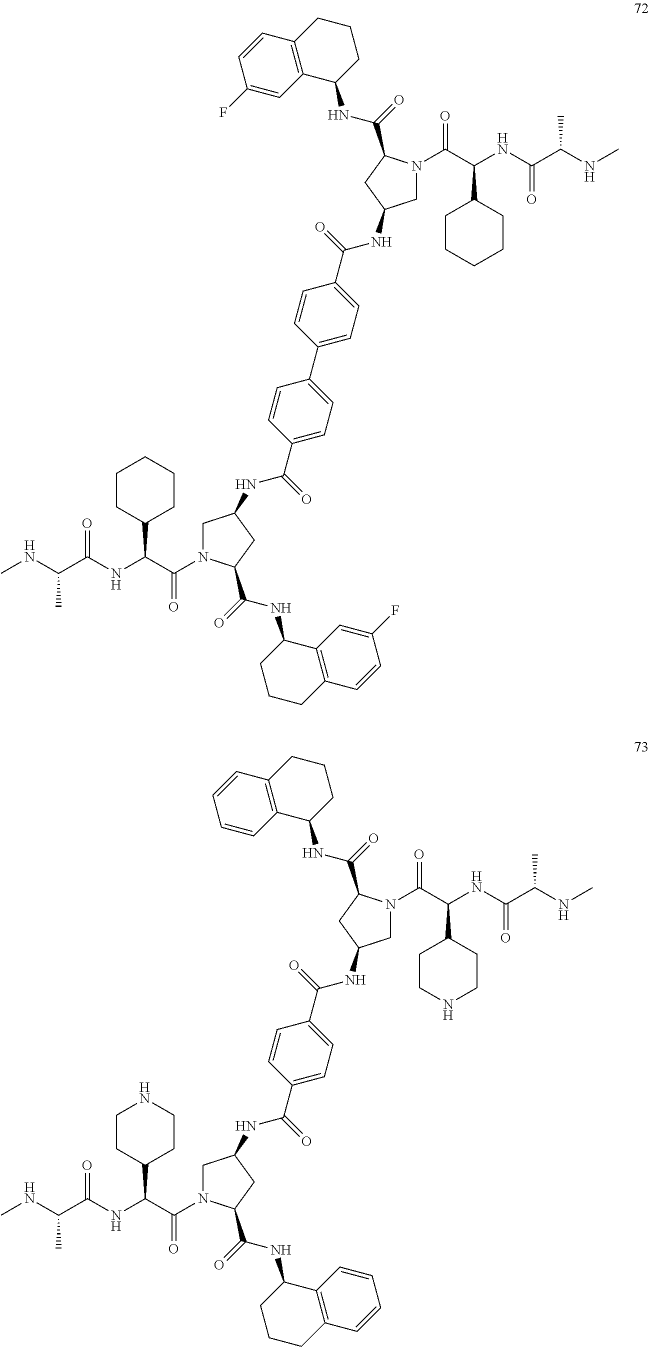

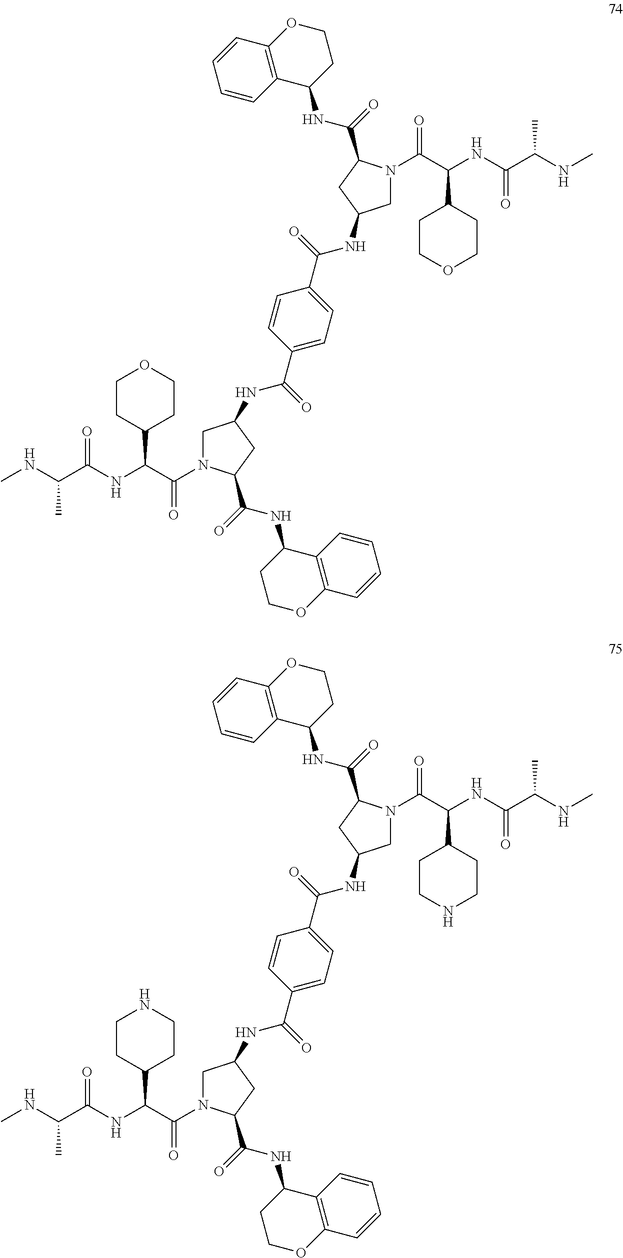

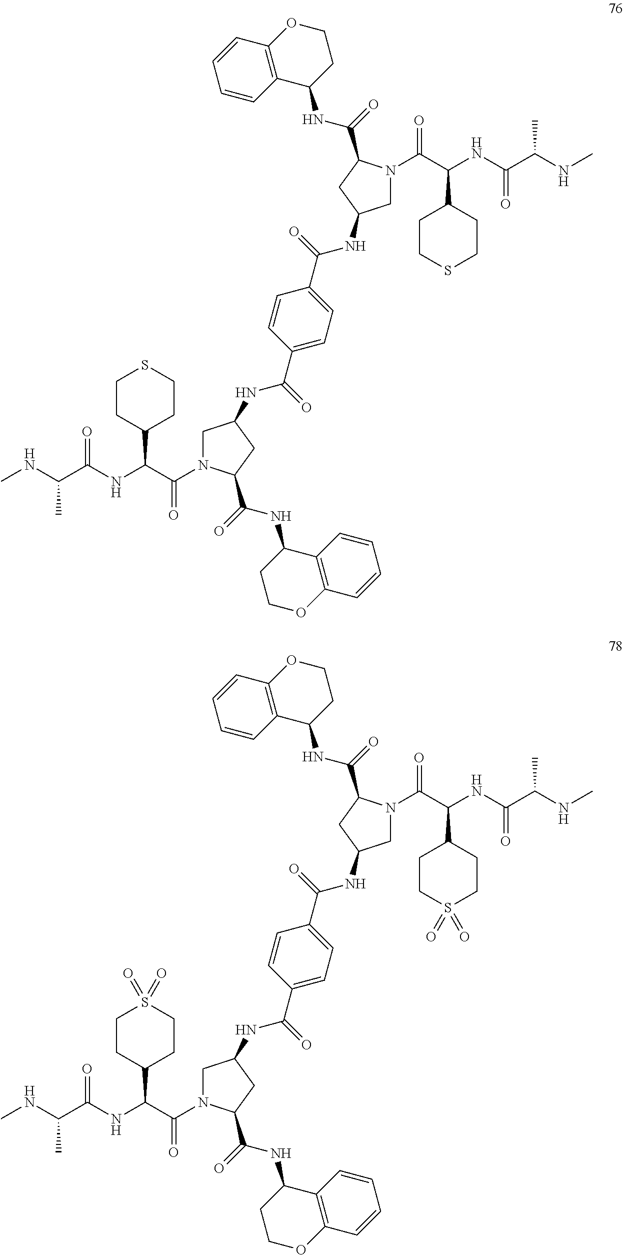

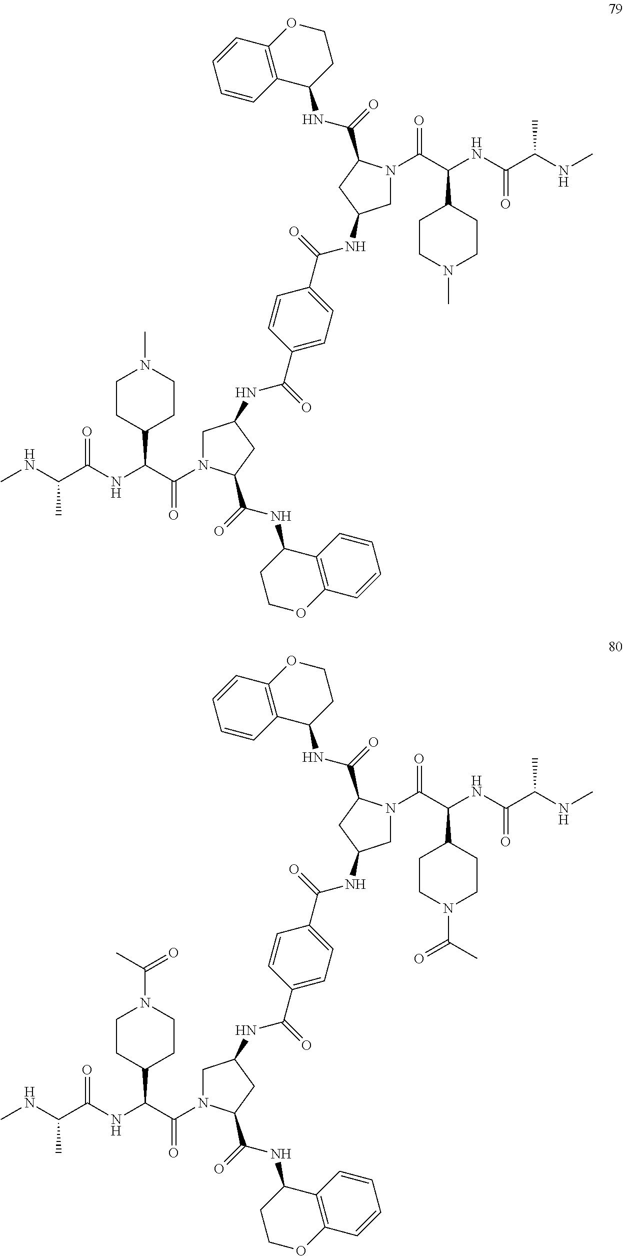

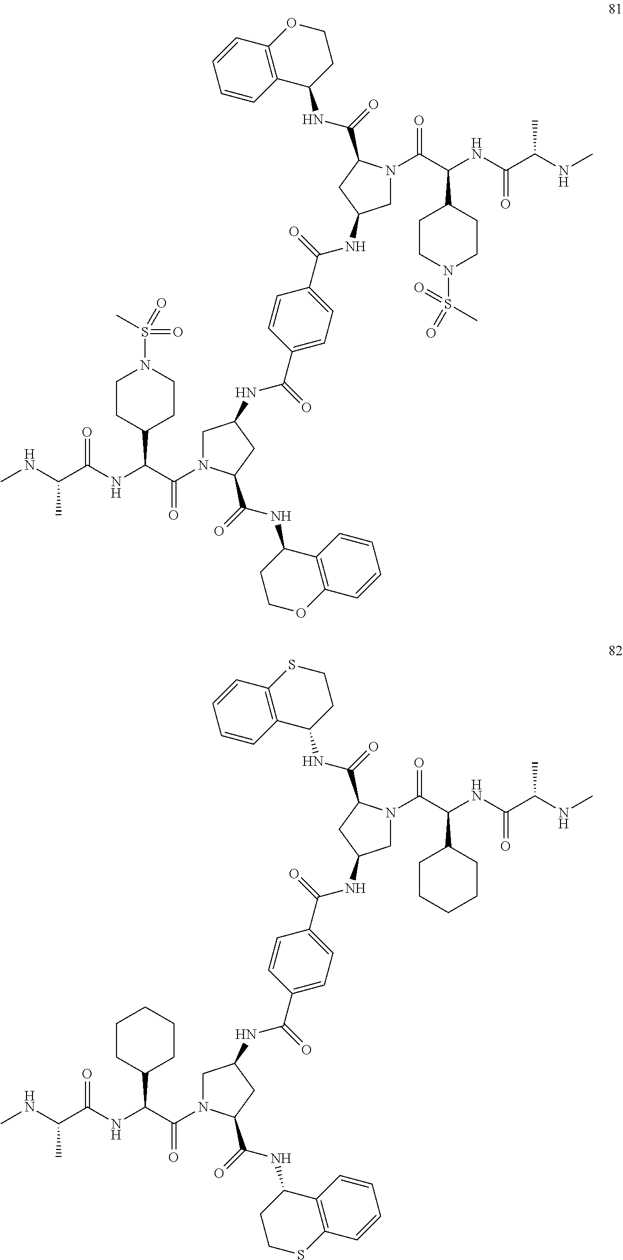

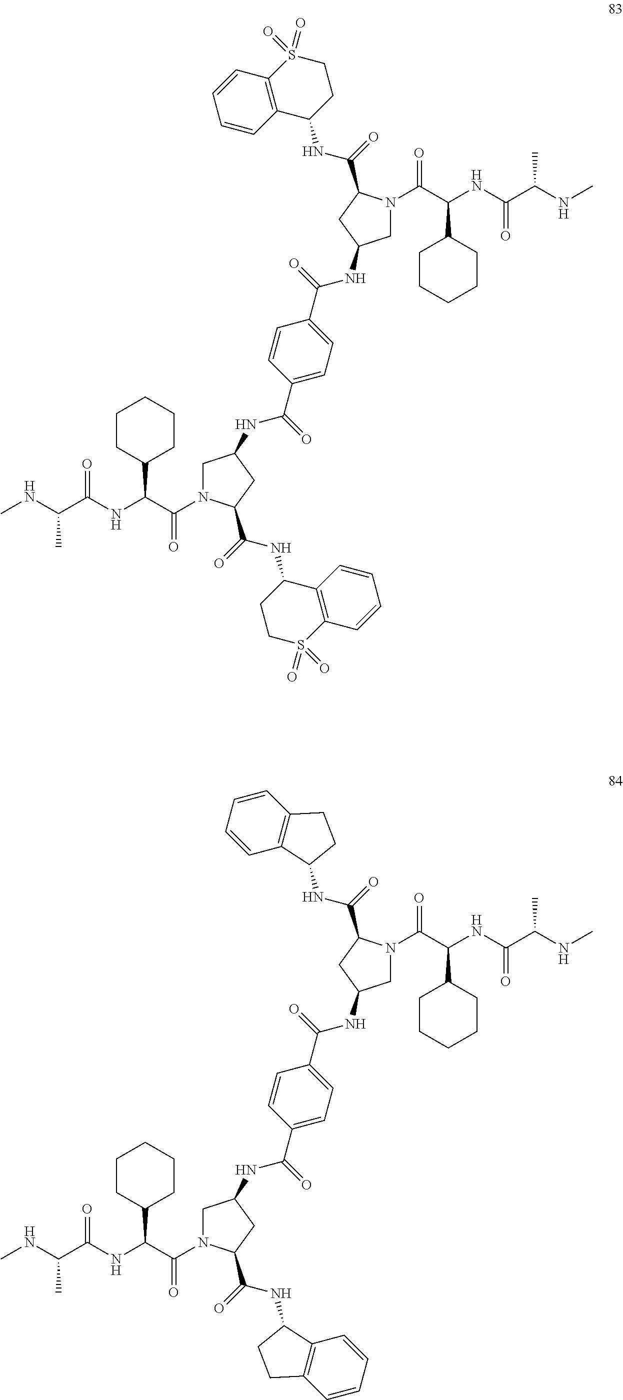

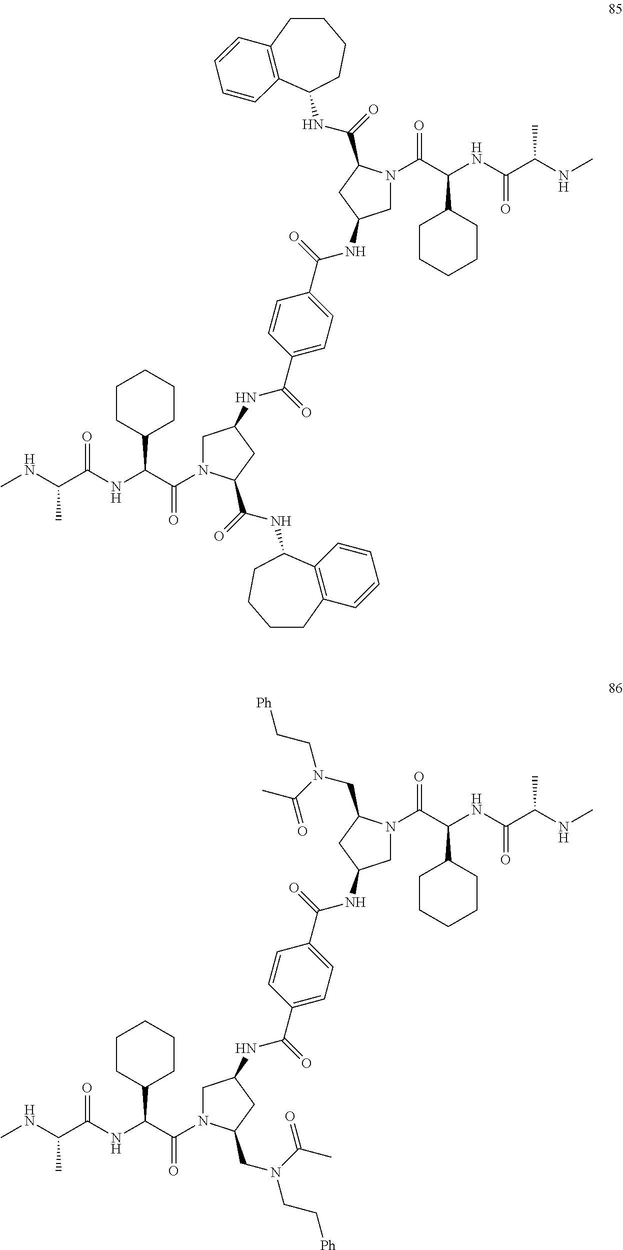

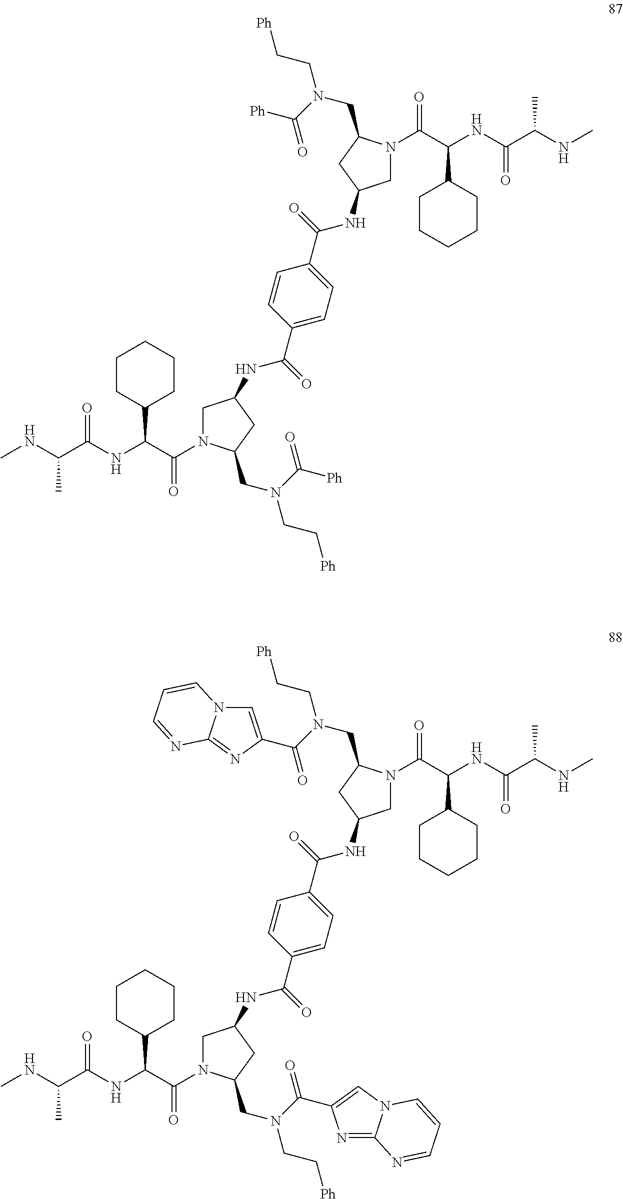

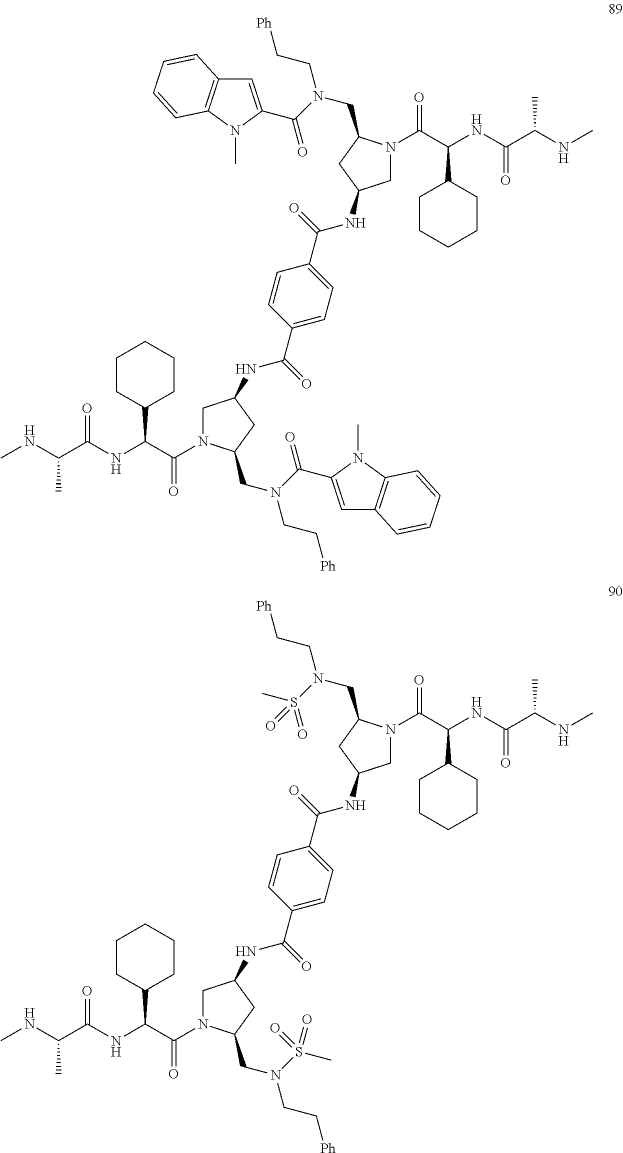

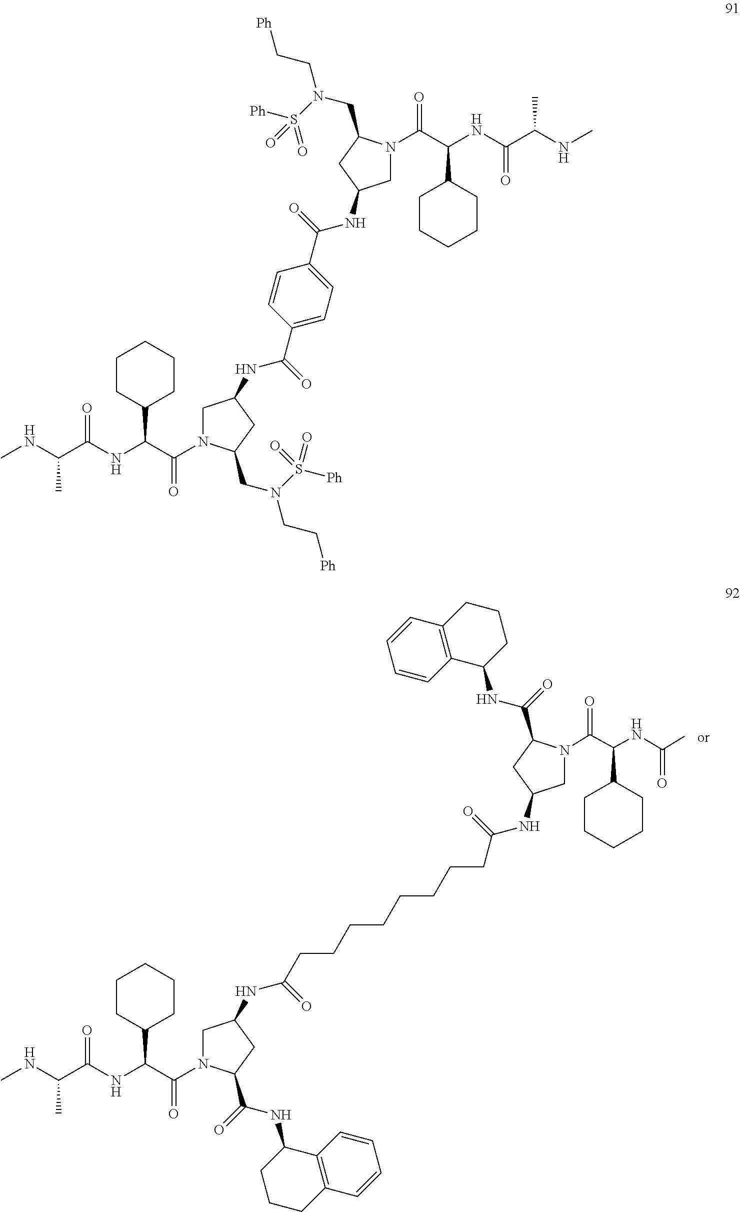

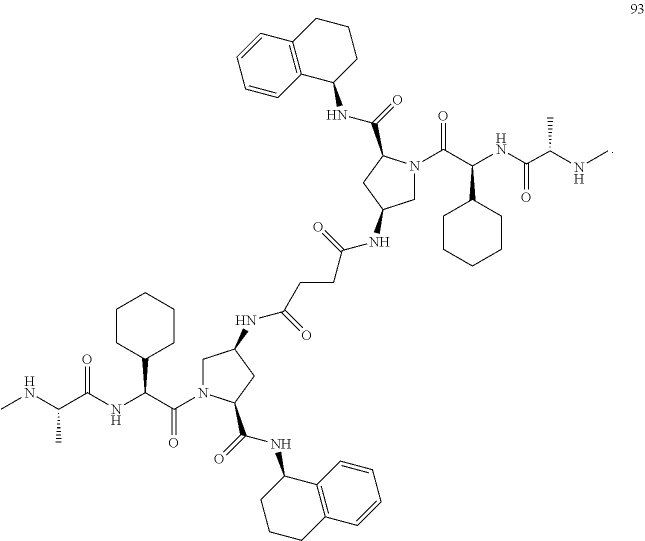

19. A compound represented by 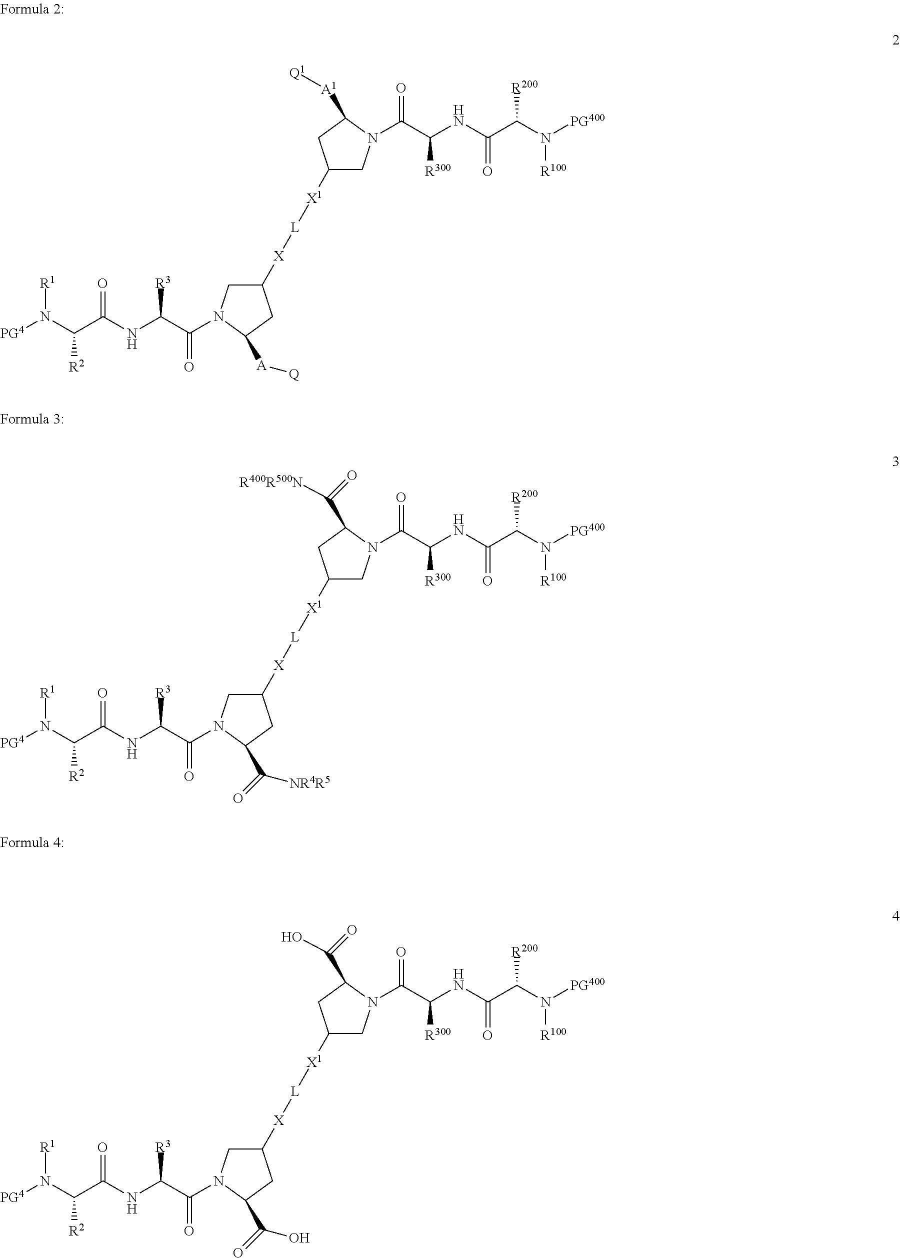 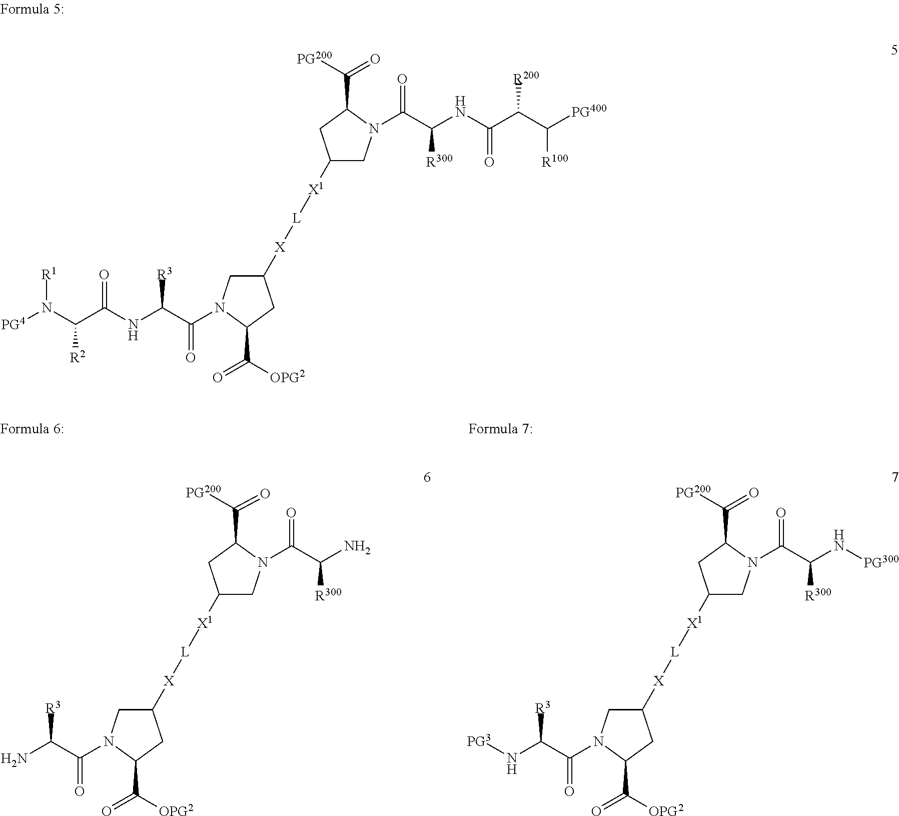 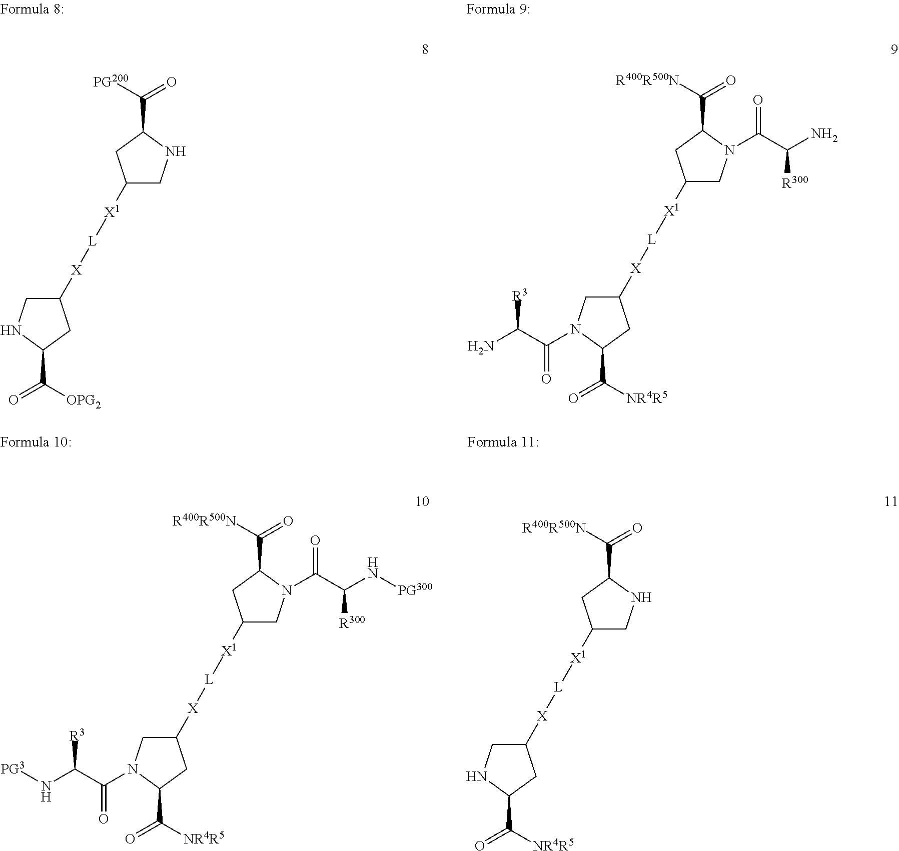 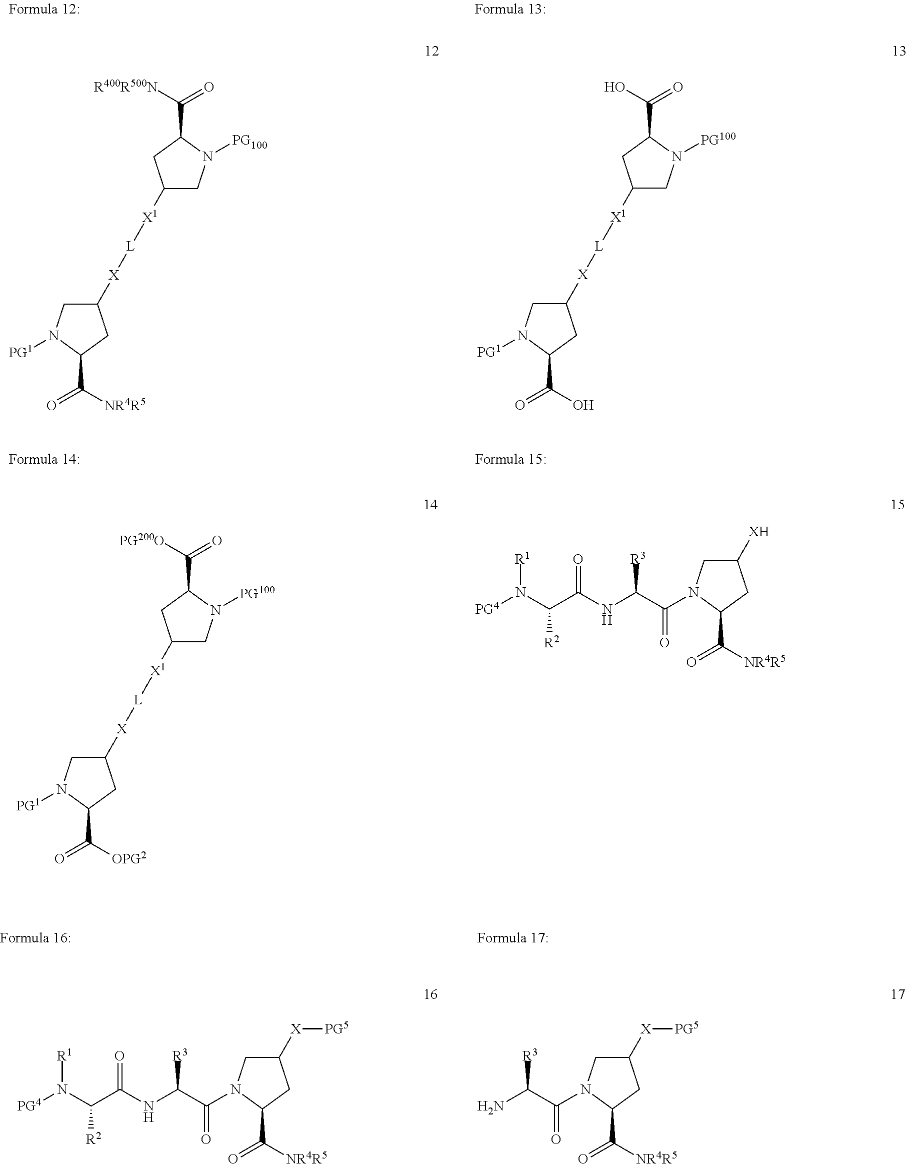 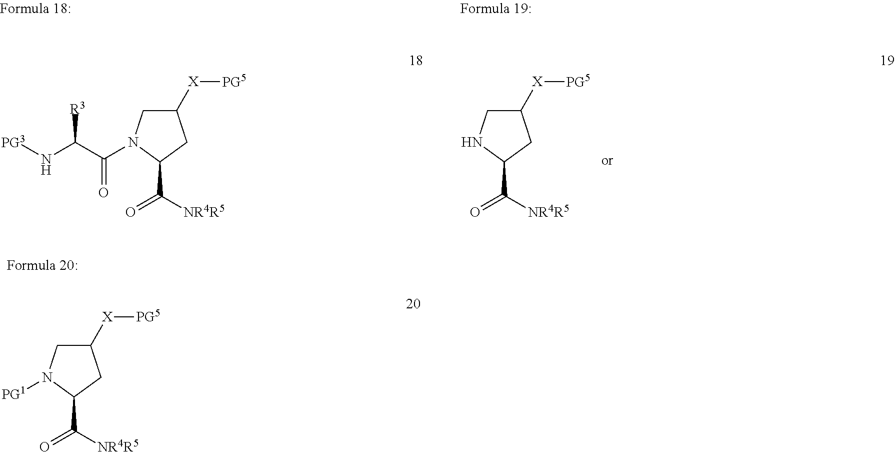
wherein PG¹, PG¹⁰⁰, PG², PG²⁰⁰, PG³, PG³⁰⁰, PG⁴, PG⁴⁰⁰, and PG⁵ are protecting groups, and L, X, X¹, R¹, R¹⁰⁰, R², R²⁰⁰, R³, R³⁰⁰, R⁴, R⁴⁰⁰, R⁵, and R⁵⁰⁰ are as defined in claim 1.

20. A method for preparing a pharmaceutically acceptable salt of a compound of formula 1, according to claim 1, comprising treating a compound of formula 1 or an intermediate compound of formula 2-ii with a pharmaceutically acceptable acid, so as to form a pharmaceutically acceptable salt of a compound of formula 1.

21. A pharmaceutical composition comprising a compound of claim 1, and a pharmaceutically acceptable carrier, diluent or excipient.

22. The pharmaceutical composition of claim 21 further comprising one or more death receptor agonists.

23. The pharmaceutical composition of claim 22, wherein the pharmaceutical composition comprises TRAIL or an anti-TRAIL receptor antibody.

24-28. (canceled)

29. A method of treating a proliferative disease or a disease state characterized by insufficient apoptosis, the method comprising administering to a subject in need thereof a therapeutically effective amount of the pharmaceutical composition of claim 21, so as to treat the proliferative disease or disease state characterized by insufficient apoptosis.

30. The method of claim 29, wherein the proliferative disease or disease state characterized by insufficient apoptosis is cancer or rheumatoid arthritis.

31-33. (canceled)

34. The method of claim 29, wherein the disease or disease state is rheumatoid arthritis, and the pharmaceutical composition is administered in combination, simultaneously or sequentially, with a non-steroidal anti-inflammatory drug (NSAID), analgesic, corticosteroid, antirheumatic, a tumor necrosis factor inhibitor, a T-cell costimulatory blocking agent, a B cell depleting agent, an Interleukin-1 (IL-1) receptor antagonist, a p38 inhibitor, a JAK inhibitor, an anti-CD20 MAb, or an anti-IL/ILR agent.

35-36. (canceled)

37. The method of claim 29, further comprising administering to the subject a therapeutically effective amount of a death receptor agonist prior to, simultaneously with, or after administration of the pharmaceutical composition, wherein the death receptor agonist is TRAIL or an anti-TRAIL receptor antibody.

38-40. (canceled)

41. A method of modulating IAP function, the method comprising contacting a cell with a compound according to claim 1 so as to prevent binding of a BIR binding protein to an IAP BIR domain, thereby modulating the IAP function.
